SEQUENCE LISTING (22 sequences)

SEQ ID NO: 1 - protein, 447 aa, Artificial Sequence
  Control mAb HC
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 219 aa, Artificial Sequence
  Control mAb LC
DIVMTQSPDSLAVSLGERNATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 5 aa, Artificial Sequence
  Anti-PSMA mAb (PSMB127) HC CDR1
SDAMH

SEQ ID NO: 4 - protein, 17 aa, Artificial Sequence
  Anti-PSMA mAb (PSMB127) HC CDR2
EISGSGGYTNYADSVKG

SEQ ID NO: 5 - protein, 15 aa, Artificial Sequence
  Anti-PSMA mAb (PSMB127) HC CDR3
DSYDSSLYVGDYFDY

SEQ ID NO: 6 - protein, 11 aa, Artificial Sequence
  Anti-PSMA mAb (PSMB127) LC CDR1
RASQSVSSYLA

SEQ ID NO: 7 - protein, 7 aa, Artificial Sequence
  Anti-PSMA mAb (PSMB127) LC CDR2
DASNRAT

SEQ ID NO: 8 - protein, 9 aa, Artificial Sequence
  Anti-PSMA mAb (PSMB127) LC CDR3
QQRSNWPLT

SEQ ID NO: 9 - protein, 451 aa, Artificial Sequence
  Anti-PSMA mAb (PSMB127) HC
EVQLLESGGGLVQPGGSLRLSCAASGFTFKSDAMHWVRQAPGKGLEWVSEISGSGGYTNYADSVKGRFTISRDNSKNTLYLQMNSLRAEDTAVYYCARDSYDSSLYVGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence
  Anti-PSMA mAb (PSMB127) LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 6 aa, Artificial Sequence
  H11B6 CDR sequence
SDYAWN

SEQ ID NO: 12 - protein, 16 aa, Artificial Sequence
  H11B6 CDR sequence
YISYSGSTTYNPSLKS

SEQ ID NO: 13 - protein, 8 aa, Artificial Sequence
  H11B6 CDR sequence
GYYYGSGF

SEQ ID NO: 14 - protein, 15 aa, Artificial Sequence
  H11B6 CDR sequence
KASESVEYFGTSLMH

SEQ ID NO: 15 - protein, 7 aa, Artificial Sequence
  H11B6 CDR sequence
AASNRES

SEQ ID NO: 16 - protein, 9 aa, Artificial Sequence
  H11B6 CDR sequence
QQTRKVPYT

SEQ ID NO: 17 - protein, 117 aa, Artificial Sequence
  H11B6 HC variable region sequence
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 18 - protein, 111 aa, Artificial Sequence
  H11B6 LC variable region sequence
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIK

SEQ ID NO: 19 - protein, 330 aa, Artificial Sequence
  H11B6 HC constant region sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 107 aa, Artificial Sequence [sequence omitted]

SEQ ID NO: 21 - protein, 447 aa, Artificial Sequence
  H11B6 HC sequence
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 218 aa, Artificial Sequence
  H11B6 LC sequence
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0143231 A1
(43) Pub. Date: May 12, 2022

(54) MACROCYCLIC COMPOUNDS AND METHODS OF USE THEREOF

(71) Applicant: Janssen Biotech, Inc., Horsham, PA (US)

(72) Inventors: Rhys Salter, Doylestown, PA (US); Vadim Y. Dudkin, Chalfont, PA (US); Fengbin Song, Doylestown, PA (US); Wei Zhang, Malvern, PA (US); Shalom Goldberg, Merion Station, PA (US); John Keith, San Diego, CA (US)

(21) Appl. No.: 17/522,144

(22) Filed: Nov. 9, 2021

Related U.S. Application Data
(60) Provisional application No. 63/111,933, filed on Nov. 10, 2020.

(51) Int. Cl. A61K 51/10; A61K 47/55; A61K 51/04; A61K 47/68; A61K 47/54

(52) U.S. Cl. CPC A61K 51/1096; A61K 47/55; A61K 47/545; A61K 47/6803; A61K 51/0497

(57) ABSTRACT

The present invention is directed to compounds and pharmaceutically acceptable salts thereof, immunoconjugates, radioimmunoconjugates thereof, pharmaceutical compositions containing said compounds and immunoconjugates, radioimmunoconjugates thereof, and the use of said compounds and immunoconjugates, radioimmunoconjugates thereof, in the treatment of neuroplastic diseases or disorders.

### CROSS-REFERENCE TO RELATED APPLICATIONS

This application claims the benefit of priority of U.S. Provisional Application No. 63/111,933, filed on Nov. 10, 2020, which is incorporated by reference herein, in its entirety and for all purposes.

### REFERENCE TO SEQUENCE LISTING SUBMITTED ELECTRONICALLY

This application contains a sequence listing, which is submitted electronically via EFS-Web as an ASCII formatted sequence listing with a file name “JBI6417WOPCT1_SeqListing.txt” and a creation date of Oct. 26, 2021 and having a size of 26 kb. The sequence listing submitted via EFS-Web is part of the specification and is herein incorporated by reference in its entirety.

### FIELD OF THE INVENTION

The present invention is directed to compounds (macrocyclic compounds) and pharmaceutically acceptable salts thereof, immunoconjugates, radioimmunoconjugates thereof, pharmaceutical compositions containing said compounds and immunoconjugates, radioimmunoconjugates thereof, and the use of said compounds and immunoconjugates, radioimmunoconjugates thereof, in the treatment of neoplastic diseases or disorders.

### BACKGROUND OF THE INVENTION

Alpha particle-emitting radionuclides show great promise for cancer therapy due to their combination of high linear energy transfer and short-range of action, providing the possibility of potent killing that is mostly localized to tumor cells (Kim, Y. S. and M. W. Brechbiel, An overview of targeted alpha therapy. Tumour Biol, 2012. 33(3): p. 573-90). Targeted delivery of alpha-emitters, using an antibody, scaffold protein, small molecule ligand, aptamer, or other binding moiety that is specific for a cancer antigen, provides a method of selective delivery of the radionuclide to tumors to enhance their potency and mitigate off-target effects. In common practice, the binding moiety is attached to a chelator which binds to the alpha-emitting radiometal to produce a radiocomplex. Many such examples use a monoclonal antibody (mAb) as the targeting vector, to produce what is known as a radioimmunoconjugate.

Actinium-225 (²²⁵Ac) is an alpha-emitting radioisotope of particular interest for medical applications (Miederer et al., Realizing the potential of the Actinium-225 radionuclide generator in targeted alpha particle therapy applications. Adv Drug Deliv Rev, 2008. 60(12):71-82). The 10-day half-life of ²²⁵Ac is long enough to facilitate radio-conjugate production, but short enough to match the circulation pharmacokinetics of delivery vehicles such as antibodies and therefore, ²²⁵Ac radioimmunoconjugates are of particular interest. Additionally, ²²⁵Ac decays in a series of steps that collectively emit 4 alpha particles for every ²²⁵Ac decay before reaching a stable isotope, ²⁰⁹Bi, thereby increasing the potency. Another radioisotope of interest for medical applications is Lutetium-177 (¹⁷⁷Lu), which emits both gamma-irradiation suitable for imaging and medium-energy beta-irradiation suitable for radiotherapy. It has been shown that ¹⁷⁷Lu-labeled peptides demonstrate reduced normal tissue damage, and that ¹⁷⁷Lu-labeling makes it possible to use a single radiolabeled agent for both therapy and imaging (Kwekkeboom D J, et al. [¹⁷⁷Lu-DOTA⁰,Tyr³]octreotate: comparison with [¹¹¹In-DTPA⁰]octreotide in patients. Eur J Nucl Med. 2001; 28: p. 1319-1325). Other radioisotopes that are used for therapeutic applications include, e.g., beta or alpha emitters, such as, e.g., ³²P, ⁴⁷Sc, ⁶⁷Cu, ⁷⁷As, ⁸⁹Sr, ⁹⁰Y, ⁹⁹Tc, ¹⁰⁵Rh, ¹⁰⁹Pd, ¹¹¹Ag, ¹³¹I, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²⁵⁵Fm and ²²⁷Th. Other radioisotopes that are used for imaging applications include gamma- and, or positron emitting radioisotopes, such as, e.g., 62Cu, ⁶⁴Cu, ⁶⁷Ga, ⁶⁸Ga, ⁸⁶Y, ⁸⁹Zr, and ¹¹¹In.

Currently, the most widely used chelator for Actinium-225 and lanthanides is DOTA (1,4,7,10-Tetraazacyclododecane-1,4,7,10-tetraacetic acid; tetraxaten), and previous clinical and pre-clinical programs have largely used 1,4,7,10-tetraazacyclododecane-1,4,7,10-tetraacetic acid (DOTA) for actinium chelation. However, it is known that DOTA chelation of actinium can be challenging (Deal, K. A., et al., Improved in vivo stability of actinium-225 macrocyclic complexes. J Med Chem, 1999. 42(15): p. 2988-92). For example, DOTA allows for a chelation ratio of at best >500:1 DOTA:Actinium-225 when attached to targeting ligands, such as proteins or antibodies, and often requires either harsh conditions or high levels of DOTA per antibody. Other macrocyclic chelators for lanthanides and actinium-225 have been described in, for example, International Patent Application Publication WO 2018/183906; Thiele et al. “An Eighteen-Membered Macrocyclic Ligand for Actinium-225 Targeted Alpha Therapy” Angew. Chem. Int. Ed. (2017) 56, 14712-14717; Roca-Sabio et al. “Macrocyclic Receptor Exhibiting Unprecedented Selectivity for Light Lanthanides” J. Am. Chem. Soc. (2009) 131, 3331-3341.

Site-specificity has become a key area of focus in the antibody-drug conjugate (ADC) field (Agarwal, P. and C. R. Bertozzi, Site-specific antibody-drug conjugates: the nexus of bioorthogonal chemistry, protein engineering, and drug development, Bioconjug Chem, 2015. 26(2): p. 176-92), as it has been demonstrated that both efficacy and safety of ADCs can be increased with site-specific methods as compared to random conjugation. It is thought that similar safety and efficacy benefits could be achieved for radioimmunoconjugates.

### BRIEF SUMMARY OF THE INVENTION

Accordingly, there is a need in the art for novel compounds that bind radiometals, preferably alpha-emitting radiometals, such as actinium-225 (²²⁵Ac), and can be used to produce stable radioimmunoconjugates with high specific activity and high yield. The invention satisfies this need by providing macrocyclic compounds capable of binding radiometals, such as alpha-emitting radiometals, for example ²²⁵Ac, irrespective of the specific activity or most common metal impurities, as well as the ability to chelate an imaging radiometal, for example ¹³⁴Ce.

Compounds of the invention can be used to produce radioimmunoconjugates having high stability in vitro and in vivo by conjugation to a targeting ligand, such as an antibody, protein, aptamer, etc., preferably in a site-specific manner using “click chemistry.” Radioimmunoconjugates produced by conjugation of the compounds of the invention to a targeting ligand can be used for targeted radiotherapy, such as for targeted radiotherapy of a neoplastic cell and/or targeted treatment of a neoplastic disease or disorder, including cancer.

The inventions encompass compounds capable of forming complexes with radiometal, radiometal complexes and radioimmunoconjugates as described herein.

In an embodiment of the invention is a compound of Formula (I):

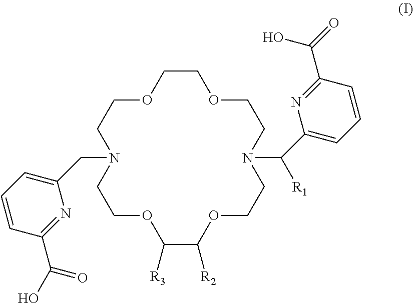

or a pharmaceutically acceptable salt thereof, wherein:
R₁ is hydrogen and R₂ is -L₁-R₄;
alternatively, R₁ is -L₁-R₄ and R₂ is hydrogen;
R₃ is hydrogen;
alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
L₁ is absent or a linker; and
R₄ is a nucleophilic moiety, an electrophilic moiety, or a targeting ligand.
In an embodiment, the present invention is directed to one or more compounds independently selected from the group consisting of

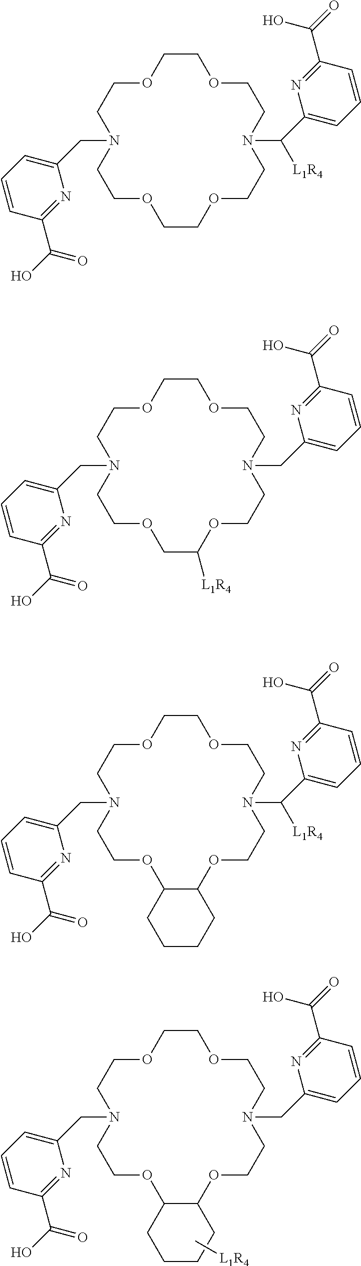

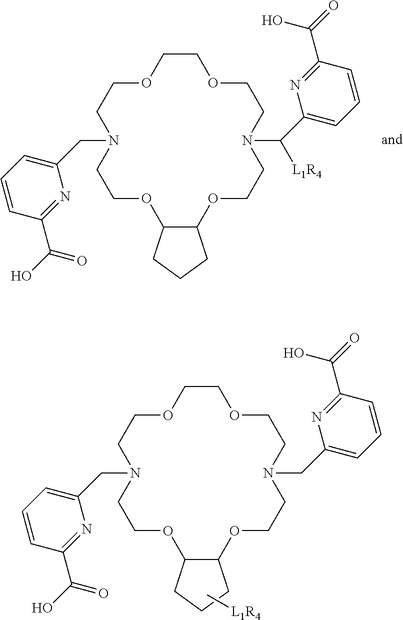

wherein
L₁ is absent or a linker; and
R₄ is a nucleophilic moiety, an electrophilic moiety, or a targeting ligand.

In certain embodiments, R₄ is —NH₂, —NCS, —NCO, —N₃, alkynyl, cycloalkynyl, —C(O)R₁₃, —COOR₁₃, —CON(R₁₃)₂, maleimido, acyl halide, tetrazine, or trans-cyclooctene.

In certain embodiments, R₄ is cyclooctynyl or a cyclooctynyl derivative selected from the group consisting of bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

In certain embodiments, R₄ is DBCO or BCN.

In certain embodiments, R₄ comprises a targeting ligand, wherein the targeting ligand is selected from the group consisting of an antibody, antibody fragment (e.g., an antigen-binding fragment), a binding peptide, a binding polypeptide (such as a selective targeting oligopeptide containing up to 50 amino acids), a binding protein, an enzyme, a nucleobase-containing moiety (such as an oligonucleotide, DNA or RNA vector, or aptamer), and a lectin.

In certain embodiments, a targeting ligand is an antibody or antigen binding fragment thereof.

In another embodiment, the invention is a radiometal complex comprising a radiometal ion complexed to a compound of Formula I.

In another embodiment, the present invention is directed to a radiometal complex of Formula (I-M⁺):

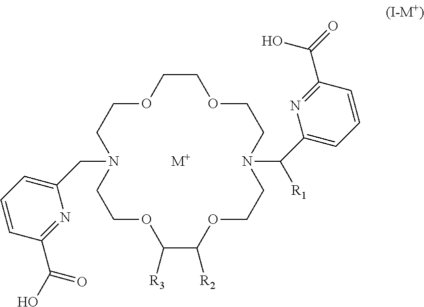

or a pharmaceutically acceptable salt thereof, wherein:
M⁺ is a radiometal ion selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
R₁ is hydrogen and R₂ is -L₁-R₄;
alternatively, R₁ is -L₁-R₄ and R₂ is hydrogen;
R₃ is hydrogen;
alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
L₁ is absent or a linker;
R₄ is a nucleophilic moiety, an electrophilic moiety, or a targeting ligand.

In some embodiments, R₄ is —NH₂, —NCS, —NCO, —N₃, alkynyl, cycloalkynyl, —C(O)R₁₃, —COOR₁₃, —CON(R₁₃)₂, maleimido, acyl halide, tetrazine, or trans-cyclooctene.

In certain embodiments, R₄ is cyclooctynyl or a cyclooctynyl derivative selected from the group consisting of bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

In certain embodiments, R₄ is DBCO or BCN.

In certain embodiments, the alpha-emitting radiometal ion is actinium-225 (²²⁵Ac).

In another embodiment, the present invention is directed to radioimmunoconjugates of formula (I-M⁺), or a pharmaceutically acceptable salt thereof, wherein
M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
   R₁ is hydrogen and R₂ is -L₁-R₄;
   alternatively, R₁ is -L₁-R₄ and R₂ is hydrogen;
   R₃ is hydrogen;
   alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
   L₁ is absent or a linker; and
   R₄ is a targeting ligand; wherein the targeting ligand is selected from the group consisting of an antibody, antibody fragment (e.g., an antigen-binding fragment), a binding moiety, a binding peptide, a binding polypeptide (such as a selective targeting oligopeptide containing up to 50 amino acids), a binding protein, an enzyme, a nucleobase-containing moiety (such as an oligonucleotide, DNA or RNA vector, or aptamer), and a lectin.

In certain embodiments, the alpha-emitting radiometal ion is actinium-225 (²²⁵Ac).

In another embodiment, the invention is directed to an immunoconjugate comprising compounds of the invention covalently linked via R₄ to a targeting ligand, preferably an antibody or antigen binding fragment thereof.

In a further embodiment, a radioimmunoconjugate comprises a radiometal complex of the invention covalently linked to an antibody or antigen binding fragment thereof, via a triazole moiety.

In another embodiment, the invention is directed methods of preparing an immunoconjugate or a radioimmunoconjugate of the invention, comprising covalently linking a compound or a radiometal complex of the invention with a targeting ligand, preferably via R₄ of the compound or radiometal complex to an antibody or antigen binding fragment thereof.

In another embodiment, the invention is directed to a pharmaceutical composition comprising a compound, immunoconjugate or radioimmunoconjugate of the invention, and a pharmaceutically acceptable carrier. The pharmaceutical composition may comprise one or more pharmaceutically acceptable excipients.

In another embodiment, the present invention also provides compositions (e.g. pharmaceutical compositions) and medicaments comprising any of one of the compounds as described herein (or a pharmaceutically acceptable salt thereof) and a pharmaceutically acceptable carrier or one or more excipients or fillers. In a similar embodiment, the present invention also provides compositions (e.g., pharmaceutical compositions) and medicaments comprising any of one of the embodiments of the modified antibody, modified antibody fragment, or modified binding peptide of the present technology disclosed herein and a pharmaceutically acceptable carrier or one or more excipients or fillers.

In another embodiment, the invention is directed to methods of using the radioimmunoconjugates and pharmaceutical compositions of the invention for targeted radiotherapy.

In an embodiment, the invention is directed to a method of selectively targeting neoplastic cells for radiotherapy in a subject in need thereof, the method comprising administering to the subject a pharmaceutical composition of the invention.

In an embodiment, the invention is directed to a method of treating a neoplastic disease or disorder in a subject in need thereof, the method comprising administering to the subject a pharmaceutical composition of the invention.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

In the drawings:

FIGS. 1A-1B shows HPLC chromatograms from a chelation test with La³⁺; FIG. 1A shows HPLC chromatograms of 6-((16-((6-carboxypyridin-2-yl)(phenyl)methyl)-1,4,10,13-tetraoxa-7,16-diazacyclooctadecan-7-yl)methyl)picolinic acid (TOPA-[C7]-phenyl) prior to mixing (top) and subsequent to mixing with La³⁺ (middle); the shift in retention time from 14.137 minutes to 12.047 minutes subsequent to mixing with La³⁺ and Ac-225 indicates rapid chelation of La³⁼; FIG. 1B shows HPLC chromatograms of TOPA-[C7]-isopentyl prior to mixing (top) and, subsequent to mixing with La³⁺ (bottom); the shift in retention time from 17.181 minutes to 15.751 minutes subsequent to mixing with La³⁺ indicates rapid chelation of La³⁺ and Ac-225 by TOPA-[C7]-isopentyl;

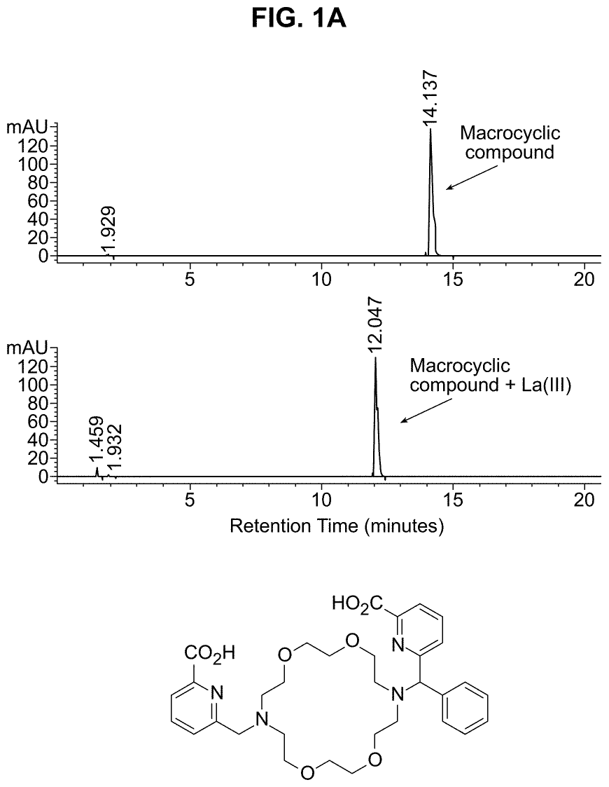

FIG. 2 shows a schematic representation of radiolabeling an antibody to produce a radioimmunoconjugate according to embodiments of the invention by random conjugation methods (e.g., methods for labeling of lysine residues, cysteine residues, etc.) or site-specific conjugation methods (e.g., glycan-specific methods, conjugation tag methods, or engineered cysteine methods); FIG. 2A schematically illustrates random conjugation via one-step direct radiolabeling; FIG. 2B schematically illustrates random conjugation via click radiolabeling; FIG. 2C schematically illustrates site-specific conjugation via one-step direct radiolabeling; and FIG. 2D schematically illustrates site-specific conjugation via click radiolabeling.

FIGS. 3A-3B shows HPLC chromatograms from a chelation test with Ac-225. FIG. 3A shows HPLC chromatogram of 6-((16-((6-carboxypyridin-2-yl)(phenyl)methyl)-1,4,10,13-tetraoxa-7,16-diazacyclooctadecan-7-yl)methyl)picolinic acid (TOPA-[C7]-phenyl) chelating with Ac-225 (RA (radioactivity) trace by cut-count-reconstruct). FIG. 3B shows HPLC chromatogram of 6-((16-(1-(6-carboxypyridin-2-yl)-4-methylpentyl)-1,4,10,13-tetraoxa-7,16-diazacyclooctadecan-7-yl)methyl)picolinic acid (TOPA-[C7]-isopentyl) chelating with Ac-225 (RA (radioactivity) trace by cut-count-reconstruct).

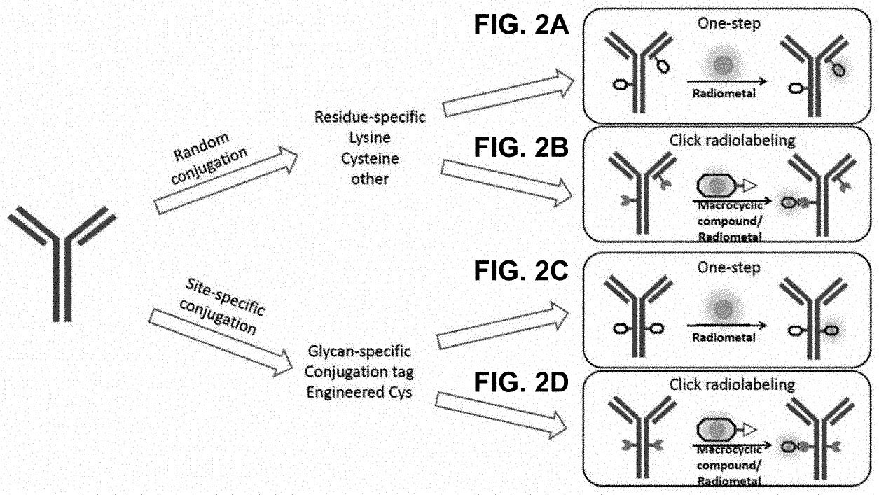

FIGS. 4A-4B shows HPLC chromatograms from the chelation test with Ac-225. FIG. 4A shows HPLC chromatogram of TOPA-[C7]-phenylthiourea-H11B6 chelated with Ac-225 (UV).

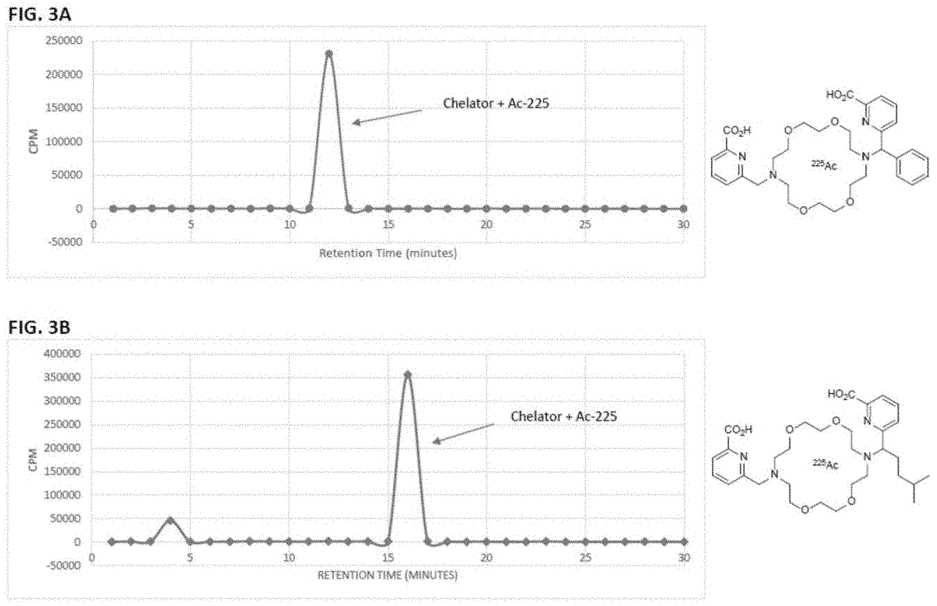

FIG. 4B shows HPLC chromatogram of TOPA-[C7]-phenylthiourea-H11B6 chelated with Ac-225 RA (radioactivity) trace by cut-count-reconstruct).

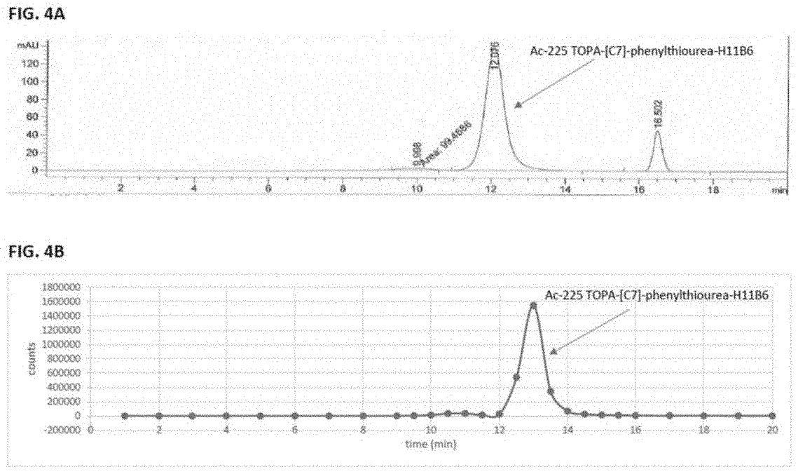

FIG. 5 shows a scan of instant thin layer chromatography (iTLC) indicating percentage of Ac-225 bound to TOPA-[C7]-phenylthiourea-h11b6 in the presence of metal impurities, as described in Example 22.

FIG. 6 shows a scan of iTLC indicating percentage of Ac-225 bound to TOPA-[C7]-phenylthiourea-h11b6 in the presence of metal impurities, as described in Example 22.

FIG. 7 shows a scan of iTLC indicating percentage of Ac-225 bound to TOPA-[C7]-phenylthiourea-h11b6 in the presence of metal impurities, as described in Example 22.

FIG. 8 shows a scan of iTLC indicating percentage of Ac-225 bound to TOPA-[C7]-phenylthiourea-h11b6 in the presence of metal impurities, as described in Example 22.

FIG. 9 shows a scan of iTLC indicating percentage of Ac-225 chelated to DOTA-h11b6 in the presence of metal impurities, as described in Example 22.

FIG. 10 shows a scan of iTLC indicating percentage of Ac-225 bound to DOTA-h11b6 in the presence of metal impurities, as described in Example 22.

FIG. 11 shows a scan of iTLC indicating percentage of Ac-225 chelated to DOTA-h11b6 in the presence of metal impurities, as described in Example 22.

FIG. 12 shows a scan of iTLC indicating percentage of Ac-225 bound to DOTA-h11b6 in the presence of metal impurities, as described in Example 22.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms cited herein have the meanings as set in the specification. All patents, published patent applications and publications cited herein are incorporated by reference as if set forth fully herein

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

The term “substituted” means that at least one hydrogen atom is replaced with a non-hydrogen group, provided that all normal valencies are maintained and that the substitution results in a stable compound. When a particular group is “substituted,” that group can have one or more substituents, preferably from one to five substituents, more preferably from one to three substituents, most preferably from one to two substituents, independently selected from the list of substituents. For example, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SFs), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like. The term “independently” when used in reference to substituents, means that when more than one of such substituents is possible, such substituents can be the same or different from each other.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

As used herein, Cm-Cn, such as C₁-C₁₁, C₁-C₈, or C₁-C₆ when used before a group refers to that group containing m to n carbon atoms.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Alkyl groups may be substituted or unsubstituted. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Cycloalkyl groups may be substituted or unsubstituted. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Cycloalkylalkyl groups may be substituted or unsubstituted. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, an alkenyl can have one carbon-carbon double bond, or multiple carbon-carbon double bonds, such as 2, 3, 4 or more carbon-carbon double bonds. Examples of alkenyl groups include, but are not limited to methenyl, ethenyl, propenyl, butenyl, etc. Alkenyl groups may be substituted or unsubstituted. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl group can be a mono- or polycyclic alkyl group having from 3 to 12, more preferably from 3 to 8 carbon atoms in the ring(s) and comprising at least one double bond between two carbon atoms. Cycloalkenyl groups may be substituted or unsubstituted. In some embodiments the cycloalkenyl group may have one, two or three double bonds or multiple carbon-carbon double bonds, such as 2, 3, 4, or more carbon-carbon double bonds, but does not include aromatic compounds. Cycloalkenyl groups have from 3 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C═CH, —C═CCH₃, —CH₂C═CCH₃, —C═CCH₂CH(CH₂CH₃)₂, among others. Alkynyl groups may be substituted or unsubstituted. A terminal alkyne has at least one hydrogen atom bonded to a triply bonded carbon atom. Representative substituted alkynyl groups may be mono-substituted or substituted more than once, such as, but not limited to, mono-, di- or trisubstituted with substituents such as those listed above. A “cyclic alkyne” or “cycloalkynyl” is a cycloalkyl ring comprising at least one triple bond between two carbon atoms. Examples of cyclic alkynes or cycloalkynyl groups include, but are not limited to, cyclooctyne, bicyclononyne (BCN), difluorinated cyclooctyne (DIFO), dibenzocyclooctyne (DIBO), keto-DIBO, biarylazacyclooctynone (BARAC), dibenzoazacyclooctyne (DIBAC), dimethoxyazacyclooctyne (DIMAC), difluorobenzocyclooctyne (DIFBO), monobenzocyclooctyne (MOBO), and tetramethoxy DIBO (TMDIBO).

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Aryl groups may be substituted or unsubstituted. The phrase “aryl groups” includes groups containing fused rings, such as fused aromatic-aliphatic ring systems (e.g., indanyl, tetrahydronaphthyl, and the like). Representative substituted aryl groups may be monosubstituted or substituted more than once. For example, monosubstituted aryl groups include, but are not limited to, 2-, 3-, 4-, 5-, or 6-substituted phenyl or naphthyl groups, which may be substituted with substituents such as those listed above. Aryl moieties are well known and described, for example, in Lewis, R. J., ed., Hawley's Condensed Chemical Dictionary, 13^{th} Edition, John Wiley & Sons, Inc., New York (1997). An aryl group can be a single ring structure (i.e., monocyclic) or comprise multiple ring structures (i.e., polycyclic) that are fused ring structures. Preferably, an aryl group is a monocyclic aryl group.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Alkoxy groups may be substituted or unsubstituted. Representative substituted alkoxy groups may be substituted one or more times with substituents such as those listed above.

Similarly, alkylthio or thioalkoxy refers to an —SR group in which R is an alkyl attached to the parent molecule through a sulfur bridge, for example, —S-methyl, —S-ethyl, etc. Representative examples of alkylthio include, but are not limited to, —SCH₃, —SCH₂CH₃, etc.

The term “halogen” as used herein refers to bromine, chlorine, fluorine, or iodine. Correspondingly, the term “halo” means fluoro, chloro, bromo, or iodo. In some embodiments, the halogen is fluorine. In other embodiments, the halogen is chlorine or bromine.

The terms “hydroxy” and “hydroxyl” can be used interchangeably and refer to —OH.

The term “carboxy” refers to —COOH.

The term “cyano” refers to —CN.

The term “nitro” refers to —NO₂.

The term “isothiocyanate” refers to —N═C═S.

The term “isocyanate” refers to —N═C═O.

The term “azido” refers to —N₃.

The term “amino” refers to —NH₂. The term “alkylamino” refers to an amino group in which one or both of the hydrogen atoms attached to nitrogen is substituted with an alkyl group.

An alkylamine group can be represented as —NR₂ in which each R is independently a hydrogen or alkyl group. For example, alkylamine includes methylamine (—NHCH₃), dimethylamine (—N(CH₃)₂), —NHCH₂CH₃, etc. The term “aminoalkyl” as used herein is intended to include both branched and straight-chain saturated aliphatic hydrocarbon groups substituted with one or more amino groups. Representative examples of aminoalkyl groups include, but are not limited to, —CH₂NH₂, —CH₂CH₂NH₂, and —CH₂CH(NH₂)CH₃.

As used herein, “amide” refers to —C(O)N(R)₂, wherein each R is independently an alkyl group or a hydrogen. Examples of amides include, but are not limited to, —C(O)NH₂, —C(O)NHCH₃, and —C(O)N(CH₃)₂.

The terms “hydroxylalkyl” and “hydroxyalkyl” are used interchangeably, and refer to an alkyl group substituted with one or more hydroxyl groups. The alkyl can be a branched or straight-chain aliphatic hydrocarbon. Examples of hydroxylalkyl include, but are not limited to, hydroxylmethyl (—CH₂OH), hydroxylethyl (—CH₂CH₂OH), etc.

As used herein, the term “heterocyclyl” includes stable monocyclic and polycyclic hydrocarbons that contain at least one heteroatom ring member, such as sulfur, oxygen, or nitrogen. As used herein, the term “heteroaryl” includes stable monocyclic and polycyclic aromatic hydrocarbons that contain at least one heteroatom ring member such as sulfur, oxygen, or nitrogen. Heteroaryl can be monocyclic or polycyclic, e.g., bicyclic or tricyclic. Each ring of a heterocyclyl or heteroaryl group containing a heteroatom can contain one or two oxygen or sulfur atoms and/or from one to four nitrogen atoms provided that the total number of heteroatoms in each ring is four or less and each ring has at least one carbon atom. Heteroaryl groups which are polycyclic, e.g., bicyclic or tricyclic must include at least one fully aromatic ring but the other fused ring or rings can be aromatic or non-aromatic. The heterocyclyl or heteroaryl group can be attached at any available nitrogen or carbon atom of any ring of the heterocyclyl or heteroaryl group. Preferably, the term “heteroaryl” refers to 5- or 6-membered monocyclic groups and 9- or 10-membered bicyclic groups which have at least one heteroatom (O, S, or N) in at least one of the rings, wherein the heteroatom-containing ring preferably has 1, 2, or 3 heteroatoms, more preferably 1 or 2 heteroatoms, selected from O, S, and/or N. The nitrogen heteroatom(s) of a heteroaryl can be substituted or unsubstituted. Additionally, the nitrogen and sulfur heteroatom(s) of a heteroaryl can optionally be oxidized (i.e., N→O and S(O)ᵣ, wherein r is 0, 1 or 2).

The term “ester” refers to —C(O)₂R, wherein R is alkyl.

The term “carbamate” refers to —OC(O)NR₂, wherein each R is independently alkyl or hydrogen.

The term “aldehyde” refers to —C(O)H.

The term “carbonate” refers to —OC(O)OR, wherein R is alkyl.

The term “maleimide” refers to a group with the chemical formula H₂C₂(CO)₂NH. The term “maleimido” refers to a maleimide group covalently linked to another group or molecule. Preferably, a maleimido group is N-linked, for example:

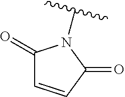 [SMILES: *N1C(=O)C=CC1=O]

The term “acyl halide” refers to —C(O)X, wherein X is halo (e.g., Br, Cl). Exemplary acyl halides include acyl chloride (—C(O)Cl) and acyl bromide (—C(O)Br).

In accordance with convention used in the art:

[symbol]

is used in structural formulas herein to depict the bond that is the point of attachment of the moiety, functional group, or substituent to the core, parent, or backbone structure, such as a compound of the invention or targeting ligand.

When any variable occurs more than one time in any constituent or formula for a compound, its definition at each occurrence is independent of its definition at every other occurrence. Thus, for example, if a group is shown to be substituted with 0-3 R groups, then said group can be optionally substituted with up to three R groups, and at each occurrence, R is selected independently from the definition of R.

When a bond to a substituent is shown to cross a bond connecting two atoms in a ring, then such substituent can be bonded to any atom on the ring.

As used herein, the term “radiometal ion” or “radioactive metal ion” refers to one or more isotopes of the elements that emit particles and/or photons. Any radiometal ion known to those skilled in the art in view of the present disclosure can be used in the invention. Examples of radiometal ions suitable for use in the invention include, but are not limited to, ⁴⁷Sc, ⁶²Cu, ⁶⁴Cu, ⁶⁷Cu, ⁶⁷Ga, ⁶⁸Ga, ⁸⁶Y, ⁸⁹Zr, ⁸⁹Sr, ⁹⁰Y, ⁹⁹Tc, ¹⁰⁵Rh, ¹⁰⁹Pd, ¹¹¹Ag, ¹¹¹In, ¹¹⁷Sn, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁷⁷Lu, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²²⁵Ac, ²²⁷Th, and ²⁵Fm. Preferably, the radiometal ion is a “therapeutic emitter,” meaning a radiometal ion that is useful in therapeutic applications. Examples of therapeutic emitters include, but are not limited to, beta or alpha emitters, such as, ¹³²La, ¹³⁵La, ¹³⁴Ce, ¹⁴⁴Nd, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁷⁷Lu, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²²⁵Ac, ²⁵⁵Fm and ²²⁷Th, ²²⁶Th, ²³⁰U. Preferably, a radiometal ion used in the invention is an alpha-emitting radiometal ion, such as actinium-225 (²²⁵Ac).

Compounds of the invention refer to a macrocycle compound to which a metal, preferably a radiometal, can be complexed to. In certain embodiments, a compound is a macrocycle or a macrocyclic ring containing one or more heteroatoms, e.g., oxygen and/or nitrogen as ring atoms. Preferably, the compound is a macrocycle that is a derivative of 4,13-diaza-18-crown-6.

A “radiometal complex” as used herein refers to a complex comprising a radiometal ion associated with a macrocyclic compound. A radiometal ion is bound to or coordinated to a macrocycle via coordinate bonding. Heteroatoms of the macrocyclic ring can participate in coordinate bonding of a radiometal ion to a macrocycle compound. A macrocycle compound can be substituted with one or more substituent groups, and the one or more substituent groups can also participate in coordinate bonding of a radiometal ion to a macrocycle compound in addition to, or alternatively to the heteroatoms of the macrocyclic ring.

As used herein, the term “TOPA” refers to a macrocycle known in the art as H₂bp18c6 and may alternatively be referred to as N,N′-bis[(6-carboxy-2-pyridil)methyl]-4,13-diaza-18-crown-6. See, e.g., Roca-Sabio et al., “Macrocyclic Receptor Exhibiting Unprecedented Selectivity for Light Lanthanides,” J. Am. Chem. Soc. (2009) 131, 3331-3341, which is incorporated by reference herein.

As used herein, the term “click chemistry” refers to a chemical philosophy introduced by Sharpless, describing chemistry tailored to generate covalent bonds quickly and reliably by joining small units comprising reactive groups together (see Kolb, et al., Angewandte Chemie International Edition (2001) 40: 2004-2021). Click chemistry does not refer to a specific reaction, but to a concept including, but not limited to, reactions that mimic reactions found in nature. In some embodiments, click chemistry reactions are modular, wide in scope, give high chemical yields, generate inert byproducts, are stereospecific, exhibit a large thermodynamic driving force to favor a reaction with a single reaction product, and/or can be carried out under physiological conditions. In some embodiments, a click chemistry reaction can be carried out under simple reaction conditions, uses readily available starting materials and reagents, uses non-toxic solvents or uses a solvent that is benign or easily removed, such as water, and/or provides simple product isolation by non-chromatographic methods, such as crystallization or distillation.

Click chemistry reactions utilize reactive groups that are rarely found in naturally-occurring biomolecules and are chemically inert towards biomolecules, but when the click chemistry partners are reacted together, the reaction can take place efficiently under biologically relevant conditions, for example in cell culture conditions, such as in the absence of excess heat and/or harsh reagents. In general, click chemistry reactions require at least two molecules comprising click reaction partners that can react with each other. Such click reaction partners that are reactive with each other are sometimes referred to herein as click chemistry handle pairs, or click chemistry pairs. In some embodiments, the click reaction partners are an azide and a strained alkyne, e.g. cycloalkyne such as a cyclooctyne or cyclooctyne derivative, or any other alkyne. In other embodiments, the click reaction partners are reactive dienes and suitable tetrazine dienophiles. For example, trans-cyclooctene, norbornene, or biscyclononene can be paired with a suitable tetrazine dienophile as a click reaction pair. In yet other embodiments, tetrazoles can act as latent sources of nitrile imines, which can pair with unactivated alkenes in the presence of ultraviolet light to create a click reaction pair, termed a “photo-click” reaction pair. In other embodiments, the click reaction partners are a cysteine and a maleimide. For example the cysteine from a peptide (e.g., GGGC) can be reacted with a maleimide that is associated with a chelating agent (e.g., NOTA). Other suitable click chemistry handles are known to those of skill in the art (see, e.g., Spicer et al., Selective chemical protein modification. Nature Communications. 2014; 5: p. 4740). In other embodiments, the click reaction partners are Staudinger ligation components, such as phosphine and azide. In other embodiments, the click reaction partners are Diels-Alder reaction components, such as dienes (e.g., tetrazine) and alkenes (e.g., trans-cyclooctene (TCO) or norbornene). Exemplary click reaction partners are described in US20130266512 and in WO2015073746, the relevant description on click reaction partners in both of which are incorporated by reference herein.

According to preferred embodiments, a click chemistry reaction utilizes an azide group and an alkyne group, more preferably a strained alkyne group, e.g., cycloalkyne such as a cyclooctyne or cyclooctyne derivative, as the click chemistry pair or reaction partners. In such embodiments, the click chemistry reaction is a Huisgen cycloaddition or 1,3-dipolar cycloaddition between the azide (—N₃) and alkyne moiety to form a 1,2,3-triazole linker. Click chemistry reactions between alkynes and azides typically require the addition of a copper catalyst to promote the 1,3-cycloaddition reaction, and are known as copper-catalyzed azide-alkyne cycloaddition (CuAAC) reactions. However, click chemistry reactions between cyclooctyne or cyclooctyne derivatives and azides typically do not require the addition of a copper catalyst, and instead proceed via strain-promoted azide-alkyne cycloaddition (SPAAC) (Debets, M. F., et al., Bioconjugation with strained alkenes and alkynes. Acc Chem Res, 2011. 44(9): p. 805-15).

As used herein the term “targeting ligand” refers to any molecule that provides an enhanced affinity for a selected target, e.g., an antigen, a cell, cell type, tissue, organ, region of the body, or a compartment (e.g., a cellular, tissue or organ compartment). Targeting ligands include, but are not limited to, antibodies or antigen binding fragments thereof, aptamers, polypeptides, and scaffold proteins. Preferably, a targeting ligand is a polypeptide, more preferably an antibody or antigen binding fragment thereof, engineered domain, or scaffold protein.

As used herein, the term “antibody” or “immunoglobulin” is used in a broad sense and includes immunoglobulin or antibody molecules including polyclonal antibodies, monoclonal antibodies including murine, human, human-adapted, humanized and chimeric monoclonal antibodies, and antigen-binding fragments thereof.

In general, antibodies are proteins or peptide chains that exhibit binding specificity to a specific antigen, referred to herein as a “target.” Antibody structures are well known. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibodies used in the invention can be of any of the five major classes or corresponding sub-classes. Antibody light chains of any vertebrate species can be assigned to one of two clearly distinct types, namely kappa and lambda, based on the amino acid sequences of their constant domains. According to particular embodiments, antibodies used in the invention include heavy and/or light chain constant regions from mouse antibodies or human antibodies. Each of the four IgG subclasses has different biological functions known as effector functions. These effector functions are generally mediated through interaction with the Fc receptor (FcγR) or by binding C1q and fixing complement. Binding to FcγR can lead to antibody dependent cell mediated cytolysis, whereas binding to complement factors can lead to complement mediated cell lysis. An antibody useful for the invention can have no or minimal effector function, but retain its ability to bind FcRn.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “scaffold” or “scaffold protein” refers to any protein that has a target binding domain and that can bind to a target. A scaffold contains a “framework”, which is largely structural, and a “binding domain” which makes contact with the target and provides for specific binding. The binding domain of a scaffold need not be defined by one contiguous sequence of the scaffold. In certain cases, a scaffold may be part of larger binding protein, which, itself, may be part of a multimeric binding protein that contains multiple scaffolds. Certain binding proteins can be bi- or multi-specific in that they can bind to two or more different epitopes. A scaffold can be derived from a single chain antibody, or a scaffold may be not antibody-derived.

As used herein, the term “aptamer” refers to a single-stranded oligonucleotide (single-stranded DNA or RNA molecule) that can bind specifically to its target with high affinity. The aptamer can be used as a molecule targeting various organic and inorganic materials.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca2⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The present technology provides new macrocyclic complexes that are substantially more stable than those of the conventional art. Thus, these new complexes can advantageously target cancer cells more effectively, with substantially less toxicity to non-targeted tissue than complexes of the art. Moreover, the new complexes can advantageously be produced at room temperature, in contrast to DOTA-type complexes, which generally require elevated temperatures (e.g., at least 80° C.) for complexation with the radionuclide. The present technology also specifically employs alpha-emitting radionuclides instead of beta radionuclides. Alpha-emitting radionuclides are of much higher energy, and thus substantially more potent, than beta-emitting radionuclides.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular embodiments described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary It is also to be understood that the terminology used herein is for the purpose of describing particular embodiments only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein, may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### COMPOUNDS (MACROCYCLIC COMPOUNDS) OF THE INVENTION

In an embodiment, the invention is directed to a compound of formula (I)

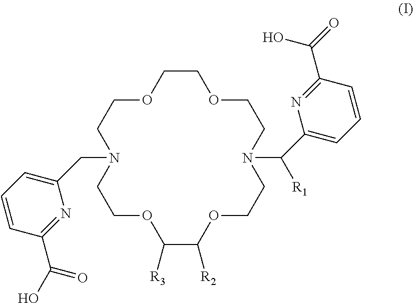

or a pharmaceutically acceptable salt thereof, wherein:
R₁ is hydrogen and R₂ is -L₁-R₄;
alternatively, R₁ is -L₁-R₄ and R₂ is hydrogen;
R₃ is hydrogen;
alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
L₁ is absent or a linker; and
R₄ is a nucleophilic moiety, an electrophilic moiety, or a targeting ligand.

In some embodiments, L₁ is absent. When L₁ is absent, R₄ is directly bound (e.g., via covalent linkage) to the compound.

In some embodiments, L₁ is a linker. As used herein, the term “linker” refers to a chemical moiety that joins a compound of the invention to a nucleophilic moiety, electrophilic moiety, or targeting ligand. Any suitable linker known to those skilled in the art in view of the present disclosure can be used in the invention. The linkers can have, for example, a substituted or unsubstituted alkyl, a substituted or unsubstituted heteroalkyl moiety, a substituted or unsubstituted aryl or heteroaryl, a polyethylene glycol (PEG) linker, a peptide linker, a sugar-based linker, or a cleavable linker, such as a disulfide linkage or a protease cleavage site such as valine-citrulline-p-aminobenzyl (PAB). Exemplary linker structures suitable for use in the invention include, but are not limited to:

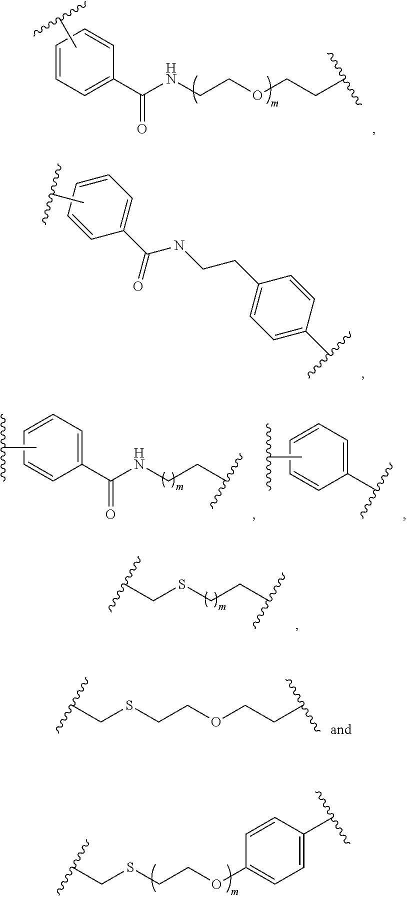

wherein m is an integer of 0 to 12.

In some embodiments, R₄ is a nucleophilic moiety or an electrophilic moiety. A “nucleophilic moiety” or “nucleophilic group” refers to a functional group that donates an electron pair to form a covalent bond in a chemical reaction. An “electrophilic moiety” or “electrophilic group” refers to a functional group that accepts an electron pair to form a covalent bond in a chemical reaction. Nucleophilic groups react with electrophilic groups, and vice versa, in chemical reactions to form new covalent bonds. Reaction of the nucleophilic group or electrophilic group of a compound of the invention with a targeting ligand or other chemical moiety (e.g., linker) comprising the corresponding reaction partner allows for covalent linkage of the targeting ligand or chemical moiety to the compound of the invention.

Exemplary examples of nucleophilic groups include, but are not limited to, azides, amines, and thiols. Exemplary examples of electrophilic groups include, but are not limited to amine-reactive groups, thiol-reactive groups, alkynyls and cycloalkynyls. An amine-reactive group preferably reacts with primary amines, including primary amines that exist at the N-terminus of each polypeptide chain and in the side-chain of lysine residues. Examples of amine-reactive groups suitable for use in the invention include, but are not limited to, N-hydroxy succinimide (NHS), substituted NHS (such as sulfo-NHS), isothiocyanate (—NCS), isocyanate (—NCO), esters, carboxylic acid, acyl halides, amides, alkylamides, and tetra- and per-fluoro phenyl ester. A thiol-reactive group reacts with thiols, or sulfhydryls, preferably thiols present in the side-chain of cysteine residues of polypeptides. Examples of thiol-reactive groups suitable for use in the invention include, but are not limited to, Michael acceptors (e.g., maleimide), haloacetyl, acyl halides, activated disulfides, and phenyloxadiazole sulfone.

In certain embodiments, R₄ is —NH₂, —NCS (isothiocyanate), —NCO (isocyanate), —N₃ (azido), alkynyl, cycloalkynyl, carboxylic acid, ester, amido, alkylamide, maleimido, acyl halide, tetrazine, or trans-cyclooctene, more particularly —NCS, —NCO, —N₃, alkynyl, cycloalkynyl, —C(O)R₁₃, —COOR₁₃, —CON(R₁₃)₂, maleimido, acyl halide (e.g., —C(O)Cl, —C(O)Br), tetrazine, or trans-cyclooctene wherein each R₁₃ is independently hydrogen or alkyl.

In some embodiments, R₄ is an alkynyl, cycloalkynyl, or azido group thus allowing for attachment of the compound of the invention to a targeting ligand or other chemical moiety (e.g., linker) using a click chemistry reaction. In such embodiments, the click chemistry reaction that can be performed is a Huisgen cycloaddition or 1,3-dipolar cycloaddition between an azido (—N₃) and an alkynyl or cycloalkynyl group to form a 1,2,4-triazole linker or moiety. In one embodiment, the compound of the invention comprises an alkynyl or cycloalkynyl group and the targeting ligand or other chemical moiety comprises an azido group. In another embodiment, the compound of the invention comprises an azido group and the targeting ligand or other chemical moiety comprises an alkynyl or cycloalkynyl group.

In certain embodiments, R₄ is an alkynyl group, more preferably a terminal alkynyl group or cycloalkynyl group that is reactive with an azide group, particularly via strain-promoted azide-alkyne cycloaddition (SPAAC). Examples of cycloalkynyl groups that can react with azide groups via SPAAC include, but are not limited to cyclooctynyl or a bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

In certain embodiments, R₄ is dibenzoazacyclooctynyl (DIBAC, DBCO, ADIBO), which has the following structure:

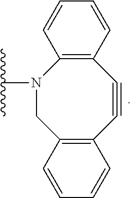 [SMILES: *N1Cc2ccccc2C#Cc2ccccc21]

In embodiments in which R₄ is DBCO, the DBCO can be covalently linked to a compound directly or indirectly via a linker, and is preferably attached to the compound indirectly via a linker.

In certain embodiments, R₄ is a targeting ligand. The targeting ligand can be linked to the compound directly via a covalent linkage, or indirectly via a linker. The targeting ligand can be a polypeptide, e.g., antibody or antigen binding fragment thereof, aptamer, or scaffold protein, etc. In preferred embodiments, the targeting ligand is an antibody or antigen binding fragment thereof, such as antibody or antigen binding fragment thereof, e.g., monoclonal antibody (mAb) or antigen binding fragment thereof, which specifically binds an antigen associated with a neoplastic disease or disorder, such as a cancer antigen, which can be prostate-specific membrane antigen (PSMA), BCMA, Her2, EGFR, KLK2, CD19, CD22, CD30, CD33, CD79b, or Nectin-4.

According to particular embodiments, the targeting ligand specifically binds to a prostate-specific antigen (e.g., PSMA or KLK2).

In another embodiment, the invention is directed to a compound of Formula (II):

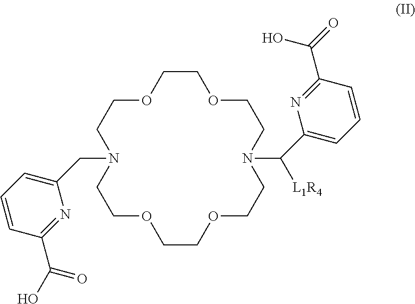

or a pharmaceutically acceptable salt thereof, wherein:
L₁ is absent or a linker; and
R₄ is a nucleophilic moiety, an electrophilic moiety, or a targeting ligand.

In another embodiment of the invention is directed to a compound of Formula (III):

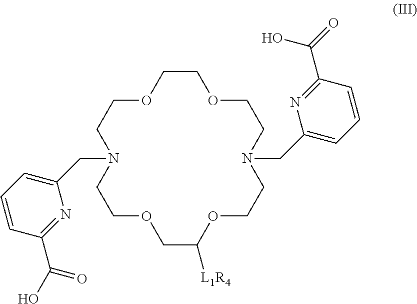

or a pharmaceutically acceptable salt thereof, wherein:
L₁ is absent or a linker; and
R₄ is a nucleophilic moiety, an electrophilic moiety, or a targeting ligand.

In another embodiment, the invention is directed to a compound, wherein: R₁ is -L₁-R₄; R₂ and R₃ are taken together with the carbon atoms to which they are attached to form a 5- or 6-membered cycloalkyl; L₁ is absent or a linker; and R₄ is a nucleophilic moiety, an electrophilic moiety, or a targeting ligand; or a pharmaceutically acceptable salt thereof.

In a further embodiment, the invention is directed to a compound, wherein R₁ is H; R₂ and R₃ are taken together with the carbon atoms to which they are attached to form a 5- or 6-membered cycloalkyl substituted with -L₁-R₄; L₁ is absent or a linker; and R₄ is a nucleophilic moiety, an electrophilic moiety, or a targeting ligand; or a pharmaceutically acceptable salt thereof.

Additional embodiments include those wherein R₄ is a targeting ligand, wherein the targeting ligand is selected from the group consisting of an antibody, antigen binding fragment of an antibody, scaffold protein, and aptamer.

In an embodiment, the compounds of the invention are any one or more independently selected from the group consisting of:

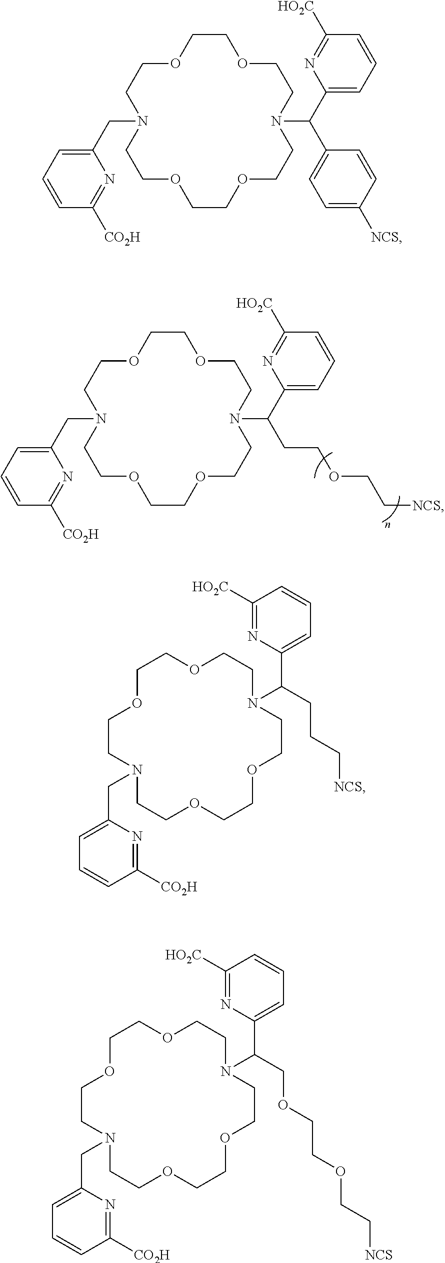

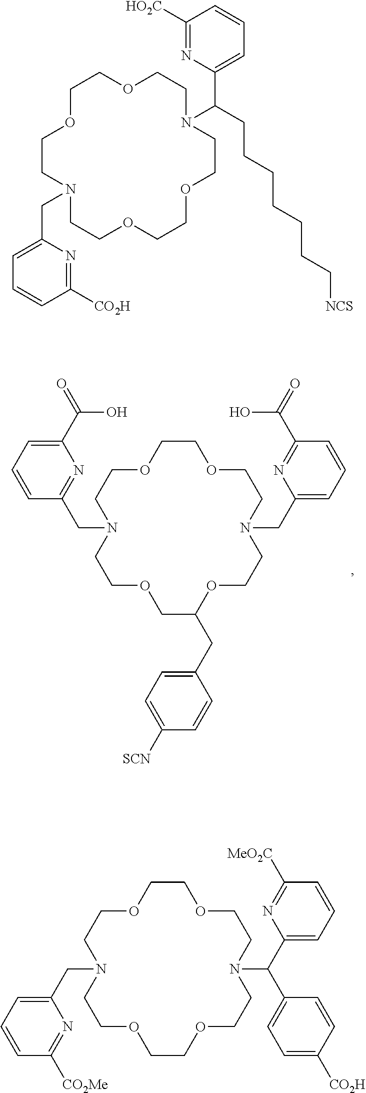

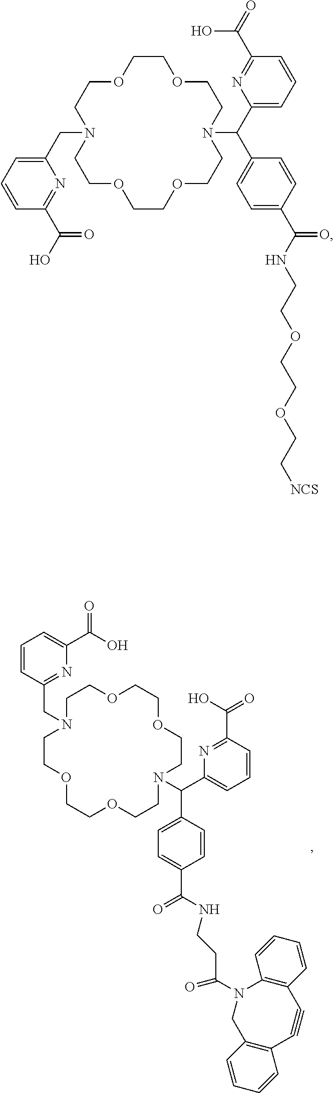

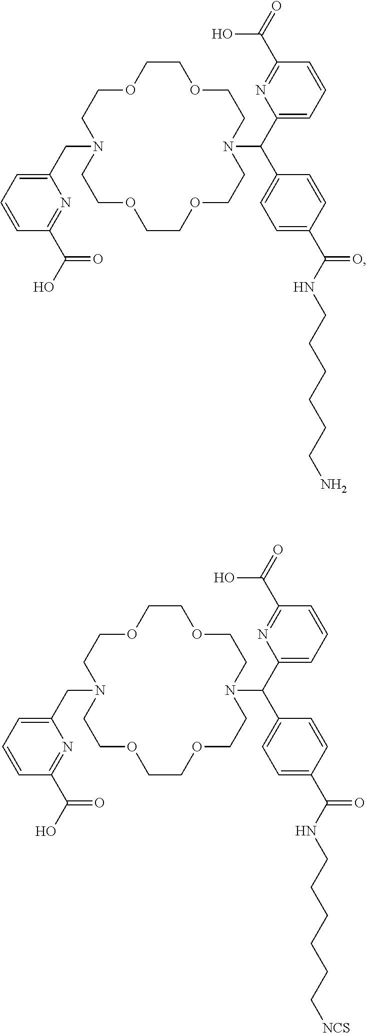

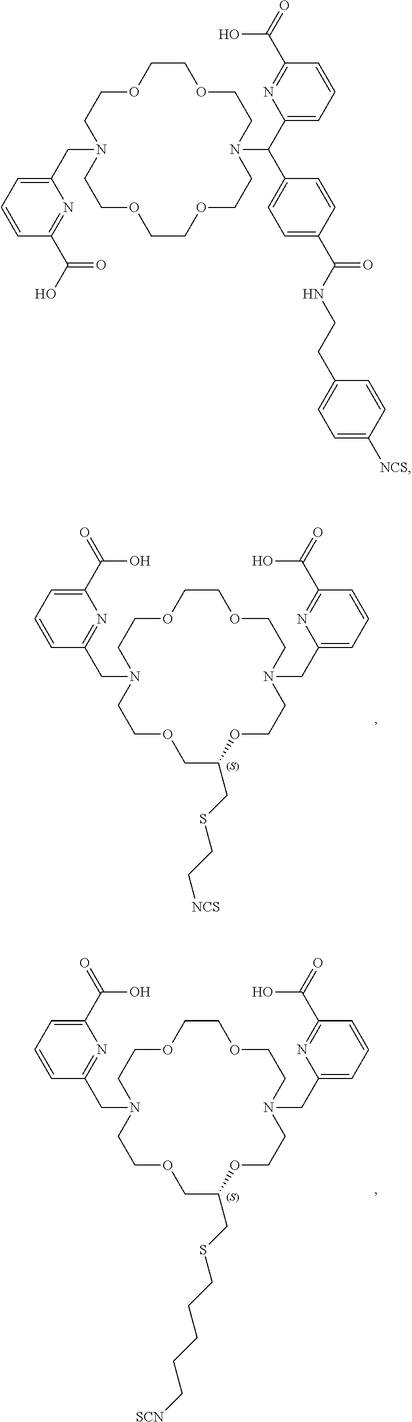

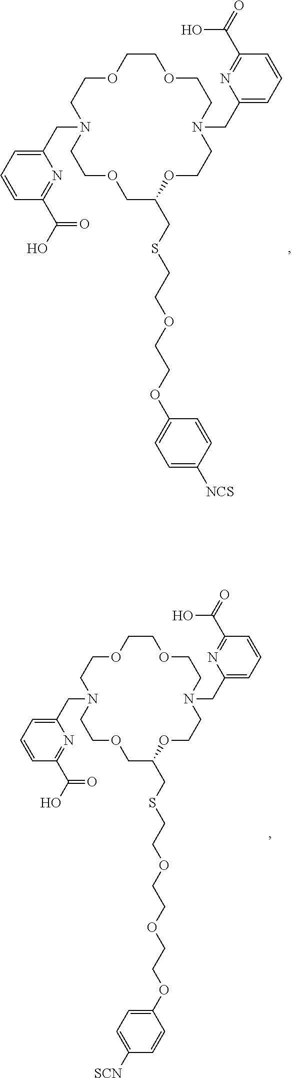

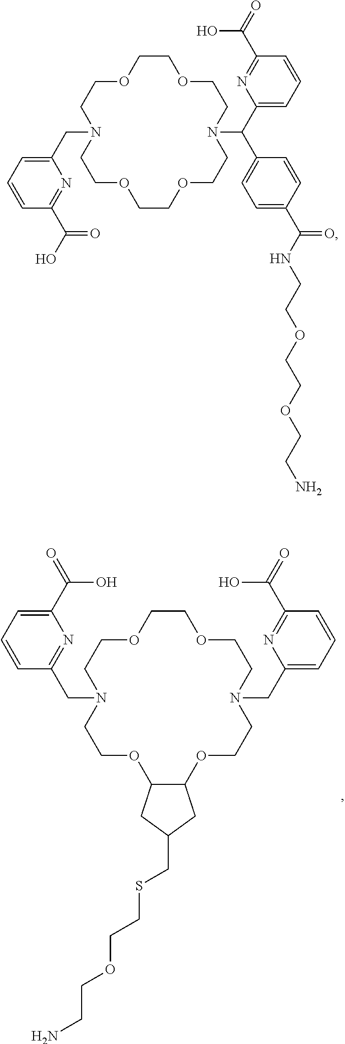

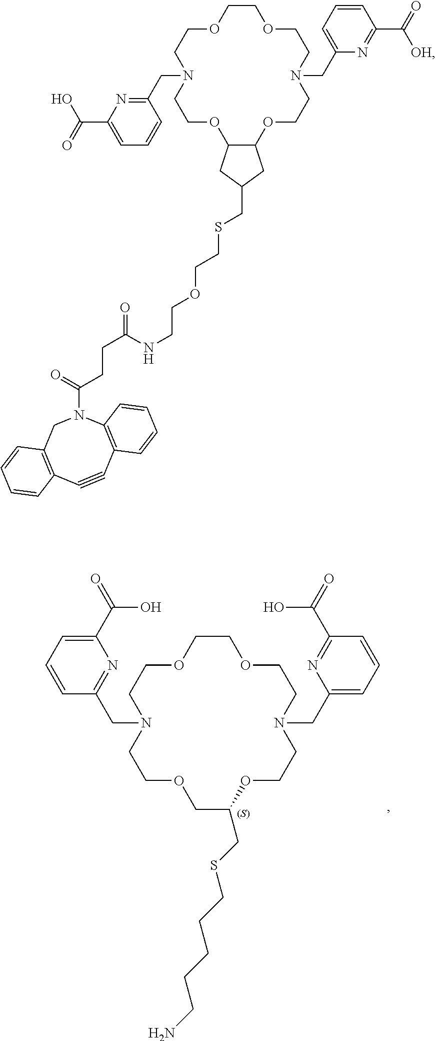

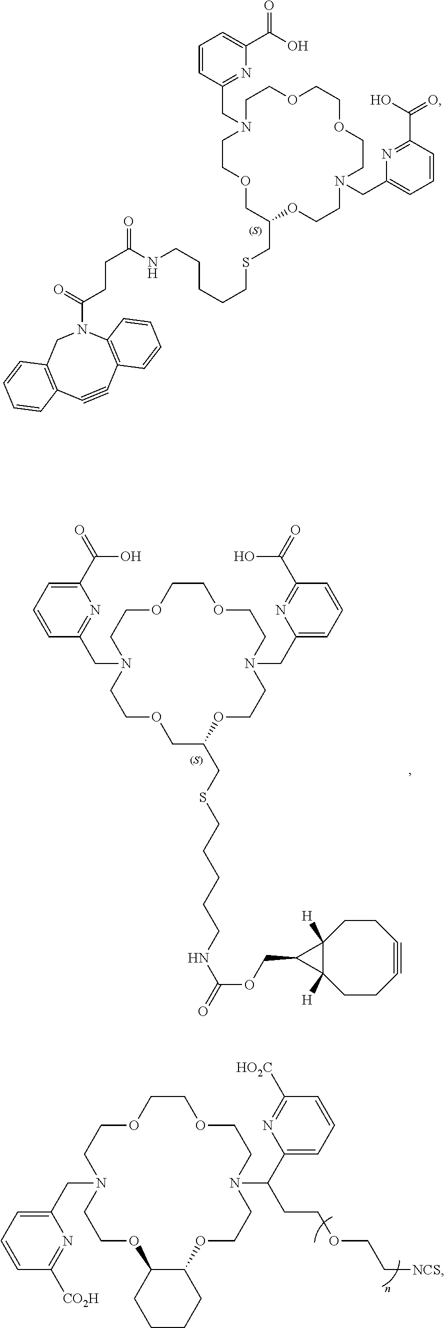

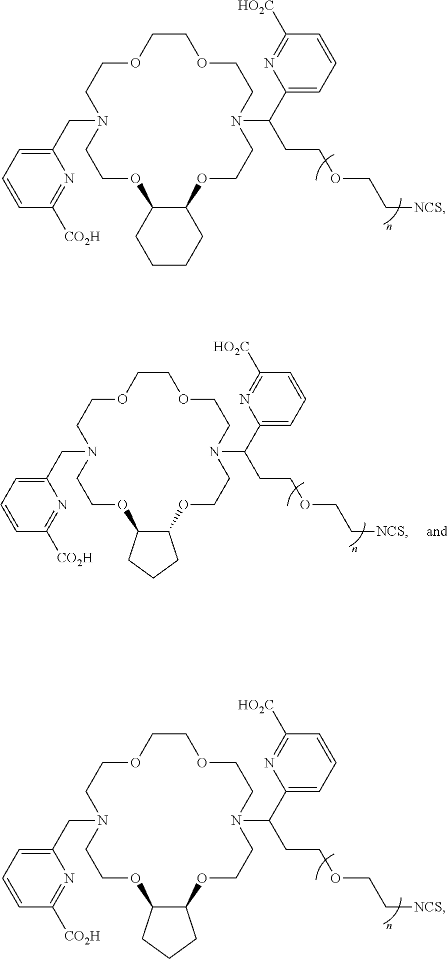

wherein n is 1-10.

Said compounds can be covalently attached to a targeting ligand (e.g., an antibody or antigen binding fragment thereof) to form immunoconjugates or radioimmunoconjugates (when complexed with a metal) by reacting the compound with an azide-labeled targeting ligand to form a 1,2,3-triazole linker via a click chemistry reaction as described in more detail below.

Compounds of the invention can be produced by any method known in the art in view of the present disclosure. For example, the pendant aromatic/heteroaromatic groups can be attached to the macrocyclic ring portion by methods known in the art, such as those exemplified and described below.

### Radiometal Complexes

In certain embodiments, the invention is directed to radiometal complexes comprising a radiometal ion complexed to a compound of the invention via coordinate bonding. Any of the compounds of the invention described herein can comprise a radiometal ion. Preferably, the radiometal ion is an alpha-emitting radiometal ion, more preferably ²²⁵Ac. Compounds of the invention can complex to radiometal ions, particularly ²²⁵Ac at any specific activity irrespective of metal impurities, thus forming a radiometal complex having high chelation stability in vivo and in vitro and which is stable to challenge agents, e.g., diethylene triamine pentaacetic acid (DTPA).

In certain embodiments, the invention is directed to a radiometal complex structure of Formula (I-M⁺):

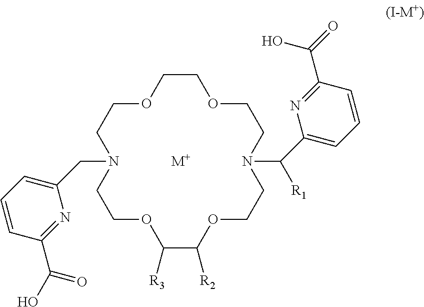

or a pharmaceutically acceptable salt thereof, wherein:
M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
R₁ is hydrogen and R₂ is -L₁-R₄;
alternatively, R₁ is -L₁-R₄ and R₂ is hydrogen;
R₃ is hydrogen;
alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
L₁ is absent or a linker;
R₄ is a nucleophilic moiety, an electrophilic moiety, or a targeting ligand.

In another embodiment, the invention is directed to a radiometal complex of Formula (II-M⁺):

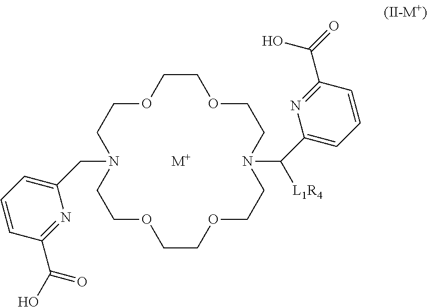

or a pharmaceutically acceptable salt thereof, wherein:
M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
L₁ is absent or a linker;
R₄ is a nucleophilic moiety, an electrophilic moiety, or a targeting ligand.

In another embodiment, the invention is directed to a radiometal complex of Formula (III-M⁺):

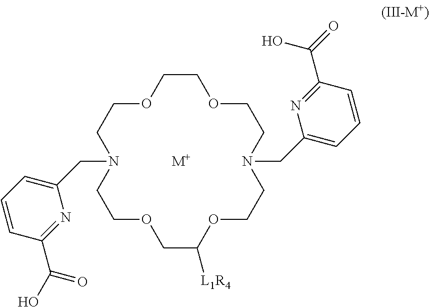

or a pharmaceutically acceptable salt thereof, wherein:
M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U); L₁ is absent or a linker; and
R₄ is a nucleophilic moiety, an electrophilic moiety, or a targeting ligand.

In another embodiment, the invention is directed to a radiometal complex wherein:
M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
R₁ is -L₁-R₄;
R₂ and R₃ are taken together with the carbon atoms to which they are attached to form a 5- or 6-membered cycloalkyl;
L₁ is absent or a linker; and
R₄ is a nucleophilic moiety, an electrophilic moiety, or a targeting ligand;

or a pharmaceutically acceptable salt thereof.

In a further embodiment, the invention is directed to a radiometal complex wherein
M⁺ is a radiometal ion, wherein M⁺ is selected from the group consisting of actinium-225(²²⁵Ac), radium-223 (²³³Ra), bismuth-213 (213Bi), lead-212 (²¹²Pb(II) and/or ²¹²Pb(IV)), terbium-149 (149Tb), terbium-152 (¹⁵²Tb), terbium-155 (155Tb), fermium-255 (²⁵⁵Fm), thorium-227 (²²⁷Th), thorium-226 (²²⁶Th⁴⁺), astatine-211 (²¹¹At), cerium-134 (134Ce), neodymium-144 (144Nd), lanthanum-132 (132La), lanthanum-135 (135La) and uranium-230 (²³⁰U);
R₁ is H;
R₂ and R₃ are taken together with the carbon atoms to which they are attached to form a 5- or 6-membered cycloalkyl substituted with -L₁-R₄;
L₁ is absent or a linker; and
R₄ is a nucleophilic moiety, an electrophilic moiety, or a targeting ligand;

or a pharmaceutically acceptable salt thereof.

In certain embodiments, the invention is directed to any one or more radiometal complexes selected from the group consisting of:

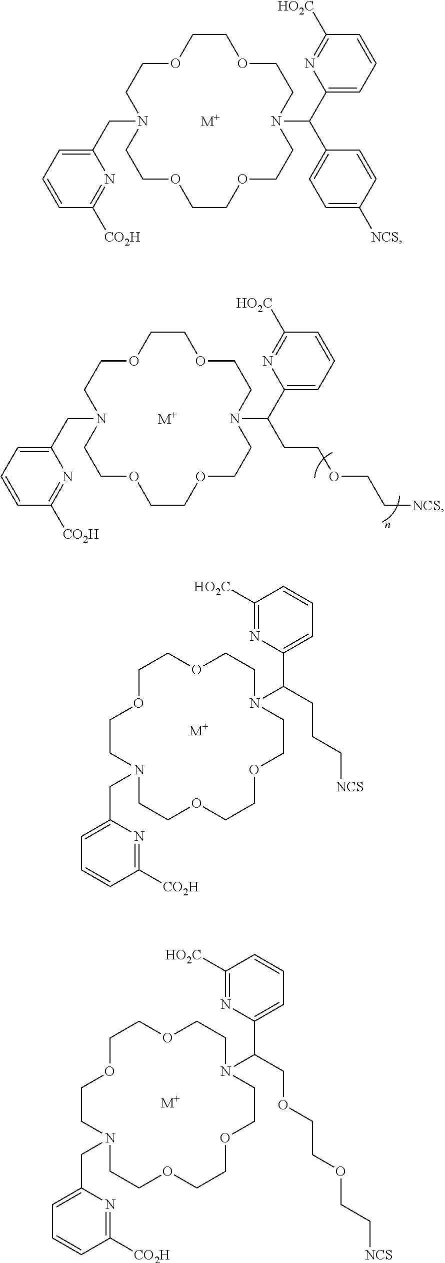

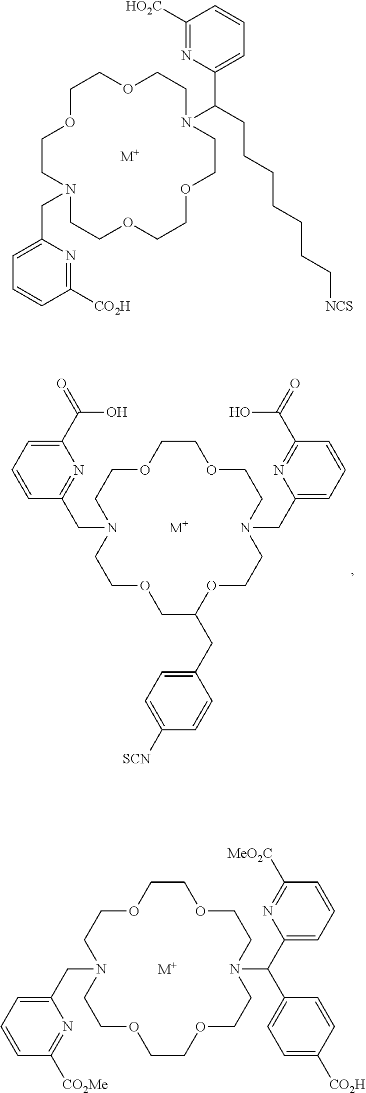

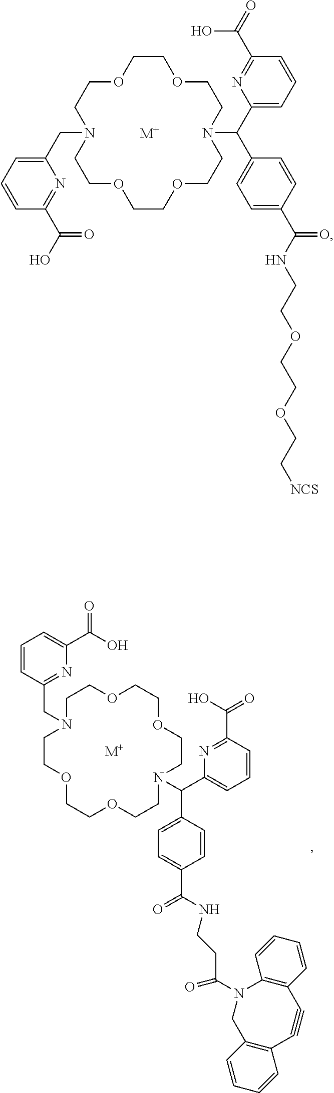

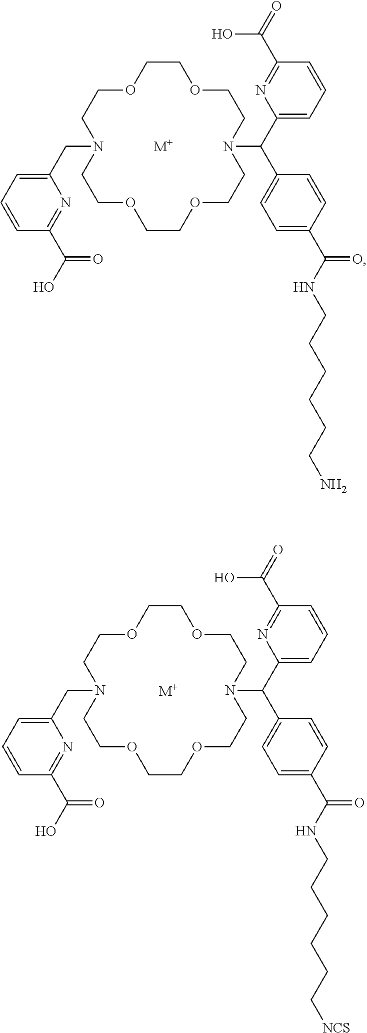

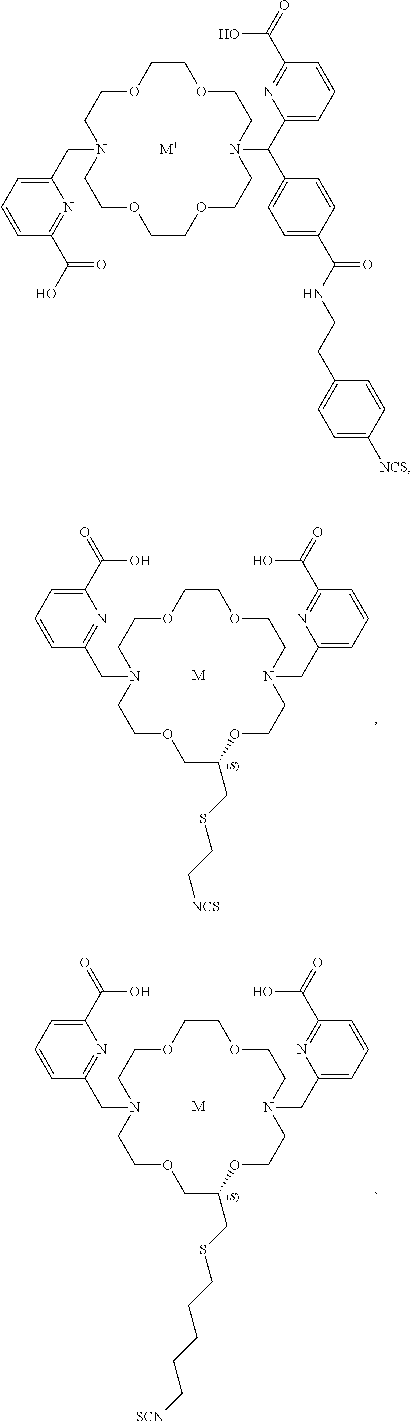

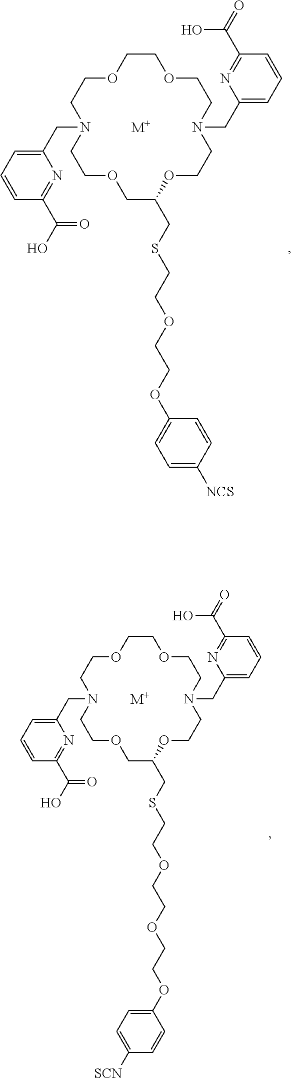

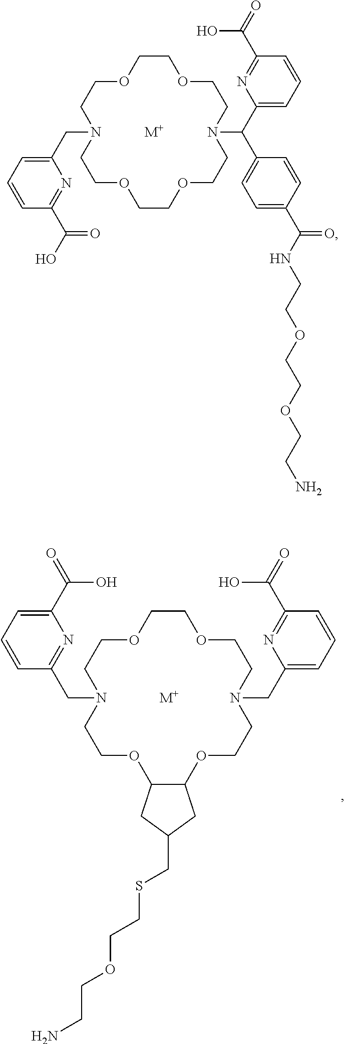

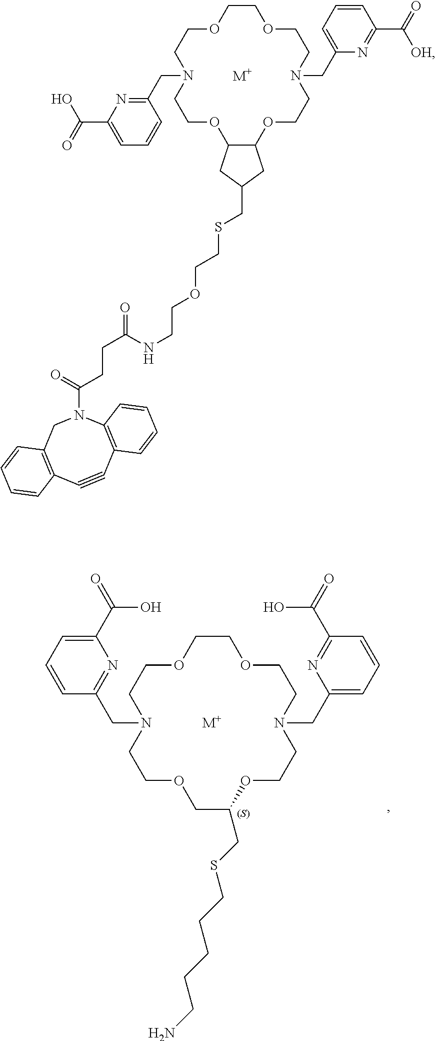

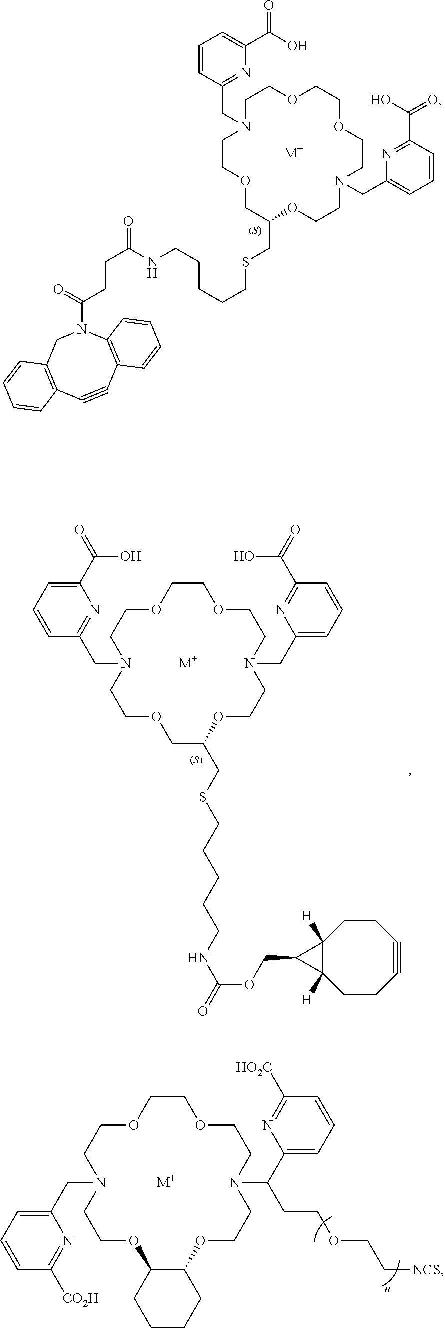

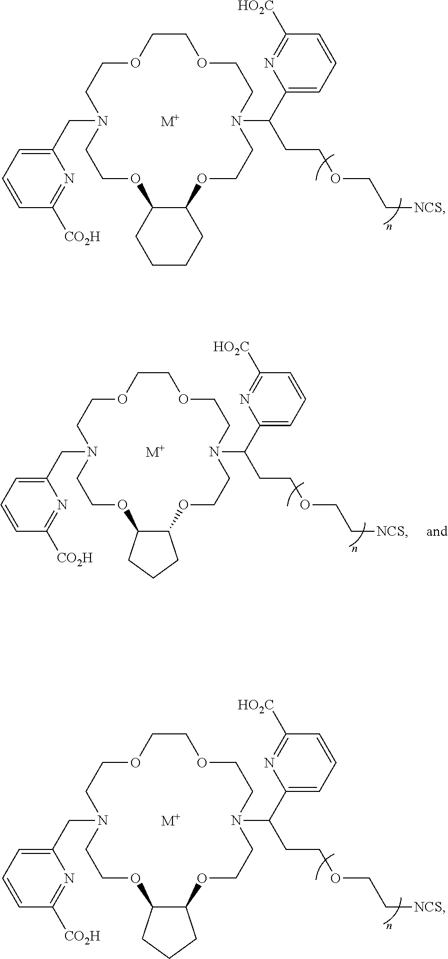

wherein n is 1-10 and M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U).

Radiometal complexes can be produced by any method known in the art in view of the present disclosure. For example, a macrocyclic compound of the invention can be mixed with a radiometal ion and the mixture incubated to allow for formation of the radiometal complex. In an exemplary embodiment, a compound is mixed with a solution of ²²⁵Ac(NO₃)₃ to form a radiocomplex comprising ²²⁵Ac bound to the compound via coordinate bonding. As described above, compounds of in the invention efficiently chelate radiometals, particularly ²²⁵Ac. Thus, in particular embodiments, a compound of the invention is mixed with a solution of ²²⁵Ac ion at a ratio by concentration of compound of the invention to ²²⁵Ac ion of 1:1000, 1:500, 1:400, 1:300, 1:200, 1:100, 1:50, 1:10, or 1:5, preferably 1:5 to 1:200, more preferably 1:5 to 1:100. Thus, in some embodiments, the ratio of a compound of the invention to ²²⁵Ac which can be used to form a radiometal complex is much lower than that which can be achieved with other known ²²⁵Ac chelators, e.g., DOTA. The radiocomplex can be characterized by instant thin layer chromatography (e.g., iTLC-SG), HPLC, LC-MS, etc. Exemplary methods are described herein, e.g., in the Examples below.

### Immunoconjugates and Radioimmunoconiumates

In another embodiment, the invention is directed to immunoconjugates and radioimmunoconjugates. Compounds of the invention and radiometal complexes of the invention can be conjugated to (i.e., covalently linked to) targeting ligands, such as an immune substance to produce immunoconjugates and/or radioimmunoconjugates that are suitable, for example, for medicinal applications in subjects, e.g., humans, such as targeted radiotherapy. Using the macrocyclic compounds, radiometal complexes and radioimmunoconjugates of the invention, targeting ligands, particularly antibodies or antigen binding fragments thereof that can bind specifically to targets of interest (such as cancer cells), can be site-specifically labeled with radiometal ions to produce radioimmunoconjugates. In particular, using the compounds of the invention and/or radiometal complexes of the invention, radioimmunoconjugates having high yield complexation of radiometal ions, particularly ²²⁵Ac, and desired compound-antibody ratio (CAR) can be produced.

According to particular embodiments, methods of the present invention provide an average CAR of less than 10, less than 8, less than 6, or less than 4; or a CAR of between about 2 to about 8, or about 2 to about 6, or about 2 to about 4, or about 2 to about 3; or a CAR of about 2, or about 3, or about 4, or about 5, or about 6, or about 7, or about 8.

As used herein, an “immunoconjugate” is an antibody or antigen binding fragment thereof conjugated to (e.g., bound via a covalent bond) to a second molecule, such as a toxin, drug, radiometal ion, radiometal complex, etc. A “radioimmunoconjugate” (which may also be referred to as a radioconjugate) in particular is an immunoconjugate in which an antibody or antigen binding fragment thereof is labeled with a radiometal or conjugated to a radiometal complex.

In certain embodiments of the invention, an immunoconjugate comprises a compound of the invention, e.g., a compound of Formula (I) as described herein, covalently linked to an antibody or antigen binding fragment thereof, preferably via a linker. Numerous modes of attachment with different linkages between the compounds of the invention and antibody or antigen binding fragment thereof are possible depending on the reactive functional groups (i.e., nucleophiles and electrophiles) on the compounds of Formula (I) and antibody or antigen binding fragment thereof.

In certain embodiments of the invention, a radioimmunoconjugate comprises a radiometal complex of the invention, e.g., a radiometal complex as described herein, covalently linked to an antibody or antigen binding fragment thereof, preferably via a linker.

Any of the compounds or radiometal complexes of the invention described herein can be used to produce immunoconjugates or radioimmunoconjugates of the invention.

In certain embodiments, a radiometal complex or radioimmunoconjugate of the invention comprises an alpha-emitting radiometal ion coordinated to the compound moiety of the radiocomplex. Preferably, the alpha-emitting radiometal ion is ²²⁵Ac.

In certain embodiments, the antibody or antigen binding fragment in an immunoconjugate or radioimmunoconjugate of the application can bind specifically to a tumor antigen. Preferably, the antibody or antigen binding fragment binds specifically to a cancer antigen. Examples of cancer antigens include, but are not limited to, prostate-specific membrane antigen (PSMA), BCMA, Her2, EGFR, KLK2, CD19, CD22, CD30, CD33, CD79b, and Nectin-4.

In one embodiment, the antibody binds specifically to PSMA. Preferably, the antibody is PSMB127. A human IgG4 antibody that binds to human prostate-specific membrane antigen (PSMA), referred to herein as “anti-PSMA mAb” with designation “PSMB127”, has a heavy chain (HC) CDR1 sequence of SEQ ID NO: 3, a HC CDR2 sequence of SEQ ID NO: 4, a HC CDR3 sequence of SEQ ID NO: 5, a light chain (LC) CDR1 sequence of SEQ ID NO: 6, a LC CDR2 sequence of SEQ ID NO: 7, and a LC CDR3 sequence of SEQ ID NO: 8, and has a HC sequence of SEQ ID NO: 9 and a LC sequence of SEQ ID NO: 10. Anti-PSMA mAb was expressed and purified using standard chromatography methods. The antibody PSMB127, its biologic activities, uses or other related information thereof are described, for example, in U.S. Patent Application Publication No. US 20200024360A1, the contents of which are hereby incorporated by reference in their entireties.

In another embodiment, the antibody binds specifically to human kallikrein-2 (KLK2). KLK2 may also be referred to as hK2. Preferably, the antibody is H11B6 (also referred to as h11B6). The H11B6 antibody, biologic activities, uses or other related information thereof are described in U.S. Pat. No. 10,100,125, the contents of which are hereby incorporated by reference in their entireties. As described therein, the H11B6 antibody polypeptide comprises a heavy chain (HC) variable region comprising the amino acid sequences of SEQ ID NO: 11 and SEQ ID NO: 12 and SEQ ID NO: 13 and a light chain (LC) variable region comprising the amino acid sequences of SEQ ID NO: 14 and SEQ ID NO: 15 and SEQ ID NO: 16.

Thus, according to particular embodiments, a radioconjugate of the present invention comprises an h11B6 antibody which comprises (a) a heavy chain variable region (VH) comprising a VH CDR1 having an amino acid sequence of SEQ ID NO:11 (SDYAWN), a VH CDR2 having an amino acid sequence of and SEQ ID NO:12 (YISYSGSTTYNPSLKS) and a VH CDR3 having an amino acid sequence of SEQ ID NO:13 (GYYYGSGF); and (b) a light chain variable region (VL) comprising a VL CDR1 having an amino acid sequence of SEQ ID NO:14 (KASESVEYFGTSLMH), a VL CDR2 having an amino acid sequence of and SEQ ID NO:15 (AASNRES) and a VL CDR3 having an amino acid sequence of SEQ ID NO:16 (QQTRKVPYT).

The H11B6 antibody can further have a heavy chain variable region which comprises the amino acid sequence of SEQ ID NO: 17 and a light chain variable region which comprises the amino acid sequence of SEQ ID NO: 18, or have a heavy chain constant region which comprises the amino acid sequence of SEQ ID NO: 19 and a light chain constant region which comprises the amino acid sequence of SEQ ID NO: 20, or have a heavy chain comprising the amino acid sequence of SEQ ID NO:21 and a light chain comprising the amino acid sequence of SEQ ID NO:22.

Kabat numbering scheme (Kabat et al, 1991) is used throughout this description (Sequences of Immunological Interest, 5th edition, NH, Bethesda, Md., the disclosures of which are incorporated herein by reference).

According to particular embodiments, an antibody of the present invention comprises a heavy chain variable region (VH) having at least 80%, at least 85%, at least 90%, at least 95%, or at least 98% sequence identity to the amino acid sequence of SEQ ID NO: 17, and/or a light chain variable region (VL) having at least 80%, at least 85%, at least 90%, at least 95%, or at least 98% sequence identity to the amino acid sequence of SEQ ID NO: 18.

According to particular embodiments, an antibody of the present invention a heavy chain constant region having at least 80%, at least 85%, at least 90%, at least 95%, or at least 98% sequence identity to the amino acid sequence of SEQ ID NO: 19, and/or a light chain constant region having at least 80%, at least 85%, at least 90%, at least 95%, or at least 98% sequence identity to the amino acid sequence of SEQ ID NO: 20.

According to particular embodiments, an antibody of the present invention comprises a heavy chain having at least 80%, at least 85%, at least 90%, at least 95%, or at least 98% sequence identity to the amino acid sequence of SEQ ID NO: 21, and/or a light chain having at least 80%, at least 85%, at least 90%, at least 95%, or at least 98% sequence identity to the amino acid sequence of SEQ ID NO: 22.

According to particular embodiments, an antibody of the present invention (e.g., h11B6) comprises or consists of an intact (i.e. complete) antibody, such as an IgA, IgD, IgE, IgG or IgM molecule.

According to particular embodiments, an antibody of the present invention (e.g., h11B6) comprises or consists of an intact IgG molecule, or a variant of the same. The IgG molecule may be of any known subtype, for example IgG1, IgG2, IgG3 or IgG4.

According to particular embodiments, an antibody of the present invention comprises an h11B6 antibody that is an IgG1 antibody. According to particular embodiments, an antibody of the present invention comprises an h11B6 antibody that is an IgG1 kappa isotype. According to particular embodiments, an antibody of the present invention comprises an h11B6 antibody that is an IgG1 antibody or a variant thereof, such as an Fc variant.

In any of the embodiments disclosed herein (for simplicity's sake, hereinafter recited as “in any embodiment disclosed herein” or the like), the antibody may include, but is not limited to, belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Ziv-aflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab. In any embodiment disclosed herein, it may be that the antibody fragment includes an antigen-binding fragment of belimumab, Mogamulizumab, Blinatumomab, Ibritumomab tiuxetan, Obinutuzumab, Ofatumumab, Rituximab, Inotuzumab ozogamicin, Moxetumomab pasudotox, Brentuximab vedotin, Daratumumab, Ipilimumab, Cetuximab, Necitumumab, Panitumumab, Dinutuximab, Pertuzumab, Trastuzumab, Trastuzumab emtansine, Siltuximab, Cemiplimab, Nivolumab, Pembrolizumab, Olaratumab, Atezolizumab, Avelumab, Durvalumab, Capromab pendetide, Elotuzumab, Denosumab, Zivaflibercept, Bevacizumab, Ramucirumab, Tositumomab, Gemtuzumab ozogamicin, Alemtuzumab, Cixutumumab, Girentuximab, Nimotuzumab, Catumaxomab, or Etaracizumab. In any embodiment disclosed herein, the binding peptide may include, but is not limited to, a prostate specific membrane antigen (“PSMA”) binding peptide, a somatostatin receptor agonist, a bombesin receptor agonist, a seprase binding compound, or a binding fragment thereof.

Immunoconjugates and radioimmunoconjugates of the invention can be prepared by any method known in the art in view of the present disclosure for conjugating ligands, e.g., antibodies, to compounds of the invention, including chemical and/or enzymatic methods. For example, immunoconjugates and radioimmunoconjugates can be prepared by a coupling reaction, including by not limited to, formation of esters, thioesters, or amides from activated acids or acyl halides; nucleophilic displacement reactions (e.g., such as nucleophilic displacement of a halide ring or ring opening of a strained ring system); azide-alkyne Huisgen cycloaddition (e.g., 1,3-dipolar cycloaddition between an azide and alkyne to form a 1,2,3-triazole linker); thiolyne addition; imine formation; Diels-Alder reactions between tetrazines and trans-cycloctene (TCO); and Michael additions (e.g., maleimide addition). Numerous other modes of attachment, with different linkages, are possible depending on the reactive functional group used. The attachment of a ligand can be performed on a compound that is coordinated to a radiometal ion, or on a compound which is not coordinated to a radiometal ion.

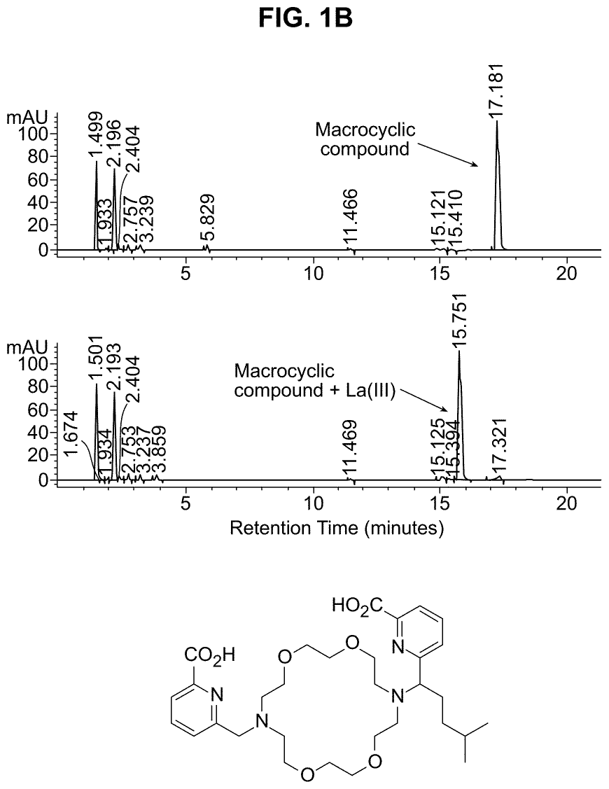

In an embodiment, a radioimmunoconjugate can be produced by covalently linking a radiometal complex of the invention to an antibody or antigen binding fragment thereof by, for example, a click chemistry reaction (see, e.g., FIGS. 2B and 2D, referred to as “click radiolabeling”). Alternatively, a radioimmunoconjugate can be produced by first preparing an immunoconjugate of the invention by covalently linking a compound of the invention to an antibody or antigen-binding fragment thereof by, for example, a click chemistry reaction; the immunoconjugate can subsequently be labeled with a radiometal ion to produce a radioimmunoconjugate (see, e.g., FIGS. 2A and 2C, referred to as “one-step direct radiolabeling”). Both residue-specific (e.g., FIGS. 2A and 2B) and site-specific methods (e.g., FIGS. 2C and 2D) of conjugation can be used to produce immunoconjugate and radioimmunoconjugates of the invention.

Residue-specific methods for conjugation to proteins are well established and most commonly involve either lysine side chains, using an activated ester or isothiocyanate, or cysteine side chains with a maleimide, haloacetyl derivative or activated disulfide (Brinkley Bioconjugate Chem 1992:2). Since most proteins have multiple lysine and cysteine residues, heterogeneous mixtures of product with different numbers of conjugated molecules at a variety of amino acid positions are typically obtained using such methods. Additional methods have been established including tyrosine-specific conjugation (Ban et al. Bioconjugate Chemistry 2013:520), methionine-specific methods (Lin et al. Science 2017 (355) 597), additional cysteine-focused approaches (Toda et al. Angew Chemie 2013:12592), and others.

More recently, site-selective and site-specific conjugation methods have been established for monoclonal antibodies and other proteins (Agarwal, P. and C. R. Bertozzi, Bioconjug Chem, 2015. 26(2): p. 176-92; Rabuka et al. Curr Opin Chem Biol 2010:790). These include incorporation of unnatural amino acids; fusion of the protein of interest to a ‘self-labeling tag’ such as SNAP or DHFR or a tag that is recognized and modified specifically by another enzyme such as sortase A, lipoic acid ligase and formylglycine-generating enzyme; enzymatic modification of the glycan to allow conjugation of payloads of interest (Hu et al. Chem Soc Rev 2016:1691); use of microbial transglutaminase to selectively recognize defined positions on the antibody; and additional methods using molecular recognition and/or chemical approaches to affect selective conjugation (Yamada et al. 2019:5592; Park et al. Bioconjugate Chem 2018:3240; Pham et al. Chembiochem 2018:799).

In certain embodiments, an immunoconjugate or radioimmunoconjugate of the invention is produced using residue specific methods for conjugation of a compound of the invention to an antibody or antigen binding fragment thereof. Such residue specific methods typically result in an immunoconjugate or radioimmunoconjugate covalently linked to a compound of the invention or radiometal complex at a variety of positions of the antibody. Any residue specific method for forming protein or antibody conjugates known to those skilled in the art in view of the present disclosure can be used. Examples of residue specific methods for conjugation that can be used include, but are not limited to, conjugation of a compound of the invention or radiometal complex to lysine residues of the antibody using a compound of the invention or radiometal complex comprising, e.g., an activated ester or isothiocyanate group; conjugation to cysteine residues of the antibody using a compound of the invention or radiometal complex comprising, e.g., a maleimide, haloacetyl derivative, acyl halide, activated disulfide group, or methylsulfonyl phenyloxadiazole group; conjugation to tyrosine resides of the antibody using a compound of the invention or radiometal complex comprising, e.g., 4-phenyl-3H-1,2,4-triazoline-3,5(4H)-diones (PTADs); and conjugation to methionine residues of the antibody using a compound of the invention or radiometal complex comprising, e.g., an oxaziridine derivative. It is also possible to label the antibody at a particular residue with a biorthogonal reactive functional group using one or more of the above described methods prior to conjugating to a compound of the invention or radiometal complex of the invention. For example, tyrosine residues can be site-specifically labeled with a biorthogonal reactive functional group using an oxaziridine derivative linked to the biorthogonal reactive functional group, e.g., azido, alkynyl, or cycloalkynyl, and then the antibody containing the labeled tyrosine residues can be conjugated to a compound of the invention or radiometal complex of the invention, using a compound of the invention or radiometal complex bearing a compatible reactive functional group.

In certain embodiments, an immunoconjugate or radioimmunoconjugate of the invention can be produced using site-specific or site-selective methods for conjugation of a compound of the invention to an antibody or antigen binding fragment thereof. In contrast to residue specific methods, “site-specific” or “site-selective” methods typically result in an immunoconjugate or radioimmunoconjugate covalently linked to a compound of the invention or radiometal complex at a specified position of the antibody. Any site-specific method for forming protein or antibody conjugates known to those skilled in the art in view of the present disclosure can be used. For example, an unnatural amino acid (e.g., azido- or alkynyl-amino acid) can be site-specifically incorporated into an antibody using a mutant aminoacyl t-RNA synthetase that can selectively aminoacylate its tRNA with an unnatural amino acid of interest. The mutant acylated tRNA together with an amber suppressor tRNA can then be used to site-specifically incorporate the unnatural amino acid into a protein in response to an amber nonsense codon. An antibody that is site-specifically labeled by one or more of the above described methods can subsequently be conjugated to a compound of the invention or radiometal complex of the invention bearing a compatible reactive functional group.

In certain embodiments, the invention is directed to a method of producing a radioimmunoconjugate comprises reacting a compound of the invention or radiocomplex of the invention, wherein R₄ is a nucleophilic or electrophilic moiety, with an antibody or antigen binding fragment thereof, or a modified antibody or antigen binding fragment thereof comprising a nucleophilic or electrophilic moiety.

In one embodiment, the invention is directed to a method comprising reacting a compound of the invention with an antibody or antigen binding fragment thereof, or a modified antibody or antigen binding fragment thereof comprising a nucleophilic or electrophilic functional group, to form an immunoconjugate having a covalent linkage between the compound of the invention and antibody or antigen binding fragment thereof, or modified antibody or antigen binding fragment thereof, and then reacting the immunoconjugate with a radiometal ion such that the radiometal ion binds the compound of the invention of the immunoconjugate via coordinate binding, thereby forming the radioimmunoconjugate. This embodiment may be referred to as a “one-step direct radiolabeling” method (e.g., as schematically illustrated in FIG. 2C) because there is only one chemical reaction step involving the radiometal.

In another embodiment, the invention is directed to a method comprising reacting a radiocomplex of the invention with an antibody or antigen binding fragment thereof, or a modified antibody or antigen binding fragment thereof comprising a nucleophilic or electrophilic functional group, thereby forming the radioimmunoconjugate. This embodiment may be referred to as a “click radiolabeling” method (e.g., as schematically illustrated in FIG. 2D). A modified antibody or antigen binding fragment thereof can be produced by any method known in the art in view of the present disclosure, e.g., by labeling an antibody at a particular residue with a biorthogonal reactive functional group using one or more of the above described methods, or by site-specifically incorporating an unnatural amino acid (e.g., azido- or alkynyl-amino acid) into an antibody using one or more of the above described methods. The degree of labeling (DOL), sometimes called degree of substitution (DOS), is a particularly useful parameter for characterizing and optimizing bioconjugates, such as antibody modified by unnatural amino acid. It is expressed as an average number of the unnatural amino acid coupled to a protein molecule (e.g. an antibody), or as a molar ratio in the form of label/protein. The DOL can be determined from the absorption spectrum of the labeled antibody by any known method in the field.

In certain embodiments of the invention, immunoconjugates and radioimmunoconjugates of the invention are prepared using a click chemistry reaction. For example, radioimmunoconjugates of the invention can be prepared using a click chemistry reaction referred to as “click radiolabeling” (see, e.g., FIGS. 2B and 2D). Click radiolabeling uses click chemistry reaction partners, preferably an azide and alkyne (e.g., cyclooctyne or cyclooctyne derivative) to form a covalent triazole linkage between the radiocomplex (radiometal ion bound to the compound of the invention) and antibody or antigen binding fragment thereof. Click radiolabeling methods of antibodies are described in, e.g., International Patent Application No. PCT/US18/65913, entitled “Radiolabeling of Polypeptides” of which the relevant description is incorporated herein by reference. In other embodiments referred to as “one-step direct radiolabeling,” an immunoconjugate is prepared using a click chemistry reaction between an antibody or antigen binding fragment thereof and a compound of the invention; the immunoconjugate is then contacted with a radiometal ion to form the radioimmunoconjugate (see, e.g., FIGS. 2A and 2C).

In an embodiment, the invention is directed to a method of preparing a radioimmunoconjugate comprises binding a radiometal ion to a compound of the invention (e.g., via coordinate bonding).

In an embodiment, the “one-step direct radiolabeling” method of preparing a radioimmunoconjugate comprises contacting an immunoconjugate (i.e., polypeptide-compound of the invention complex) with a radiometal ion to form a radioimmunoconjugate, wherein the immunoconjugate comprises a compound of the present invention. According to particular embodiments, the immunoconjugate is formed via a click chemistry reaction between the compound of the present invention and the polypeptide. According to particular embodiments, the radioimmunoconjugate is formed without metal-free conditions (e.g., without any step(s) of removing or actively excluding common metal impurities from the reaction mixture). This is contrary to certain conventional methods in which it is necessary to radiolabel an antibody under strict metal-free conditions to avoid competitive (non-productive) chelation of common metals such as iron, zinc and copper, which introduce significant challenges into the production process.

In an embodiment, the invention is directed to a method of preparing a radioimmunoconjugate (comprising a “one-step direct radiolabeling” method) comprising:
(i) reacting a modified polypeptide with a compound of the invention, wherein the modified polypeptide is an antibody or antigen binding fragment thereof consisting of an azido group to yield an immunoconjugate; and
(ii) reacting the immunoconjugate with a radiometal ion to yield a radioimmunoconjugate.

In another embodiment, the invention is directed to a method of preparing a radioimmunoconjugate (comprising a “one-step direct radiolabeling” method) comprising:
(i) reacting a modified antibody or antigen binding fragment thereof consisting of an azido group with a compound Formula I to yield an immunoconjugate; and
(ii) reacting the immunoconjugate with a radiometal ion to yield a radioimmunoconjugate.

In certain embodiments, the invention is directed to a method of preparing a radioimmunoconjugate (comprises a “click radiolabeling” method as for example, illustrated in FIG. 2D) comprising:
(i) reacting a modified antibody or antigen binding fragment thereof with the radiocomplex, under a condition wherein the azido group reacts with the alkynyl group or cycloalkynyl group to yield a radioimmunoconjugate.

Conditions for carrying out click chemistry reactions are known in the art, and any conditions for carrying out click chemistry reactions known to those skilled in the art in view of the present disclosure can be used in the invention. Examples of conditions include, but are not limited to, incubating the modified polypeptide and the radiocomplex at a ratio of 1:1 to 1000:1 at a pH of 4 to 10 and a temperature of 20° C. to 70° C.

The click radiolabeling methods described above allow for complexation of the radiometal ion under low or high pH and/or high temperature conditions to maximize efficiency, which can be accomplished without the risk of inactivating the alkyne reaction partner. The efficient complexation and efficient SPAAC reaction between an azide-labeled antibody or antigen binding fragment thereof and the radiocomplex allows radioimmunoconjugates to be produced with high radiochemical yield even with low azide:antibody ratios. The only step in which trace metals must be excluded is the radiometal ion complexation to the macrocycle compound moiety; the antibody production, purification, and conjugation steps do not need to be conducted under metal free conditions.

Compounds of the invention and radiometal complexes of the invention can also be used in the production of site-specific radiolabeled polypeptides, e.g., antibodies. The click radiolabeling methods described herein facilitate site-specific production of radioimmunoconjugates by taking advantage of established methods to install azide groups site-specifically on antibodies (Li, X., et al. Preparation of well-defined antibody-drug conjugates through glycan remodeling and strain-promoted azide-alkyne cycloadditions. Angew Chem Int Ed Engl, 2014. 53(28): p. 7179-82; Xiao, H., et al., Genetic incorporation of multiple unnatural amino acids into proteins in mammalian cells. Angew Chem Int Ed Engl, 2013. 52(52): p. 14080-3). Methods of attaching molecules to proteins or antibodies in a site-specific manner are known in the art, and any method of site-specifically labeling an antibody known to those skilled in the art can be used in the invention in view of the present disclosure. Examples of methods to site-specifically modify antibodies suitable for use in the invention include, but are not limited to, incorporation of engineered cysteine residues (e.g., THIOMAB™), use of non-natural amino acids or glycans (e.g., seleno cysteine, p-AcPhe, formylglycine generating enzyme (FGE, SMARTag™), etc.), and enzymatic methods (e.g., use of glycotransferase, endoglycosidase, microbial or bacterial transglutaminase (MTG or BTG), sortase A, etc.).

In certain embodiments, a modified antibody or antigen binding fragment thereof for use in producing an immunoconjugate or radioimmunoconjugate of the invention is obtained by trimming the antibody or antigen binding fragment thereof with a bacterial endoglycosidase specific for the β-1,4 linkage between a core GlcNac residue in an Fc-glycosylation site of the antibody, such as GlycINATOR (Genovis), which leaves the inner most GlcNAc intact on the Fc, allowing for the site-specific incorporation of azido sugars at that site. The trimmed antibody or antigen binding fragment thereof can then be reacted with an azide-labeled sugar, such as UDP-N-azidoacetylgalactosamine (UDP-GalNAz) or UDP-6-azido 6-deoxy GalNAc, in the presence of a sugar transferase, such as GalT galactosyltransferase or GalNAc transferase, to thereby obtain the modified antibody or antigen binding fragment thereof.

In other embodiments, a modified antibody or antigen binding fragment thereof for use in producing an immunoconjugate or radioimmunoconjugate of the invention is obtained by deglycosylating the antibody or antigen binding fragment thereof with an amidase. The resulting deglycosylated antibody or antigen binding fragment thereof can then be reacted with an azido amine, preferably 3-azido propylamine, 6-azido hexylamine, or any azido-linker-amine or any azido-alkyl/heteroalkyl-amine, such as an azido-polyethylene glycol (PEG)-amine, for example, O-(2-aminoethyl)-O′-(2-azidoethyl)tetraethylene glycol, O-(2-aminoethyl)-O′-(2-azidoethyl)pentaethylene glycol, O-(2-aminoethyl)-O′-(2-azidoethyl)triethylene glycol, etc., or in the presence of a microbial transglutaminase to thereby obtain the modified antibody or antigen binding fragment thereof.

Any radiometal complex described herein can be used to produce a radioimmunoconjugate of the invention. In particular embodiments, the radiometal complex has the structure of Formula (I-M⁺).

In certain embodiments, the radioimmunoconjugate is any one or more structures independently selected from the group consisting of:

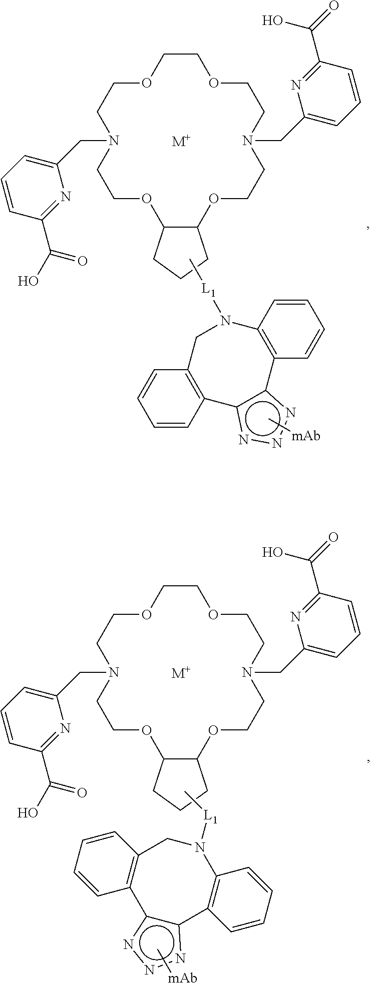

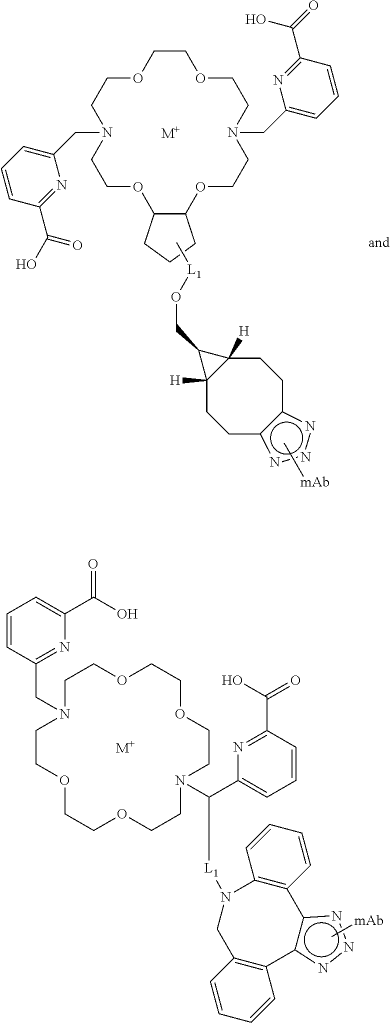

wherein:
M⁺ is a radiometal ion, wherein M⁺ is selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
L₁ is absent or a linker; and
mAb is an antibody or antigen binding fragment thereof.

In another embodiment, the radioimmunoconjugate is any one or more selected from the group consisting of:

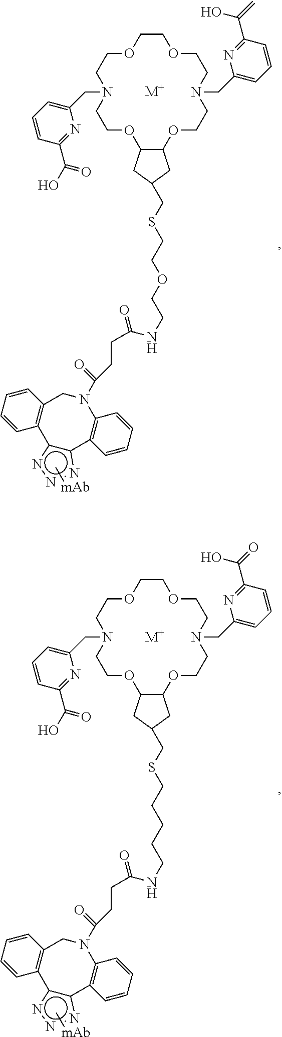

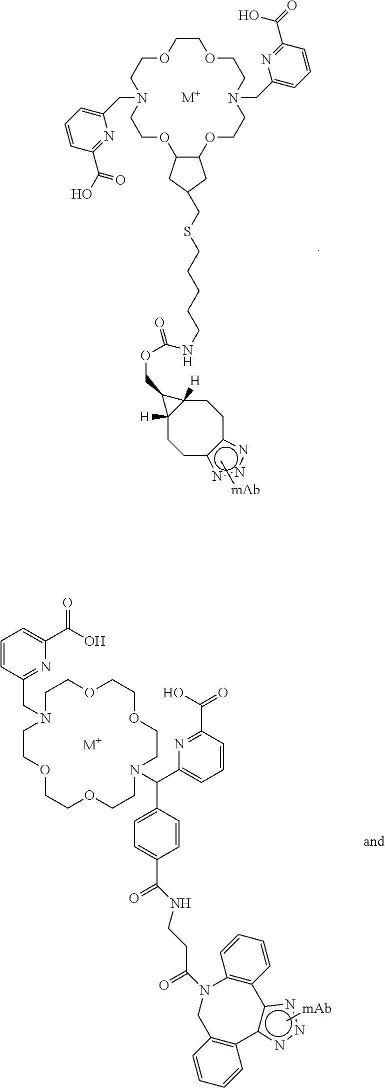

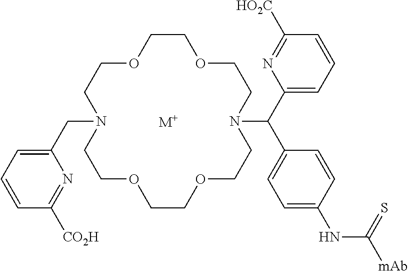

wherein mAb is an antibody or antigen binding fragment thereof.

It is noted that, in radioimmunoconugate structures depicted herein comprising “mAb,” the structures do not show the residue of the mAb (e.g., the lysine residue of the mAb) that is linked to the radiometal complex.

An embodiment of the present invention provides a radioimmunoconjugate having the following structure:

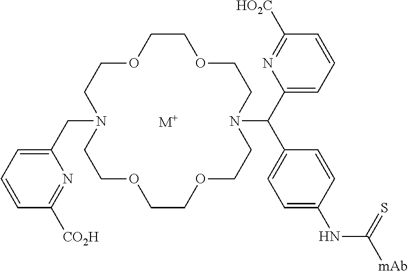

(when mAb is h11B6, the compound is also referred to herein as TOPA-[C7]-phenylthiourea-h11B6 Antibody Conjugate),

wherein M⁺ is actinium-225(²²⁵Ac), and

wherein the mAb has binding specificity for hK2; for example,

(i) wherein the mAb is an h11B6 antibody comprising a heavy chain (HC) variable region comprising the amino acid sequences of SEQ ID NO: 11 and SEQ ID NO: 12 and SEQ ID NO: 13 and a light chain (LC) variable region comprising the amino acid sequences of SEQ ID NO: 14 and SEQ ID NO: 15 and SEQ ID NO: 16; and/or

(ii) wherein the mAb comprises a heavy chain variable region (VI) having at least 80%, at least 85%, at least 90%, at least 95%, or at least 98%, or 100% sequence identity to the amino acid sequence of SEQ ID NO: 17, and/or a light chain variable region (VL) having at least 80%, at least 85%, at least 90%, at least 95%, or at least 98%, or 100% sequence identity to the amino acid sequence of SEQ ID NO: 18.

An embodiment of the present invention provides a radioimmunoconjugate having the following structure:

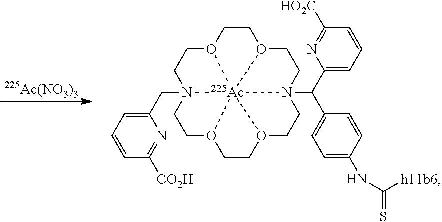 [SMILES: C=C(C)Cc1ccc(C(c2cccc(C)c2)C23CCCCCCCCC(Cc4cccc(C)c4)(CCCCCCCC2)C3)cc1]

(i) wherein the mAb is an h11B6 antibody comprising a heavy chain (HC) variable region comprising the amino acid sequences of SEQ ID NO: 11 and SEQ ID NO: 12 and SEQ ID NO: 13 and a light chain (LC) variable region comprising the amino acid sequences of SEQ ID NO: 14 and SEQ ID NO: 15 and SEQ ID NO: 16; and/or

(ii) wherein the mAb comprises a heavy chain variable region (VH) having at least 80%, at least 85%, at least 90%, at least 95%, or at least 98%, or 100% sequence identity to the amino acid sequence of SEQ ID NO: 17, and/or a light chain variable region (VL) having at least 80%, at least 85%, at least 90% at least 95%, or at least 98%, or 100% sequence identity to the amino acid sequence of SEQ ID NO: 18.

Radioimmunoconjugates produced by the methods described herein can be analyzed using methods known to those skilled in the art in view of the present disclosure. For example, LC/MS analysis can be used to determine the ratio of the compound to the labeled polypeptide, e.g., antibody or antigen binding fragment thereof, analytical size-exclusion chromatography can be used to determine the oligomeric state of the polypeptides and polypeptide conjugates, e.g., antibody and antibody conjugates; radiochemical yield can be determined by instant thin layer chromatography (e.g., iTLC-SG), and radiochemical purity can be determined by size-exclusion HPLC. Exemplary methods are described herein, e.g., in the Examples below.

### Pharmaceutical Compositions and Methods of Use

In another embodiment, the invention is directed to a pharmaceutical composition comprising a compound of the invention, radiometal complex, an immunoconjugate, or radioimmunoconjugate of the invention, and a pharmaceutically acceptable carrier. The pharmaceutical composition may comprise one or more pharmaceutically acceptable excipients.

In one embodiment, a pharmaceutical composition comprises a compound of the invention, and a pharmaceutically acceptable carrier.

In one embodiment, a pharmaceutical composition comprises a radiometal complex of the invention, and a pharmaceutically acceptable carrier.

In another embodiment, a pharmaceutical composition comprises an immunoconjugate of the invention, and a pharmaceutically acceptable carrier.

In another embodiment, a pharmaceutical composition comprises a radioimmunoconjugate of the invention, and a pharmaceutically acceptable carrier.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody-based, or a radiocomplex-based pharmaceutical composition can be used in the invention.

According to particular embodiments, the compositions described herein are formulated to be suitable for the intended route of administration to a subject. For example, the compositions described herein can be formulated to be suitable for parenteral administration, e.g., intravenous, subcutaneous, intramuscular or intratumoral administration.

In certain embodiments, the invention is directed to methods of selectively targeting neoplastic cells for radiotherapy and treating neoplastic diseases or disorders. Any of the radiocomplexes or radioimmunoconjugates, and pharmaceutical compositions thereof described herein can be used in the methods of the invention.

A “neoplasm” is an abnormal mass of tissue that results when cells divide more than they should or do not die when they should. Neoplasms can be benign (not cancer) or malignant (cancer). A neoplasm is also referred to as a tumor. A neoplastic disease or disorder is a disease or disorder associated with a neoplasm, such as cancer. Examples of neoplastic disease or disorders include, but are not limited to, disseminated cancers and solid tumor cancers.

In certain embodiments, the invention is directed to a method of treating prostate cancer (e.g., metastatic prostate cancer, or metastatic castration-resistant prostate cancer) in a subject in need thereof comprises administering to the subject a therapeutically effective amount of an immunoconjugate or radioimmunoconjugate as described herein, wherein the immunoconjugate or radioimmunoconjugate comprises a radiometal complex as described herein conjugated to H11B6.

Embodiments of the present invention are particularly useful in treating patients that have been diagnosed with prostate cancer; for example, patients that have late-stage prostate cancer. According to an embodiment, the cancer is non-localized prostate cancer. According to another embodiment, the cancer is metastatic prostate cancer. According to another embodiment, the cancer is castration-resistant prostate cancer (CRPC). According to another embodiment, the cancer is metastatic castration-resistant prostate cancer (mCRPC). According to another embodiment, the cancer is mCRPC with adenocarcinoma.

Other examples of diseases to be treated or targeted for radiotherapy by the methods of the invention described herein include, but are not limited to, hypertrophy, a coronary disease, or a vascular occlusive disease, a disease or disorder associated with an infected cell, a microbe or a virus, or a disease or disorder associated with an inflammatory cell, such as rheumatoid arthritis (RA).

In an embodiment, the invention is directed to a method of selectively targeting neoplastic cells for radiotherapy comprises administering to a subject in need thereof a radioimmunoconjugate or pharmaceutical composition of the invention to the subject.

In an embodiment the invention is directed to a method of treating a neoplastic disease or disorder comprises administering to a subject in need thereof a radioimmunoconjugate or pharmaceutical composition of the invention to the subject.

In an embodiment the invention is directed to a method of treating cancer in a subject in need thereof comprises administering to the subject in need thereof a radioimmunoconjugate or pharmaceutical composition of the invention to the subject.

Additionally, embodiments of the invention relate to a chelator as described herein (e.g., a compound of formula (I), (II), or (III), or a pharmaceutically acceptable salt thereof), for the manufacture of a medicament for the treatment of any one of the diseases, disorders or medical conditions mentioned herein, such as prostate cancer (e.g., CRPC or mCRPC).

Additionally, embodiments of the invention relate to a radiometal complex as described herein (e.g., a compound of formula (I-M⁺), (II-M⁺), or (III-M⁺), or a pharmaceutically acceptable salt thereof), for the manufacture of a medicament for the treatment of any one of the diseases, disorders or medical conditions mentioned herein, such as prostate cancer (e.g., CRPC or mCRPC).

Additionally, embodiments of the invention relate to a radioimmunoconjugate as described herein conjugated to an antibody (e.g., a radiometal complex of formula (I-M⁺), (II-M⁺), or (III-M⁺), wherein M⁺ is Ac²²⁵, and wherein the radiometal complex is conjugated to h11B6), for the manufacture of a medicament for the treatment of any one of the diseases, disorders or medical conditions mentioned herein, such as prostate cancer (e.g., CRPC or mCRPC).

Additionally, embodiments of the invention relate to a radioimmunoconjugate as described herein (e.g., a radiometal complex of formula (I-M⁺), (II-M⁺), or (III-M⁺), wherein M⁺ is Ac²²⁵, and wherein the radiometal complex is conjugated to h11B6), for use as a medicament for the treatment of any one of the diseases, disorders or medical conditions mentioned herein, such as prostate cancer (e.g., CRPC or mCRPC).

Radioimmunoconjugates carry radiation directly to, for example, cells, etc., targeted by the targeting ligand. Preferably, the radioimmunoconjugates carry alpha-emitting radiometal ions, such as ²²⁵Ac. Upon targeting, alpha particles from the alpha-emitting radiometal ions, e.g., ²²⁵Ac and daughters thereof, are delivered to the targeted cells and cause a cytotoxic effect thereto, thereby selectively targeting neoplastic cells for radiotherapy and/or treating the neoplastic disease or disorder.

The present invention further includes Pre-targeting approaches for selectively targeting neoplastic cells for radiotherapy and for treating a neoplastic disease or disorder. According to a pre-targeting approach, an azide-labeled antibody or antigen binding fragment thereof is dosed, binds to cells bearing the target antigen of the antibody, and is allowed to clear from circulation over time or removed with a clearing agent. Subsequently, a radiometal complex of the invention, preferably a radiometal complex comprising a cyclooctyne or cyclooctyne derivative, e.g., DBCO, is administered and undergoes a SPAAC reaction with azide-labeled antibody bound at the target site, while the remaining unbound radiometal complex clears rapidly from circulation. The pre-targeting technique provides a method of enhancing radiometal ion localization at a target site in a subject.

In other embodiments, a modified polypeptide, e.g., azide-labeled antibody or antigen binding fragment thereof, and a radiometal complex of the invention are administered to a subject in need of targeted radiotherapy or treatment of a neoplastic disease or disorder in the same composition, or in different compositions.

As used herein, the term “therapeutically effective amount” refers to an amount of an active ingredient or component that elicits the desired biological or medicinal response in a subject. A therapeutically effective amount can be determined empirically and in a routine manner, in relation to the stated purpose. For example, in vitro assays can optionally be employed to help identify optimal dosage ranges. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the route of administration, and the severity of disease, and should be decided according to the judgment of the practitioner and each patient's circumstances. Effective doses can be extrapolated from dose-response curves derived from in vitro or animal model test systems.

As used herein, the terms “treat,” “treating,” and “treatment” are all intended to refer to an amelioration or reversal of at least one measurable physical parameter related to a disease, disorder, or condition in which administration of a radiometal ion would be beneficial, such as a neoplastic disease or disorder, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition in which administration of a radiometal ion would be beneficial, such as a neoplastic disease or disorder. In a particular embodiment, “treat,” “treating,” and “treatment” refer to prevention of the recurrence of a neoplastic disease, disorder, or condition. In a particular embodiment, “treat,” “treating,” and “treatment” refer to an increase in the survival of a subject having a neoplastic disease, disorder, or condition. In a particular embodiment, “treat,” “treating,” and “treatment” refer to elimination of a neoplastic disease, disorder, or condition in the subject.

In some embodiments, a therapeutically effective amount of a radioimmunoconjugate or pharmaceutical composition of the invention is administered to a subject to treat a neoplastic disease or disorder in the subject, such as cancer.

In other embodiments of the invention, radioimmunoconjugates and pharmaceutical compositions of the invention can be administered in combination with other agents that are effective for treatment of neoplastic diseases or disorders.

In additional embodiments, the invention is directed to radioimmunoconjugates and pharmaceutical compositions as described herein for use in selectively targeting neoplastic cells for radiotherapy and/or for treating a neoplastic disease or disorder; and use of a radioimmunoconjugate or pharmaceutical compositions as described herein in the manufacture of a medicament for selectively targeting neoplastic cells for radiotherapy and/or for treating a neoplastic disease or disorder.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

### EXAMPLES

The following Examples are set forth to aid in the understanding of the invention and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

Abbreviations used in the specification, particularly the Schemes and Examples, are as listed in the Table A, below:

TABLE A
Abbreviations

| | |
|---|---|
| ADP = | Adenosine Diphosphate |
| Alexa633 tracer = | Alexa Fluor ® 633 Hydrazide Tracer |
| | (ThermoFisher) |
| BSA = | Bovine Serum Albumin |
| ACN or MeCN = | Acetonitrile |
| ATP = | Adenosine Triphosphate |
| BINAP = | (2,2′-Bis(diphenylphosphino)-1,1′-binaphthyl) |
| Brij ™-35 = | Polyethylene glycol hexadecyl ether |
| DBCO = | Dibenzocyclooctyl |
| DCM = | Dichloromethane |
| DIPEA or DIEA = | Diisopropylethylamine |
| DMF = | N,N-Dimethylformamide |
| DMSO = | Dimethylsulfoxide |
| DPPF or dppf = | 1,1′-Bis(diphenylphosphino)ferrocene |
| DTPA = | Diethylene triamine pentaacetic acid |
| DTT = | Dithiothrietol |
| EDTA = | Ethylenediaminetetracetic acid |
| eGFR = | Estimated Glomular Filtration Rate |
| EtOH = | Ethanol |
| F12 medium = | Gibco ® F12 Nutrient Medium (ThermoFisher) |
| FBS = | Fetal Bovine Serum |
| G418 = | Geneticin ® (G418) Sulfate |
| GFR = | Glomular Filtration Rate |
| GLP-1 = | Glucagon-like peptide 1 |
| GRK2 = | G protein-coupled Receptor Kinase 2 |
| HATU = | (1-[Bis(dimethylamino)methylene-1H-1,2,3- |
| | triazolo[4,5-b]pyridinium 3-oxid |
| | hexafluorophosphate |
| HBSS = | GIBCO ® Hank's Balanced Salt Solution |
| HEPES = | 4-(2-Hydroxyethyl)-1-Piperizine Ethane Sulfonic |
| | Acid |
| HPLC = | High Pressure Liquid Chromatography |
| HTRF = | Homogeneous Time Resolved Fluorescence |
| IFG = | Impaired fasting glucose |
| IGT = | Impaired glucose tolerance |
| LCMS or LC/MS = | Liquid chromatography-mass spectrometry |
| LDA = | Lithium diisopropylamide |
| LiHMDS = | Lithium bis(trimethylsilyl)amide |
| MeOH = | Methanol |
| mesyl or Ms = | Methylsulfonyl (i.e. —SO₂—CH₃) |
| mesylate or OMs = | Methanesulfonate (i.e. —O—SO₂—CH₃) |
| MOM = | Methoxy methyl |
| Ms or mesyl = | —SO₂—CH₃ |
| MsCl = | Mesyl Chloride (i.e. CH₃—SO₂—Cl) |
| NaBH(OAc)₃ = | Sodium triacetoxyborohydride |
| NAFLD = | Non-alcoholic fatty liver disease |
| Na₂SO₄ = | Sodium Sulfate |
| NASH = | Non-alcoholic steatohepatitis |
| NBS = | N-Bromosuccinimide |
| NH(PMB)₃ = | tris(4-methoxybenzyl)-λ⁴-azane |
| NMO = | 4-Methylmorpholine N-oxide |
| NMP = | N-Methyl-2-pyrrolidone |
| NMR = | Nuclear Magnetic Resonance |
| OMs or mesylate = | Methanesulfonate (i.e. —O—SO₂—CH₃) |
| OTf or triflate = | Trifluoromethanesulfonate |
| OTs or tosylate = | p-Toluenesulfonate |
| Pd/C = | Palladium on carbon |
| Pd(dba)₂ = | Bis(dibenzylideneacetone)dipalladium(0) |
| Pd(dppf)Cl₂ = | [1,1′-Bis(diphenylphosphino)ferrocene] Palladium |
| | (II) Dichloride |
| Pd(dppf)Cl₂•CHCl₃ = | [1,1′-Bis(diphenylphosphino)ferrocene] |
| | chloropalladium complex with chloroform |
| (1-nap)₃P or | Tri(1-naphthyl)phosphine |
| P(1-nap)₃ = | |
| Pd(OAc)₂ = | Palladium (II) acetate |
| Pd(OH)₂ = | Palladium hydroxide |
| Pd(OH)₂/C = | Palladium hydroxide on carbon (Pearlman's |
| | Catalyst) |
| Pd(PPh₃)₄ = | Tetrakis(triphenylphosphine) palladium (0) |
| PEG = | Polyethylene Glycol |
| PMB = | 4-Methoxybenzyl ether |
| PPh₃ = | Triphenylphosphine |
| SNS = | Sympathetic Nervous System |
| TBAB = | Tetra-n-butylammonium bromide |
| TBAF = | Tetra-n-butylammonium fluoride |
| TBAI = | Tetra-n-butylammonium iodide |
| TBSOTf = | Tert-butyldimethylsilyl trifluoromethanesulfonate |
| TEA = | Triethylamine |
| Tf or trifyl = | Trifluoromethylsulfonyl (i.e. —SO₂—CF₃) |
| TFA = | Trifluoroacetic Acid |
| TEIF = | Tetrahydrofuran |
| THP = | 2-Tetrahydropyranyl |
| TLC = | Thin Layer Chromatography |
| TMS = | Trimethylsilyl |
| TMSN₃ = | Trimethylsilyl azide |
| Tosylate or OTs = | p-Toluenesulfonate |
| TOPA = | 6,6′-((1,4,10,13-tetraoxa-7,16- |
| | diazacyclooctadecane-7,16-diyl)bis |
| | (methylene))dipicolinic acid = H2bp18c6 |
| Ts or tosyl = | -p-Toluenesulfonyl chloride |
| p-TsCl = | p-Toluenesulfonyl chloride |
| Tween-20 ® = | Nonionic detergent (Sigma Aldrich) |

As used herein, unless otherwise noted, the term “isolated form” shall mean that the compound is present in a form which is separate from any solid mixture with another compound(s), solvent system or biological environment. In an embodiment of the present invention, any of the compounds as herein described are present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s).

### Example 1

### 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (TOPA-[C-7]-Phenyl-carboxylic acid)

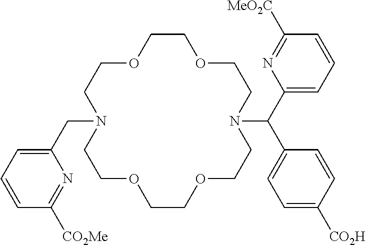 [SMILES: COC(=O)c1cccc(CN2CCOCCOCCN(C(c3ccc(C(=O)O)cc3)c3cccc(C(=O)OC)n3)CCOCCOCC2)n1]

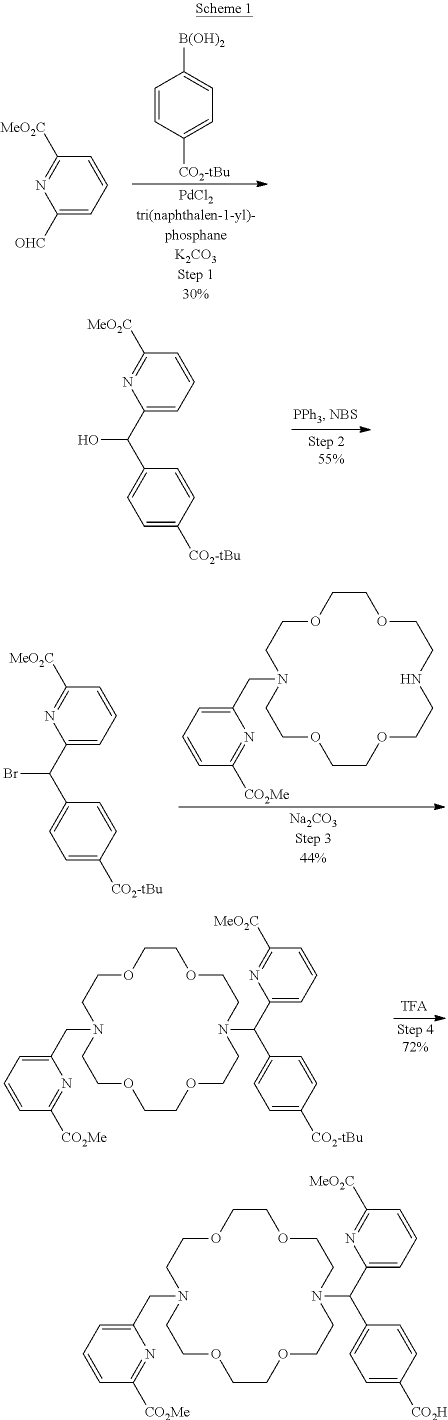

Step 1: To a mixture of methyl 6-formylpicolinate (4.00 g, 24.2 mmol), (4-(tert-butoxycarbonyl)phenyl)boronic acid (10.7 g, 48.5 mmol), PdCl2 (0.21 g, 1.2 mmol), tri(naphthalen-1-yl)phosphine (0.50 g, 1.2 mmol) and potassium carbonate (10.0 g, 72.7 mmol) under nitrogen at −78° C. in a 500 mL three neck round bottom flask was added tetrahydrofuran (100 mL) in one portion. The mixture was purged with nitrogen and stirred at room temperature for 30 min, then heated at 65° C. for 24 h. The reaction mixture was cooled room temperature and filtered through a pad of Celite and the filtrate was concentrated to dryness. The crude product was purified by silica gel chromatography (0-50% EtOAc/petroleum ether) to afford methyl 6-((4-(tert-butoxycarbonyl)phenyl)(hydroxy)methyl)picolinate as a yellow oil (2.5 g, 30% yield).

Step 2: A stir bar, methyl 6-((4-(tert-butoxycarbonyl)phenyl)(hydroxy)methyl)picolinate (2.50 g, 7.30 mmol), PPh₃ (3.43 g, 13.1 mmol), N-bromosuccinimide (2.13 g, 12.0 mmol) and dichloromethane (30 mL) were added to a 250 mL three neck round bottom flask under nitrogen atmosphere at room temperature and stirred for 1 h. The reaction solution was loaded onto a silica gel column and chromatography (0-30% EtOAc/petroleum ether) gave compound methyl 6-(bromo(4-(tert-butoxycarbonyl)phenyl)methyl)picolinate (1.65 g, 56% yield) as a yellow oil.

Step 3: A stir bar, methyl 6-(bromo(4-(tert-butoxycarbonyl)phenyl)methyl)picolinate (1.52 g, 3.69 mmol), methyl 6-((1,4,10,13-tetraoxa-7,16-diazacyclooctadecan-7-yl)methyl)picolinate (1.50 g, 3.69 mmol), Na₂CO₃ (1.17 g, 11.1 mmol), and acetonitrile (30 mL) were added to a 250 mL three neck round-bottomed flask, and the resultant heterogeneous mixture was heated at 90° C. for 16 h under nitrogen atmosphere. Subsequently reaction mixture was cooled to room temperature, filtered through a pad of Celite, and concentrated to dryness in vacuo to give the crude product. The crude product was purified by silica gel chromatography (0-10% MeOH/dichloromethane) to afford methyl 6-((4-(tert-butoxycarbonyl)phenyl)(16-((6-(methoxycarbonyl)pyridin-2-yl)methyl)-1,4,10,13-tetraoxa-7,16-diazacyclooctadecan-7-yl)methyl)picolinate as a brown oil (1.2 g, 44%).

Step 4: A stir bar, methyl 6-((4-(tert-butoxycarbonyl)phenyl)(16-((6-(methoxycarbonyl)pyridin-2-yl)methyl)-1,4,10,13-tetraoxa-7,16-diazacyclooctadecan-7-yl)methyl)picolinate (1.2 g, 1.6 mmol), TFA (0.62 mL, 8.1 mmol) and DCM (20 mL) were added to a 100 mL three neck round bottom flask at r.t. and stirred for 1 h. Reaction mixture was concentrated to dryness and the resultant crude product was subjected to preparative HPLC (Column: XBRIDGE C18 (19×150 mm) 5.0 μm; Mobile phase: 0.1% TFA in water/ACN; Flow Rate: 15.0 mL/min) to give TOPA-[C-7]-Phenyl-carboxylic acid (0.8 g, 72%) as brown oil. LC-MS APCI: Calculated for C₃₅H₄₄N₄O₁₀ 680.31; Observed m/z [M+H]⁺ 681.5. Purity by LC-MS: 99.87%. Purity by HPLC: 97.14% (97.01% at 210 nm, 97.20% at 254 nm and 97.21% at 280 nm; Column: Atlantis dC18 (250×4.6 mm), 5 μm; Mobile phase A: 0.1% TFA in water, Mobile phase B: acetonitrile; Flow rate: 1.0 mL/min.%. ¹H NMR (400 MHz, DMSO-d6): δ 8.12-8.07 (m, 4H), 8.00-7.98 (m, 2H), 7.75-7.73 (m, 4H), 6.10 (s, 1H), 4.67 (s, 2H), 3.96 (s, 3H), 3.91 (s, 3H), 3.82 (s, 8H), 3.56 (s, 8H), 3.52 (s, 8H).

### Example 2

### 6-((16-((6-Carboxypyridin-2-yl)(4-((2-(2-(2-isothiocyanatoethoxy)ethoxy)ethyl)carbamoyl)phenyl)methyl)-1,4,10,13-tetraoxa-7,16-diazacyclooctadecan-7-yl)methyl)picolinic acid

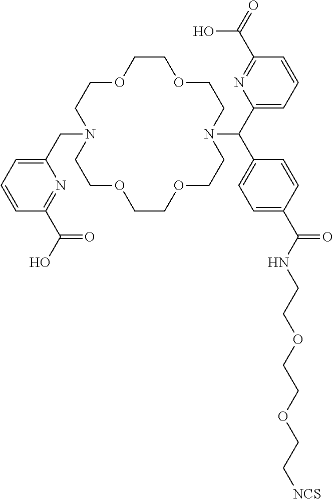

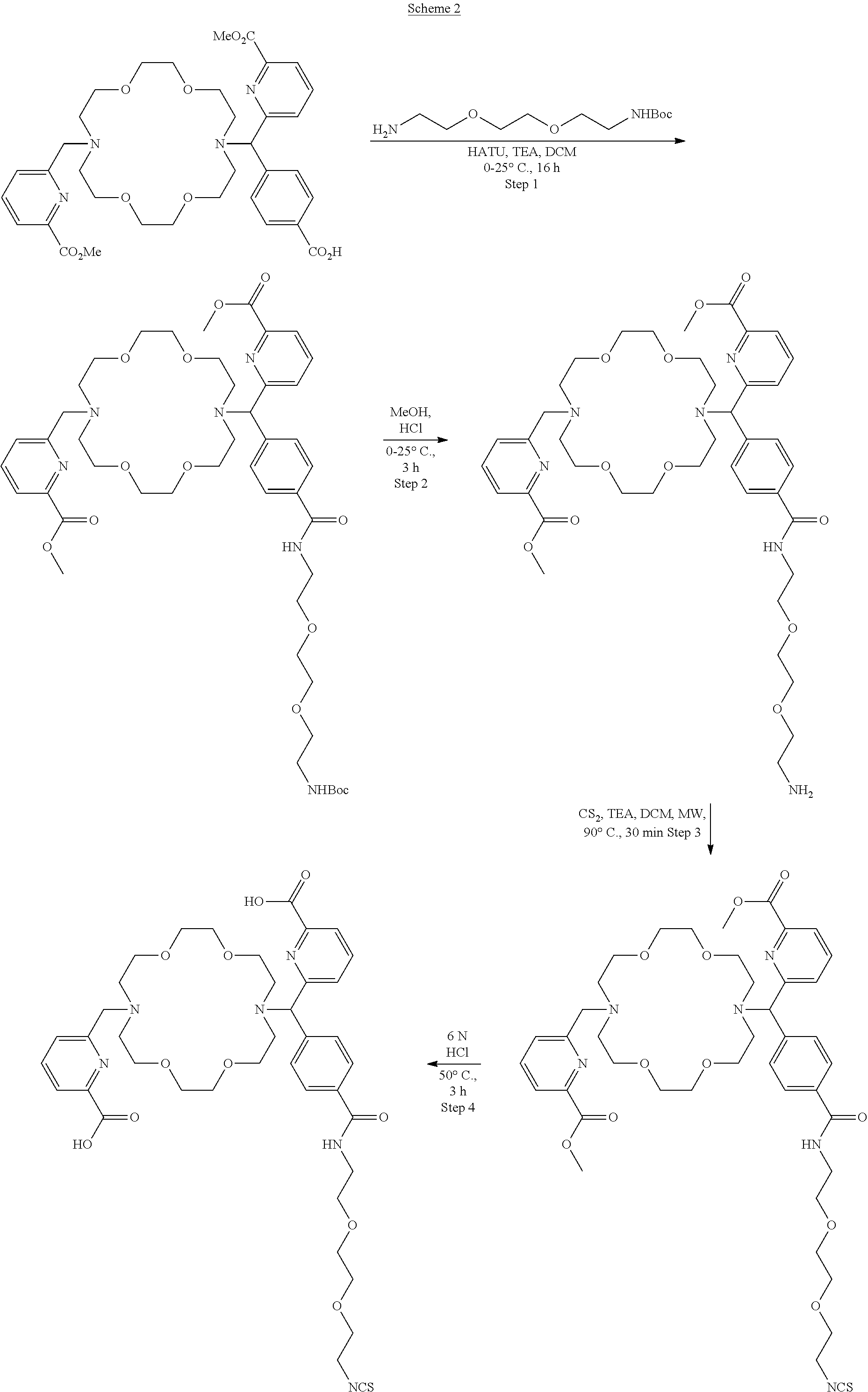

Step 1: A stir bar, 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.40 g, 0.60 mmol), tert-butyl (2-(2-(2-aminoethoxy)ethoxy)ethyl)carbamate (0.15 g, 0.60 mmol), triethylamine (0.18 g, 0.76 mmol), HATU (0.33 g, 0.90 mmol), and DCM (4.0 mL) were added to a 25 mL three neck round-bottomed flask at 0° C. under nitrogen atmosphere. The mixture was stirred overnight at room temperature. The reaction was treated with water (10 mL) and extracted with dichloromethane (10 mL×3). The combined extracts were washed with 10% aqueous NaHCO₃(10 mL), brine (10 mL), dried over anhydrous Na₂SO₄, filtered, and concentrated to dryness to yield an oil, which was purified by silica gel chromatography (0-10% MeOH/DCM) to yield methyl 6-((4-((2,2-dimethyl-4-oxo-3,8,11-trioxa-5-azatridecan-13-yl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.18 g).

Step 2: A stir bar, methyl 6-((4-((2,2-dimethyl-4-oxo-3,8,11-trioxa-5-azatridecan-13-yl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.18 g, 0.20 mmol), MeOH (1.8 mL), and HCl in methanol (4 M, 1.0 mL, 4.0 mmol) were added to a 10 mL single-neck round-bottomed flask at 0° C., then warmed to room temperature and stirred for 2 h. The volatiles were removed in vacuo to yield methyl 6-((4-((2-(2-(2-aminoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.15 g), which was used without purification.

Step 3: A stir bar, methyl 6-((4-((2-(2-(2-aminoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.10 g, 0.12 mmol), triethylamine (37 mg, 0.37 mmol), dry DCM (2 mL), and carbon disulfide (14 mg, 0.18 mmol) were added to a pressure vial at room temperature under a nitrogen atmosphere. The vial was subjected to microwave-irradiation (150 W power) at 90° C. for 30 min. The vial was then cooled to room temperature, the reaction mixture diluted with dichloromethane (10 mL), and then washed successively with water (5 mL), 1 M HCl (5 mL), and water (5 mL), dried over anhydrous Na₂SO₄, filtered, and concentrated to dryness to yield methyl 6-((4-((2-(2-(2-isothiocyanatoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (100 mg), which was used without purification.

Step 4: A stir bar, methyl 6-((4-((2-(2-(2-isothiocyanatoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.10 g, 0.12 mmol), and aqueous HCl (6 N, 0.4 mL, 2.34 mmol) were added to a 10 mL single-neck round-bottomed flask, and stirred at 50° C. for 3 h. The reaction mixture was cooled to room temperature, concentrated to dryness in vacuo to yield an oil, which was purified by preparative HPLC (Column: XBRIDGE C18 19×150 mm, 5.0 μm; Mobile phase: 0.1% TFA in water/acetonitrile; Flow Rate: 15.0 mL/min) to yield 6-((16-((6-carboxypyridin-2-yl)(4-((2-(2-(2-isothiocyanatoethoxy)ethoxy)ethyl)carbamoyl)phenyl)methyl)-1,4,10,13-tetraoxa-7,16-diazacyclooctadecan-7-yl)methyl)picolinic acid (5.0 mg). LC-MS APCI: Calculated for C₄₀H₅₂N₆O₁₁S: 824.34; Observed m/z [M+H]⁺ 824.8. ¹H NMR (400 MHz, CD₃OD): δ 8.22-8.20 (m, 2H), 8.14-8.05 (m, 2H), 7.94 (d, J=8.00 Hz, 2H), 7.79 (d, J=8.00 Hz, 2H), 7.73-7.67 (m, 2H), 6.16 (s, 1H), 4.77 (s, 2H), 3.93-4.00 (m, 8H), 3.59-3.70 (m, 27H), 3.47-3.44 (m, 2H).

### Example 3

### 6-((4-((6-Aminohexyl)carbamoyl)phenyl)(16-((6-carboxypyridin-2-yl)methyl)-1,4,10,13-tetraoxa-7,16-diazacyclooctadecan-7-yl)methyl)picolinic acid and

### Example 4

### 6-((16-((6-Carboxypyridin-2-yl)(4-((6-isothiocyanatohexyl)carbamoyl)phenyl)methyl)-1,4,10,13-tetraoxa-7,16-diazacyclooctadecan-7-yl)methyl)picolinic acid

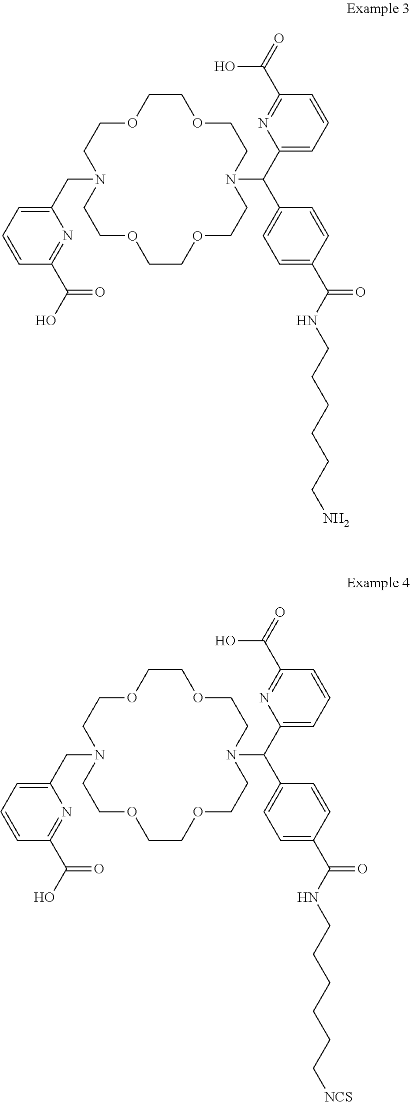

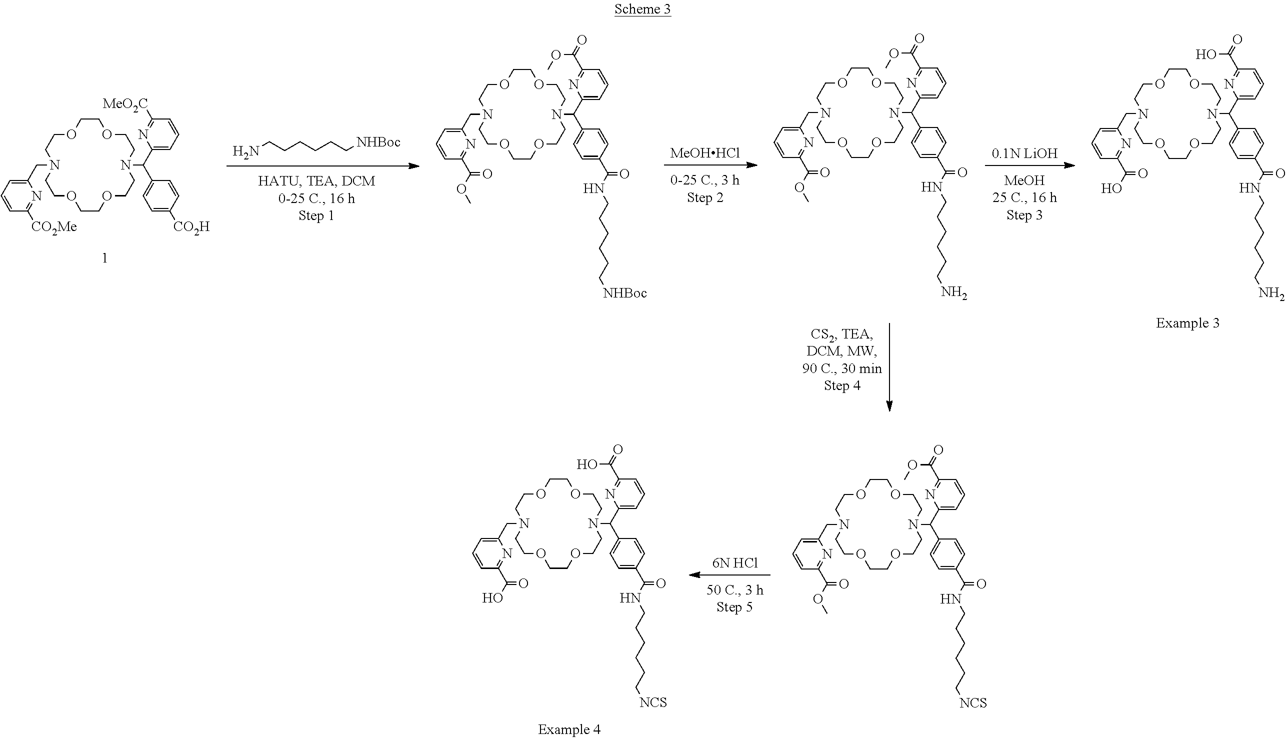

Step 1: A stir bar, 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.12 g, 0.18 mmol), tert-butyl (6-aminohexyl)carbamate (38 mg, 0.18 mmol), triethylamine (54 mg, 0.54 mmol), HATU (0.10 g, 0.27 mmol), and DCM (4.0 mL) were added to a 25 mL three-neck round-bottomed flask at 0° C. under a nitrogen atmosphere. The reaction mixture was then brought to room temperature and stirred overnight. The reaction mixture was then treated with water (10 mL) and extracted with dichloromethane (10 mL×3). The combined extracts were washed with 10% aqueous NaHCO₃(10 mL) and brine (10 mL), dried over anhydrous Na₂SO₄, filtered, and concentrated to dryness to yield an oil. The oil was purified via silica gel chromatography (0-10% MeOH/DCM) to yield methyl 6-((4-((6-((tert-butoxycarbonyl)amino)hexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (70 mg) as a gummy oil.

Step 2: A stir bar, methyl 6-((4-((6-((tert-butoxycarbonyl)amino)hexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (70 mg, 0.080 mmol), MeOH (1.5 mL), and HCl in methanol (4 M, 0.4 mL, 1.6 mmol) were added to a 25 mL round-bottomed flask at 0° C., which was subsequently brought to room temperature and stirred for 2 h. The volatiles were removed in vacuo to yield methyl 6-((4-((6-aminohexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (30 mg), which was used without purification.

Step 3: A stir bar, methyl 6-((4-((6-aminohexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (30 mg, 0.038 mmol), aqueous LiOH (1.1 mL, 0.1 N, 0.11 mmol), and MeOH (1.0 mL) were added to an 8 mL reaction vial and stirred overnight at room temperature. The reaction mixture was then treated with acetic acid until pH˜6.5, and subsequently concentrated to dryness in vacuo at room temperature. The resultant product was subjected to preparative HPLC (Column: XBRIDGE C18 19×150 mm, 5.0 μm; Mobile phase: 10 mM ammonium acetate in water/ACN; Flow Rate: 15.0 mL/min) to yield Example 3: 6-((4-((6-aminohexyl)carbamoyl)phenyl)(16-((6-carboxypyridin-2-yl)methyl)-1,4,10,13-tetraoxa-7,16-diazacyclooctadecan-7-yl)methyl)picolinic acid (10 mg). LC-MS APCI: Calculated for C₃₉H₅₄N₆O₉; 750.40; Observed m/z [M+H]⁺ 751.3. ¹H NMR (400 MHz, CD₃OD): δ 8.22 (d, J=1.60 Hz, 2H), 8.21-8.06 (m, 2H), 7.92 (d, J=8.40 Hz, 2H), 7.80 (d, J=8.40 Hz, 2H), 7.75-7.69 (m, 2H), 6.20 (s, 1H), 4.70 (s, 2H), 4.02-3.92 (m, 8H), 3.76-3.62 (m, 14H), 3.51-3.32 (m, 4H), 2.93 (t, J=8.00 Hz, 2H), 1.67-1.64 (m, 4H), 1.46-1.45 (m, 4H).

Step 4: A stir bar, methyl 6-((4-((6-aminohexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.10 g, 0.13 mmol), triethylamine (39 mg, 0.38 mmol), dry DCM (2 mL), and carbon disulfide (15 mg, 0.19 mmol) were added to a pressure vial at room temperature under a nitrogen atmosphere. The vial was subjected to microwave irradiation (150 W power) at 90° C. for 30 min. The vial was then cooled to room temperature and the reaction mixture diluted with dichloromethane (10 mL), washed with water (5 mL), 1 M HCl (5 mL), and water (5 mL), dried over anhydrous Na₂SO₄, filtered, and concentrated to dryness to yield methyl 6-((4-((6-isothiocyanatohexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.1 g), which was used without purification.

Step 5: A stir bar, methyl 6-((4-((6-isothiocyanatohexyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.10 g, 0.12 mmol), and aqueous HCl (6 N, 0.4 mL, 2.4 mmol) were added to a 10 mL round-bottomed flask, and then stirred at 50° C. for 3 h. The reaction mixture was then cooled to room temperature and concentrated to dryness in vacuo to yield a residue, which was purified by preparative HPLC (Column: XBRIDGE C18 19×150 mm, 3.5 μm; Mobile phase: 0.1% TFA in water/acetonitrile; Flow Rate: 2.0 mL/min) to yield Example 4: 6-((16-((6-carboxypyridin-2-yl)(4-((6-isothiocyanatohexyl)carbamoyl)phenyl)methyl)-1,4,10,13-tetraoxa-7,16-diazacyclooctadecan-7-yl)methyl)picolinic acid (15 mg). LC-MS APCI: Calculated for C₄₀H₅₂N₆O₉S: 792.35; Observed m/z [M+H]⁺ 792.8. ¹H NMR (400 MHz, CD₃OD): δ 8.23-8.20 (m, 2H), 8.15-8.06 (m, 2H), 7.92 (d, J=8.40 Hz, 2H), 7.79 (d, J=8.40 Hz, 2H), 7.74-7.68 (m, 2H), 6.17 (s, 1H), 4.77 (s, 2H), 4.01-3.93 (m, 8H), 3.75-3.56 (m, 16H), 3.42-3.33 (m, 5H), 1.74-1.64 (m, 4H), 1.50-1.44 (m, 4H).

### Example 5

### 6-((16-((6-Carboxypyridin-2-yl)(4-((4-isothiocyanatophenethyl)carbamoyl)phenyl)methyl)-1,4,10,13-tetraoxa-7,16-diazacyclooctadecan-7-yl)methyl)picolinic acid

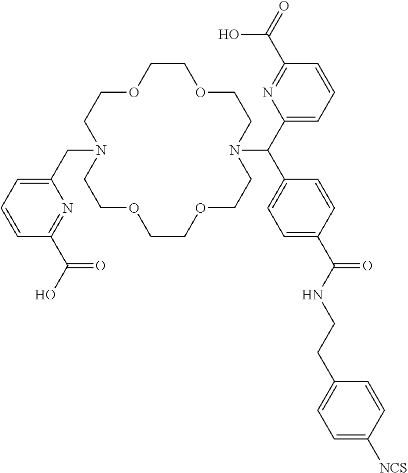

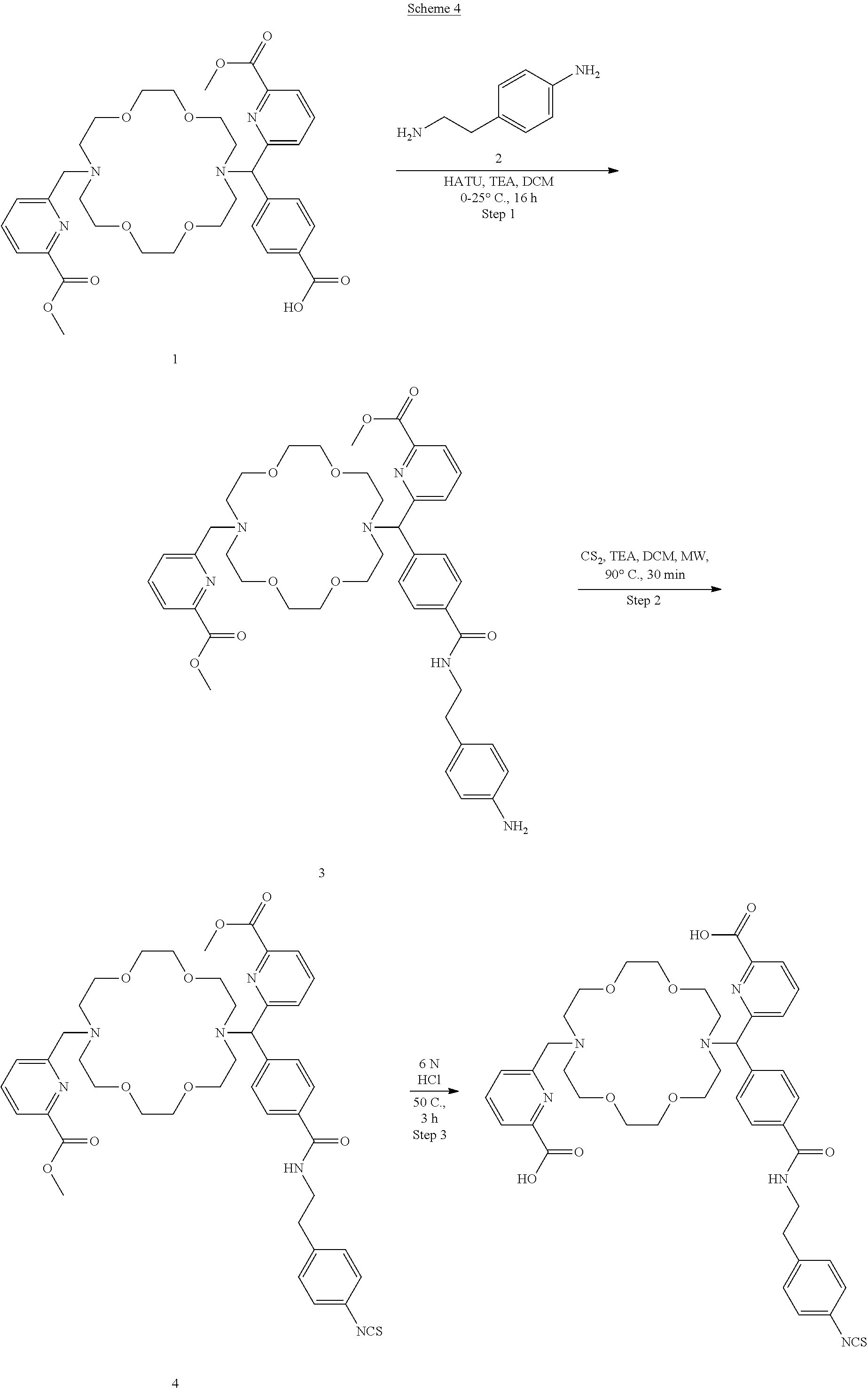

Step 1: A stir bar, 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.25 g, 0.37 mmol), 4-(2-aminoethyl)aniline (60 mg, 0.37 mmol), TEA (0.11 g, 0.15 mL, 1.1 mmol), HATU (0.21 g, 0.55 mmol), and DCM (5 mL) were added to a 25 mL three neck round-bottomed flask at 0° C. under a nitrogen atmosphere. The reaction mixture was stirred overnight at room temperature, and then treated with water (10 mL), and extracted with dichloromethane (10 mL×3). The combined extracts were washed with 10% aqueous NaHCO₃(10 mL) and brine (10 mL), dried over anhydrous Na₂SO₄, filtered, and concentrated to dryness to yield a product which was purified by silica gel chromatography (0-10% MeOH/DCM) to yield methyl 6-((4-((4-aminophenethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.12 g).

Step 2: A stir bar, methyl 6-((4-((4-aminophenethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.12 g, 0.15 mmol), TEA (45 mg, 65 μL, 0.45 mmol), DCM (3 mL), and CS₂ (17 mg, 0.23 mmol) were added to a 10 mL microwave pressure vial at room temperature under a nitrogen atmosphere. The reaction mixture was subjected to microwave-irradiation (150 W power) at 90° C. for 30 min. The reaction mixture was then cooled to room temperature, diluted with dichloromethane (10 mL), washed successively with water (5 mL), 1 M HCl (5 mL), and water (5 mL), dried over anhydrous Na₂SO₄, and concentrated to dryness to yield methyl 6-((4-((4-isothiocyanatophenethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.12 g), which was used without purification.

Step 3: A stir bar, methyl 6-((4-((4-isothiocyanatophenethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.12 g, 0.14 mmol), and aqueous HCl (0.50 mL, 6 N, 2.8 mmol) were added to a 10 mL single-neck round-bottomed flask and stirred at 50° C. for 3 h. The reaction mixture was cooled to room temperature, concentrated to dryness in vacuo, and the crude product was subjected to preparative HPLC (Column: XBRIDGE C18 19×150 mm, 5.0 μm; Mobile phase: 0.1% TFA in water/acetonitrile; Flow Rate: 15.0 mL/min) to yield 6-((16-((6-carboxypyridin-2-yl)(4-((4-isothiocyanatophenethyl)carbamoyl)phenyl)methyl)-1,4,10,13-tetraoxa-7,16-diazacyclooctadecan-7-yl)methyl)picolinic acid (30 mg). LC-MS APCI: Calculated for C₄₂H₄₈N₅O₁₀S; 812.32; Observed m/z [M+H]⁺ 812.9. ¹H NMR (400 MHz, CD₃OD): δ 8.22 (d, J=0.80 Hz, 2H), 8.06-8.21 (m, 2H), 7.85 (d, J=8.40 Hz, 2H), 7.68-7.78 (m, 4H), 7.31 (d, J=8.40 Hz, 2H), 7.21 (d, J=2.00 Hz, 2H), 6.18 (s, 1H), 4.77 (s, 2H), 3.70-4.00 (m, 7H), 3.60-3.67 (m, 16H), 3.44-3.49 (m, 2H), 2.90-3.10 (m, 3H).

### Example 6

### (S)-6,6′-((2-(((2-Isothiocyanatoethyl)thio)methyl)-1,4,10,13-tetraoxa-7,16-diazacyclooctadecane-7,16-diyl)bis(methylene))dipicolinic acid

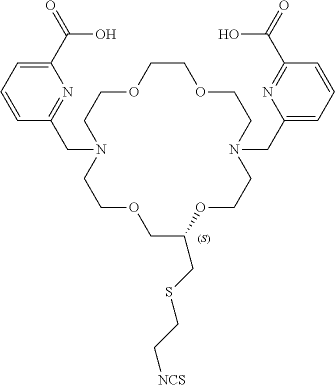

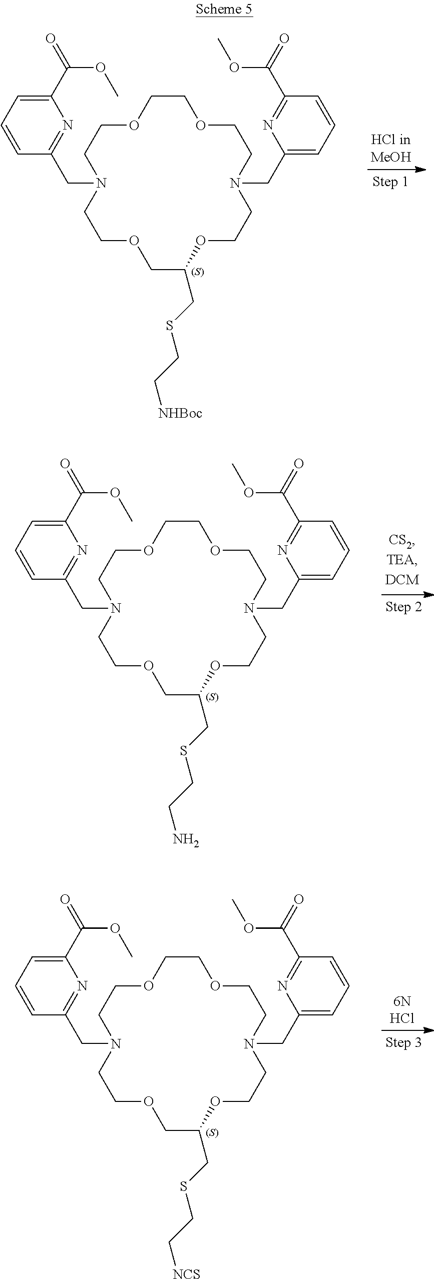

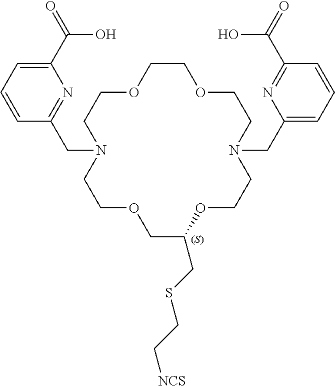

Step 1: A stir bar, 1 (methyl 6-((4-((tert-butoxycarbonyl)amino)phenyl)(16-((6-(methoxycarbonyl)pyridin-2-yl)methyl)-1,4,10,13-tetraoxa-7,16-diazacyclooctadecan-7-yl)methyl)picolinate) (0.10 g, 0.15 mmol), MeOH (0.5 mL) and HCl in methanol (4 M, 0.6 mL, 4.0 mmol) were added to a 25 mL single-neck round-bottomed flask at 0° C. and then brought to room temperature and stirred for 2 h. The volatiles were removed in vacuo to yield dimethyl 6,6′-((2-(((2-aminoethyl)thio)methyl)-1,4,10,13-tetraoxa-7,16-diazacyclooctadecane-7,16-diyl)bis(methylene))(S)-dipicolinate (55 mg), which was used in the next step without purification.

Step 2: A stir bar, dimethyl 6,6′-((2-(((2-aminoethyl)thio)methyl)-1,4,10,13-tetraoxa-7,16-diazacyclooctadecane-7,16-diyl)bis(methylene))(S)-dipicolinate (50 mg, 0.10 mmol), triethylamine (24 mg, 0.24 mmol), DCM (2 mL) and carbon disulfide (12 mg, 0.16 mmol) were added to a microwave vial at room temperature under a nitrogen atmosphere. The vial was subjected to microwave-irradiation (150 W power) at 90° C. for 30 min. The vial was then cooled to room temperature and the reaction mixture diluted with dichloromethane (10 mL), washed successively with water (5 mL), 1M HCl (5 mL), water (5 mL), dried over anhydrous Na₂SO₄, concentrated to dryness and was subjected to silica gel chromatography (0-10% MeOH/DCM) to yield dimethyl 6,6′-((2-(((2-isothiocyanatoethyl)thio)methyl)-1,4,10,13-tetraoxa-7,16-diazacyclooctadecane-7,16-diyl)bis(methylene))(S)-dipicolinate as a yellow solid (20 mg).

Step 3: A stir bar, dimethyl 6,6′-((2-(((2-isothiocyanatoethyl)thio)methyl)-1,4,10,13-tetraoxa-7,16-diazacyclooctadecane-7,16-diyl)bis(methylene))(S)-dipicolinate (20 mg, 0.030 mmol) and aqueous HCl (6 N, 0.1 mL, 0.6 mmol) were added to a 10 mL single-neck round-bottomed flask and stirred at room temperature overnight. The reaction mixture was concentrated to dryness in vacuo, and the resultant residue was subjected to preparative HPLC (Column: XBRIDGE C18 (19×150 mm) 5.0 μm; Mobile phase: 0.1% TFA in water/acetonitrile; Flow Rate: 15.0 mL/min) to yield (S)-6,6′-((2-(((2-isothiocyanatoethyl)thio)methyl)-1,4,10,13-tetraoxa-7,16-diazacyclooctadecane-7,16-diyl)bis(methylene))dipicolinic acid (6 mg). LC-MS APCI: Calculated for C₃₀H₄₁N₅O₈S₂: 663.24; Observed m/z [M+H]⁺ 664.2. ¹H NMR (400 MHz, DMSO-d₆): δ 9.78 (s, 1H), 8.10 (s, 4H), 7.78 (d, J=6.00 Hz, 2H), 4.69 (s, 4H), 3.96-3.52 (m, 23H), 2.85 (t, J=6.40 Hz, 2H), 2.70 (t, J=8.00 Hz, 2H).

### Example 7

### (S)-6,6′-((2-(((5-Isothiocyanatopentyl)thio)methyl)-1,4,10,13-tetraoxa-7,16-diazacyclooctadecane-7,16-diyl)bis(methylene))dipicolinic acid

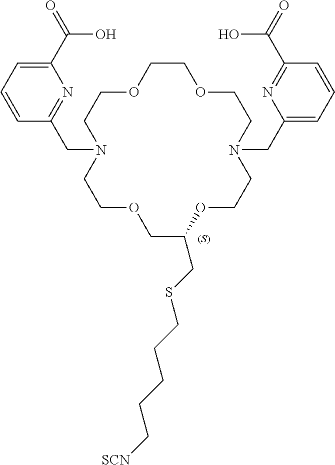

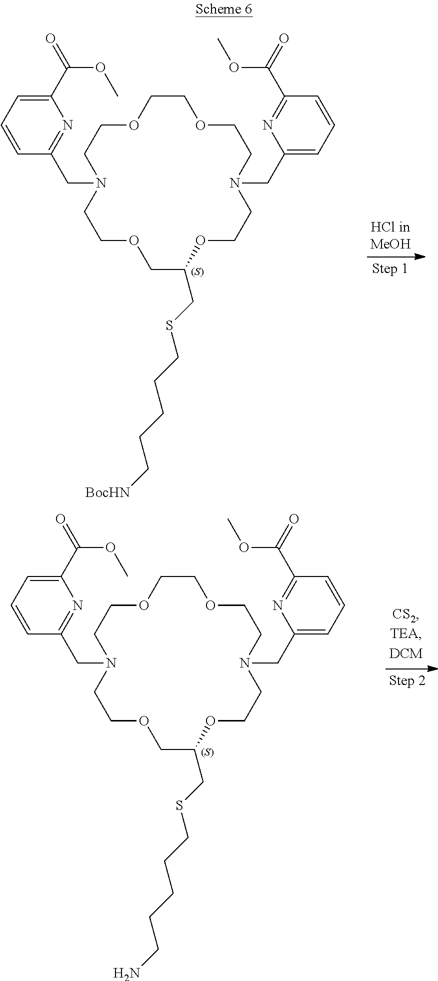

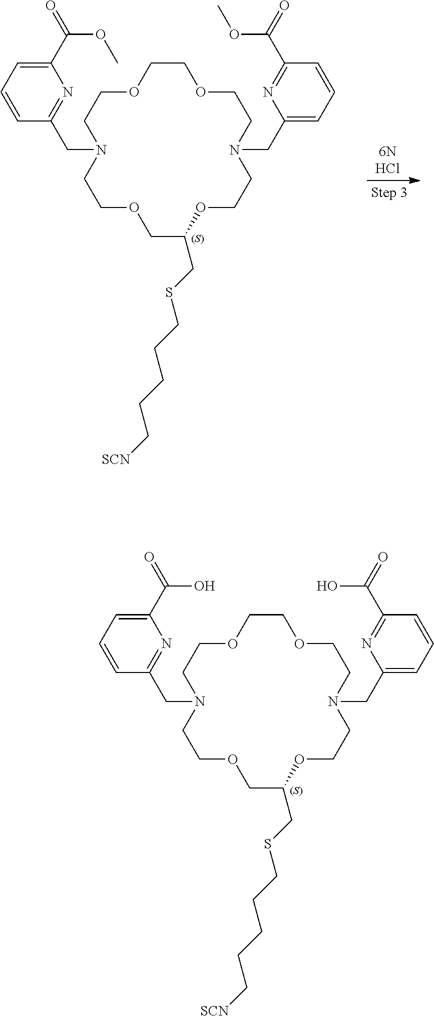

Step 1: A stir bar, dimethyl 6,6′-((2-(((5-((tert-butoxycarbonyl)amino)pentyl)thio)methyl)-1,4,10,13-tetraoxa-7,16-diazacyclooctadecane-7,16-diyl)bis(methylene))(S)-dipicolinate (0.12 g, 0.15 mmol), MeOH (0.5 mL), and HCl in methanol (4 M, 0.6 mL, 4.0 mmol) were added to a 25 mL single-neck round-bottomed flask at 0° C. and brought to room temperature and stirred for 2 h. The volatiles were then removed in vacuo to yield dimethyl 6,6′-((2-(((5-aminopentyl)thio)methyl)-1,4,10,13-tetraoxa-7,16-diazacyclooctadecane-7,16-diyl)bis(methylene))(S)-dipicolinate (70 mg), which was used without purification.

Step 2: A stir bar, dimethyl 6,6′-((2-(((5-aminopentyl)thio)methyl)-1,4,10,13-tetraoxa-7,16-diazacyclooctadecane-7,16-diyl)bis(methylene))(S)-dipicolinate (70 mg, 0.10 mmol), triethylamine (20 mg, 0.20 mmol) dry DCM (2 mL) and carbon disulfide (15 mg, 0.20 mmol) were added to a microwave vial at room temperature under a nitrogen atmosphere. The reaction mixture was subjected to microwave-irradiation (150 W power) at 90° C. for 30 min. The vial was brought to room temperature and the reaction mixture was diluted with dichloromethane (10 mL), washed successively with water (5 mL), 1M HCl (5 mL), and water (5 mL), dried over anhydrous sodium sulphate (Na₂SO₄), filtered and concentrated to dryness to yield a residue. The residue was subjected to silica gel chromatography (0-10% MeOH/DCM) to yield dimethyl 6,6′-((2-(((5-isothiocyanatopentyl)thio)methyl)-1,4,10,13-tetraoxa-7,16-diazacyclooctadecane-7,16-diyl)bis(methylene))(S)-dipicolinate as a yellow solid (30 mg).

Step 3: A stir bar, dimethyl 6,6′-((2-(((5-isothiocyanatopentyl)thio)methyl)-1,4,10,13-tetraoxa-7,16-diazacyclooctadecane-7,16-diyl)bis(methylene))(S)-dipicolinate (30 mg, 0.040 mmol), and aqueous HCl (6 N, 0.2 mL, 0.8 mmol) were added to a 10 mL single-neck round-bottomed flask and stirred overnight at room temperature. The reaction mixture was concentrated to dryness in vacuo, and the concentrate was purified by HPLC (Column: XBRIDGE C18 19×150 mm, 5.0 μm; Mobile phase: 0.1% TFA in water/acetonitrile; Flow Rate: 15.0 mL/min) to yield (S)-6,6′-((2-(((5-isothiocyanatopentyl)thio)methyl)-1,4,10,13-tetraoxa-7,16-diazacyclooctadecane-7,16-diyl)bis(methylene))dipicolinic acid (12 mg). LC-MS APCI: Calculated for C₃₃H₄₇N₅O₈S₂: 705.29; Observed m/z [M+H]⁺ 706.2. ¹H NMR (400 MHz, DMSO-d₆): δ 13.40 (s, 1H), 9.90 (s, 1H), 8.17-8.09 (m, 4H), 7.78 (d, J=6.80 Hz, 2H), 4.70 (s, 4H), 3.93-3.17 (m, 27H), 2.68-2.67 (m, 2H), 1.64-1.60 (m, 2H), 1.53-1.49 (m, 2H), 1.40-1.38 (m, 2H).

### Example 8

### (S)-6,6′-((2-(((2-(2-(4-Isothiocyanatophenoxy)ethoxy)ethyl)thio)methyl)-1,4,10,13-tetraoxa-7,16-diazacyclooctadecane-7,16-diyl)bis(methylene))dipicolinic acid

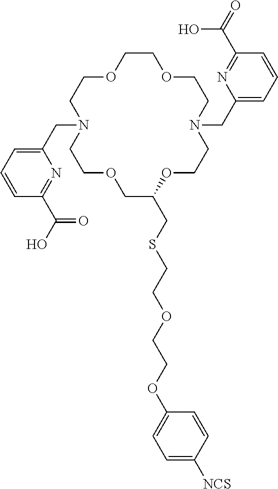

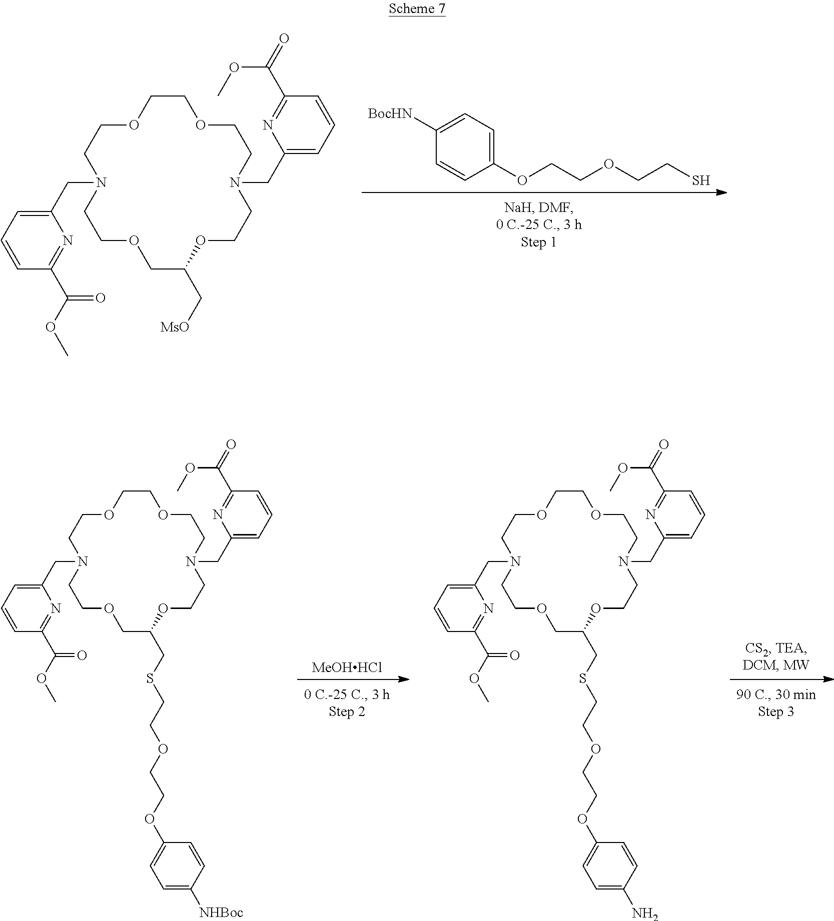

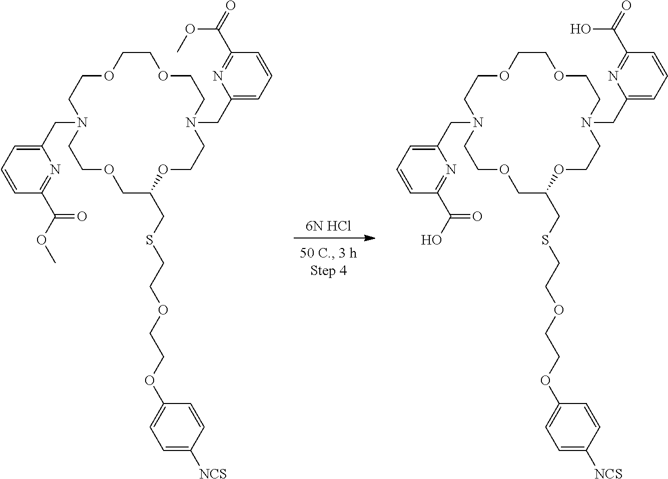

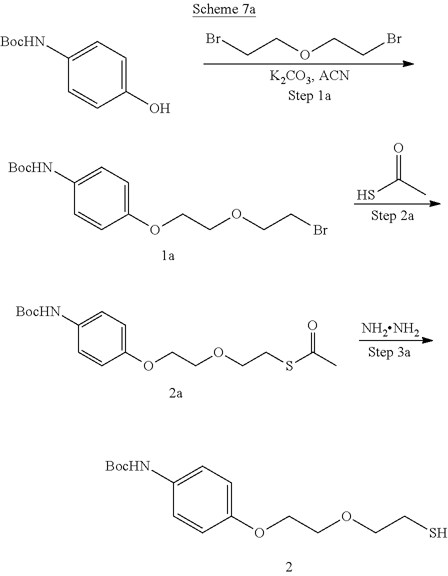

Scheme 7a, Step 1a: A stir bar, tert-butyl (4-hydroxyphenyl)carbamate (4.5 g, 22 mmol), 1-bromo-2-(2-bromoethoxy)ethane (5.0 g, 22 mmol), K₂CO₃ (4.6 g, 43 mmol) and ACN (45 mL) were added to a 250 mL three-neck round-bottomed flask under nitrogen atmosphere, and the resultant reaction mixture was heated at 80° C. for 16 h under nitrogen atmosphere. Reaction mixture was cooled to room temperature, filtered through Celite®, and concentrated to dryness in vacuo to yield a concentrate which was purified by silica gel chromatography (0-20% EtOAc/pet ether) to afford product tert-butyl (4-(2-(2-bromoethoxy)ethoxy)phenyl)carbamate (2.0 g,).

Step 2a: A stir bar, tert-butyl (4-(2-(2-bromoethoxy)ethoxy)phenyl)carbamate (2.0 g, 5.6 mmol), ethanethioic S-acid (0.42 g, 5.6 mmol), K₂CO₃ (1.5 g, 11 mmol) and ACN (50 mL) were added to a 250 mL three-neck round-bottomed flask under nitrogen atmosphere. The reaction mixture stirred at 80° C. for 2 h, and then cooled to room temperature, filtered through Celite®, and concentrated to dryness in vacuo. The concentrate was purified using neutral alumina chromatography (0-50% EtOAc/pet ether) to yield S-(2-(2-(4-((tert-butoxycarbonyl)amino)phenoxy)ethoxy)ethyl) ethanethioate (1.8 g).

Step 3a: A stir bar, S-(2-(2-(4-((tert-butoxycarbonyl)amino)phenoxy)ethoxy)ethyl) ethanethioate (1.8 g, 5.1 mmol), ethanol (20 mL) and hydrazine monohydrate (0.24 g, 0.24 mL, 7.6 mmol) were added to a 250 mL single-neck round-bottomed flask under nitrogen, and stirred at 80° C. for 1 h. The reaction mixture was then cooled to room temperature and concentrated to dryness in vacuo, to yield a concentrate which was purified via silica gel chromatography (5-10% EtOAc/pet ether) to yield tert-butyl (4-(2-(2-mercaptoethoxy)ethoxy)phenyl)carbamate (0.5 g) as a colorless oil.

Scheme 7, Step 1: A solution consisting of tert-butyl (4-(2-(2-mercaptoethoxy)ethoxy)phenyl)carbamate (0.40 g, 1.0 mmol) and DMF (3.0 mL) was added dropwise over 5 minutes to a 50 mL three-neck round-bottomed flask containing a suspension of sodium hydride (0.060 g, 60% in mineral oil, 1.5 mmol) in DMF (3.0 mL) at 0° C. and a under nitrogen atmosphere. Once addition was complete, the reaction mixture was warmed to room temperature and stirred for 15 minutes. The mixture was re-cooled to 0° C. and a solution consisting of dimethyl 6,6′-((2-(((methylsulfonyl)oxy)methyl)-1,4,10,13-tetraoxa-7,16-diazacyclooctadecane-7,16-diyl)bis(methylene))(S)-dipicolinate (0.5 g, 0.7 mmol) and DMF (3.0 mL) was added dropwise. Once addition was complete, the reaction mixture was slowly warmed to room temperature and stirred 1.5 h. The reaction was slowly treated with sat. NH₄Cl (0.2 mL) and then concentrated to dryness to yield an oil. The oil was purified by preparative HPLC (Column: XBRIDGE C18 19×150 mm 5.0 μm; Mobile phase: 0.1% TFA in water/acetonitrile; Flow Rate: 15.0 mL/min) to yield dimethyl 6,6′-((2-(((2-(2-(4-((tert-butoxycarbonyl)amino)phenoxy)ethoxy)ethyl)thio)methyl)-1,4,10,13-tetraoxa-7,16-diazacyclooctadecane-7,16-diyl)bis(methylene))(S)-dipicolinate (0.15 g) as a brown oil.

Step 2: A stir bar, dimethyl 6,6′-((2-(((2-(2-(4-((tert-butoxycarbonyl)amino)phenoxy)ethoxy)ethyl)thio)methyl)-1,4,10,13-tetraoxa-7,16-diazacyclooctadecane-7,16-diyl)bis(methylene))(S)-dipicolinate (0.15 g, 0.16 mmol), MeOH (1.0 mL) and HCl in methanol (4 M, 0.80 mL, 3.2 mmol) were added to a 25 mL single-neck round-bottomed flask at 0° C. and then brought to room temperature. and stirred for 3 h. The volatiles were removed in vacuo to yield dimethyl 6,6′-((2-(((2-(2-(4-aminophenoxy)ethoxy)ethyl)thio)methyl)-1,4,10,13-tetraoxa-7,16-diazacyclooctadecane-7,16-diyl)bis(methylene))(S)-dipicolinate (0.12 g), which was used without purification.

Step 3: A stir bar, dimethyl 6,6′-((2-(((2-(2-(4-aminophenoxy)ethoxy)ethyl)thio)methyl)-1,4,10,13-tetraoxa-7,16-diazacyclooctadecane-7,16-diyl)bis(methylene))(S)-dipicolinate (0.12 g, 0.15 mmol), triethylamine (46 mg, 0.46 mmol), dry DCM (3 mL) and carbon disulfide (17 mg, 0.22 mmol) were added to a pressure vial at room temperature under nitrogen atmosphere. The reaction mixture was subjected to microwave-irradiation (150 W power) at 90° C. for 30 min. The reaction mixture was cooled to room temperature and was diluted with dichloromethane (10 mL), washed successively with water (5 mL), 1M HCl (5 mL), and water (5 mL), dried over anhydrous Na₂SO₄ and concentrated to dryness to yield dimethyl 6,6′-((2-(((2-(2-(4-isothiocyanatophenoxy)ethoxy)ethyl)thio)methyl)-1,4,10,13-tetraoxa-7,16-diazacyclooctadecane-7,16-diyl)bis(methylene))(S)-dipicolinate (0.12 g), which was used in the without purification.

Step 4: A stir bar, dimethyl 6,6′-((2-(((2-(2-(4-isothiocyanatophenoxy)ethoxy)ethyl)thio)methyl)-1,4,10,13-tetraoxa-7,16-diazacyclooctadecane-7,16-diyl)bis(methylene))(S)-dipicolinate (0.12 g, 0.15 mmol) and aqueous HCl (6 N, 0.51 mL, 3.1 mmol) were added to a 10 mL single-neck round-bottomed flask and stirred at 50° C. for 3 h. The reaction mixture was cooled to room temperature, concentrated to dryness in vacuo, and the concentrate was purified via preparative HPLC (Column: XBRIDGE C18 19×150 mm 5.0 μm; Mobile phase: 0.1% TFA in water/acetonitrile; Flow Rate: 15.0 mL/min) to yield (S)-6,6′-((2-(((2-(2-(4-isothiocyanatophenoxy)ethoxy)ethyl)thio)methyl)-1,4,10,13-tetraoxa-7,16-diazacyclooctadecane-7,16-diyl)bis(methylene))dipicolinic acid (40 mg, 37%). LC-MS APCI: Calculated for C₃₈H₄₉N₅O₁₀S₂: 799.29; Observed m/z [M+H]⁺ 799.9. ¹H NMR (400 MHz, CD₃OD): δ 8.23-8.20 (m, 2H), 8.15-8.09 (m, 2H), 7.74-7.71 (m, 2H), 7.19 (d, J=8.80 Hz, 2H), 6.92 (d, J=9.20 Hz, 2H), 4.81 (s, 2H), 4.77 (s, 2H), 4.09-4.11 (m, 4H), 3.92-3.95 (m, 6H), 3.79 (t, J=4.00 Hz, 3H), 3.66-3.71 (m, 16H), 2.70-2.76 (m, 4H).

### Example 9

### (S)-6,6′-((2-(((2-(2-(2-(4-Isothiocyanatophenoxy)ethoxy)ethoxy)ethyl)thio)methyl)-1,4,10,13-tetraoxa-7,16-diazacyclooctadecane-7,16-diyl)bis(methylene))dipicolinic acid

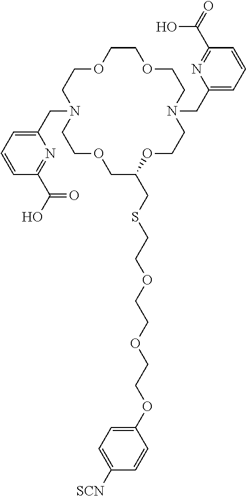

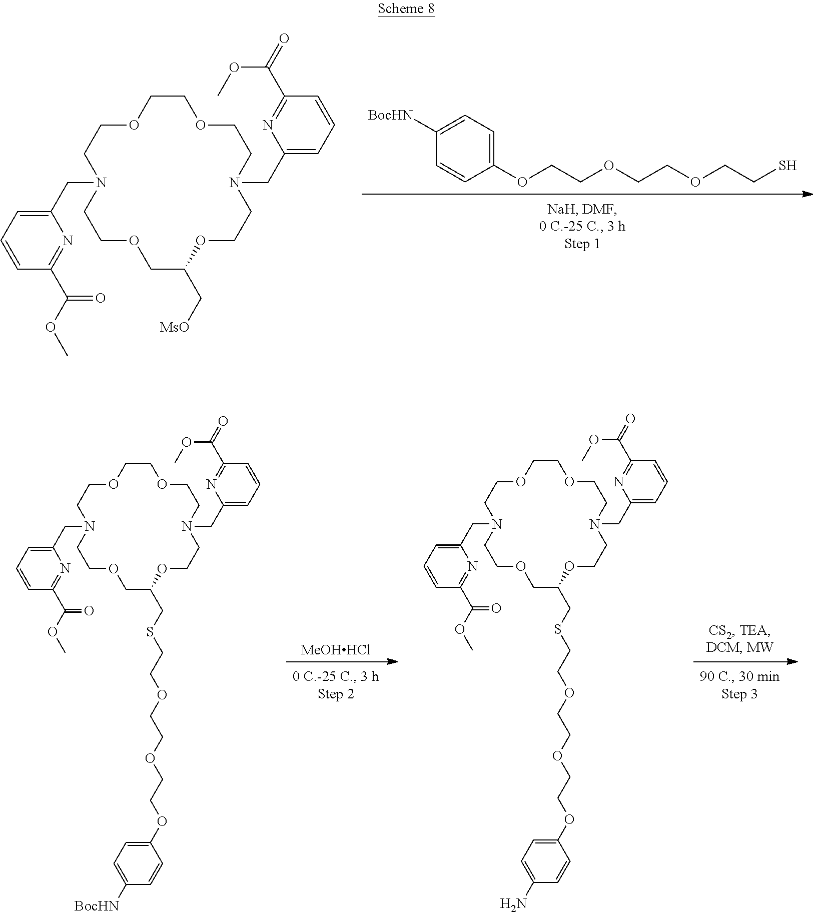

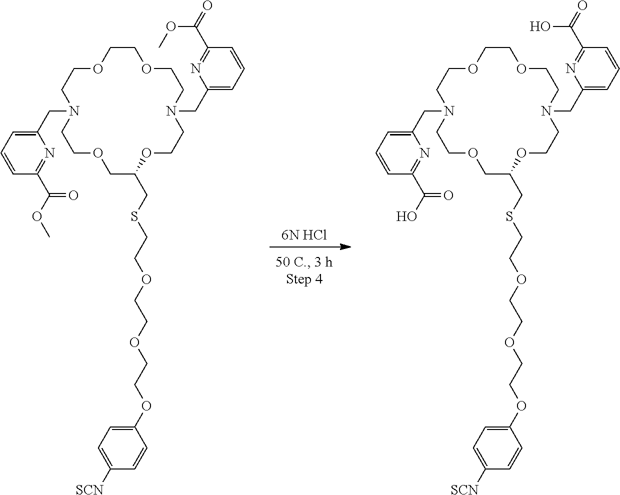

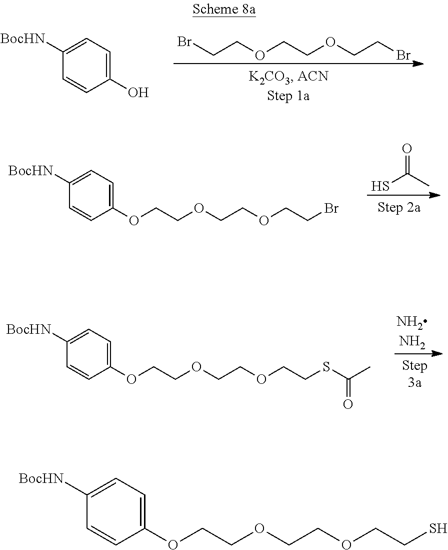

Scheme 8a, Step 1a: A stir bar, tert-butyl (4-hydroxyphenyl)carbamate (3.5 g, 17 mmol), 1,2-bis(2-bromoethoxy)ethane (4.6 g, 17 mmol), K₂CO₃ (4.6 g, 33 mmol) and ACN (40 mL) were added to a 250 mL three-neck round-bottomed flask, and then stirred at 80° C. for 48 h under a nitrogen atmosphere. The reaction mixture was cooled to room temperature, filtered through Celite® and concentrated to dryness in vacuo to yield a concentrate, which was purified via silica gel chromatography (0-10% MeOH/DCM) to yield tert-butyl (4-(2-(2-(2-bromoethoxy)ethoxy)ethoxy)phenyl)carbamate (4.0 g) as a brown oil.

Step 2a: A stir bar, tert-butyl (4-(2-(2-(2-bromoethoxy)ethoxy)ethoxy)phenyl)carbamate (4.0 g, 9.9 mmol), ethanethioic S-acid (0.75 g, 9.9 mmol), K₂CO₃ (2.7 g, 20 mmol) and ACN (50 mL) were added to a 250 mL three-neck round-bottomed flask under a nitrogen atmosphere, and the reaction mixture was heated at 60° C. for 2 h under a nitrogen atmosphere. The reaction mixture was cooled to room temperature, filtered through Celite®, concentrated to dryness in vacuo and the concentrate was purified by alumina chromatography (0-5₀% EtOAc/Pet Ether) to yield S-(2-(2-(2-(4-((tert-butoxycarbonyl)amino)phenoxy)ethoxy)ethoxy)ethyl) ethanethioate (3.0 g) as brown oil.

Step 3a: A stir bar, S-(2-(2-(2-(4-((tert-butoxycarbonyl)amino)phenoxy)ethoxy)ethoxy)ethyl) ethanethioate (3.0 g, 7.5 mmol), ethanol (50 mL) and hydrazine monohydrate (0.36 g, 0.36 mL, 11 mmol) were added to a 250 mL single-neck round-bottomed flask under nitrogen, and stirred at 80° C. for 1 h. The reaction mixture was cooled to room temperature, concentrated to dryness in vacuo, and the concentrate was purified by silica gel chromatography (5-10% EtOAc/pet ether) to yield tert-butyl (4-(2-(2-(2-mercaptoethoxy)ethoxy)ethoxy)phenyl)carbamate (1.0 g) as a colorless oil.

Scheme 7, Step 1: A solution consisting of tert-butyl (4-(2-(2-(2-mercaptoethoxy)ethoxy)ethoxy)phenyl)carbamate (0.40 g, 1.0 mmol) and DMF (3.0 mL) was added dropwise over 5 minutes to a 50 mL three-neck round-bottomed flask containing a suspension of sodium hydride (0.060 g, 60% in mineral oil, 1.5 mmol) in DMF (3.0 mL) at 0° C. under nitrogen atmosphere. Once addition was complete, the reaction mixture brought to room temperature and stirred continuously for 15 minutes. The mixture was re-cooled to 0° C. and a solution consisting of dimethyl 6,6′-((2-(((methylsulfonyl)oxy)methyl)-1,4,10,13-tetraoxa-7,16-diazacyclooctadecane-7,16-diyl)bis(methylene))(S)-dipicolinate (0.5 g, 0.7 mmol) and DMF (3.0 mL) was added dropwise over 10 minutes. Once addition was complete, the reaction mixture was slowly warmed to room temperature and stirred for 1.5 h. The reaction mixture was then slowly treated with sat. aqueous NH₄Cl (0.2 mL) and concentrated to dryness to yield an oil. The oil was purified by preparative HPLC (Column: XBRIDGE C18 (19×150 mm) 5.0 μm; Mobile phase: 0.1% TFA in water/acetonitrile; Flow Rate: 15.0 mL/min) to yield dimethyl 6,6′-((2-(((2-(2-(2-(4-((tert-butoxycarbonyl)amino)phenoxy)ethoxy)ethoxy)ethyl)thio)methyl)-1,4,10,13-tetraoxa-7,16-diazacyclooctadecane-7,16-diyl)bis(methylene))(S)-dipicolinate (0.15 g, 21%) as a brown oil.

Step 2: A stir bar, dimethyl 6,6′-((2-(((2-(2-(2-(4-((tert-butoxycarbonyl)amino)phenoxy)ethoxy)ethoxy)ethyl)thio)methyl)-1,4,10,13-tetraoxa-7,16-diazacyclooctadecane-7,16-diyl)bis(methylene))(S)-dipicolinate (0.15 mg, 0.16 mmol), MeOH (1.0 mL) and HCl in methanol (4 M, 0.80 mL, 3.2 mmol) were added to a 25 mL single-neck round-bottomed flask at 0° C. The reaction mixture was allowed to warm to room temperature and stirred for 3 h. The volatiles were removed in vacuo to give yield dimethyl 6,6′-((2-(((2-(2-(2-(4-aminophenoxy)ethoxy)ethoxy)ethyl)thio)methyl)-1,4,10,13-tetraoxa-7,16-diazacyclooctadecane-7,16-diyl)bis(methylene))(S)-dipicolinate (0.12 g), which was used in the next step without purification.

Step 3: A stir bar, dimethyl 6,6′-((2-(((2-(2-(2-(4-aminophenoxy)ethoxy)ethoxy)ethyl)thio)methyl)-1,4,10,13-tetraoxa-7,16-diazacyclooctadecane-7,16-diyl)bis(methylene))(S)-dipicolinate (0.12 g, 0.14 mmol), triethylamine (44 mg, 0.43 mmol) dry DCM (5 mL) and carbon disulfide (17 mg, 0.22 mmol) were added to a microwave vial at room temperature under a nitrogen atmosphere. The reaction mixture subjected to microwave irradiation (150 W power) at 90° C. for 30 min. The reaction mixture was then cooled to room temperature, diluted with dichloromethane (10 mL), washed successively with water (5 mL), 1M HCl (5 mL), and water (5 mL), dried over anhydrous Na₂SO₄ and concentrated to dryness to yield dimethyl 6,6′-((2-(((2-(2-(2-(4-isothiocyanatophenoxy)ethoxy)ethoxy)ethyl)thio)methyl)-1,4,10,13-tetraoxa-7,16-diazacyclooctadecane-7,16-diyl)bis(methylene))(S)-dipicolinate (0.12 g), which was used in the next step without purification.

Step 4: A stir bar, dimethyl 6,6′-((2-(((2-(2-(2-(4-isothiocyanatophenoxy)ethoxy)ethoxy)ethyl)thio)methyl)-1,4,10,13-tetraoxa-7,16-diazacyclooctadecane-7,16-diyl)bis(methylene))(S)-dipicolinate (0.12 mg, 0.14 mmol) and aqueous HCl (6 N, 0.50 mL, 2.8 mmol) were added to a 10 mL single-neck round-bottomed flask and stirred at 50° C. for 3 h. The reaction mixture was cooled to room temperature, concentrated to dryness in vacuo to yield a residue which was purified by preparative HPLC (Column: XBRIDGE C18 19×150 mm 5.0 μm; Mobile phase: 0.1% TFA in water/acetonitrile; Flow Rate: 15.0 mL/min) to yield (S)-6,6′-((2-(((2-(2-(2-(4-isothiocyanatophenoxy)ethoxy)ethoxy)ethyl)thio)methyl)-1,4,10,13-tetraoxa-7,16-diazacyclooctadecane-7,16-diyl)bis(methylene))dipicolinic acid (50 mg). LC-MS APCI: Calculated for C₄₀H₅₃N₅O₁₁S₂: 843.32; Observed m/z [M+H]⁺ 843.9. ¹H NMR (400 MHz, CD₃OD): δ 8.24-8.21 (m, 2H), 8.21-8.11 (m, 2H), 7.74 (d, J=7.60 Hz, 2H), 7.23-7.20 (m, 2H), 6.97-6.95 (m, 2H), 4.84-4.79 (m, 5H), 4.14-4.12 (m, 4H), 3.97-3.94 (m, 6H), 3.83-3.59 (m, 23H), 2.75-2.67 (m, 4H).

### Example 10

### 6-((4-((2-(2-(2-Aminoethoxy)ethoxy)ethyl)carbamoyl)phenyl)(16-((6-carboxypyridin-2-yl)methyl)-1,4,10,13-tetraoxa-7,16-diazacyclooctadecan-7-yl)methyl)picolinic acid

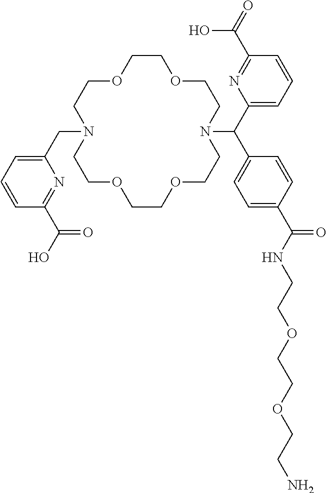 [SMILES: NCCOCCOCCNC(=O)c1ccc(C(c2cccc(C(=O)O)n2)N2CCOCCOCCN(Cc3cccc(C(=O)O)n3)CCOCCOCC2)cc1]

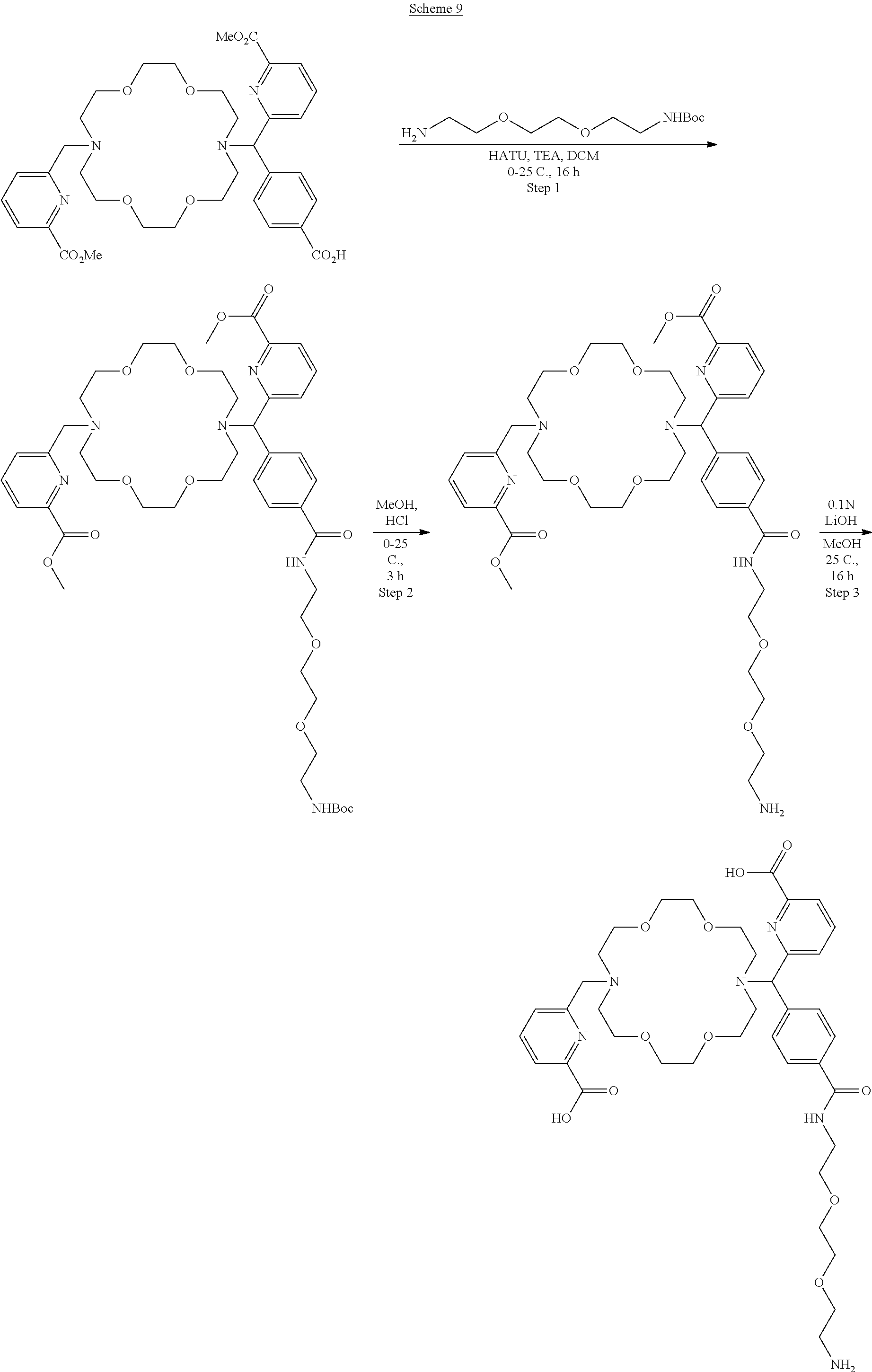

Step 1: A stir bar, 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.40 g, 0.60 mmol), tert-butyl (2-(2-(2-aminoethoxy)ethoxy)ethyl)carbamate (0.15 g, 0.60 mmol), triethylamine (0.18 g, 0.76 mmol), HATU (0.33 g, 0.90 mmol), and DCM (4.0 mL) were added to a 25 mL three-neck round-bottomed flask at 0° C. under a nitrogen atmosphere. The mixture was stirred overnight at room temperature and diluted with water (10 mL), and extracted with dichloromethane (10 mL×3). The combined extracts were washed with 10% aqueous NaHCO₃ (10 mL) and brine (10 mL), dried over anhydrous Na₂SO₄, filtered, and concentrated to dryness to yield a concentrate, which was purified via silica gel chromatography (0-10% MeOH/DCM) to yield methyl 6-((4-((2,2-dimethyl-4-oxo-3,8,11-trioxa-5-azatridecan-13-yl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.18 g).

Step 2: A stir bar, methyl 6-((4-((2,2-dimethyl-4-oxo-3,8,11-trioxa-5-azatridecan-13-yl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.18 g, 0.20 mmol), MeOH (1.8 mL), and HCl in methanol (4 M, 1.0 mL, 4.0 mmol) were added to a 10 mL single-neck round-bottomed flask at 0° C., and then brought to room temperature and stirred for 2 h. The volatiles were removed in vacuo to yield methyl 6-((4-((2-(2-(2-aminoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.15 g), which was used without purification.

Step 3: A stir bar, methyl 6-((4-((2-(2-(2-aminoethoxy)ethoxy)ethyl)carbamoyl)phenyl)(16-((6-(methoxycarbonyl)pyridin-2-yl)methyl)-1,4,10,13-tetraoxa-7,16-diazacyclooctadecan-7-yl)methyl)picolinate (0.1 g, 0.1 mmol), aqueous LiOH (3 mL, 0.1 N, 0.3 mmol), and MeOH (1.0 mL) were added to an 8 mL reaction vial at room temperature and stirred overnight. The reaction mixture was adjusted to pH˜6.5 with acetic acid, and then concentrated to dryness in vacuo at room temperature to yield a concentrate, which was purified via preparative HPLC (Column: XBRIDGE C18 19×150 mm, 5.0 μm; Mobile phase: 0.1% TFA in water/ACN; Flow Rate: 15.0 mL/min) to yield 6-((4-((2-(2-(2-aminoethoxy)ethoxy)ethyl)carbamoyl)phenyl)(16-((6-carboxypyridin-2-yl)methyl)-1,4,10,13-tetraoxa-7,16-diazacyclooctadecan-7-yl)methyl)picolinic acid (40 mg) LC-MS APCI: Calculated for C₃₉H₅₄N₆O₁; 782.39; Observed m/z [M+H]⁺ 783.0.

### Example 11

### 6,6′-((18-(((2-(2-Aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinic acid and

### Example 12

### N-acyl-DBCO tagged 6,6′-((18-(((2-(2-Aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinic acid

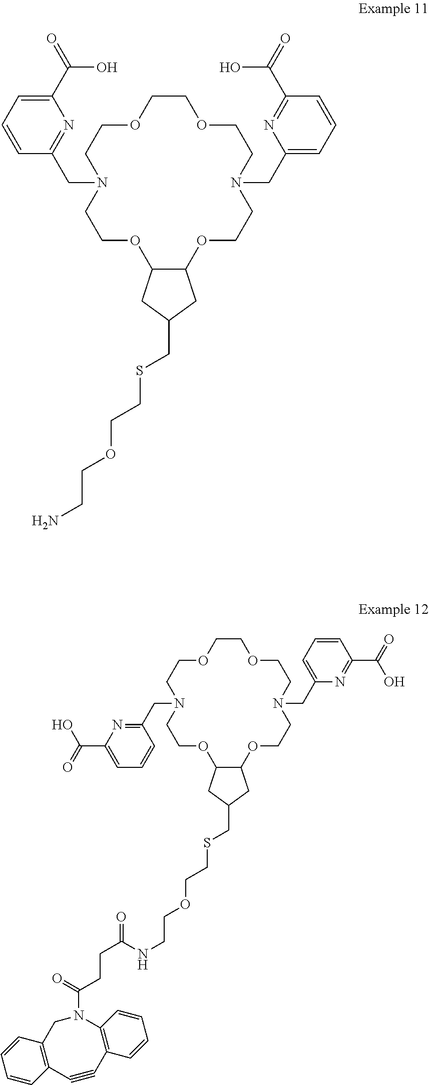 [SMILES: NCCOCCSCC1CC2OCCN(Cc3cccc(C(=O)O)n3)CCOCCOCCN(Cc3cccc(C(=O)O)n3)CCOC2C1 ; O=C(CCC(=O)N1Cc2ccccc2C#Cc2ccccc21)NCCOCCSCC1CC2OCCN(Cc3cccc(C(=O)O)n3)CCOCCOCCN(Cc3cccc(C(=O)O)n3)CCOC2C1]

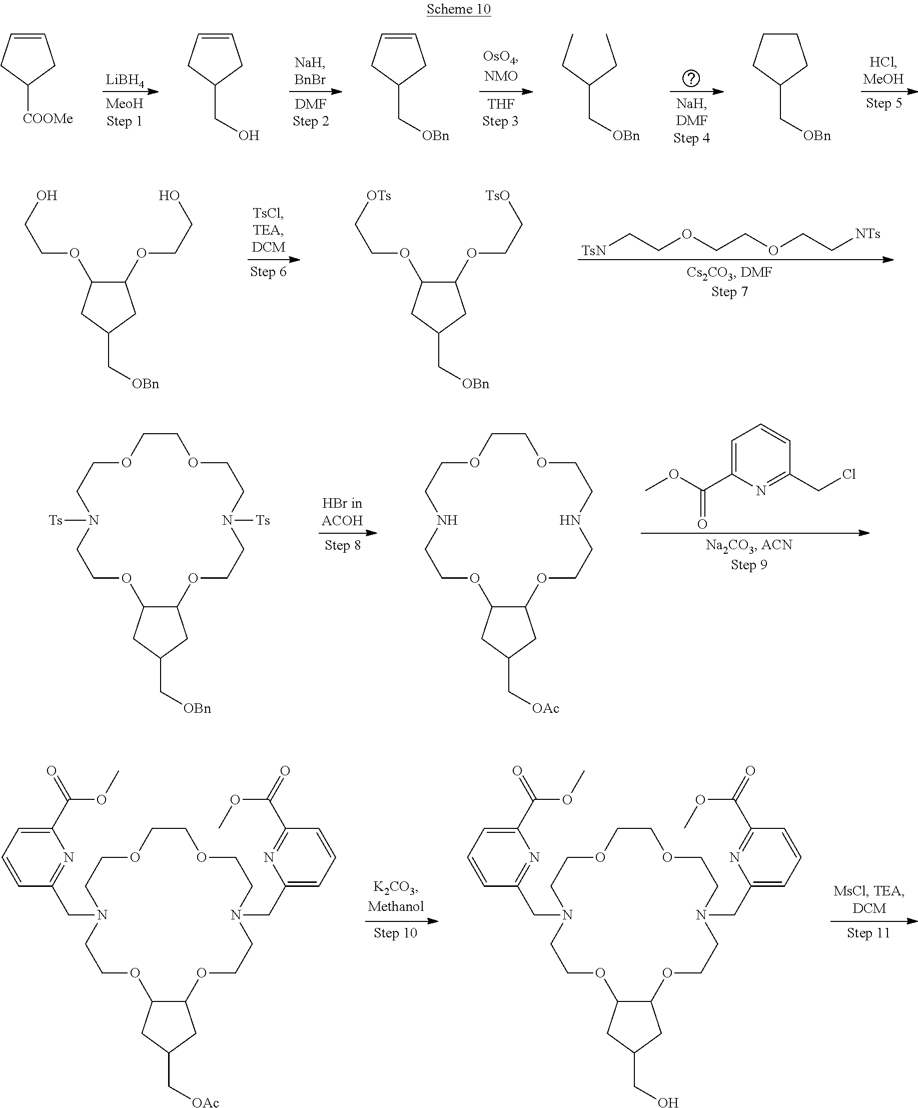

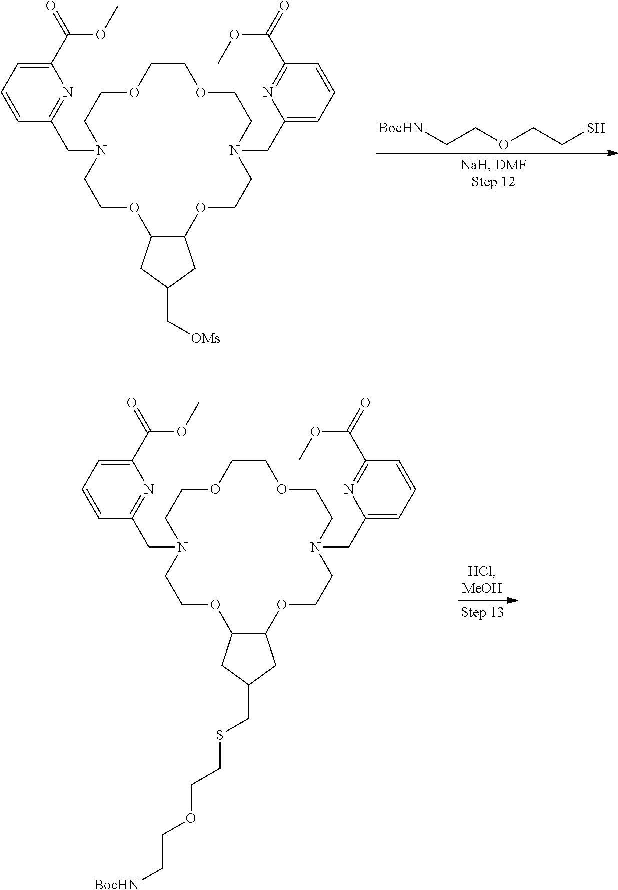 [SMILES: COC(=O)c1cccc(CN2CCOCCOCCN(Cc3cccc(C(=O)OC)n3)CCOC3CC(COS(C)(=O)=O)CC3OCC2)n1 ; CC(C)(C)OC(=O)NCCOCCS ; COC(=O)c1cccc(CN2CCOCCOCCN(Cc3cccc(C(=O)OC)n3)CCOC3CC(CSCCOCCNC(=O)OC(C)(C)C)CC3OCC2)n1]

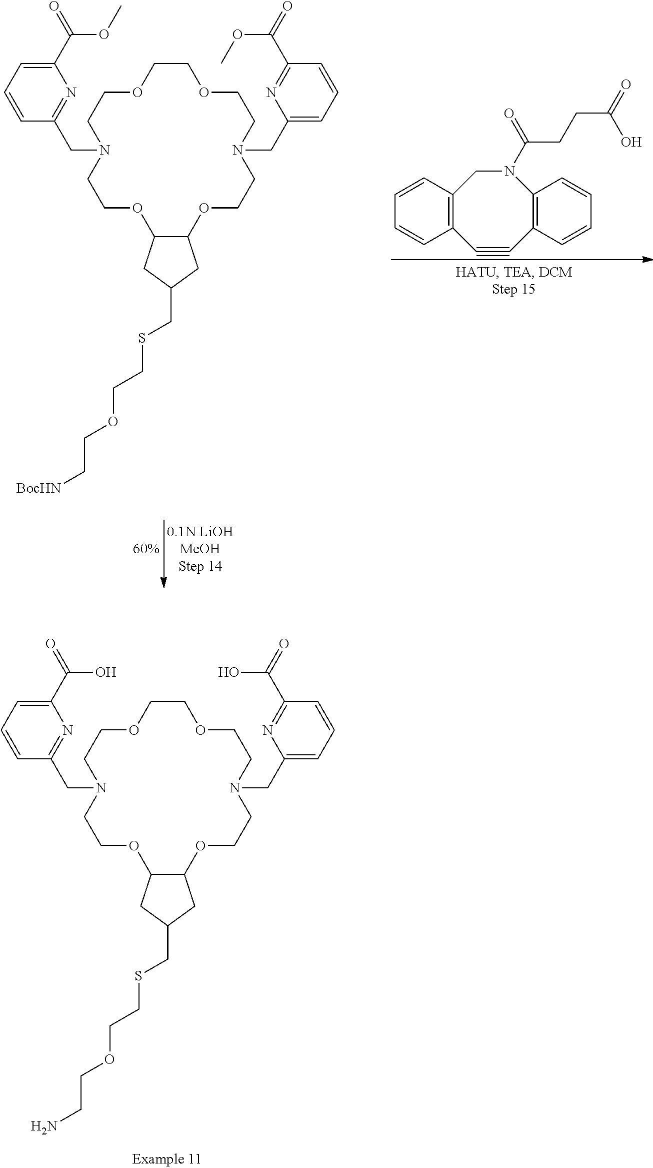 [SMILES: COC(=O)c1cccc(CN2CCOCCOCCN(Cc3cccc(C(=O)OC)n3)CCOC3CC(CSCCOCCNC(=O)OC(C)(C)C)CC3OCC2)n1 ; O=C(O)CCC(=O)N1Cc2ccccc2C#Cc2ccccc21 ; NCCOCCSCC1CC2OCCN(Cc3cccc(C(=O)O)n3)CCOCCOCCN(Cc3cccc(C(=O)O)n3)CCOC2C1]

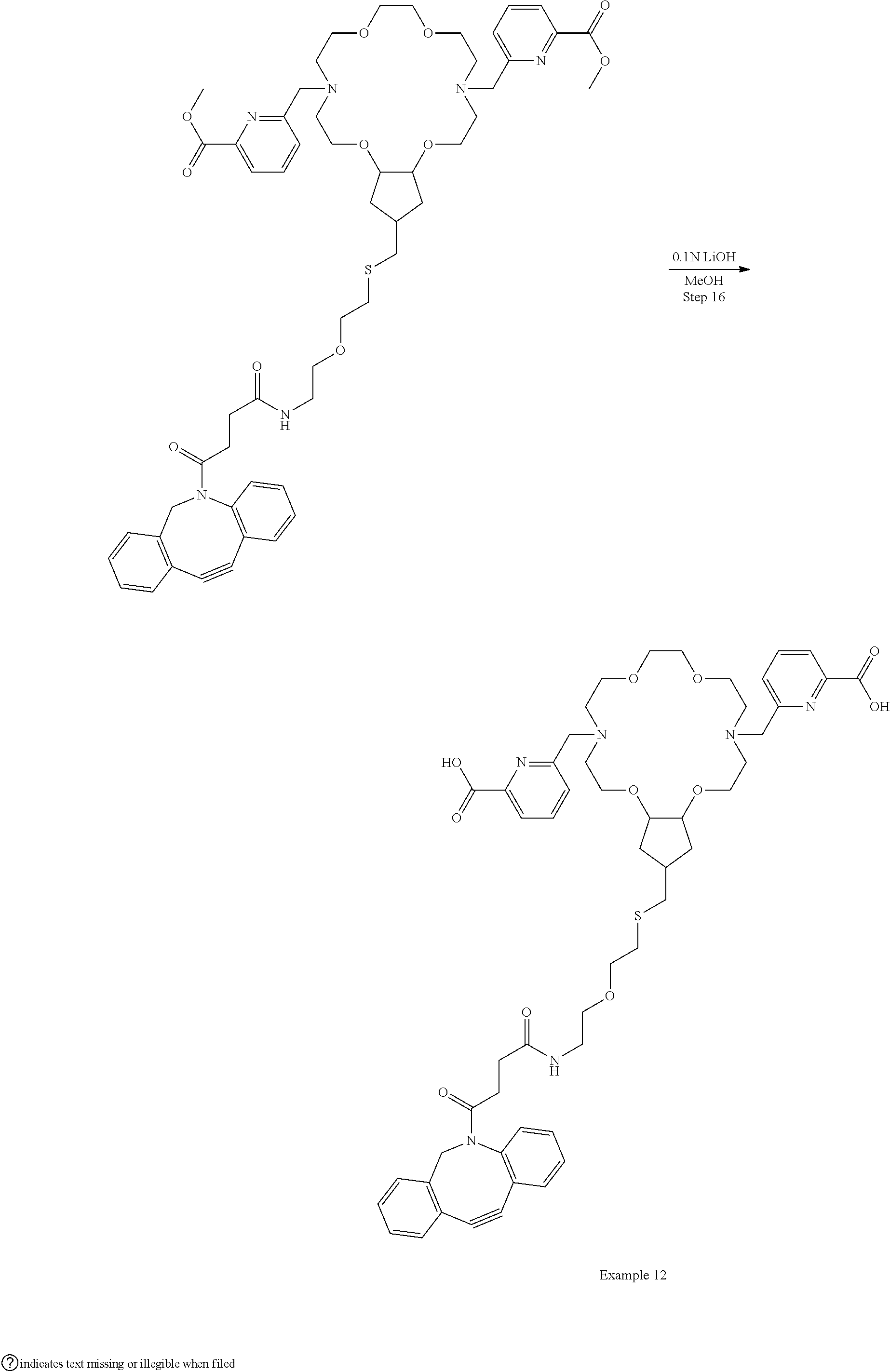 [SMILES: COC(=O)c1cccc(CN2CCOCCOCCN(Cc3cccc(C(=O)OC)n3)CCOC3CC(CSCCOCCNC(=O)CCC(=O)N4Cc5ccccc5C#Cc5ccccc54)CC3OCC2)n1 ; O=C(CCC(=O)N1Cc2ccccc2C#Cc2ccccc21)NCCOCCSCC1CC2OCCN(Cc3cccc(C(=O)O)n3)CCOCCOCCN(Cc3cccc(C(=O)O)n3)CCOC2C1]

Step 1: A stir bar, methyl cyclopent-3-ene-1-carboxylate (25.0 g, 198 mmol), THF (600 mL), methanol (12.6 g, 16.0 mL, 397 mmol) and lithium borohydride (198 mL, 2.0 M in THF, 397 mmol) were added to a 3000 mL three-neck round-bottomed flask at 0° C. Once addition was complete, the reaction mixture was stirred at 70° C. for 6 h. The reaction mixture was then cooled to room temperature, slowly treated with ice water (250 mL), cooled further to 0° C., brought to pH˜2 with 1.5 N HCl (pH˜2) and then extracted with DCM (1000 mL×3). The combined extracts were washed with water (500 mL), dried over anhydrous Na₂SO₄, filtered and concentrated to dryness to yield a concentrate which was purified by silica gel chromatography (50-80% EtOAc/pet ether) to yield cyclopent-3-en-1-ylmethanol (13.8 g).

Step 2: A solution consisting of cyclopent-3-en-1-ylmethanol (13.7 g, 139 mmol) and DMF (50 mL) was added dropwise over 30 min into a 1000 mL three-neck round-bottomed flask containing a suspension of sodium hydride (6.69 g, 60% in mineral oil, 167 mmol) in DMF (50 mL) at 0° C. under nitrogen atmosphere. Once addition was complete, the reaction mixture was slowly warmed to room temperature and stirring continued for 30 min. The mixture was then re-cooled to 0° C. and treated dropwise over 15 min with a solution consisting of benzyl bromide (19.8 g, 167 mmol) and DMF (50 mL). Once addition was complete, the reaction mixture was slowly warmed to room temperature and then stirred for 16 h. The reaction mixture was slowly treated with sat. aqueous NH₄Cl (50 mL) and then extracted with ethyl acetate (1000 mL×3). The combined extracts were washed with water (500 mL×3), dried over anhydrous Na₂SO₄, filtered, and concentrated to dryness to yield a concentrate. The concentrate was purified by silica gel chromatography (0-20% EtOAc/pet ether) to yield ((cyclopent-3-en-1-ylmethoxy)methyl)benzene (21.0 g).

Step 3: A stir bar, NMO (38.0 g, 50% wt in H₂O, 158 mmol), THF (180 mL) and osmium tetroxide (16.2 g, 3.21 mL, 2.5% wt % in t-butanol, 0.158 mmol) were added to a 1000 mL three-neck round-bottomed flask at 0° C. The reaction mixture was brought to room temperature, stirred for 10 min and re-cooled to 0° C. Once cooled, the mixture was treated dropwise over 15 min with a solution of ((cyclopent-3-en-1-ylmethoxy)methyl)benzene (20.0 g, 158 mmol) and THF (180 mL). The reaction was brought to room temperature and stirred for 16 h before it was slowly treated with sat. aqueous NaHCO₃(100 mL) and extracted with DCM (1000 mL×3). The combined extracts were washed with water (500 mL), dried over anhydrous Na₂SO₄, filtered, and concentrated to dryness to yield a concentrate, which was purified via silica gel chromatography (0-20% EtOAc/pet ether) to yield an isomeric mixture of 4-((benzyloxy)methyl)cyclopentane-1,2-diol as a colorless oil. The isomers were separated via SFC (Instrument: PIC 100; Column: Chiralpak OXH (250×30) mm, 5 m; Mobile phase: CO₂: 0.5% isopropyl amine in IPA (60:40); Total flow: 70 g/min; Back pressure: 100 bar; Wave length: 220 nm; Cycle time: 8.0 min) yielded both cis-1,2 isomers of 4-((benzyloxy)methyl)cyclopentane-1,2-diol: 1^{st} eluting isomer (10 g) and 2^{nd} eluting isomer (5 g).

Step 4: A solution consisting of the 1^{st}-eluting isomer of 4-((benzyloxy)methyl)cyclopentane-1,2-diol (10.0 g, 45.0 mmol) and DMF (60 mL) was added dropwise over 1 h to a 250 mL three-neck round-bottomed flask containing a suspension of sodium hydride (8.62 g, 60% in mineral oil, 225 mmol) in DMF (60 mL) at 0° C. under a nitrogen atmosphere. Once addition was complete, the reaction mixture brought to room temperature and stirred for 30 min. The mixture was then re-cooled to 0° C. and treated dropwise over 15 min with a solution consisting of 2-(2-bromoethoxy)tetrahydro-2H-pyran (47.0 g, 225 mmol) and DMF (60 mL). Once addition was complete, the reaction mixture was slowly warmed to room temperature and stirred for 2 h. The mixture was then slowly treated with sat. aqueous NH₄Cl (50 mL) and then extracted with ethyl acetate (500 mL×3). The combined extracts were washed with water (500 mL), dried over anhydrous Na₂SO₄, filtered, and concentrated to dryness to yield an oil, which was purified by silica gel chromatography (0-30% EtOAc/pet ether) to yield 2,2′-((((4-((benzyloxy)methyl)cyclopentane-1,2-diyl)bis(oxy))bis(ethane-2,1-diyl))bis(oxy))bis(tetrahydro-2H-pyran) (21.0 g).

Step 5: A stir bar, 2,2′-((((4-((benzyloxy)methyl)cyclopentane-1,2-diyl)bis(oxy))bis(ethane-2,1-diyl))bis(oxy))bis(tetrahydro-2H-pyran) (29.0 g, 61.0 mmol), MeOH (200 mL) and HCl in 1,4-dioxane (4 M, 3.0 mL, 12.0 mmol) were added to a 1000 mL three-neck round-bottomed flask and then heated at reflux for 1 h. The flask was then cooled to room temperature and the volatiles removed in vacuo to yield 2,2′-((4-((benzyloxy)methyl)cyclopentane-1,2-diyl)bis(oxy))bis(ethan-1-01) (20.0 g) as a residue, which was used without purification.

Step 6: A stir bar, 2,2′-((4-((benzyloxy)methyl)cyclopentane-1,2-diyl)bis(oxy))bis(ethan-1-ol) (20.0 g) (20.0 g, 64.4 mmol), DCM (200 mL) and triethylamine (32.6 mL, 322 mmol) were added to a 1000 mL round-bottomed flask under a nitrogen atmosphere, and the resulting mixture was cooled to 10° C. The mixture was then treated with pTsCl (36.9 g, 193 mmol) which was added portion-wise and then brought to room temperature. Once addition was complete the reaction mixture was stirred for 16 h during which time a precipitate formed. The mixture was then diluted with DCM (500 mL), washed with cold aq. HCl (1 M, 500 mL×3) and ice-cold water (500 mL×2), dried over anhydrous Na₂SO₄, filtered, and concentrated to dryness to yield a residue which was purified via silica gel chromatography (0-30% EtOAc/pet ether) to yield ((4-((benzyloxy)methyl)cyclopentane-1,2-diyl)bis(oxy))bis(ethane-2,1-diyl) bis(4-methylbenzenesulfonate) (26.0 g).

Step 7: A stir-bar, N,N′-((ethane-1,2-diylbis(oxy))bis(ethane-2,1-diyl))bis(4-methylbenzenesulfonamide) (21.0 g, 42.0 mmol), Cs₂CO₃ (41.3 g, 126 mmol) and dry DMF (250 mL) were added to a 2000 mL three-neck round-bottomed flask under nitrogen atmosphere, and the resultant heterogeneous mixture stirred at room temperature for 1.5 h. The mixture was then treated dropwise with a solution consisting of ((4-((benzyloxy)methyl)cyclopentane-1,2-diyl)bis(oxy))bis(ethane-2,1-diyl) bis(4-methylbenzenesulfonate) (26.0 g, 42.0 mmol) and DMF (250 mL) over a period of 2 h. Stirring was continued for 20 h, before the mixture was concentrated to dryness in vacuo to yield a paste-like solid. The paste was suspended in DCM (1000 mL), stirred for 30 min, and filtered by vacuum filtration. The filtrate was concentrated to dryness in vacuo to yield a concentrate, which was purified by silica gel chromatography (0-40% EtOAc/pet ether) to yield 18-((benzyloxy)methyl)-4,13-ditosyltetradecahydro-2H,11H,17H-cyclopenta[b][1,4,10,13]tetraoxa[7,16]diazacyclooctadecine (24 g).

Step 8: A HOAc solution of HBr (50%, 112 mL, 695 mmol) was added to a 500 mL round-bottomed flask containing a stir bar and 18-((benzyloxy)methyl)-4,13-ditosyltetradecahydro-2H,11H,17H-cyclopenta[b][1,4,10,13]tetraoxa[7,16]diazacyclooctadecine (24.0 g, 32.8 mmol) under a nitrogen atmosphere. The mixture was stirred at room temperature until homogeneous and then treated with phenol (16.3 g, 174 mmol). The reaction mixture was then heated at 60° C. for 6 h, before cooling to room temperature and concentrating to dryness in vacuo to yield a concentrate. The concentrate was purified via reverse-phase column chromatography (Column: Revelries C18-330 g; Mobile phase A: 0.1% TFA in water, Mobile phase B: acetonitrile; Flow rate: 60 mL/min) to yield (tetradecahydro-2H,11H,17H-cyclopenta[b][1,4,10,13]tetraoxa[7,16]diazacyclooctadecin-18-yl)methyl acetate (8.0 g).

Step 9: A stir bar, (tetradecahydro-2H,11H,17H-cyclopenta[b][1,4,10,13]tetraoxa[7,16]diazacyclooctadecin-18-yl)methyl acetate (8.0 g, 21 mmol), methyl 6-(chloromethyl)picolinate (12.2 g, 53.2 mmol), Na₂CO₃ (11.1 g, 106 mmol) and acetonitrile (100 mL) were added to a 500 mL three-neck round-bottomed flask under a nitrogen atmosphere, and the resultant heterogeneous mixture heated at 90° C. for 16 h under a nitrogen atmosphere. The resulting mixture was then cooled to room temperature, filtered through a pad of Celite®, and the filtrate concentrated to dryness in vacuo to yield a concentrate. The concentrate was subjected to silica gel chromatography (0-10% MeOH/DCM) to yield dimethyl 6,6′-((18-(acetoxymethyl)tetradecahydro-4H,13H,17H-cyclopenta[b][1,4,10,13]tetraoxa[7,16]diazacyclooctadecine-4,13-diyl)bis(methylene))dipicolinate (5.0 g).

Step 10: A stir bar, dimethyl 6,6′-((18-(acetoxymethyl)tetradecahydro-4H,13H,17H-cyclopenta[b][1,4,10,13]tetraoxa[7,16]diazacyclooctadecine-4,13-diyl)bis(methylene))dipicolinate (5.0 g, 7.4 mmol), K₂CO₃ (0.10 g, 0.74 mmol) and methanol (50 mL) were added to a 250 mL round-bottomed flask under nitrogen atmosphere, and the resulting mixture was stirred at room temperature for 10 min. The mixture was then concentrated to dryness in vacuo and the resulting residue purified by silica gel chromatography (0-10% MeOH/DCM) to yield 6,6′-((18-(hydroxymethyl)tetradecahydro-4H,13H,17H-cyclopenta[b][1,4,10,13]tetraoxa[7,16]diazacyclooctadecine-4,13-diyl)bis(methylene))dipicolinate (3.0 g).

Step 11: A stir bar, 6,6′-((18-(hydroxymethyl)tetradecahydro-4H,13H,17H-cyclopenta[b][1,4,10,13]tetraoxa[7,16]diazacyclooctadecine-4,13-diyl)bis(methylene))dipicolinate (2.0 g, 3.1 mmol), DCM (20 mL) and triethylamine (1.2 g, 9.5 mmol) were added to a 100 mL three-neck round-bottomed flask under a nitrogen atmosphere, and the resulting mixture cooled to 10° C. The mixture was treated with MsCl (0.48 g, 6.3 mmol) portion wise, and once addition was complete, the reaction vessel was brought to room temperature and stirred for 30 minutes, during which time a precipitate formed. The heterogeneous mixture was then diluted with DCM (50 mL), washed with cold aq. HCl (1 M, 50 mL×3) and ice-cold water (50 mL×2), dried over anhydrous Na₂SO₄, filtered, and concentrated to dryness to yield a gummy solid. The gummy solid was purified by neutral alumina column chromatography (0-10% MeOH/DCM) to yield dimethyl 6,6′-((18-(((methylsulfonyl)oxy)methyl)tetradecahydro-4H,13H,17H-cyclopenta[b][1,4,10,13]tetraoxa[7,16]diazacyclooctadecine-4,13-diyl)bis(methylene))dipicolinate (1.5 g).

Step 12: A solution consisting of dimethyl 6,6′-((18-(((methylsulfonyl)oxy)methyl)tetradecahydro-4H,13H,17H-cyclopenta[b][1,4,10,13]tetraoxa[7,16]diazacyclooctadecine-4,13-diyl)bis(methylene))dipicolinate (0.69 g, 3.2 mmol) and DMF (5 mL) was added dropwise over 5 minutes to a 25 mL three-neck round-bottomed flask containing a suspension of sodium hydride (162 mg, 60% in mineral oil, 4.22 mmol) in DMF (0.5 mL), at 0° C. under nitrogen atmosphere. Once addition was complete, the reaction mixture was brought to room temperature and stirred 15 minutes. The reaction mixture was then re-cooled to 0° C. and treated dropwise over 5 minutes with a solution consisting of tert-butyl (2-(2-mercaptoethoxy)ethyl)carbamate (1.50 g, 2.11 mmol) and DMF (3 mL). Once addition was complete, the reaction mixture was slowly warmed to room temperature and then stirred for 1 h. The reaction was then slowly treated with sat. NH₄Cl and subsequently extracted with ethyl acetate (10 mL×3). The combined extracts were washed with water (10 mL), dried over anhydrous Na₂SO₄, filtered, and concentrated to dryness to yield an oil. The oil was purified via preparative HPLC (Column: XBRIDGE C18 19×150 m) 5.0 μm; Mobile phase: 0.1% TFA in water/acetonitrile; Flow Rate: 15.0 mL/min) to yield cyclopenta[b][1,4,10,13]tetraoxa[7,16]diazacyclooctadecine-4,13-diyl)bis(methylene))dipicolinate (0.2 g).

Step 13: A stir bar, cyclopenta[b][1,4,10,13]tetraoxa[7,16]diazacyclooctadecine-4,13-diyl)bis(methylene))dipicolinate (0.20 g, 0.24 mmol), MeOH (1.0 mL), and HCl in methanol (4 M, 1.2 mL, 4.8 mmol) were added to a 25 mL single-neck round-bottomed flask at 0° C. and the resulting mixture brought to room temperature, and stirred for 2 h. The volatiles were removed in vacuo to yield dimethyl 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinate (150 mg), which was used without purification.

Step 14: A stir bar, dimethyl 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinate (40 mg, 0.054 mmol), aqueous LiOH (1.6 mL, 0.1 N, 0.16 mmol) and MeOH (0.5 mL) were added to an 8 mL reaction vial at room temperature and the resulting mixture was stirred overnight. The pH of the reaction mixture was adjusted with acetic acid to pH˜6.5 and then concentrated to dryness in vacuo at room temperature, and the resultant concentrate was purified by preparative HPLC (Column: XBRIDGE C18 19×150 mm 5.0 μm; Mobile phase: 10 Mm Ammonium Acetate in water/ACN; Flow Rate: 15.0 mL/min) to yield Example 11: 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinic acid (23 mg). LC-MS APCI: Calculated for C₃₄H₅₁N₅O_{9S}; 705.34; Observed m/z [M+H]>706.4.

Step 15: A stir bar, dimethyl 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinate (70 mg, 0.95 mmol), 11,12-Didehydro-γ-oxodibenz[b,f]azocine-5(6H)-butanoic acid (29 mg, 0.95 mmol), triethylamine (29 mg, 0.76 mmol), HATU (54 mg, 0.14 mmol) and DCM (0.5 mL) were added to a 25 mL three-neck round-bottomed flask at 0° C. under a nitrogen atmosphere. The resulting mixture was brought to room temperature and stirred overnight. The reaction mixture was diluted with water (10 mL) and the extracted with dichloromethane (10 mL×3). The combined extracts were washed with 10% aqueous NaHCO₃ (10 mL) and brine (10 mL), dried over anhydrous Na₂SO₄, filtered, and concentrated to dryness to yield an oil. The oil was purified via silica gel chromatography (0-10% MeOH/DCM) to yield N-acyl-DBCO tagged dimethyl 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinate (10 mg).

Step 16: A stir bar, N-acyl-DBCO tagged dimethyl 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinate (10 mg, 0.01 mmol), aqueous LiOH (0.3 mL, 0.1 N, 0.03 mmol) and methanol (0.25 mL) were added to an 8 mL reaction vial at room temperature and the resultant mixture stirred overnight. The reaction mixture was adjusted to pH-6.5 with acetic acid, concentrated to dryness in vacuo at room temperature, and the resultant concentrate was purified by preparative HPLC (Column: XBRIDGE C18 (19×150 mm) 5.0 μm; Mobile phase: 10 Mm Ammonium Acetate in water/ACN; Flow Rate: 15.0 mL/min) to yield Example 12: N-acyl-DBCO tagged 6,6′-((18-(((2-(2-aminoethoxy)ethyl)thio)methyl)tetradecahydro-4H,13H,17H-cyclopenta[b][1,4,10,13]tetraoxa[7,16]diazacyclooctadecine-4,13-diyl)bis(methylene))dipicolinic acid (3 mg). LC-MS APCI: Calculated for C₅₃H₆₄N₆O₁₁S; 992.44; Observed m/z [M−H]⁻: 991.4.

### Example 13

### 6-((16-(1-(6-carboxypyridin-2-yl)-8-isothiocyanatooctyl)-1,4,10,13-tetraoxa-7,16-diazacyclooctadecan-7-yl)methyl)picolinic acid

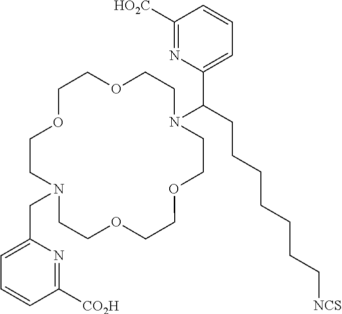

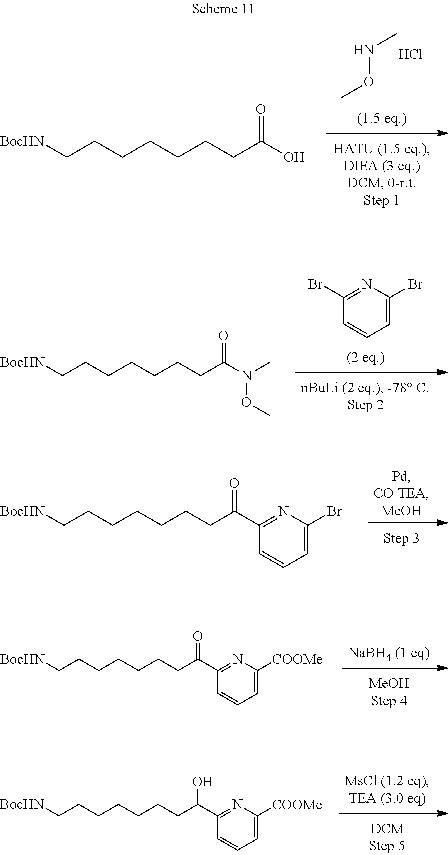

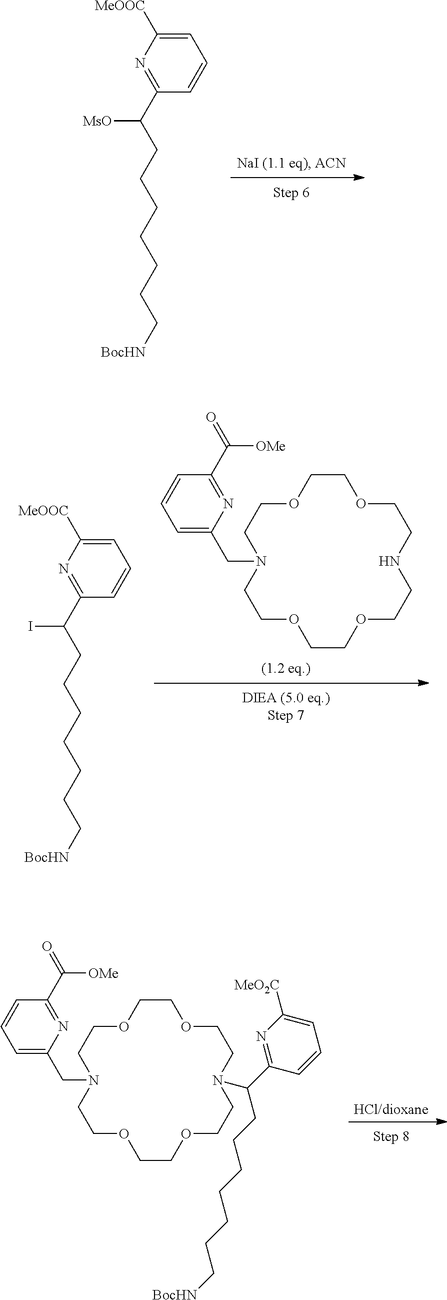

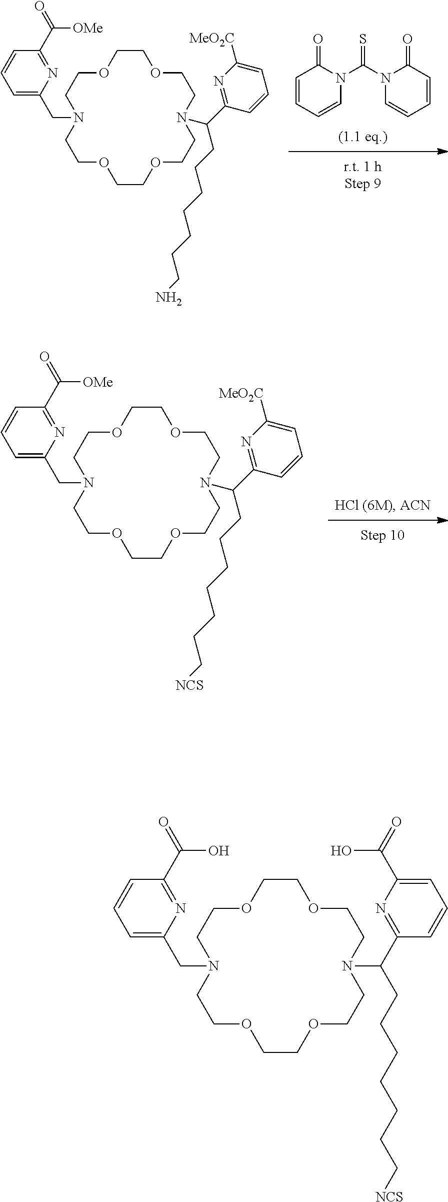

Step 1: Into a 500-mL 3-necked round-bottom flask, purged and maintained under an inert atmosphere of nitrogen, was placed a solution of 8-((tert-butoxycarbonyl)amino)octanoic acid (20.0 g, 77.1 mmol) in dichloromethane (200 mL), N, O-dimethylhydroxylamine (7.0 g, 115 mmol), diisopropylethylamine (29.90 g, 231 mmol). This was followed by the addition of HATU (43.9 g, 115 mmol) with stirring at 0° C. The resulting solution was stirred for 1 h. at room temperature. The reaction was then quenched by the addition of 200 mL of water. The resulting solution was extracted with dichloromethane (100 mL×2). The combined organic layers were washed sequentially with HCl (1 M) (300 mL×2), NH₄CO₃ aqueous solution (400 mL×3) and bine (400 mL). After it was dried over anhydrous Na₂SO₄, it was concentrated to give tert-butyl (8-(methoxy(methyl)amino)-8-oxooctyl)carbamate (15.4 g, 66% yield) as light-yellow oil.

Step 2: Into a 500-mL 3-necked round-bottom flask, purged and maintained under an inert atmosphere of nitrogen, was placed a solution of 2,6-dibromopyridine (23.0 g, 927 mmol) in THF (400 mL). It was cooled to −78° C. and n-BuLi (60.4 mL, 927 mmol) was added dropwise quickly.

After stirring for 10 min, an addition of tert-butyl (8-(methoxy(methyl)amino)-8-oxooctyl)carbamate (14.0 g, 463.5 mmol) in THF (40 mL) was added dropwise with stirring at −78° C. The resulting solution was stirred for 30 min. at room temperature. The reaction was quenched by the addition of 500 mL of water. The resulting solution was extracted with ethyl acetate (200 mL×2). The combined organic layers were washed with brine (400 mL), dried over anhydrous sodium sulfate and concentrated to give the crude product. Chromatography on silica gel ((0-10% ethyl acetate in petroleum ether) gave tert-butyl (8-(6-bromopyridin-2-yl)-8-oxooctyl)carbamate (11.8 g, 50% yield) as light yellow solid.

Step 3: Into a 1-L high pressure reactor, maintained with an inert atmosphere of nitrogen, was placed a solution of tert-butyl (8-(6-bromopyridin-2-yl)-8-oxooctyl)carbamate (11.5 g, 28.8 mmol, 1.0 eq.) in MeOH (500 mL), followed by Pd(dppf)Cl₂ (2.1 g, 2.88 mmol), TEA (8.7 g, 86.4 mmol). Then CO (20 atm) was introduced in. The resulting solution was stirred for 16 h at 100° C. The reaction solution was filtered and used for next step directly.

Step 4: The MeOH solution received from above was cooled to 0° C. and NaBH₄ (1.08 g, 28.8 mmol) was added. The resulting solution was stirred for 1 h. at room temperature. The reaction was quenched by the addition of 500 mL of NH₄CO₃ aqueous solution and extracted with ethyl acetate (300 mL×2). The combined organic layers were washed with brine (600 mL), dried over anhydrous Na₂SO₄ and concentrated to give methyl 6-(8-((tert-butoxycarbonyl)amino)-1-hydroxyoctyl)picolinate (10 g) as brown oil.

Step 5: Into a 250-mL 3-necked round-bottom flask, purged and maintained under an inert atmosphere of nitrogen, was placed a solution of methyl 6-(8-((tert-butoxycarbonyl)amino)-1-hydroxyoctyl)picolinate (10 g) in DCM (100 mL). After it was cooled to 0° C., TEA (7.9 g, 78.9 mmol) and mesyl chloride (3.6 g, 31.5 mmol) were added. The resulting solution was stirred for 1 h. at room temperature. The mixture was concentrated under vacuum. MeCN (100 mL) was added and concentrated under vacuum. The crude product methyl 6-(8-((tert-butoxycarbonyl)amino)-1-((methylsulfonyl)oxy)octyl)picolinate went straight to the next step.

Step 6: To a solution of the above crude product methyl 6-(8-((tert-butoxycarbonyl)amino)-1-((methylsulfonyl)oxy)octyl)picolinate in ACN (100 mL) was added NaI (4.3 g, 28.9 mmol). The resulting solution was stirred for 1 h at 80° C. The mixture was filtered and concentrated. The crude product was purified by Flash-Prep-HPLC: Column C18; mobile phase, H₂O/ACN=50/50% to H₂O/ACN=20/80% in 30 min; It gave 4 g of methyl 6-(8-((tert-butoxycarbonyl)amino)-1-iodooctyl)picolinate as brown oil.

Step 7: To a solution of methyl 6-(8-((tert-butoxycarbonyl)amino)-1-iodooctyl)picolinate (3.0 g, 6.12 mmol,) in DCM (200 mL) were added methyl 6-((1,4,10,13-tetraoxa-7,16-diazacyclooctadecan-7-yl)methyl)picolinate (3.0 g, 7.34 mmol), diisopropylethylamine (3.9 g, 30.61 mmol). The resulting solution was stirred for 16 h at 80° C. The reaction was concentrated. The crude product was purified by Flash-Prep-HPLC: Column C18; mobile phase, A: H₂O (0.05% TFA), B: CAN; 20% B to 40% B in 20 min. It gave 1.9 g of methyl 6-(8-((tert-butoxycarbonyl)amino)-1-(16-((6-(methoxycarbonyl)pyridin-2-yl)methyl)-1,4,10,13-tetraoxa-7,16-diazacyclooctadecan-7-yl)octyl)picolinate as brown oil.

Step 8: To a stirred solution of methyl 6-(8-((tert-butoxycarbonyl)amino)-1-(16-((6-(methoxycarbonyl)pyridin-2-yl)methyl)-1,4,10,13-tetraoxa-7,16-diazacyclooctadecan-7-yl)octyl)picolinate (1.7 g, 2.19 mmol, 77% on LCMS) in DCM (8.5 mL) at 0° C. was added HCl/dioxane dropwise. The resulting solution was stirred for 1 h. at room temperature. The reaction was quenched by the portion wise addition of NH₄CO₃ aqueous solution (20 mL×3). The resulting solution was extracted with dichloromethane (100 mL×2). The combined organic layers were washed brine (400 mL), dried over anhydrous Na₂SO₄ and concentrated to give 1.3 g of methyl 6-(8-amino-1-(16-((6-(methoxycarbonyl)pyridin-2-yl)methyl)-1,4,10,13-tetraoxa-7,16-diazacyclooctadecan-7-yl)octyl)picolinate as brown oil.

Step 9: To a solution of methyl 6-(8-amino-1-(16-((6-(methoxycarbonyl)pyridin-2-yl)methyl)-1,4,10,13-tetraoxa-7,16-diazacyclooctadecan-7-yl)octyl)picolinate (1.0 g, 1.48 mmol) in DCM (17 mL) under N₂ was added 1,1′-thiocarbonylbis(pyridin-2(1H)-one) (0.38 g, 1.63 mmol). The resulting solution was stirred for 1 h at room temperature. It was concentrated to give 1.6 g of methyl 6-((16-(1-(6-(methoxycarbonyl)pyridin-2-yl)-8-thiocyanatooctyl)-1,4,10,13-tetraoxa-7,16-diazacyclooctadecan-7-yl)methyl)picolinate as brown oil.

Step 10: To a solution of methyl 6-((16-(1-(6-(methoxycarbonyl)pyridin-2-yl)-8-thiocyanatooctyl)-1,4,10,13-tetraoxa-7,16-diazacyclooctadecan-7-yl)methyl)picolinate (1.40 g, 1.42 mmol) in ACN (4 mL) was added HCl (6 M) (7 mL). The resulting solution was stirred for 5 h at 50° C. in an oil bath. It was diluted with 10 mL of H₂O. The crude product was purified by Flash-Prep-HPLC: Column, C18; mobile phase, A: H₂O (0.05% TFA), B: ACN, 20% B to 36% B in 20 min; Detector UV@210 nm. The product fractions were concentrated to remove ACN. The aqueous was adjust to pH to 7˜8 with NaHCO₃ aqueous solution. It was purified again on Flash-Prep-HPLC: Column, C18; mobile phase, A: H₂O, B: ACN, 95% B to 100% B in 20 min.

The product solution was concentrated to remove CAN and then lyophilized. It gave 190 mg of 6-((16-(1-(6-carboxypyridin-2-yl)-8-thiocyanatooctyl)-1,4,10,13-tetraoxa-7,16-diazacyclooctadecan-7-yl)methyl)picolinic acid as a brown solid. ¹H NMR (300 MHz, D20) δ 7.91 (s, 4H), 7.54 (s, 2H), 4.52 (d, J=17.9 Hz, 3H), 3.77 (d, J=9.3 Hz, 8H), 3.56-3.41 (m, 18H), 2.11 (s, 2H), 1.51 (s, 2H), 1.17 (s, 7H), 0.97 (s, 1H). MS (ES, m z): 688.3 (M+H+).

### Example 14

### 6-((16-(1-(6-carboxypyridin-2-yl)-2-(2-(2-isothiocyanatoethoxy)ethoxy)ethyl)-1,4,10,13-tetraoxa-7,16-diazacyclooctadecan-7-yl)methyl)picolinic acid

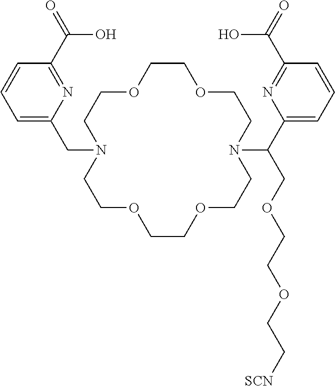

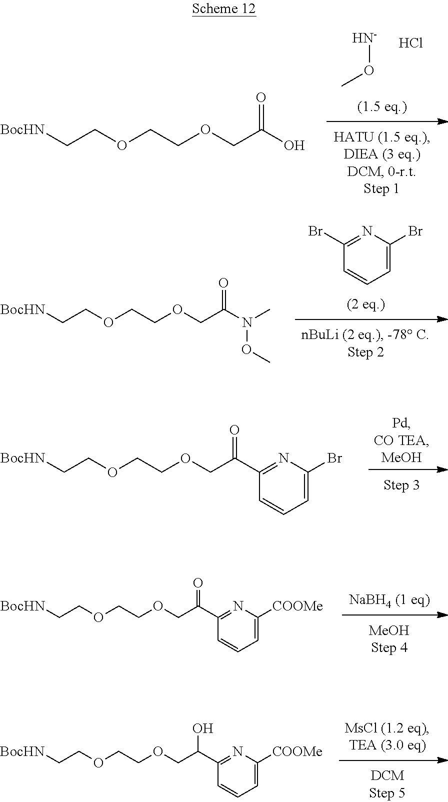

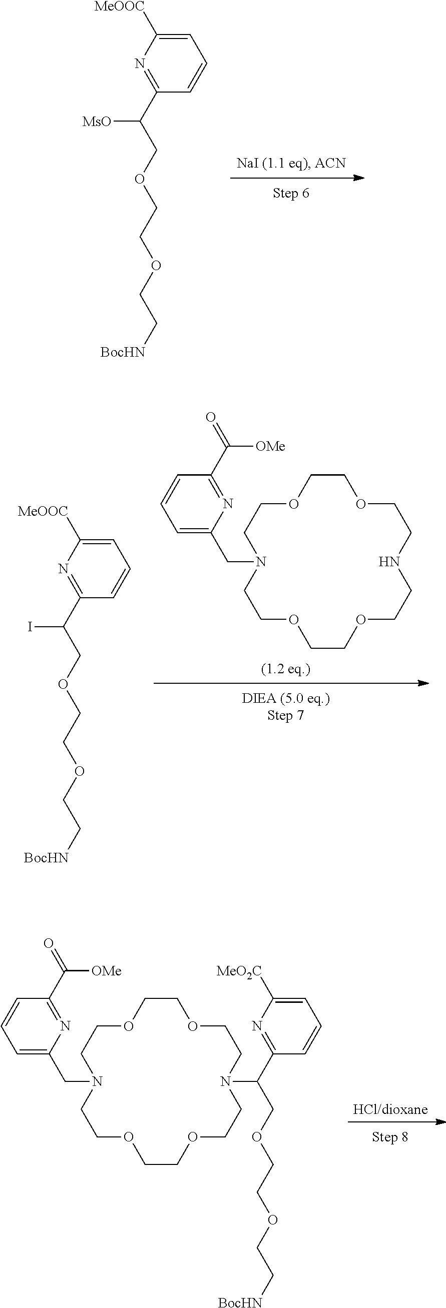

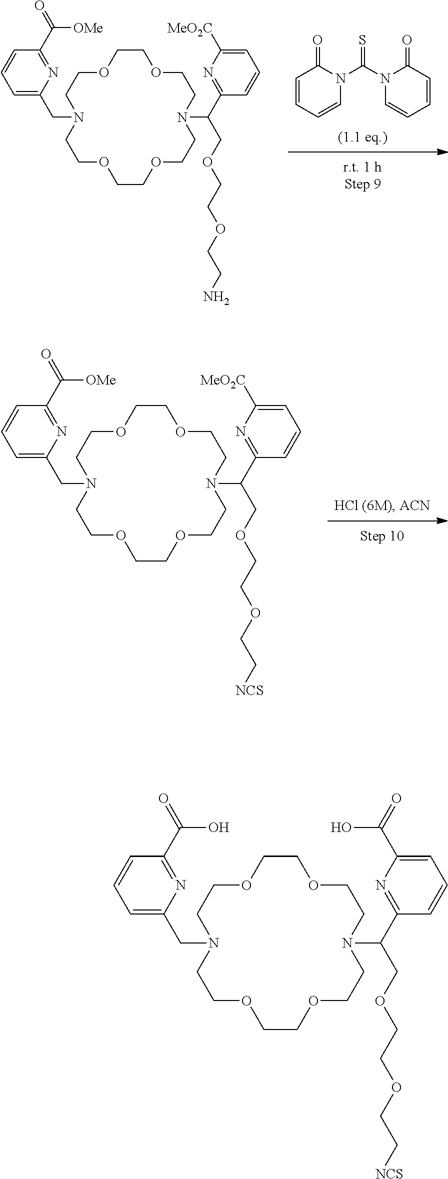

Step 1: To a stirred solution of 2,2-dimethyl-4-oxo-3,8,11-trioxa-5-azatridecan-13-oic acid (10.00 g, 37.98 mmol) and diisopropylethylamine (14.73 g, 113.94 mmol) in dichloromethane (100 mL) at 0° C. under nitrogen atmosphere was added [Bis(dimethylamino)methylene]-1H-1,2,3-triazolo[4,5-b]pyridinium 3-oxide hexafluorophosphate (15.16 g, 39.88 mmol), N,O-dimethyl hydroxylamine (5.55 g, 56.97 mmol) dropwise. After the resulting mixture was stirred for 1 h at room temperature, it was poured to saturated NH₄Cl (aq.). The resulting mixture was extracted with dichloromethane (100 mL×2). The combined organic layers were washed with brine, dried over anhydrous Na₂SO₄. After filtration, the filtrate was concentrated under reduced pressure. The residue was purified by chromatography: Column, C18; mobile phase A: H₂O with 0.05% TFA, B: ACN; gradient 20% B to 40% B in 20 minutes; Detector: UV@210 nm. It gave tert-butyl (3-methyl-4-oxo-2,6,9-trioxa-3-azaundecan-11-yl)carbamate (9.90 g. 85% yield) as light yellow oil.

Step 2: To a solution of 2,6-dibromo-pyridine (13.3 g, 56.1 mmol) in THF (260 mL) in a 500 ml 3-necked round-bottom flask at −78° C. under nitrogen atmosphere was added n-BuLi (28.0 mL, 56.1 mmol) dropwise. The solution was stirred at −78° C. for 10 min. A solution of tert-butyl (3-methyl-4-oxo-2,6,9-trioxa-3-azaundecan-11-yl)carbamate (7.0 g, 28.0 mmol) in THF (30 mL) was added dropwise to the reaction solution at −78° C. and the mixture was stirred at room temperature for 30 min. The reaction was quenched by the addition of water/ice (200 mL) at 0° C. The aqueous layer was extracted with ethyl acetate (100 mL×3). The combined extracts were dried over Na₂SO₄ and concentrated under vacuum. The residue was purified by chromatography: Column, C18; mobile phase, mobile phase A: H₂O with 0.05% TFA, B: ACN; gradient 38% B to 58% B in 20 minutes; Detector: UV@210 nm. It gave to tert-butyl (2-(2-(2-(6-bromopyridin-2-yl)-2-oxoethoxy)ethoxy)ethyl)carbamate (4.6 g, 50% yield) as a yellow solid. MS (ES, m z): 425, 427 (M+Na⁺).

Step 3: To a 250-mL high pressure reactor were added tert-butyl (2-(2-(2-(6-bromopyridin-2-yl)-2-oxoethoxy)ethoxy)ethyl)carbamate (4.0 g, 18.1 mmol), triethylamine (5.5 g, 54.3 mmol), Pd(dppf)Cl₂ (1.3 g, 1.8 mmol) and MeOH (40 mL). The reaction solution was evacuated and backfilled with N₂. Then CO (10 atm) was introduced in. The resulting solution was stirred at 100° C. for overnight. The reaction mixture was filtered, and the filtrate was concentrated to dryness. The residue was purified by chromatography: Column, C18; mobile phase A: H₂O with 0.05% TFA, B: ACN; gradient 38% B to 58% B in 20 minutes; Detector: UV@210 nm. It gave methyl 6-(2,2-dimethyl-4-oxo-3,8,11-trioxa-5-azatridecan-13-oyl)picolinate (2.4 g, 63% yield) as a brown oil. MS (ES, m z): 405 (M+Na⁺).

Step 4: To a solution of methyl 6-(2,2-dimethyl-4-oxo-3,8,11-trioxa-5-azatridecan-13-oyl)picolinate (2.30 g, 6.01 mmol) in MeOH (46 mL) under N₂ atmosphere at 0° C. was added NaBH₄ (0.23 g, 6.01 mmol). The resulting solution was stirred for 1 h at room temperature and quenched by the addition of 50 mL of saturated NH₄HCO₃ (aq.). The resulting solution was extracted with ethyl acetate (30 mL×2). The combined organic layers were washed with brine (60 mL), dried over Na₂SO₄ and concentrated. It gave 2.2 g of the crude product methyl 6-(13-hydroxy-2,2-dimethyl-4-oxo-3,8,11-trioxa-5-azatridecan-13-yl)picolinate as brown oil.

Step 5: To a solution of methyl 6-(13-hydroxy-2,2-dimethyl-4-oxo-3,8,11-trioxa-5-azatridecan-13-yl)picolinate (2.2 g, 5.72 mmol) in dichloromethane (22 mL) at 0° C. under N₂ atmosphere were added triethylamine (1.74 g, 17.16 mmol) and MsCl (0.79 g, 6.86 mmol). The resulting solution was stirred for 1 h at room temperature and quenched with H₂O (22 mL). The resulting mixture was extracted with dichloromethane (20 mL×2). The combined organic layers were washed with brine (40 mL), dried over Na₂SO₄ and concentrated. It gave 2.2 g of the crude product methyl 6-(2,2-dimethyl-13-((methylsulfonyl)oxy)-4-oxo-3,8,11-trioxa-5-azatridecan-13-yl)picolinate as brown oil.

Step 6: To a solution of methyl 6-(2,2-dimethyl-13-((methylsulfonyl)oxy)-4-oxo-3,8,11-trioxa-5-azatridecan-13-yl)picolinate (2.2 g, 4.75 mmol) in ACN (22 mL) under N₂ atmosphere was added NaI (0.78 g, 5.23 mmol). The resulting solution was stirred for 1 h at 80° C. The mixture was filtered and concentrated. The crude product was purified by chromatography: Column, C18; mobile phase A: H₂O, B: ACN; gradient 50% B to 80% B in 30 min; Detector: UV@210 nm. It gave methyl 6-(13-iodo-2,2-dimethyl-4-oxo-3,8,11-trioxa-5-azatridecan-13-yl)picolinate (1.2 g) as brown oil. MS (ES, m z): 517 (M+Na⁺), 495 (M+H+).

Step 7: A solution of methyl 6-(13-iodo-2,2-dimethyl-4-oxo-3,8,11-trioxa-5-azatridecan-13-yl)picolinate (840 mg, 1.69 mmol) and methyl 6-((1,4,10,13-tetraoxa-7,16-diazacyclooctadecan-7-yl)methyl)picolinate (839 mg, 2.03 mmol) in ACN (16.8 mL) was stirred for overnight at 80° C. under nitrogen atmosphere. The cooled reaction mixture was filtered, and the filtrate was concentrated under reduced pressure. The crude product was purified by chromatography: Column, C18; mobile phase A: H₂O, B: ACN; gradient 40% B to 60% B in 20 min; Detector: UV@210 nm. It gave methyl 6-((16-(13-(6-(methoxycarbonyl)pyridin-2-yl)-2,2-dimethyl-4-oxo-3,8,11-trioxa-5-azatridecan-13-yl)-1,4,10,13-tetraoxa-7,16-diazacyclooctadecan-7-yl)methyl)picolinate (450 mg) as a brown oil. MS (ES, m z): 700 (M+Na⁺), 678 (M+H+).

Step 8: To a solution of methyl 6-((16-(13-(6-(methoxycarbonyl)pyridin-2-yl)-2,2-dimethyl-4-oxo-3,8,11-trioxa-5-azatridecan-13-yl)-1,4,10,13-tetraoxa-7,16-diazacyclooctadecan-7-yl)methyl)picolinate (450 mg, 579 mmol) in dichloromethane (2.5 mL) at 0° C. was added HCl/dioxane (2.5 ml, 4 M). The resulting solution was stirred for 20 min at room temperature. The reaction was quenched by the addition of saturated Na₂CO₃ (aq.). The aqueous layer was extracted with DCM:IPA (5:1) (30 mL×2). The combined organic layers were dried over anhydrous Na₂SO₄ and concentrated under reduced pressure to afford the crude product methyl 6-(2-(2-(2-aminoethoxy)ethoxy)-1-(16-((6-(methoxycarbonyl)pyridin-2-yl)methyl)-1,4,10,13-tetraoxa-7,16-diazacyclooctadecan-7-yl)ethyl)picolinate (330 mg). The crude product was used directly in the next step.

Step 9: A solution of methyl 6-(2-(2-(2-aminoethoxy)ethoxy)-1-(16-((6-(methoxycarbonyl)pyridin-2-yl)methyl)-1,4,10,13-tetraoxa-7,16-diazacyclooctadecan-7-yl)ethyl)picolinate (300 mg, 0.44 mmol) and 1-(2-oxopyridine-1-carbothioyl) pyridin-2-one (113.08 mg, 0.48 mmol) in dichloromethane (3 mL) was stirred for 1 h at room temperature under nitrogen atmosphere. The resulting mixture was concentrated under reduced pressure to afford the crude product methyl 6-(2-(2-(2-isothiocyanatoethoxy)ethoxy)-1-(16-((6-(methoxycarbonyl)pyridin-2-yl)methyl)-1,4,10,13-tetraoxa-7,16-diazacyclooctadecan-7-yl)ethyl)picolinate (380 mg). The crude product was used directly in the next step.

Step 10: A solution of methyl 6-(2-(2-(2-isothiocyanatoethoxy)ethoxy)-1-(16-((6-(methoxycarbonyl)pyridin-2-yl)methyl)-1,4,10,13-tetraoxa-7,16-diazacyclooctadecan-7-yl)ethyl)picolinate (380 mg, 0.52 mmol) and HCl (1.9 mL, 6 M) in dichloromethane (1.9 mL) was stirred for 3 h at 50° C. under nitrogen atmosphere. The resulting mixture was concentrated under reduced pressure and was basified to pH 6-7 with saturated NaHCO₃ (aq.). The residue was purified by chromatography: Column, C18; mobile phase A: H₂O with 0.05% TFA, B: ACN, gradient 20% B to 36% B in 20 min; Detector: UV@210 nm. Then, the product fractions were concentrated under vacuum to remove MeCN. The solution was purified again by chromatography: Column, C18; mobile phase A: H₂O, B: ACN, gradient 95% B to 100% B in 20 min. The solution was concentrated to remove most MeCN and the aqueous solution was lyophilized to give 6-((16-(1-(6-carboxypyridin-2-yl)-2-(2-(2-isothiocyanatoethoxy)ethoxy)ethyl)-1,4,10,13-tetraoxa-7,16-diazacyclooctadecan-7-yl)methyl)picolinic acid (130 mg) as brown solid. ¹H NMR (300 MHz, D20) 8.03-7.84 (m, 2H), 7.57 (dd, J=22.3, 7.4 Hz, 1H), 5.00 (s, OH), 4.59 (s, 1H), 4.20 (dd, J=23.4, 9.5 Hz, 1H), 3.82 (d, J=15.4 Hz, 4H), 3.70-3.58 (m, 6H), 3.58-3.49 (m, 6H). MS (ES, m z): 692.3 (M+H+).

### Example 15

### 6,6′-(((S)-2-(((5-(((((1R,8S,9r)-Bicyclo[6.1.0]non-4-yn-9-yl)methoxy)carbonyl)amino)pentyl)thio)methyl)-1,4,10,13-tetraoxa-7,16-diazacyclooctadecane-7,16-diyl)bis(methylene))dipicolinic acid

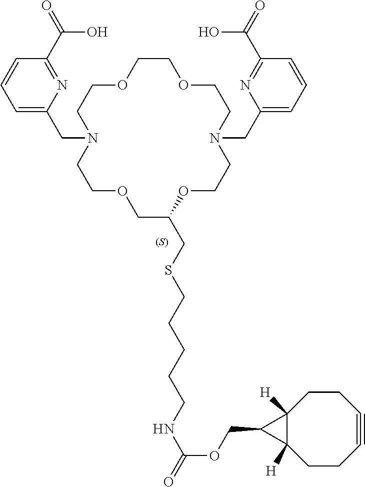

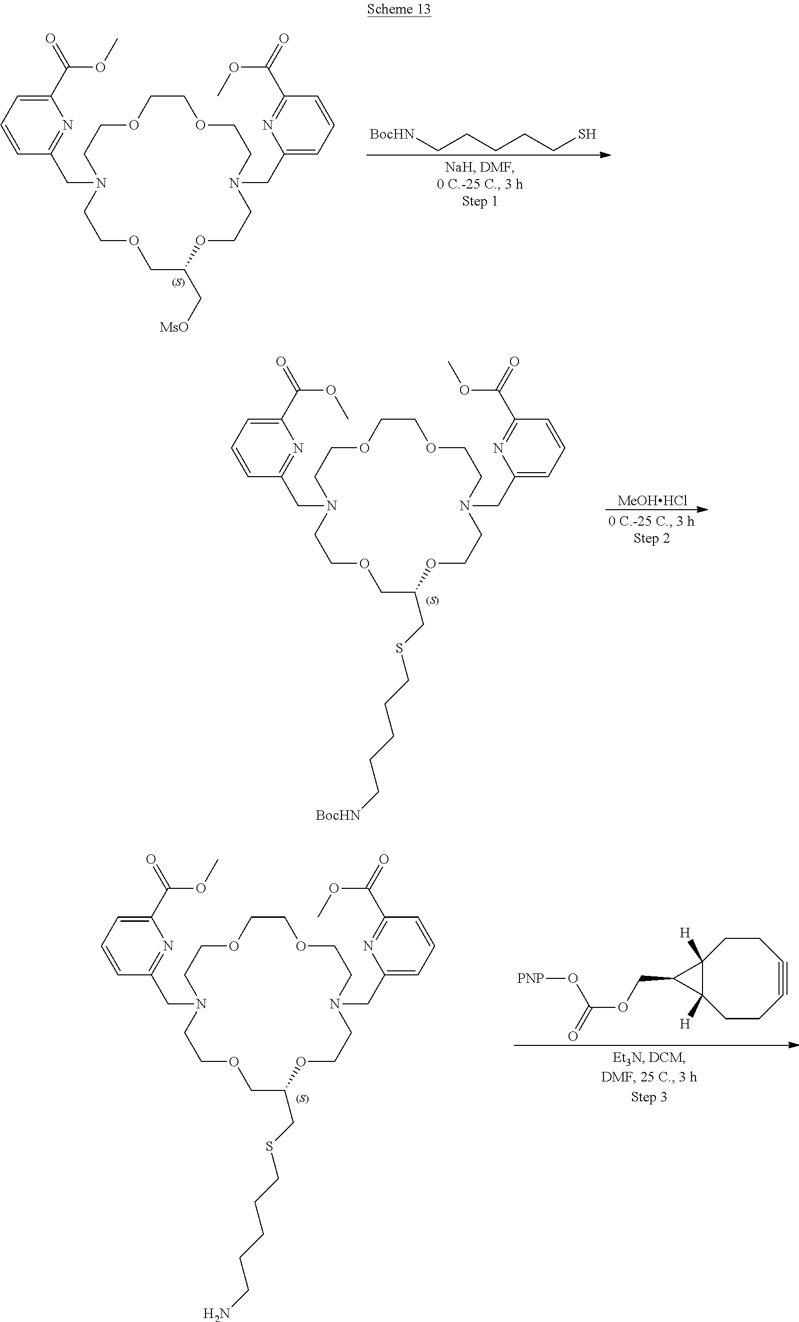

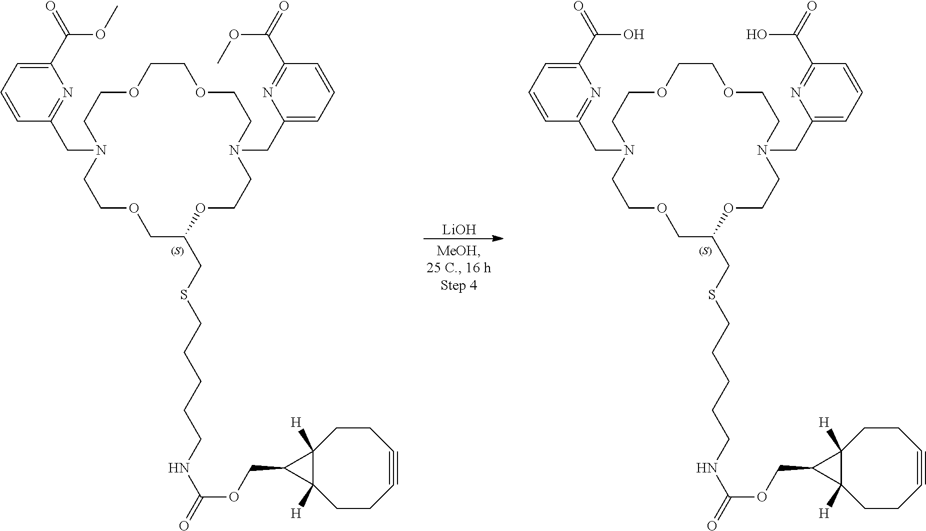

Step 1: A solution consisting of tert-butyl (5-mercaptopentyl)carbamate (0.30 g, 1.0 mmol) and DMF (3.0 mL) was added dropwise over 5 minutes to a 50 mL three-neck round-bottomed flask containing a suspension of sodium hydride (0.07 g, 60% in mineral oil, 2 mmol) in DMF (3.0 mL) at 0° C. and under a nitrogen atmosphere. Once addition was complete, the reaction mixture was brought to room temperature and stirring continued for 15 minutes. The reaction mixture was then re-cooled to 0° C. and treated dropwise over 10 minutes with a solution consisting of dimethyl 6,6′-((2-(((methylsulfonyl)oxy)methyl)-1,4,10,13-tetraoxa-7,16-diazacyclooctadecane-7,16-diyl)bis(methylene))(S)-dipicolinate (0.6 g, 0.9 mmol) and DMF (3.0 mL). Once addition was complete, the reaction mixture was slowly warmed to room temperature and stirring continued for 1.5 h. The reaction mixture was then carefully treated with sat. aqueous NH₄Cl (1.0 mL) and concentrated to dryness to give an oil. The oil was subjected to preparative HPLC (Column: XBRIDGE C18 19×150 mm, 5.0 μm; Mobile phase: 0.1% TFA in water/acetonitrile; Flow Rate: 15.0 mL/min) to yield dimethyl 6,6′-((2-(((5-((tert-butoxycarbonyl)amino)pentyl)thio)methyl)-1,4,10,13-tetraoxa-7,16-diazacyclooctadecane-7,16-diyl)bis(methylene))(S)-dipicolinate (0.25 g).

Step 2: A stir bar, dimethyl 6,6′-((2-(((5-((tert-butoxycarbonyl)amino)pentyl)thio)methyl)-1,4,10,13-tetraoxa-7,16-diazacyclooctadecane-7,16-diyl)bis(methylene))(S)-dipicolinate (0.25 g, 0.32 mmol), MeOH (1.0 mL), and HCl in methanol (4 M, 1.5 mL, 6.3 mmol) were added to a 25 mL round-bottomed flask at 0° C., which was subsequently brought to room temperature and the mixture stirred for 3 h. The volatiles were then removed in vacuo to yield dimethyl 6,6′-((2-(((5-aminopentyl)thio)methyl)-1,4,10,13-tetraoxa-7,16-diazacyclooctadecane-7,16-diyl)bis(methylene))(S)-dipicolinate (0.21 g), which was used without purification.

Step 3: A stir bar, dimethyl 6,6′-((2-(((5-aminopentyl)thio)methyl)-1,4,10,13-tetraoxa-7,16-diazacyclooctadecane-7,16-diyl)bis(methylene))(S)-dipicolinate (0.15 g, 0.22 mmol), ((1R,8S,9r)-bicyclo[6.1.0]non-4-yn-9-yl)methyl 4-nitrophenyl carbonate (68 mg, 0.22 mmol), triethylamine (66 mg, 0.65 mmol), and a mixture of DCM (2 mL) and DMF (0.1 mL) were added to a 25 mL three-neck round-bottomed flask at 0° C. under a nitrogen atmosphere. The resultant mixture was gradually warmed to room temperature and stirred overnight. The mixture was then concentrated to dryness to give dimethyl 6,6′-(((S)-2-(((5-(((((1R,8S,9r)-bicyclo[6.1.0]non-4-yn-9-yl)methoxy)carbonyl)amino)pentyl)thio)methyl)-1,4,10,13-tetraoxa-7,16-diazacyclooctadecane-7,16-diyl)bis(methylene))dipicolinate (0.1 g), which was used without purification.

Step 4: A stir bar, dimethyl 6,6′-(((S)-2-(((5-(((((1R,8S,9r)-bicyclo[6.1.0]non-4-yn-9-yl)methoxy)carbonyl)amino)pentyl)thio)methyl)-1,4,10,13-tetraoxa-7,16-diazacyclooctadecane-7,16-diyl)bis(methylene))dipicolinate (0.10 g, 0.12 mmol), aqueous LiOH (3.5 mL, 0.1 N, 0.35 mmol), and MeOH (0.5 mL) were added to a 8 mL reaction vial and the resultant mixture stirred overnight at room temperature. The reaction mixture was then treated with acetic acid until pH˜6.5, and subsequently concentrated to dryness in vacuo at room temperature to yield an oil, which was purified via preparative HPLC (Column: XBRIDGE C18 19×150 mm, 5.0 μm; Mobile phase: 0.1% Formic acid in H₂O/ACN; Flow Rate: 15.0 mL/min) to yield 6,6′-(((S)-2-(((5-(((((1R, 8S,9r)-bicyclo[6.1.0]non-4-yn-9-yl)methoxy)carbonyl)amino)pentyl)thio)methyl)-1,4,10,13-tetraoxa-7,16-diazacyclooctadecane-7,16-diyl)bis(methylene))dipicolinic acid (42 mg).

### Example 16

### N-acyl-DBCO tagged 6,6′-((2-(((5-Aminopentyl)thio)methyl)-1,4,10,13-tetraoxa-7,16-diazacyclooctadecane-7,16-diyl)bis(methylene))(S)-dipicolinic acid

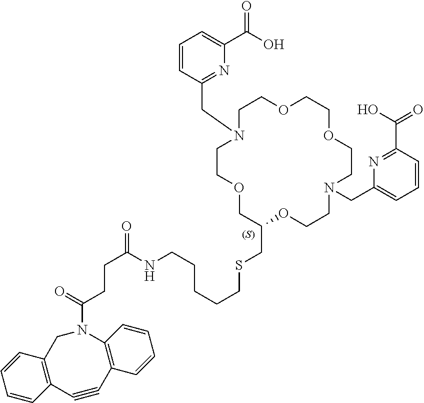

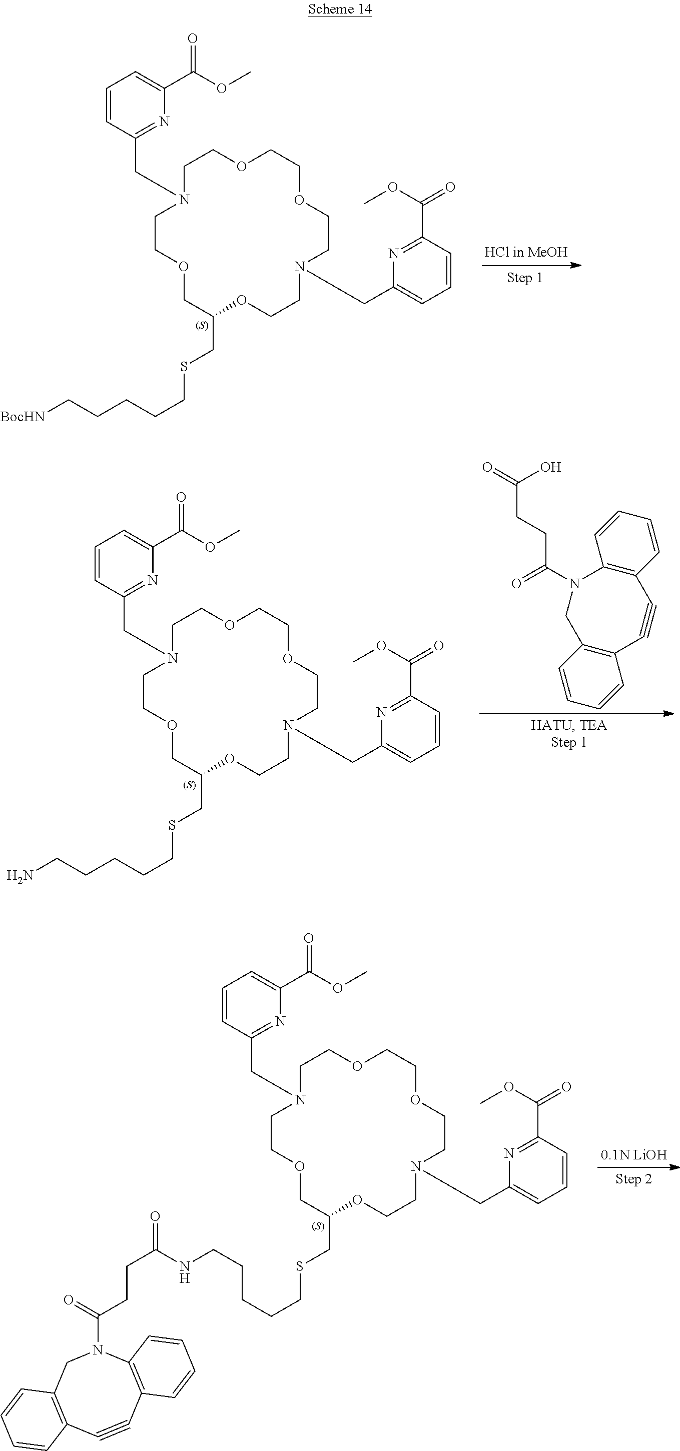

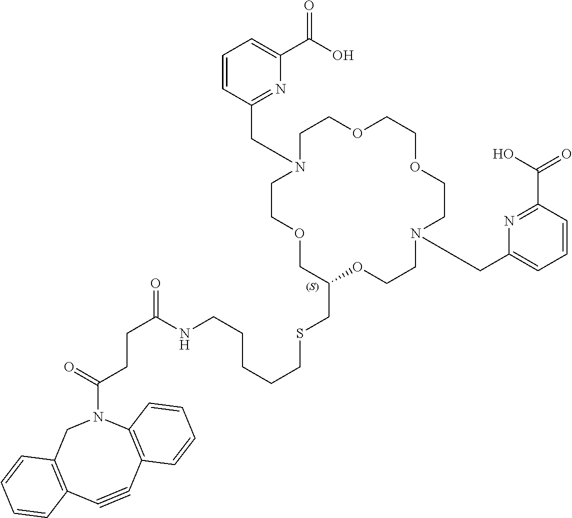

Step 1: A stir bar, dimethyl 6,6′-((2-(((5-((tert-butoxycarbonyl)amino)pentyl)thio)methyl)-1,4,10,13-tetraoxa-7,16-diazacyclooctadecane-7,16-diyl)bis(methylene))(S)-dipicolinate (0.12 g, 0.15 mmol), MeOH (0.5 mL), and HCl in methanol (4 M, 0.75 mL, 3.0 mmol) were added to a 25 mL round-bottomed flask at 0° C. and then brought to room temperature and stirred for 2 h. The volatiles were removed in vacuo to yield dimethyl 6,6′-((2-(((5-aminopentyl)thio)methyl)-1,4,10,13-tetraoxa-7,16-diazacyclooctadecane-7,16-diyl)bis(methylene))(S)-dipicolinate (70 mg), which was used without purification.

Step 2: A stir bar, dimethyl 6,6′-((2-(((5-aminopentyl)thio)methyl)-1,4,10,13-tetraoxa-7,16-diazacyclooctadecane-7,16-diyl)bis(methylene))(S)-dipicolinate (50 mg, 0.070 mmol), 11,12-Didehydro-γ-oxodibenz[b,f]azocine-5(6H)-butanoic acid (20 mg, 0.070 mmol), triethylamine (21 mg, 0.21 mmol), HATU (38 mg, 0.10 mmol), and DCM (0.5 mL) were added to a 25 mL three-neck round-bottomed flask at 0° C. under a nitrogen atmosphere, and subsequently brought to room temperature and stirred overnight. The reaction mixture was treated with water (10 mL) and extracted with dichloromethane (10 mL×3), and the combined extracts washed with 10% aqueous NaHCO₃(10 mL) and brine (10 mL), dried over anhydrous Na₂SO₄, filtered, and concentrated to dryness to yield an oil. The oil was purified via silica gel chromatography (0-10% MeOH/DCM) to yield N-acyl-DBCO tagged dimethyl 6,6′-((2-(((5-aminopentyl)thio)methyl)-1,4,10,13-tetraoxa-7,16-diazacyclooctadecane-7,16-diyl)bis(methylene))(S)-dipicolinate (16 mg).

Step 3: A stir bar, N-acyl-DBCO tagged dimethyl 6,6′-((2-(((5-aminopentyl)thio)methyl)-1,4,10,13-tetraoxa-7,16-diazacyclooctadecane-7,16-diyl)bis(methylene))(S)-dipicolinate (16 mg, 0.016 mmol), aqueous LiOH (0.49 mL, 0.1 N, 0.049 mmol), and MeOH (0.25 mL) were added to an 8 mL reaction vial and the mixture stirred at room temperature overnight. The reaction mixture was then treated with acetic acid until pH˜6.5, and concentrated to dryness in vacuo at room temperature to yield a concentrate, which was purified via preparative HPLC (Column: XBRIDGE C18 19×150 mm 5.0 μm; Mobile phase: 10 mM Ammonium Acetate in water/ACN; Flow Rate: 15.0 mL/min) to yield N-acyl-DBCO tagged 6,6′-((2-(((5-aminopentyl)thio)methyl)-1,4,10,13-tetraoxa-7,16-diazacyclooctadecane-7,16-diyl)bis(methylene))(S)-dipicolinic acid (5 mg) as an off-white solid. LC-MS APCI: Calculated for C₅₁H₆₂N₆O₁₀S; 950.42; Observed m/z [M+H]⁺ 951.4.

¹H NMR (400 MHz, D₂O): δ 7.81-7.75 (m, 4H), 7.52-7.17 (m, 10H), 4.97-4.90 (m, 1H), 4.80 (s, 4H), 4.14 (s, 3H), 3.77-3.46 (m, 16H), 3.10 (s, 7H), 2.83-2.80 (m, 2H), 2.55-2.53 (m, 2H), 2.42-2.38 (m, 3H), 2.11-2.08 (m, 3H), 1.39-1.35 (m, 2H), 1.20-1.10 (m, 4H).

### Example 17

### N-acyl-DBCO tagged 6-((4-((6-Aminoethyl)carbamoyl)phenyl)16-((6-carboxypyridin-2-yl)-1,4,10,13-tetraoxa-7,16-diazacyclooctadecan-7-yl)methyl)picolinic acid (TOPA-[C7]-benzimido-DBCO)

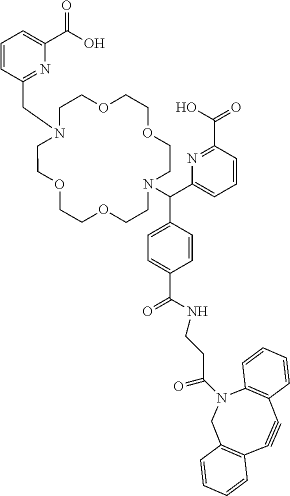 [SMILES: O=C(NCCC(=O)N1Cc2ccccc2C#Cc2ccccc21)c1ccc(C(c2cccc(C(=O)O)n2)N2CCOCCOCCN(Cc3cccc(C(=O)O)n3)CCOCCOCC2)cc1]

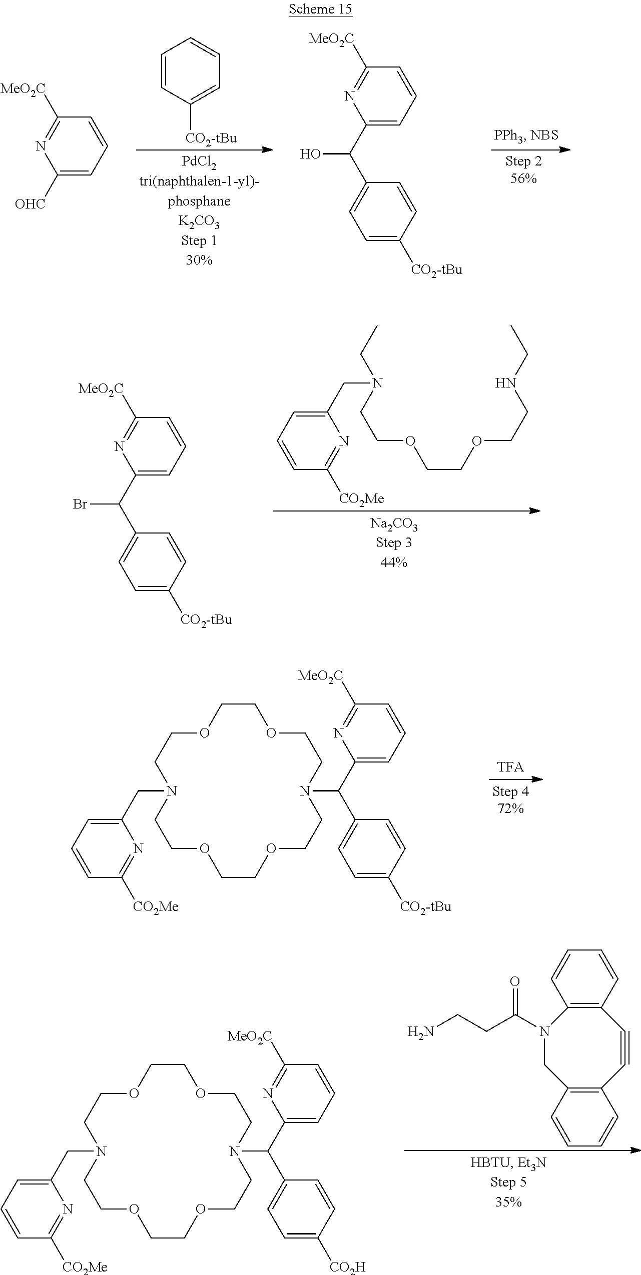

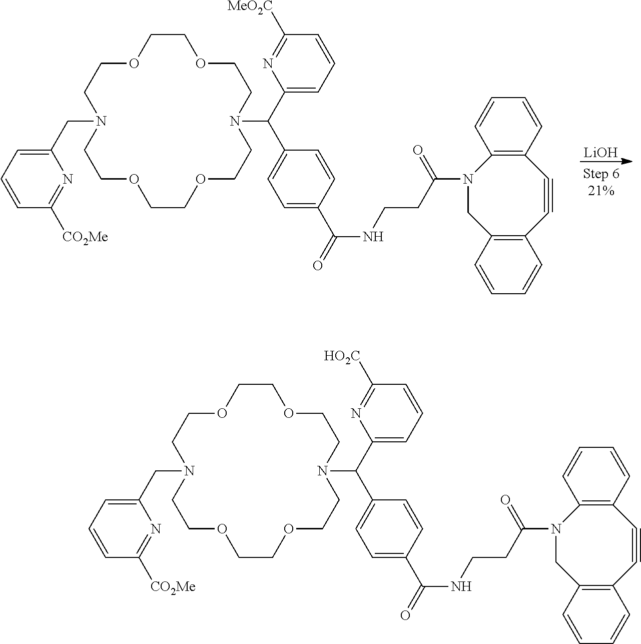

Step 1: To a mixture of methyl 6-formylpicolinate (4.00 g, 24.2 mmol), (4-(tert-butoxycarbonyl)phenyl)boronic acid (10.7 g, 48.5 mmol), PdCl2 (0.21 g, 1.2 mmol), tri(naphthalen-1-yl)phosphine (0.50 g, 1.2 mmol) and potassium carbonate (10.0 g, 72.7 mmol) under nitrogen at −78° C. in a 500 mL three neck round bottom flask was added tetrahydrofuran (100 mL) in one portion. The mixture was purged with nitrogen and stirred at r.t. for 30 min, then heated at 65° C. for 24 h. The reaction mixture was cooled r.t. and filtered through a pad of Celite® and the filtrate was concentrated to dryness. The crude product was subjected to silica gel chromatography (0-50% EtOAc/petether) to afford methyl 6-((4-(tert-butoxycarbonyl)phenyl)(hydroxy)methyl)picolinate as a yellow oil (2.5 g, 30%).

Step 2: A stir bar, methyl 6-((4-(tert-butoxycarbonyl)phenyl)(hydroxy)methyl)picolinate (2.50 g, 7.30 mmol), PPh₃ (3.43 g, 13.1 mmol), N-bromosuccinimide (2.13 g, 12.0 mmol) and DCM (30 mL) were taken in a 250 mL three neck round bottom flask under nitrogen atmosphere at r.t. and stirred for 1 h. The reaction solution was loaded onto a silica gel column and purified using 0-30% ethyl acetate in petroleum ether to get compound methyl 6-(bromo(4-(tert-butoxycarbonyl)phenyl)methyl)picolinate (1.65 g, 56%) as a yellow oil.

Step 3: A stir bar, methyl 6-((1,4,10,13-tetraoxa-7,16-diazacyclooctadecan-7-yl)methyl)picolinate (1.52 g, 3.69 mmol), 6-(bromo(4-(tert-butoxycarbonyl)phenyl)methyl)picolinate (1.50 g, 3.69 mmol), Na₂CO₃ (1.17 g, 11.1 mmol), and acetonitrile (30 mL) were added to a 250 mL three neck round-bottomed flask, and the resultant heterogeneous mixture was heated at 90° C. for 16 h under nitrogen atmosphere. Subsequently reaction mass was cooled to r.t., filtered through a pad of Celite®, and concentrated to dryness in vacuo to give the crude product. The crude product was subjected to silica gel chromatography (0-10% MeOH/DCM) to afford methyl 6-((4-(tert-butoxycarbonyl)phenyl)(16-((6-(methoxycarbonyl)pyridin-2-yl)methyl)-1,4,10,13-tetraoxa-7,16-diazacyclooctadecan-7-yl)methyl)picolinate as a brown oil (1.2 g, 44%).

Step 4: A stir bar, methyl 6-((4-(tert-butoxycarbonyl)phenyl)(16-((6-(methoxycarbonyl)pyridin-2-yl)methyl)-1,4,10,13-tetraoxa-7,16-diazacyclooctadecan-7-yl)methyl)picolinate (1.2 g, 1.6 mmol), TFA (0.62 mL, 8.1 mmol) and DCM (20 mL) were added to a 100 mL three neck round bottom flask at r.t. and stirred for 1 h. Reaction mixture was concentrated to dryness and the resultant crude product was subjected to preparative HPLC (Column: XBRIDGE C18 (19×150 mm) 5.0 μm; Mobile phase: 0.1% TFA in water/ACN; Flow Rate: 15.0 mL/min) to give 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.8 g, 72%) as brown oil. LC-MS APCI: Calculated for C₃₅H₄₄N₄O₁₀ 680.31; Observed m/z [M+H]⁺ 681.5. Purity by LC-MS: 99.87%. Purity by HPLC: 97.14% (97.01% at 210 nm, 97.20% at 254 nm and 97.21% at 280 nm; Column: Atlantis dC18 (250×4.6 mm), 5 μm; Mobile phase A: 0.1% TFA in water, Mobile phase B: acetonitrile; Flow rate: 1.0 mL/min.%. ¹H NMR (400 MHz, DMSO-d6): δ 8.12-8.07 (m, 4H), 8.00-7.98 (m, 2H), 7.75-7.73 (m, 4H), 6.10 (s, 1H), 4.67 (s, 2H), 3.96 (s, 3H), 3.91 (s, 3H), 3.82 (s, 8H), 3.56 (s, 8H), 3.52 (s, 8H).

Step 5: A stir bar, 4-((6-(methoxycarbonyl)pyridin-2-yl)(16-((6-(methoxycarbonyl)pyridin-2-yl)methyl)-1,4,10,13-tetraoxa-7,16-diazacyclooctadecan-7-yl)methyl)benzoic acid (0.25 g, 0.37 mmol), DBCO (0.10 g, 0.37 mmol), triethylamine (0.16 mL, 1.1 mmol), HBTU (0.21 g, 0.55 mmol) and DCM (10 mL) were added to a 25 mL three neck round-bottom flask at 0° C. under nitrogen atmosphere at r.t. and stirred for 16 h. The reaction was quenched with water (20 mL) and it was extracted with DCM (3×20 mL). The combined extracts were washed with 10% aqueous NaHCO₃ solution (20 mL), brine (20 mL), dried over anhydrous Na₂SO₄, filtered, and concentrated to dryness to afford the crude product as an oil. The crude product was subjected to silica gel chromatography (0-10% MeOH/DCM) to give TOPA dimethyl ester-[C7]-phenyl-DBCO (0.12 g, 35%) as a colorless gummy oil.

Step 6: A stir bar, TOPA dimethyl ester-[C7]-phenyl-DBCO (0.1 g, 0.1 mmol), aqueous LiOH.H₂O (3 mL, 0.1 N, 0.3 mmol) and THF/MeOH/H₂O (4:1:1 v/v/v, 2 mL) were added to a 8 mL reaction vial at r.t. and it was allowed to stir for 2 h. The reaction mixture was neutralized with aqueous HCl (1N) to P^{H}˜6.5. The reaction mixture was concentrated to dryness in vacuo at room temperature, and the resultant crude product was subjected to preparative HPLC (Column: XBRIDGE C18 (19×150 mm) 5.0 μm; Mobile phase: 10 Mm Ammonium Acetate in water/ACN; Flow Rate: 15.0 mL/min) to give TOPA-[C7]-phenyl-DBCO (20 mg, 21%) as an off-white solid. LC-MS APCI: Calculated for C₅₁H₅₄N₆O₁₀ 910.39; Observed m/z [M−H]⁺ 909.3. Purity by LC-MS: 92.47%. Purity by HPLC: 90.68% (88.04% at 210 nm, 90.43% at 254 nm and 93.56% at 280 nm; Column: XBRIDGE C8 (50×4.6 mm), 3.5 μm; Mobile phase A: 10 mM Ammonium bicarbonate in water, Mobile phase B: acetonitrile; Flow rate: 1.0 mL/min. ¹H NMR (400 MHz, DMSO-d6): δ 7.84-7.82 (m, 4H), 7.60-7.29 (m, 12H), 7.13-7.10 (m, 2H), 5.12-5.02 (m, 2H), 3.97 (s, 2H), 3.59-3.44 (m, 20H), 2.85 (s, 4H), 2.73-2.68 (m, 6H).

### Example 18

TOPA-[C7]-benzyimido-DBCO-triazole-PSMB-127 Antibody Conjugate

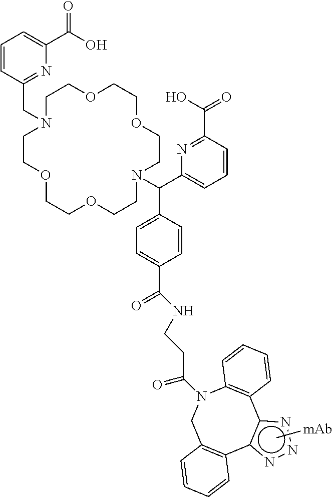

Step 1. Azide modification of mAb and Click reaction: PSMB127 was site-selectively modified with 100× molar excess of 3-azido propylamine and microbial transglutaminase (MTG; Activa TI) at 37° C. The addition of two azides on the heavy chains of the mAb was monitored by intact mass ESI-TOF LC-MS on an Agilent G224 instrument. Excess 3-azido propylamine and MTG was removed and azide modified mAb (azido-mAb) was purified using a 1 mL GE Healthcare MabSelect column. Azido-mAb is eluted from the resin using 100 mM sodium citrate pH 3.0 and subsequently exchanged into 20 mM Hepes, 100 mM NaCl pH 7.5 using 7K Zeba® desalting columns. 10× molar excess of TOPA-[C7]-phenyl-DBCO was reacted with site specific azide-PSMB127 (DOL=2) at 37° C. for 1 hour without shaking. Completion of the DBCO-azide click reaction was monitored by intact mass spectrometry. Excess free chelator was removed by desalting the conjugate over a Zeba®7K desalting column into 20 mM Hepes, 100 mM NaCl pH 7.5 followed by three sequential 15× dilution and concentration steps in 20 mM Hepes, 100 mM NaCl pH 7.5 using a 30K MWCO Amicon concentrator device by spinning at 3800×g. This provided the final site specific TOPA-[C7]-phenyl-DBCO-PSMB127 conjugate with CAR=2. The final conjugate was confirmed to be monomeric by analytical size exclusion chromatography on a Tosoh TSKgel G3000SWxl 7.8 mm×30 cm, 5 u column; column temperature: room temperature; the column was eluted with DPBS buffer (1×, without calcium and magnesium); flow rate: 0.7 mL/min; 18 min run; injection volume: 18 μL.

Step 2. Chelation: Stock solutions of the following metal salts were prepared in pure water:

| Salt | Catalog # | Concentration |
|---|---|---|
| Cerium (III) Chloride | Sigma Cat # 429406 | 10 mM |
| Neodymium (III) chloride hexahydrate | Sigma Cat # 289183 | 10 mM |
| Terbium (III) chloride | Sigma Cat # 451304 | 10 mM |
| Lutetium (III) chloride | Sigma Cat # 450960 | 10 mM |
| Thulium (III) chloride | Sigma Cat # 451304 | 10 mM |
| Yttrium (III) chloride | Sigma Cat # 451363 | 10 mM |
| Holmium (III) chloride | Sigma Cat # 450901 | 10 mM |

Metal solutions were added to the TOPA-[C7]-phenyl-DBCO-PSMB127 in 5× molar excess (6.8 uM antibody, 34 uM metal ion) in 10 mM sodium acetate buffer pH 5.2 and incubated for 2 hours at 37 C. Excess metal was removed by desalting with a Zeba® column (ThermoFisher®) followed by two cycles of 10× dilution and concentration in a 50K MWCO Amicon concentrator (EMD Millipore®). Chelation was assessed by intact and reduced mass LC-MS.

Step 3. Stability Determination: To determine stability of the chelate, DTPA challenge was performed. 50 uL of the sample (6.3 uM antibody) was combined with 50 uL of 10 mM DTPA pH 6.5 and incubated at 37 C overnight. Chelation was assessed by intact and reduced mass LC-MS. LC-MS was performed on an Agilent 1260 HPLC system connected to an Agilent G6224 MS-TOF Mass Spectrometer. LC was run on an Agilent RP-mAb C4 column (2.1×50 mm, 3.5 micron) at a flow rate of 1 mL/min with the mobile phase 0.1% formic acid in water (A) and 0.1% formic acid in acetonitrile (Sigma-Aldrich Cat #34688) (B) and a gradient of 20% B (0-2 min), 20-60% B (2-3 min), 60-80% B (3-5.5 min). The instrument was operated in positive electro-spray ionization mode and scanned from m/z 600 to 6000. Mass to charge spectrum was deconvoluted using the Maximum Entropy algorithm, and relative amounts of the relevant species were estimates by peak heights of the deconvoluted masses. Instrument settings included: capillary voltage 3500V; fragmentor 175V; skimmer 65V; gas temperature 325 C; drying gas flow 5.0 L/min; nebulizer pressure 30 psig; acquisition mode range 100-7000 with 0.42 scan rate.

Changes in MW relative to the TOPA-[C7]-phenyl-DBCO-PSMB127 were observed for the cerium and neodymium samples. The intact mass of the conjugate incubated with cerium showed an increase in MW of 139 (20% by peak area) or 276 (77%) Da corresponding to the addition of 1 or 2 cerium ions. After DTPA challenge, the masses remained similar with similar abundance (30 and 67% for the +138 and +274 species).

### Example 19

### 6-((16-((6-carboxypyridin-2-yl)(4-isothiocyanatophenyl)methyl)-1,4,10,13-tetraoxa-7,16-diazacyclooctadecan-7-yl)methyl)picolinic acid (H2bp18c6-benzyl-phenyl) (TOPA-[C7]-phenylisothiocyanate and Sodium salt forms

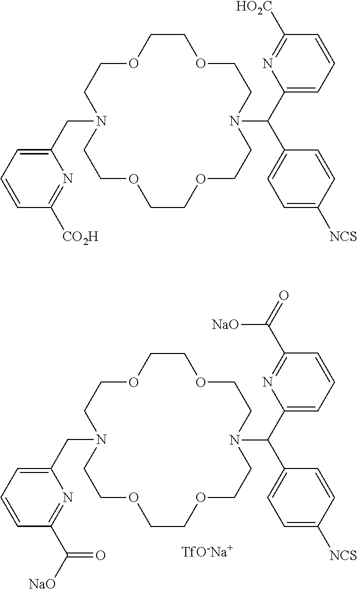

Compound 2 was prepared in an analogous manner to existing literature methods see. J. Org. Chem; 1987, 52, 5172.

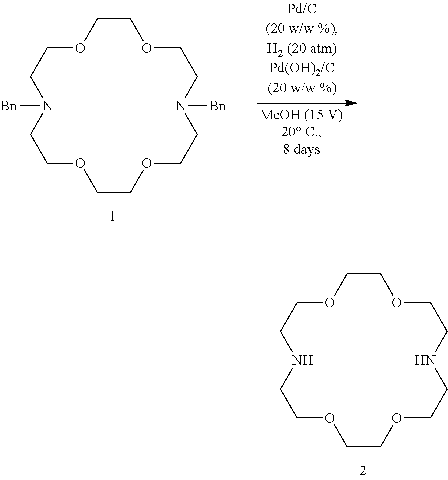 [SMILES: c1ccc(CN2CCOCCOCCN(Cc3ccccc3)CCOCCOCC2)cc1 ; C1COCCOCCNCCOCCOCCN1]

Compound 3 was prepared in an analogous manner to existing literature methods see. Chemistry—a European Journal; 2015, 21, 10179.

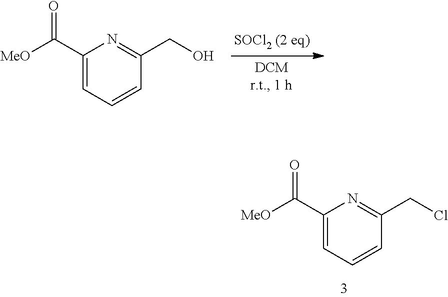 [SMILES: COC(=O)c1cccc(CO)n1 ; COC(=O)c1cccc(CCl)n1]

### Preparation of Compound 4

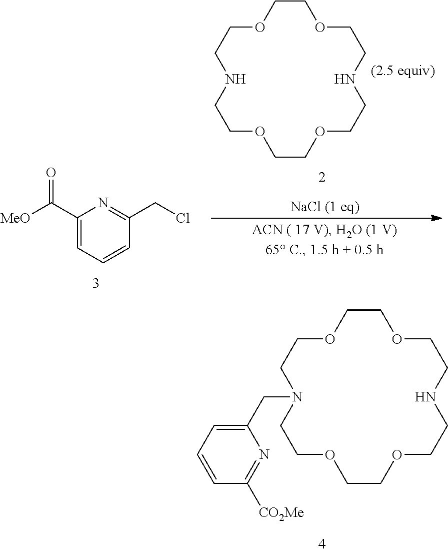 [SMILES: C1COCCOCCNCCOCCOCCN1 ; COC(=O)c1cccc(CCl)n1 ; COC(=O)c1cccc(CN2CCOCCOCCNCCOCCOCC2)n1]

1,4,10,13-tetraoxa-7,16-diazacyclooctadecane (494 g, 1.88 mol, 2.5 equiv.), NaCl (44.1 g, 0.75 mol, 1.0 equiv.), H₂O (140 mL, 1 volume with respect to compound 3) and acetonitrile (2.1 L, 15 volumes) were charged to a 10 L reactor under N₂ atmosphere at 15-20° C. the heated to 65° C. To the resulting mixture was added a solution of compound 3 (140 g, 0.75 mol) in acetonitrile (280 ml, 2 volumes) dropwise over 1 hour 65° C. The solution was aged at 65° C. for 0.5 hours.

LCMS analysis of the mixture showed the reaction was completed. The mixture was cooled to room temperature and concentrated under vacuum. Acetone (700 ml, 5 volumes) was added to the mixture and the suspension was stirred for an additional 1 hour. The mixture was filtered (the filtered solid was unreacted compound 2). The filtrate was concentrated under vacuum, then dissolved in DCM (1.4 L, 10 volumes). The organic phase was washed with water (3×750 mL) and the organic phase was dried over Na₂SO₄ then concentrated under vacuum to yield compound 4, 212 g (63% yield, assay: 85% w/w). LCMS: (ES, m/z): 412.15 [M+H]^{+ 1}H-NMR (300 MHz, DMSO-d₆, ppm): δ 7.98-7.87 (m, 2H), 7.81 (dd, J=6.4, 2.6 Hz, 1H), 3.87 (s, 3H), 3.81 (s, 2H), 3.61-3.38 (m, 16H), 2.77 (dt, J=19.0, 5.2 Hz, 8H).

### Preparation of Compound 7

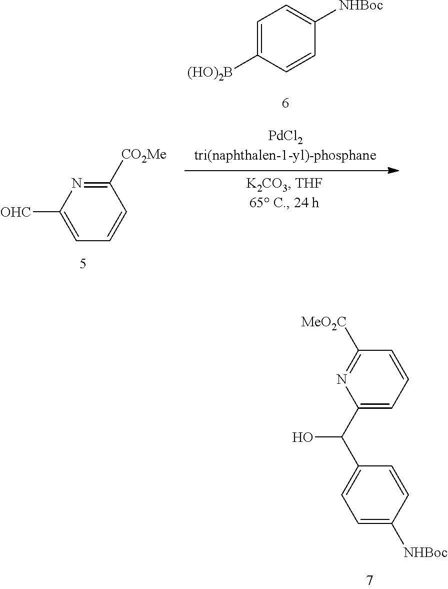 [SMILES: CC(C)(C)OC(=O)Nc1ccc(B(O)O)cc1 ; COC(=O)c1cccc(C=O)n1 ; COC(=O)c1cccc(C(O)c2ccc(NC(=O)OC(C)(C)C)cc2)n1]

Methyl 6-formylpicolinate 5 (250 g, 1.0 equiv.), (4-((tert-butoxycarbonyl)amino)phenyl)boronic acid 6 (538 g, 1.5 equiv.) and degassed THF (6.5 L, 26 volumes with respect to 5) were charged into a 10 L reactor under N₂ atmosphere at 15-20° C. This was followed by the addition of PdCl2 (14.0 g, 0.05 equiv.), tri(naphthalen-1-yl)-phosphane (31 g, 0.05 equiv.) and K₂CO₃ (650 g, 3.1 equiv.). The resulting solution was stirred at 20° C. for 0.5 hours. The Mixture was then heated to 65° C. and aged for 17 hours. Analysis by LCMS showed this reaction was complete. The resulting solution was cooled at room temperature and was diluted with ice water (2.5 L, 10 volumes) and ethyl acetate (5 L, 20 volumes). The mixture was stirred then filtered through a celite pad. The solution was allowed to separate, and the aqueous lower layer was discarded. The organic phase was washed with the water (2×1.5 L, 12 volumes). The layers were separated, and the organic layer was dried over Na₂SO₄ and concentrated under vacuum. The resulting residue was treated with heptane (1.25 L, 5 Volumes) and the resulting suspension was stirred for 0.5 hours. The mixture was filtered, and the filter cake was washed with n-heptane (500 ml, 2 volumes) to yield 530 g (98% yield, LCAP purity: 90%) of desired product 7 as yellow solid, which was used directly in the next step without further purification. LCMS: (ES, m/z): 381.10 [M+Na]^{+ 1}H-NMR (300 MHz, DMSO-d₆, ppm): δ 9.27 (s, 1H), 8.03-7.85 (m, 2H), 7.79 (dd, J=7.7, 1.4 Hz, 1H), 7.39 (d, J=8.4 Hz, 2H), 7.26 (d, J=8.4 Hz, 2H), 6.13 (d, J=4.0 Hz, 1H), 5.72 (d, J=3.9 Hz, 1H), 3.87 (s, 3H), 1.46 (s, 9H).

### Preparation of Compound 8

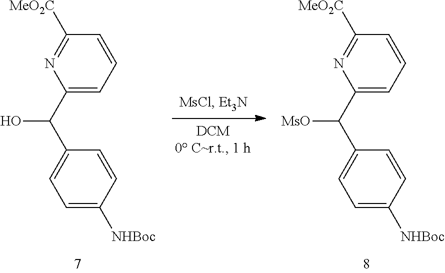 [SMILES: COC(=O)c1cccc(C(O)c2ccc(NC(=O)OC(C)(C)C)cc2)n1 ; COC(=O)c1cccc(C(OS(C)(=O)=O)c2ccc(NC(=O)OC(C)(C)C)cc2)n1]

Methyl 6-((4-((tert-butoxycarbonyl)amino)phenyl)(hydroxy)methyl)picolinate 7 (310 g, 1.0 equiv.), triethylamine (219 g, 2.5 equiv.) and DCM (6.2 L, 20 volumes with respect to 7) were charged into a 10 L reactor under nitrogen atmosphere at 15-20° C. and the solution was cooled to 0° C. Methanesulfonyl chloride (99.2 g, 1.0 equiv.) was added dropwise over 30 min maintaining the temperature at 0° C. The cooling bath was removed, and the temperature was allowed to reach ambient temperature and was then aged for 1 hour at this temperature. The solution was concentrated under vacuum at 10-15° C. and the residue was then dissolved in acetonitrile (438 ml, 2 volumes). The resulting solution was concentrated under vacuum to yield 518 g (crude) of desired product 8. This crude product was used for the next step directly without further purification.

### Preparation of Compound 9

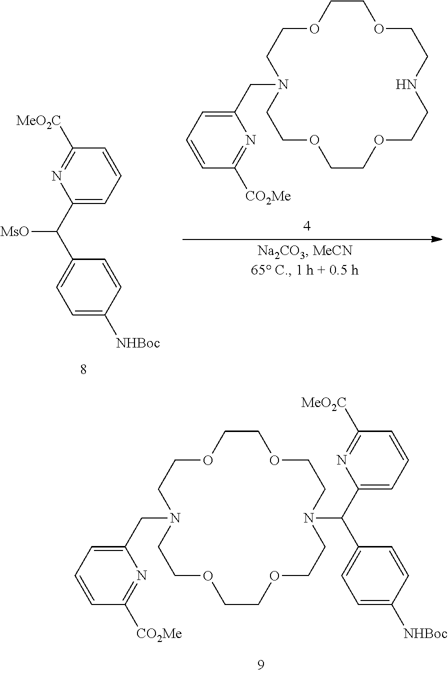 [SMILES: COC(=O)c1cccc(C(OS(C)(=O)=O)c2ccc(NC(=O)OC(C)(C)C)cc2)n1 ; COC(=O)c1cccc(CN2CCOCCOCCNCCOCCOCC2)n1 ; COC(=O)c1cccc(CN2CCOCCOCCN(C(c3ccc(NC(=O)OC(C)(C)C)cc3)c3cccc(C(=O)OC)n3)CCOCCOCC2)n1]

Methyl 6-((4-((tert-butoxycarbonyl)amino)phenyl)-((methylsulfonyl)oxy)methyl)picolinate 8 (212 g, 1.0 equiv. 85% purity by Q-NMR), Na₂CO₃ (137.6 g, 3.0 equiv.) and acetonitrile (3.56 L, 20 volumes with respect to 8) were charged into a 10 L reactor under a nitrogen atmosphere at room temperature then the mixture was heated to 65° C. and aged for 1 hour. A solution of methyl 6-((1,4,10,13-tetraoxa-7,16-diazacyclooctadecan-7-yl)methyl)picolinate 4 (377.8 g, 2.0 equiv.) in acetonitrile (3 L, 10 volumes) was added dropwise over 0.5 hours at 65° C. The mixture was aged at this temperature until HPLC analysis showed this reaction was completed. The resulting solution was cooled at room temperature then filtered, and the filter cake was washed by MeOH (2×1 volume). The filtrate was concentrated under vacuum and the resulting residue was dissolved in EA (700 mL), then silica gel (800 g, type: ZCX-2, 100-200 mesh, 2.11 w/w) was added. The mixture was concentrated under vacuum whilst maintaining the temperature below 35° C. Silica gel (9.6 kg, type: ZCX-2, 100-200 mesh, 26.3 w/w) was charged to the column, followed by the prepared dry silica gel containing adsorbed crude 9. The column was eluted with ethyl acetate:petroleum ether:dichloromethane (3:3:1)/methanol:dichloromethane (1:1) (gradient from 100:0 to 90:10 with sample collection every 4 L+0.5 L). The fractions were analyzed by TLC (ethyl acetate:petroleum ether:dichloromethane:methanol=4:4:1:1). The product bearing fractions were combined and concentrated to yield 260 g of compound 9 as yellow solid (HPLC: 94%, QNMR: 92%). An additional 70 g of compound 9 was afforded as yellow oil (HPLC: 75%, QNMR: 60%). LCMS (ES, m/z): 752.30 [M+H]⁺ Observed m/z ¹H-NMR (400 MHz, CDCl3, ppm): δ 7.53-7.32 (m, 3H), 7.28-7.18 (m, 3H), 6.86 (d, J=8.4 Hz, 2H), 6.76 (d, J=8.4 Hz, 2H), 6.09 (s, 1H), 4.63 (s, 1H), 3.48 (s, 3H), 3.44 (bs, 5H), 3.17-2.92 (m, 16H), 2.38 (dq, J=25.0, 7.2, 6.8 Hz, 8H), 0.97 (s, 9H).

### Preparation of Compound 10

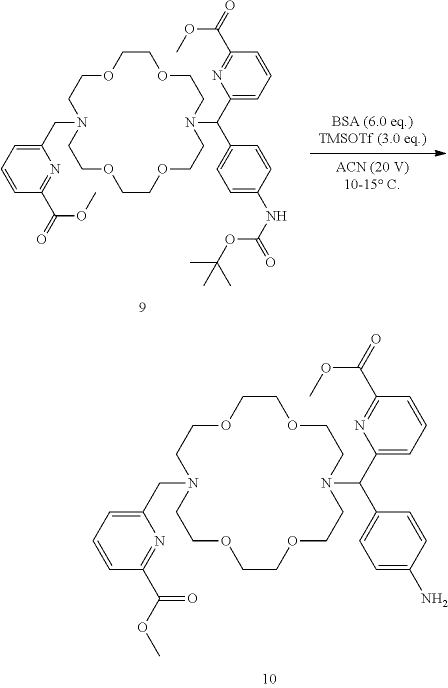 [SMILES: COC(=O)c1cccc(CN2CCOCCOCCN(C(c3ccc(NC(=O)OC(C)(C)C)cc3)c3cccc(C(=O)OC)n3)CCOCCOCC2)n1 ; COC(=O)c1cccc(CN2CCOCCOCCN(C(c3ccc(N)cc3)c3cccc(C(=O)OC)n3)CCOCCOCC2)n1]

Compound 9 (260 g, QNMR: 92%, 1.0 equiv.), N,O-bis(trimethylsilyl)acetamide (BSA, 6.0 equiv.) and acetonitrile (4 L, 15 volumes) were charged into a 10 L reactor under nitrogen atmosphere at 15-20° C. The mixture was stirred for 40 min at 20° C. A solution of TMSOTf (212.9 g, 3.0 equiv.) in acetonitrile (1.3 L, 5 volumes) was charged dropwise over 0.5 hours maintain the internal temperature between 15-20° C. The solution was aged for 1 hour at 15-20° C. When process analysis (sample preparation 0.1 mL system+0.9 mL ACN+one drop of diisopropylethylamine) showed complete conversion of staring material the mixture was quenched with diisoproylethylamine (617 g, 15.0 equiv.) maintain a temperature between 5-10° C. The mixture was stirred for 20 minutes at 5-10° C., then a saturated aqueous NH₄Cl solution (2.6 L, 10 volumes) was charged maintaining a temperature between 5-10° C. The mixture was aged for an additional 30 minutes at this temperature. The aqueous phase (contained solids) was collected and was extracted with 2-MeTHF (520 ml, 2 volumes). The organic phases were combined and checked for water content by KF (KF: 9.18%), then dried with anhydrous Na₂SO₄ (500 g, 10.0 equiv.). The solids were removed by filtration and the filter cake was washed by acetonitrile (2×520 ml, 2 volumes). The filtrates were then dried with anhydrous Na₂SO₄ (500 g, 10.0 equiv.). After filtration, the filter cake was washed by acetonotrile (2×520 ml, 2 volumes) and water content was checked by KF (KF: 8.15%). The acetonitrile/2-MeTHF stream of 10 was used for next step directly. (The product was not stable to LCMS conditions).

### Preparation of Compound 14 (Free Acid)

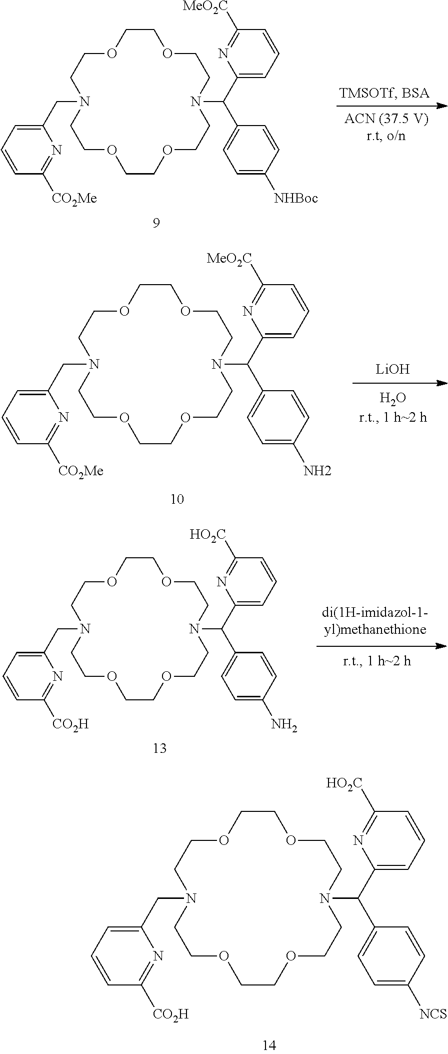

Methyl 6-((4-((tert-butoxycarbonyl)amino)phenyl)(16-((6-(methoxycarbonyl)pyridin-2-yl)methyl)-1,4,10,13-tetraoxa-7,16-diazacyclooctadecan-7-yl)methyl)picolinate (6.0 g, 1.0 equiv.) and BSA (9.7 g, 6.0 equiv.) and MeCN (120 mL, 20 volumes with respect to 9) were charged to a 500 mL reactor under nitrogen atmosphere at room temperature. A solution of TMSOTf (5.4 g, 2.3 equiv.) in MeCN (120 mL, 20 volumes) was added dropwise over 30 min at room temperature. The mixture was aged for overnight at room temperature. Analysis of the mixture (sample preparation 0.1 mL system+0.9 mL ACN+one drop of diisopropylethylamine) showed the reaction had reached completion. The mixture was quenched with diisoproylethylamine (15.4 g, 15.0 equiv.) maintain a temperature between 0-5° C. The mixture was stirred for 5 minutes at 0-5° C., then a saturated aqueous NH₄Cl solution (60 mL, 10 volumes) was charged dropwise maintaining a temperature between 0-5° C. The aqueous phase was removed by extraction and the organic phase was collected and used for next step directly. The organic phase was charged to 500 mL 3-necked round bottle bottom bottle, a solution of LiOH (1.15 g, 6.0 equiv.) in water (60 mL, 10 V) was added to the solution at room temperature. The solution was stirred for 1 hour at this temperature. Analysis of the mixture (sample preparation, 0.1 mL system+0.9 mL acetonitrile) showed not fully conversion. Another portion of LiOH (576 mg, 3.0 equiv.) was added and the solution was stirred for another 1 hour at room temperature. Analysis of the mixture (sample preparation, 0.1 mL system+0.9 mL acetonitrile) showed the reaction had reached completion. Then, TCDI (5.6 g, 3.9 equiv.) was added and the solution was stirred for 1 hour at room temperature. Analysis of the mixture (sample preparation, 0.1 mL system+0.9 mL acetonitrile) showed not fully conversion. Another portion of TCDI (2.8 g, 2.0 equiv.) was added and the solution was stirred for another 1 hour at room temperature. Analysis of the mixture (sample preparation, 0.1 mL system+0.9 mL acetonitrile) showed the reaction had reached completion. The reaction solution was separated by reversal phase Combi-Flash. Method: column C18, A solution H₂O (Containing 0.01% formic acid), B solution ACN. 5% to 35% in 40 min, flow (100 mL/min), product in 20 min-25 min. Collect a solution. The solution was concentrated to remove ACN and separated by reversal phase Combi-Flash again. Method: column C18, A solution H₂O, B solution ACN. 5% 10 min, 5% to 35% in 5 min 95% 10 min, flow (100 mL/min), product in 13 min-25 min. Collect a solution. The solution was concentrated under vacuum at <20° C. and dried by lyophilization. This result in 2.5 g (47% yield in 3 steps) compound 14 as a yellow solid. Compound 14 (6-((16-((6-carboxypyridin-2-yl)(4-isothiocyanatophenyl)methyl)-1,4,10,13-tetraoxa-7,16-diazacyclooctadecan-7-yl)methyl)picolinic acid) required storage at −80° C. LCMS: (ES, m z): 666.3 [M+H]^{+ 1}H-NMR: (400 MHz, D₂O, ppm): 7.94-7.84 (m, 4H), 7.56-7.40 (m, 4H), 7.16-7.14 (m, 2H), 5.83 (s, 1H), 4.56 (s, 2H), 3.80-3.75 (m, 8H), 3.60-3.49 (m, 14H), 3.36-3.33 (m, 2H).

### Preparation of Compound 11 (Sodium Salt)

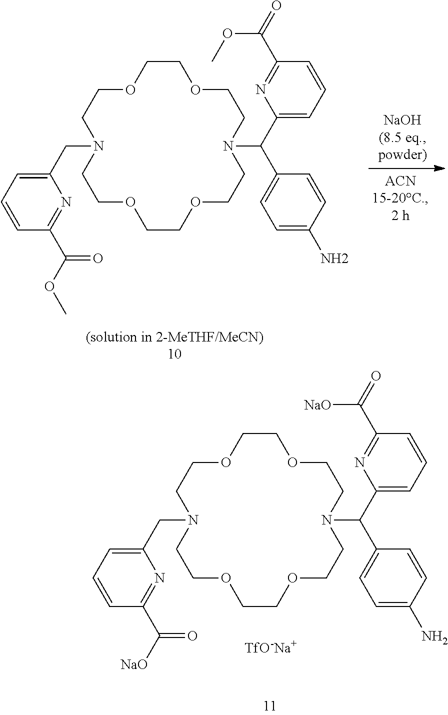

The prepared solution of compound 10 in ACN and 2-MeTHF was charged to a 10 L 4-necked reaction and the solution was cooled to 5-10° C. Powdered NaOH (56.9 g, 4.5 equiv.) was added maintaining the temperature between 5-10° C. The solution was stirred for 0.5 hours at 15-20° C. Analysis of the mixture (sample preparation, 0.1 mL system+0.9 mL acetonitrile) showed no conversion. Additional powdered NaOH (25.3 g, 2.0 equiv.) was added at 5-10° C. The solution was aged for an additional 0.5 hours at 15-20° C. A second IPC was analyzed and showed there was 50% conversion. A final charge of powdered NaOH (25.3 g, 2.0 equiv.) was added at 5-10° C. The mixture was stirred for additional 0.5 hours at 15-20° C. Analysis showed complete conversion of the starting material 10. The mixture was filtered, and the filter cake was washed by acetonitrile (2×520 ml, 2 volumes). The final solution (˜7.5 L, 28.8 volumes) was concentrated to 1-2 volumes maintaining a temperature between 15-20° C. The residue was then treated with acetonitrile (2 L, 7.7 volumes) and the water content was checked by KF (KF: 5.7%). The mixture was filtered, and the filter cake was washed by ACN (2×520 ml, 2 volumes). The solution was then concentrated to 1-2 V under vacuum at 15-20° C. The water content was again checked by KF (KF: 5.5%). The solution was diluted with acetonitrile (390 ml, 1.5 volumes), and was added dropwise over 0.5 hours into MTBE (2.6 L, 10 volumes) maintaining a temperature between 15-20° C. The solvents were decanted to leave a viscous oil which was redissolved in acetonitrile (520 ml, 2 volumes) and added into MTBE (2.6 L, 10 volumes). This process was repeated a further four times. To yield a viscous oil which was finally dissolved in acetonitrile (520 ml, 2 volumes) and dried, then concentrated at 15-20° C. under reduced pressure. Residual solvents were then removed by evaporation with an oil pump at 15-20° C. After drying 335 g of compound 11 was afforded as an off-yellow solid (QNMR: 70%, 87% overall yield over the two steps). LCMS (ES, m/z): 624.3 [M-TfONa-2Na+3H]^{+ 1}H-NMR (300 MHz, Methanol-d₄, ppm): δ 7.97 (dd, J=7.8, 2.1 Hz, 2H), 7.84 (t, J=7.7 Hz, 1H), 7.75 (t, J=7.8 Hz, 1H), 7.36 (dd, J=7.8, 1.1 Hz, 1H), 7.23 (d, J=7.7 Hz, 1H), 7.11 (d, J=8.5 Hz, 2H), 6.72 (d, J=8.5 Hz, 2H), 3.96 (s, 1H), 3.83-3.36 (m, 18H), 3.03-2.62 (m, 6H), 2.55 (d, J=14.3 Hz, 2H).

### Preparation of Compound 12 (TOPA-[C7]-phenylisothiocyanate Sodium Salt)

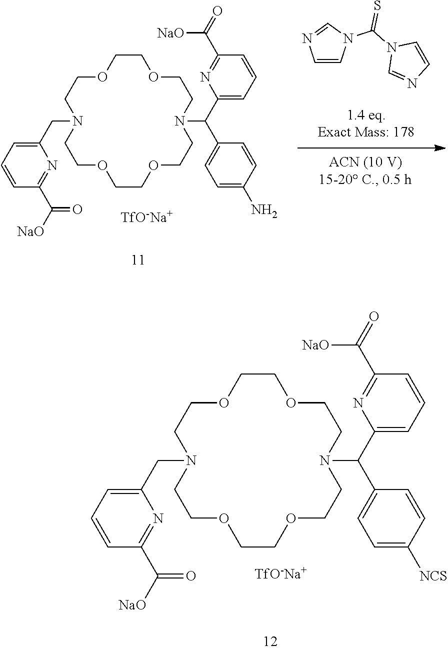

TCDI (68.7 g, 1.4 equiv.) and acetonitrile (2.6 L, 8 volumes) were charged to a 10 L reactor under nitrogen atmosphere at 15-20° C. A solution of compound 11 (330 g, Na⁺ salt, QNMR: 70%, 1.0 equiv.) in acetonitrile (660 mL, 2 volumes) was added dropwise over 30 min maintaining a temperature between 15-20° C. The mixture was aged for 0.5 hours at 15-20° C. Analysis of the mixture (sample preparation:30 μL system+300 μL ACN+a drop of water) showed the reaction had reached completion. The water content was checked by KF (KF: 0.19%). The system was dried and concentrated at 15-20° C. under reduced pressure. The resulting residue was dissolved in acetonitrile (945 ml, 2.9 volumes) and the water content was measured by KF (KF: 0.34%). isopropyl acetate (660 ml, 2 volumes) was charged to the solution over 40 minutes at 15-20° C. No nucleation was observed, and additional isopropyl acetate (6.6 L, 18 volumes) was charged dropwise slowly in 40 min at 15-20° C. leading to precipitation of the product 12 which was collected by filtration as an off yellow solid. The solid was dissolved in acetonitrile (330 ml, 1 volume) and IPAc (6.6 L, 20 volumes) was charged dropwise slowly in 40 min at 15-20° C. The mixture was filtered to yield 230 g of product as an off-yellow solid (LCAP: 80.99%, QNMR: 59%, 10% IPAc). The wet cake was dried under vacuum in 2 hours at 15-20° C., to give 224 g of crude 12 as an off-yellow solid (LCAP: 80.9%, QNMR: 60.4%, ˜6% IPAc). The crude 12 was redissolved in acetonitrile (330 ml, 1 volumes) and isopropyl acetate (412 ml, 1.25 volumes) was charged dropwise slowly in 40 min at 15-20° C. The resulting mixture was filtered, and 12 was collected (30.5 g, HPLC=60.9%, assay: 25.5%). The mother liquors were diluted with isopropyl acetate (6.6 L, 20 volumes) added over 40 minutes at 15-20° C. The mixture was filtered, and the cake was dried to afford 173.5 g of crude product 12 as an off-yellow solid (LCAP: 85.4%, QNMR: 66%, 3.9% IPAc, RRT1.19=3.9%). 190 g of crude 12 product was dissolved in 760 mL of acetonitrile:isopropyl acetate (2:1) and the mixture was passed through a silica gel column (380 g, 2×). The silica was flushed with acetonitrile:isopropyl acetate (2:1, 5.7 L) and then 12 L acetonitrile (very little product). Product containing fraction were the concentrated to afford 118 g of product 12 as an off-yellow solid (LCAP: 95%). The silica pad was then flushed with MeCN/H₂O (12 L, 10:1). The solvents were removed in vacuo to afford and additional 60 g crude 12 as an off-yellow solid which was dissolved in acetonitrile (1.5 L), stirred for 30 min then filtered. The mother liquor were then concentrated to afford 24 g of crude 12 as an off-yellow solid (LCAP=92%). crude 12 (118 g) and crude 12 (24 g) prepared as above were dissolved in acetonitrile (330 ml, 1 volume) and isopropyl acetate (6.6 L, 20 volumes) was charge dropwise over 40 min 15-20° C. The mixture was then filtered to afford 133 g of 12 product as off-yellow solid of suitable purity (LCAP: 95%, QNMR: 60.8%, 7.8% IPAc). Note, compound 12 required storage at −20° C. LCMS: (ES, m/z): 666.61 [M-TfONa-2Na+3H]^{+ 1}H-NMR: (400 MHz, methanol-d₄, ppm): δ 8.00 (ddd, J=13.8, 7.7, 1.0 Hz, 2H), 7.84 (dt, J=20.4, 7.7 Hz, 2H), 7.58-7.49 (m, 2H), 7.40 (dd, J=7.6, 1.0 Hz, 1H), 7.36-7.28 (m, 2H), 7.28-7.20 (m, 1H), 4.96 (hept, J=6.3 Hz, 1H), 3.96-3.88 (m, 1H), 3.83 (d, J=15.1 Hz, 1H), 3.70-3.52 (m, 11H), 3.55-3.39 (m, 4H), 3.07-2.73 (m, 6H), 2.62 (dt, J=15.1, 3.6 Hz, 2H).

### Example 20

### TOPA-[C7]-phenylthiourea-h11B6 Antibody Conjugate

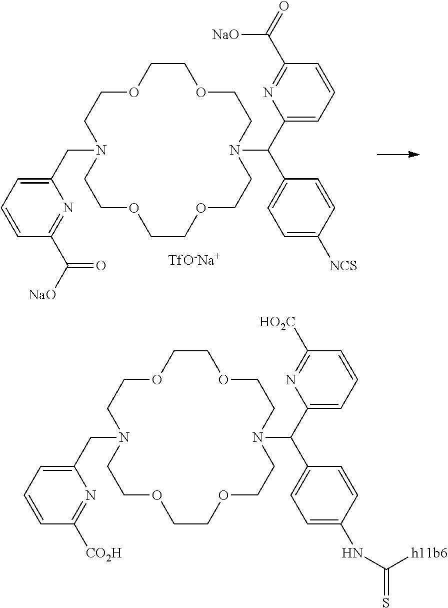

(In the TOPA-[C7]-phenylthiourea-h11B6 Antibody Conjugate depicted above, the structure does not show the lysine residue of h11b6 that is linked to the phenylthiourea moiety.)

TOPA-[C7]-phenylthiourea Modification of mAb:

h1b6 mAb (10.2 mg/ml) was diluted to 1 mg/ml in 10 mM sodium acetate pH 5.2 buffer. Directly prior to conjugation, pH was adjusted to pH 9 with sodium bicarbonate buffer (VWR 144-55-8). pH was confirmed with pH paper. Then, 10× molar excess of disodium salt TOPA-[C7]-phenylisothiocyanate sodium salt (50 mM stock dissolved in water) was added to the h1b6 mAb, and the mixture of antibody and TOPA-[C7]-phenylisothiocyanate sodium salt was incubated at room temperature without shaking for approximately 1 hour. The addition of TOPA-[C7]-phenylisothiocyanate sodium salt was monitored by intact mass ESI-TOF LC-MS on an Agilent® G224 instrument until the CAR value was between 1.5-2.0. The mixture was then immediately quenched by addition of 1M Tris pH 8.5 (Teknova T1085) to a final concentration of 100 mM. Excess free chelator was removed by desalting the reaction into 10 mM sodium acetate pH 5.2 using a 7K Zeba® desalting column. To confirm no excess chelator was present, 3× rounds of sample dilution to 15 mls followed by concentration to 1 ml using a 50,000MWCO Amicon concentrator device was performed. Sample was then concentrated to its final concentration for radiolabeling. The final conjugate was confirmed to be monomeric by analytical size exclusion chromatography on a Tosoh TSKgel G3000SWxl 7.8 mm×30 cm, 5 u column; column temperature: room temperature; the column was eluted 0.2M sodium phosphate pH 6.8; flow rate: 0.8 mL/min; 18 min run; injection volume: 18 ul.

### Example 21

Ac-225 labeled TOPA-[C7]-phenylthiourea-h11B6 Antibody Conjugate

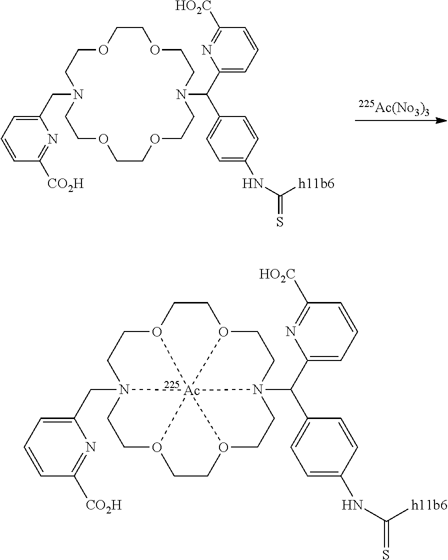

(In the Ac-225 labeled TOPA-[C7]-phenylthiourea-h11B6 Antibody Conjugate depicted above, the structure does not show the lysine residue of h11b6 that is linked to the phenylthiourea moiety.)

(i) Labeling of TOPA-[C7]-phenylthiourea-h11B6 with Ac-225 in 3M NaOAc Buffer:

To a solution of NaOAc (3 M in H₂O, 60 μL) in a plastic vial were added sequentially Ac-225 (10 mCi/mL in 0.1 N HCl, 15 μL) and TOPA-[C7]-phenylthiourea-h11B6 (1.13 mg/mL in 10 mM NaOAc pH=5.5, 441 uL, 0.5 mg). After mixing, the pH was ˜6.5 by pH paper. The vial was left standing still at 37° C. for 2 hr.

iTLC of the Labeling Reaction Mixture:

0.5 μL of the labeling reaction mixture was loaded onto an iTLC-SG, which was developed with 10 mM EDTA (pH 5-6). The dried iTLC-SG was left at room temperature for overnight before it was scanned on a Bioscan AR-2000 radio-TLC scanner. Under the elution conditions described herein, TOPA-[C7]-phenylthiourea-h11B6 bound Ac-225 stayed at the origin and any free Ac-225 would migrate with the solvent to the solvent front. Scanning of the iTLC showed 99.9% TOPA-[C7]-phenylthiourea-h11B6 bound Ac-225.

DTPA Challenge of the Labeling Reaction Mixture:

0.5 uL of the labeling reaction mixture was also mixed with 10 mM DTPA (pH=6.5, 15 μL) at 37° C. After 30 min, 10 uL of the mixture was spotted on iTLC-SG and developed by 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 chelated Ac-225 stayed at the origin and any free Ac-225 would migrate with the solvent to the solvent front. Scanning of the iTLC showed 99.7% TOPA-[C7]-phenylthiourea-h11B6 chelated Ac-225.

Purification on PD10 Column:

The PD-10 resin was conditioned in NaOAc buffer solution by passing 5 mL×3 of NaOAc buffer (25 mM NaOAc, 0.04% PS-20, pH 5.5) through column and discarding the washings. The entire labeling reaction mixture was applied to the reservoir of the column and the eluate collected in pre-numbered plastic tubes. The reaction vial was washed with 0.2 mL×3 NaOAc buffer (25 mM NaOAc, 0.04% PS-20, pH 5.5) and the washings pipetted into the reservoir of the PD-10 column and the eluate collected. Each tube contained ˜1 mL of the eluate. Continued application of NaOAc buffer (25 mM NaOAc, 0.04% PS-20, pH 5.5) into the reservoir of the PD-10 column occurred until a total elution volume of 10 mL was reached. The radiochemical purity of fractions collected were checked by iTLC: 10 uL of each collected fraction was spotted on iTLC-SG and developed with 10 mM EDTA. The dried iTLC-SG was left at room temperature for overnight before it was scanned on a Bioscan AR-2000 radio-TLC scanner. Pure fraction should have no radioactivity signal at the solvent front of the iTLC-SG.

DTPA Challenge of the Purified ²²⁵Ac-TOPA-[C7]-phenylthiourea-h11B6:

10 μL of fraction #3 collected after PD-10 column was mixed with 15 μL of 10 mM DTPA solution (pH 6.5), and incubated for 30 min. 10 μL of the mixture was loaded onto an iTLC-SG, which was developed with 10 mM EDTA and dried overnight. It was scanned on a Bioscan AR-2000 radio-TLC scanner. No radioactivity signal was observed at the solvent front of the iTLC-SG indicating that there was no free Ac-225 in the fraction #3.

HPLC Analysis of the Purified ²²⁵Ac-TOPA-[C7]-phenylthiourea-h11B6:

The fraction #3 collected after PD-10 column was analyzed by HPLC. HPLC method: Tosoh TSKgel G3000SWxl 7.8 mm×30 cm, 5 μm column; column temperature: room temperature; the column was eluted with DPBS buffer (X1, without calcium and magnesium); flow rate: 0.7 mL/min; 20 min run; injection volume: 40 μL. After HPLC, the fractions were collected in time intervals of 30 seconds or 1 minute. The collected HPLC fractions were left at room temperature overnight. The radioactivity in each of the collected fractions was counted in a gamma counter. The HPLC radio trace was constructed from the radioactivity in each HPLC fraction. HPLC radio trace showed a radioactive peak corresponding to the TOPA-[C7]-phenylthiourea-h11B6 peak on HPLC UV trace.

(ii) Labeling of TOPA-[C7]-phenylthiourea-h11B6 at Higher Concentration with Ac-225 in 1.5 M NaOAc Buffer:

To a solution of NaOAc (1.5 M in H₂O with 0.04% PS-20, 63 μL) in a plastic vial were added sequentially Ac-225 (10 mCi/mL in 0.1 N HCl, 10 μL) and TOPA-[C7]-phenylthiourea-h11B6 (9.36 mg/mL in 10 mM NaOAc pH=5.2, 0.04% PS-20, 36 uL, 337 μg). After mixing, the pH was ˜6.5 by pH paper. The vial was left standing still at 37° C. for 2 hr.

iTLC of the Labeling Reaction Mixture:

0.5 μL of labeling reaction mixture was then loaded onto an iTLC-SG, which was developed with 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 bounded Ac-225 stayed at the origin and any free Ac-225 would migrate with the solvent to the solvent front. Scanning of the iTLC showed 99.9% TOPA-[C7]-phenylthiourea-h11B6 bonded Ac-225.

DTPA Challenge of the Labeling Reaction Mixture:

0.5 uL of the labeling reaction mixture was also mixed with 10 mM DTPA (pH=6.5, 15 μL) at 37° C. After 30 min, 10 uL of the mixture was spotted on iTLC-SG and developed by 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 chelated Ac-225 stayed at the origin and any free Ac-225 would migrate with the solvent to the solvent front. Scanning of the iTLC showed 99.9% TOPA-[C7]-phenylthiourea-h11B6 chelated Ac-225.

(iii) Labeling of TOPA-[C7]-phenylthiourea-h11B6 at Higher Concentration with Ac-225 in 1 M NaOAc Buffer:

To a solution of NaOAc (1.0 M in H₂O with 0.04% PS-20, 63 μL) in a plastic vial were added sequentially Ac-225 (10 mCi/mL in 0.1 N HCl, 10 μL) and TOPA-[C7]-phenylthiourea-h11B6 (9.36 mg/mL in 10 mM NaOAc pH=5.2, 0.04% PS-20, 36 uL, 337 μg). After mixing, the pH was ˜6.5 by pH paper. The vial was left standing still at 37° C. for 2 hr.

iTLC of the Labeling Reaction Mixture:

0.5 μL of labeling reaction mixture was then loaded onto an iTLC-SG, which was developed with 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 bound Ac-225 stayed at the origin and any free Ac-225 would migrate with the solvent to the solvent front. Scanning of the iTLC showed 99.9% TOPA-[C7]-phenylthiourea-h11B6 bonded Ac-225.

DTPA Challenge of the Labeling Reaction Mixture:

0.5 uL of the labeling reaction mixture was also mixed with 10 mM DTPA (pH=6.5, 15 μL) at 37° C. After 30 min, 10 uL of the mixture was spotted on iTLC-SG and developed by 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 chelated Ac-225 stayed at the origin and any free Ac-225 would migrate with the solvent to the solvent front. Scanning of the iTLC showed 99.9% TOPA-[C7]-phenylthiourea-h11B6 chelated Ac-225.

Labeling of TOPA-[C7]-phenylthiourea-h11B6 at Higher Concentration with Ac-225 in 25 mM NaOAc with 0.4% Tween-20, pH 5.5:

To a solution of NaOAc (25 mM in H₂O with 0.04% PS-20, pH 5.5, 10 μL) in a plastic vial were added sequentially Ac-225 (10 mCi/mL in 0.1 N HCl, 5 μL), TOPA-[C7]-phenylthiourea-h11B6 (10.4 mg/mL in 10 mM NaOAc pH=5.2, 16 uL, 166 μg) and NaOH (0.1 M, 5 μL). After mixing, the pH was ˜6.0 by pH paper. The vial was left standing still at 37° C. for 2 hr.

iTLC of the Labeling Reaction Mixture:

0.5 μL of labeling reaction mixture was then loaded onto an iTLC-SG, which was developed with 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 bound Ac-225 stayed at the origin and any free Ac-225 would migrate with the solvent to the solvent front. Scanning of the iTLC showed 99.9% TOPA-[C7]-phenylthiourea-h11B6 bonded Ac-225.

DTPA Challenge of the Labeling Reaction Mixture:

0.5 uL of the labeling reaction mixture was also mixed with 10 mM DTPA (pH=6.5, 15 μL) at 37° C. After 30 min, 10 uL of the mixture was spotted on iTLC-SG and developed by 10 mM EDTA. The dried iTLC-SG was left at room temperature for overnight before it was scanned on a Bioscan AR-2000 radio-TLC scanner. Under the elution conditions described herein, TOPA-[C7]-phenylthiourea-h11B6 chelated Ac-225 stayed at the origin and any free Ac-225 would migrate with the solvent to the solvent front. Scanning of the iTLC showed 99.8% TOPA-[C7]-phenylthiourea-h11B6 chelated Ac-225.

Reaction Conditions for Labeling of TOPA-[C7]-phenylthiourea-h11B6 with Ac-225

| TOPA-[C7]- | | Radiochemical | |
|---|---|---|---|
| phenylthiourea- | Buffer for Labeling | yield | DTPA |
| h11B6 in buffer | reaction | (iTLC) | challenge |
| 1.13 mg/mL in 10 | 3M NaOAc | >99% | >99% |
| mM NaOAc pH = 5.5 | | | |
| 9.36 mg/mL in 10 | 1.5M NaOAc, | >99% | >99% |
| mM NaOAc, pH = 5.2; | 0.04% PS-20 | | |
| 0.04% PS-20 | | | |
| 9.36 mg/mL in 10 | 1.0M NaOAc, | >99% | >99% |
| mM NaOAc, pH = 5.2; | 0.04% PS-20 | | |
| 0.04% PS-20 | | | |
| 10.4 mg/mL in 10 | 25 mM in NaOAc, | >99% | >99% |
| mM NaOAc, pH = 5.2 | 0.04% PS-20, pH 5.5 | | |

### Example 22

The following set of experiments were conducted to examine the effects of the presence of non-radioactive metal contaminants, that accompany Actinium sources, on TOPA and DOTA chelating macrocycles. The four most comment contaminants found in the ORNL source of ²²⁵Ac(NO₃)₃ by ICP-MS; Al³⁺, Ca²⁺, Zn²⁺, Mg²⁺ were used as spiking standards in chelation reactions of DOTA and TOPA conjugated to h11b6. The outcome of chelation was monitored by iTLC and thereafter challenged with DTPA.

Chelation of TOPA-[C7]-phenylthiourea-h11b6 with Ac-225 in Presence of Metal Impurities (Lower-Level Impurities).

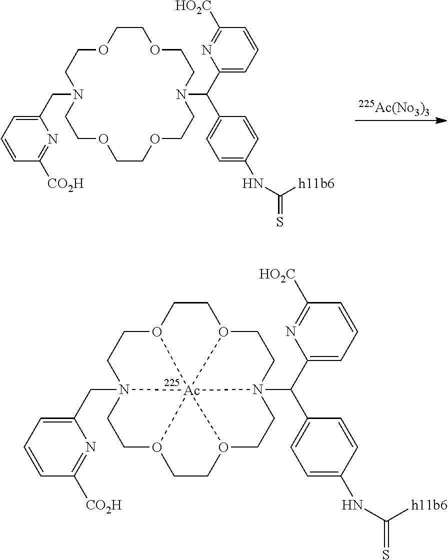

(In the Ac-225 labeled TOPA-[C7]-phenylthiourea-h11B6 Antibody Conjugate depicted above, the structure does not show the lysine residue of h11b6 that is linked to the phenylthiourea moiety.)

Ac-225 was dissolved in 0.1 M HCl and mixed with AlCl₃, CaCl₂, ZnCl₂ and MgCl₂ to form a 5 mCi/mL solution. The concentrations of aluminum, calcium, zinc and magnesium are 9.76 μg/mCi, 3.83 μg/mCi, 0.61 μg/mCi and 0.27 μg/mCi, respectively. To a solution of NaOAc (3 M in H₂O, 20 μL) in a plastic vial were added sequentially Ac-225 (5 mCi/mL in 0.1 N HCl, 10 μL, containing added metal impurities) and TOPA-[C7]-phenylthiourea-h11b6 (1.17 mg/mL in 10 mM NaOAc pH=5.5, 143 μL, 0.167 mg). After mixing, the pH was ˜6.5 by pH paper. The vial was left to stand at 37° C. for 2 hr.

iTLC of the Labeling Reaction Mixture:

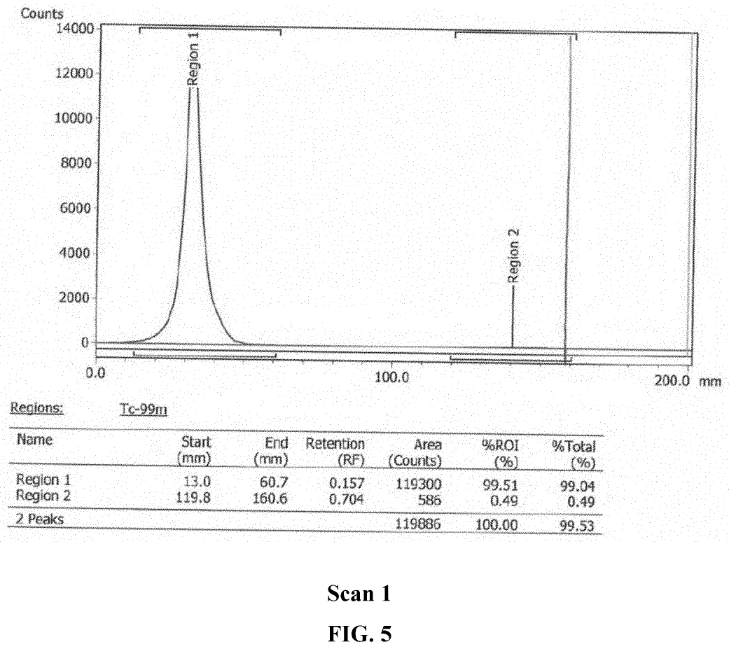

0.5 μL of labeling reaction mixture was in turn loaded onto an iTLC-SG, and developed with 10 mM EDTA solution. The iTLC-SG was allowed to dry at room temperature overnight and thereafter scanned on a Bioscan AR-2000 radio-TLC scanner. Under the elution conditions described herein, Ac-225 bound to TOPA-[C7]-phenylthiourea-h11b6 Ac-225 would remain at the origin (baseline) of the TLC and any free Ac-225 would migrate with the solvent to the solvent front. A scan of the iTLC showed 99.5% of Ac-225 bound to TOPA-[C7]-phenylthiourea-h11b6 (Scan 1, shown in FIG. 5).

DTPA Challenge of the Labeling Reaction Mixture:

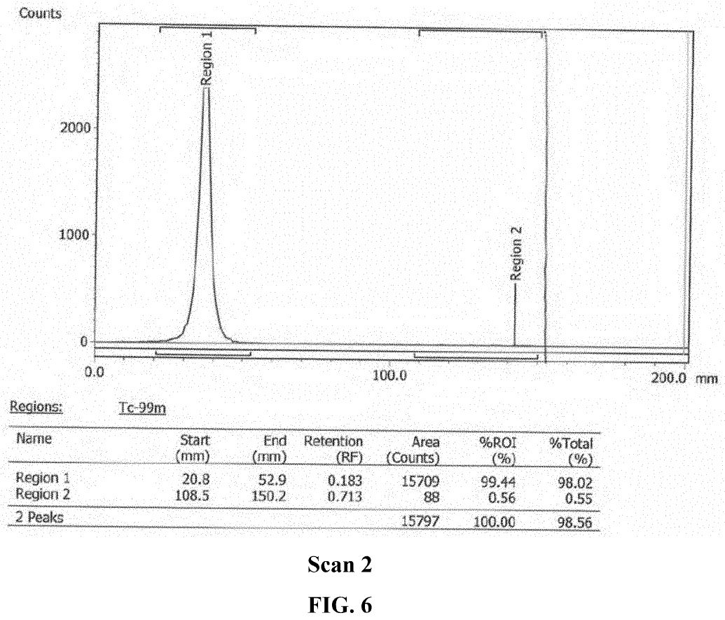

0.5 μL of the labeling reaction mixture was also mixed with 10 mM DTPA (pH=6.5, 15 μL) at 37° C. After 30 min, 10 μL of the mixture was spotted on iTLC-SG and developed in 10 mM EDTA eluent. The iTLC-SG was allowed to air-dry and left at room temperature for overnight before it was scanned on a Bioscan AR-2000 radio-TLC scanner. Under the elution conditions described herein, TOPA-[C7]-phenylthiourea-h11b6 chelated Ac-225 would remain at the origin and any free Ac-225 would migrate with the solvent to the solvent front. A scan of the iTLC showed 99.4% of Ac-225 bound to TOPA-[C7]-phenylthiourea-h11b6 (Scan 2, shown in FIG. 6).

Metal Spiking Experiments: Labeling of TOPA-[C7]-phenylthiourea-h11b6 with Ac-225 in Presence of Metal Impurities (Higher-Level Impurities, 5-Fold Increase in Concentrations Comparing to the Lower-Level Impurities).

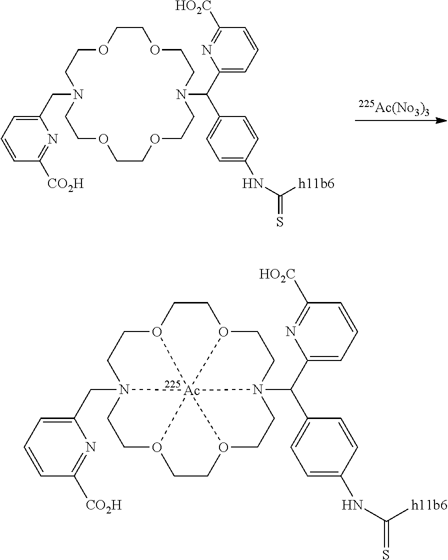

Ac-225 was dissolved in 0.1 M HCl and mixed with AlCl₃, CaCl₂, ZnCl₂ and MgCl₂ to form a 5 mCi/mL solution. The concentrations of aluminum, calcium, zinc and magnesium are 45.0 μg/mCi, 17.3 μg/mCi, 3.01 μg/mCi and 1.15 μg/mCi, respectively. To a solution of NaOAc (3 M in H₂O, 20 μL) in a plastic vial were added sequentially Ac-225 (5 mCi/mL in 0.1 N HCl, 10 μL, containing added metal impurities) and TOPA-[C7]-phenylthiourea-h11b6 (1.17 mg/mL in 10 mM NaOAc pH=5.5, 143 μL, 0.167 mg). After mixing, the pH was ˜6.5 by pH paper. The vial was left to stand at 37° C. for 2 hr.

iTLC of the Labeling Reaction Mixture:

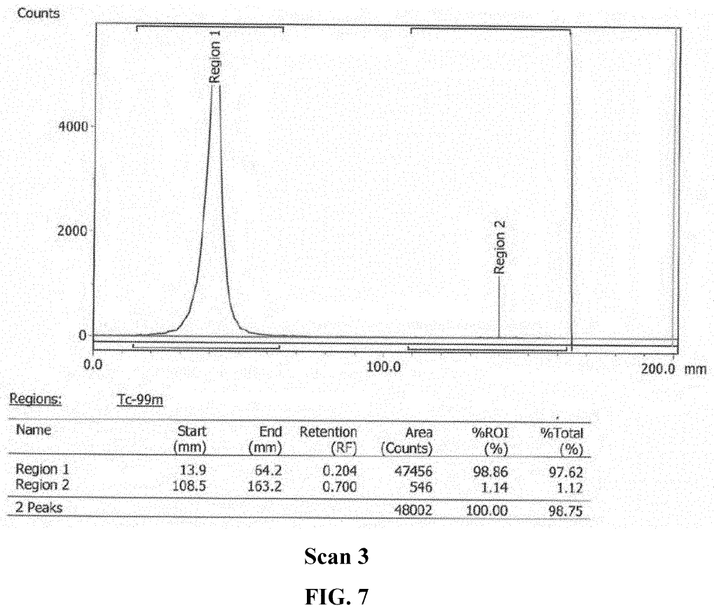

0.5 μL of labeling reaction mixture was in turn loaded onto an iTLC-SG, and developed with 10 mM EDTA solution. The iTLC-SG was allowed to dry at room temperature overnight and thereafter scanned on a Bioscan AR-2000 radio-TLC scanner. Under the elution conditions described herein, Ac-225 bound to TOPA-[C7]-phenylthiourea-h11b6 Ac-225 would remain at the origin (baseline) of the TLC and any free Ac-225 would migrate with the solvent to the solvent front. A scan of the iTLC showed 98.9% of Ac-225 bound to TOPA-[C7]-phenylthiourea-h11b6 (Scan 3, shown in FIG. 7).

DTPA Challenge of the Labeling Reaction Mixture:

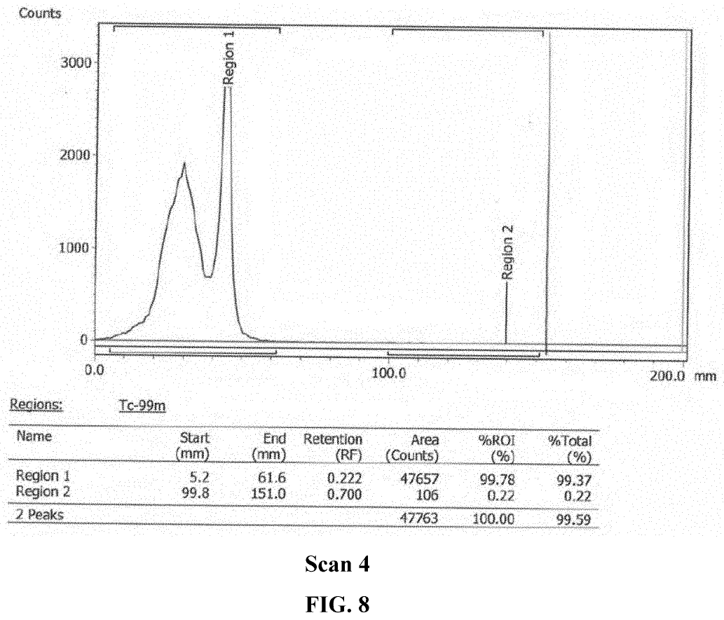

0.5 μL of the labeling reaction mixture was also mixed with 10 mM DTPA (pH=6.5, 15 μL) at 37° C. After 30 min, 10 μL of the mixture was spotted on iTLC-SG and developed in 10 mM EDTA eluent. The iTLC-SG was allowed to air-dry and left at room temperature for overnight before it was scanned on a Bioscan AR-2000 radio-TLC scanner. Under the elution conditions described herein, TOPA-[C7]-phenylthiourea-h11b6 chelated Ac-225 would remain at the origin and any free Ac-225 would migrate with the solvent to the solvent front. A scan of the iTLC showed 99.8% of Ac-225 bound to TOPA-[C7]-phenylthiourea-h11b6 (Scan 4, shown in FIG. 8).

Labeling of DOTA-h11b6 with Ac-225 in Presence of Metal Impurities (Lower-Level Impurities).

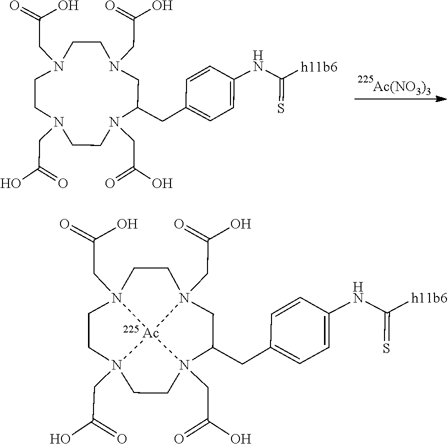

Ac-225 was dissolved in 0.1 M HCl and mixed with AlCl₃, CaCl₂, ZnCl₂ and MgCl₂ to form a 5 mCi/mL solution. The concentrations of aluminum, calcium, zinc and magnesium are 9.76 μg/mCi, 3.83 μg/mCi, 0.61 μg/mCi and 0.27 μg/mCi, respectively. To a solution of NaOAc (3 M in H₂O, 20 μL) in a plastic vial were added sequentially Ac-225 (5 mCi/mL in 0.1 N HCl, 10 μL, containing added metal impurities) and DOTA-h11b6 (10 mg/mL in 25 mM NaOAc pH=5.5, 16.7 μL, 0.167 mg). After mixing, the pH was ˜6.5 by pH paper. The vial was left to stand at 37° C. for 2 hr.

iTLC of the Labeling Reaction Mixture:

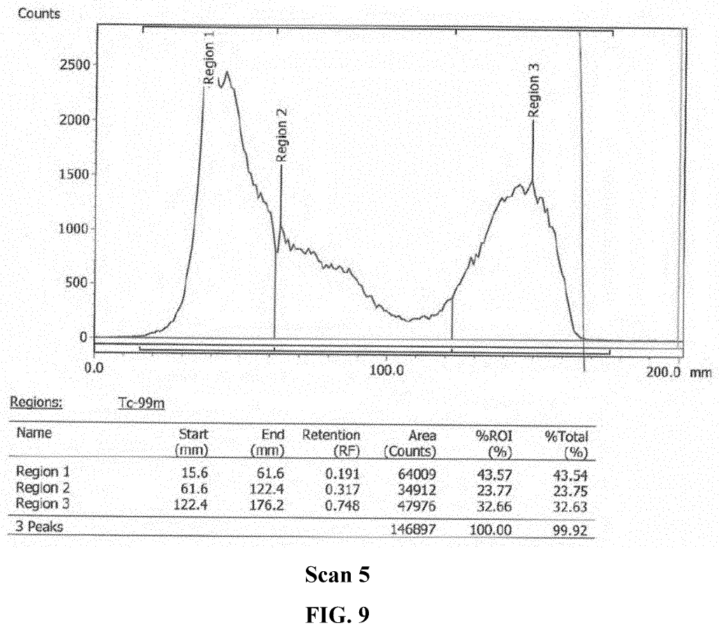

0.5 μL of labeling reaction mixture was in turn loaded onto an iTLC-SG, and developed with 10 mM EDTA solution. The iTLC-SG was allowed to dry at room temperature overnight and thereafter scanned on a Bioscan AR-2000 radio-TLC scanner. Under the elution conditions described herein, Ac-225 bound to DOTA-h11b6 Ac-225 would remain at the origin (baseline) of the TLC and any free Ac-225 would migrate with the solvent to the solvent front. A scan of the iTLC showed 43.6% of Ac-225 chelated to DOTA-h11b6 (Scan 5, shown in FIG. 9).

DTPA Challenge of the Labeling Reaction Mixture:

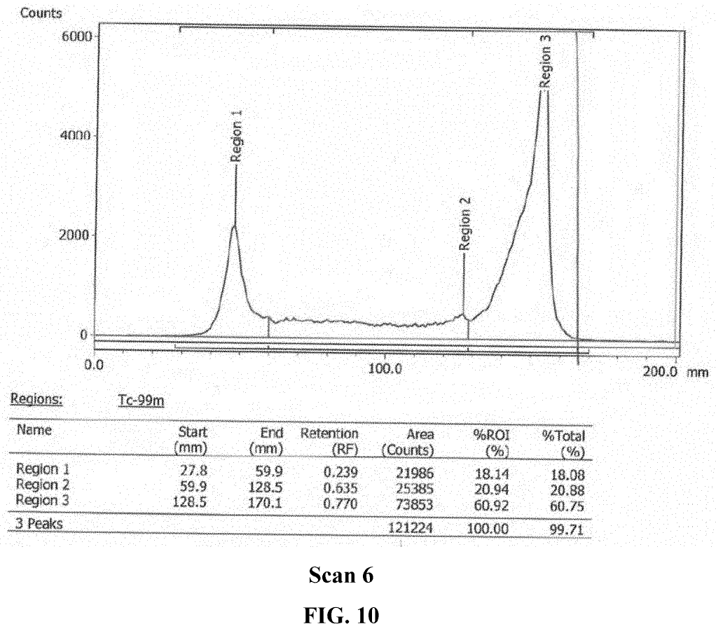

0.5 μL of the labeling reaction mixture was also mixed with 10 mM DTPA (pH=6.5, 15 μL) at 37° C. After 30 min, 10 μL of the mixture was spotted on iTLC-SG and developed in 10 mM EDTA eluent. The iTLC-SG was allowed to air-dry and left at room temperature for overnight before it was scanned on a Bioscan AR-2000 radio-TLC scanner. Under the elution conditions described herein, DOTA-h11b6 chelated Ac-225 would remain at the origin and any free Ac-225 would migrate with the solvent to the solvent front. A scan of the iTLC showed 18.1% of Ac-225 chelated to DOTA-h11b6 (Scan 6, shown in FIG. 10).

Metal Spiking Experiments: Labeling of DOTA-h11b6 with Ac-225 in Presence of Metal Impurities (Higher-Level Impurities, 5-Fold Increase in Concentrations Comparing to the Lower-Level Impurities).

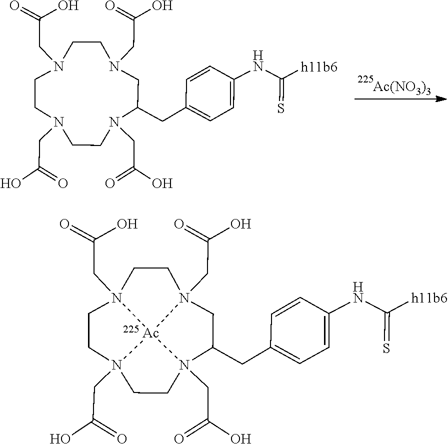

Ac-225 was dissolved in 0.1 M HCl and mixed with AlCl₃, CaCl₂, ZnCl₂ and MgCl₂ to form a 5 mCi/mL solution. The concentrations of aluminum, calcium, zinc and magnesium 45.0 μg/mCi, 17.3 μg/mCi, 3.01 μg/mCi and 1.15 μg/mCi, respectively. To a solution of NaOAc (3 M in H₂O, 20 μL) in a plastic vial were added sequentially Ac-225 (5 mCi/mL in 0.1 N HCl, 10 μL, containing added metal impurities) and DOTA-h11b6 (10 mg/mL in 25 mM NaOAc pH=5.5, 16.7 μL, 0.167 mg). After mixing, the pH was ˜6.5 by pH paper. The vial was left to stand at 37° C. for 2 hr.

iTLC of the Labeling Reaction Mixture:

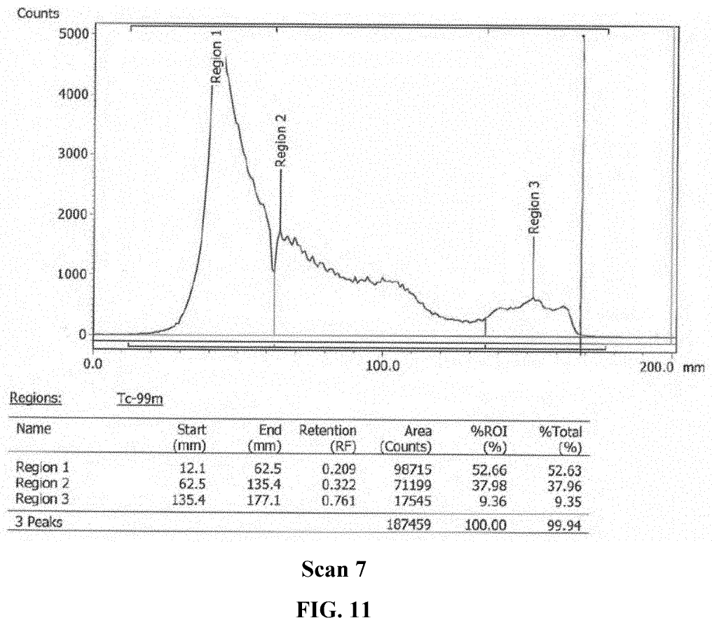

0.5 μL of labeling reaction mixture was in turn loaded onto an iTLC-SG, and developed with 10 mM EDTA solution. The iTLC-SG was allowed to dry at room temperature overnight and thereafter scanned on a Bioscan AR-2000 radio-TLC scanner. Under the elution conditions described herein, Ac-225 bound to DOTA-h11b6 Ac-225 would remain at the origin (baseline) of the TLC and any free Ac-225 would migrate with the solvent to the solvent front. A scan of the iTLC showed 52.7% of Ac-225 chelated to DOTA-h11b6 (Scan 7, shown in FIG. 11).

DTPA Challenge of the Labeling Reaction Mixture:

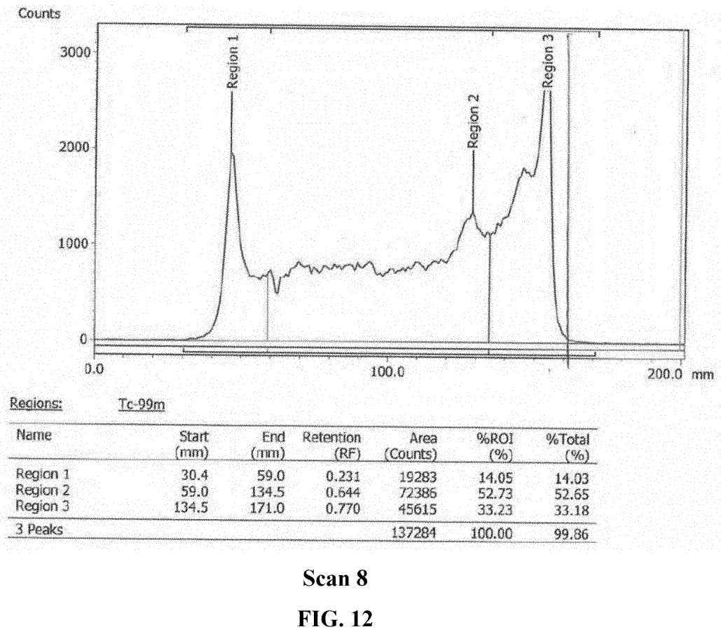

0.5 μL of the labeling reaction mixture was also mixed with 10 mM DTPA (pH=6.5, 15 μL) at 37° C. After 30 min, 10 μL of the mixture was spotted on iTLC-SG and developed in 10 mM EDTA eluent. The iTLC-SG was allowed to air-dry and left at room temperature for overnight before it was scanned on a Bioscan AR-2000 radio-TLC scanner. Under the elution conditions described herein, DOTA-h11b6 chelated Ac-225 would remain at the origin and any free Ac-225 would migrate with the solvent to the solvent front. A scan of the iTLC showed 14.0% of Ac-225 chelated to DOTA-h11b6 (Scan 8, shown in FIG. 12).

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

We claim:

1. A compound of formula (I): 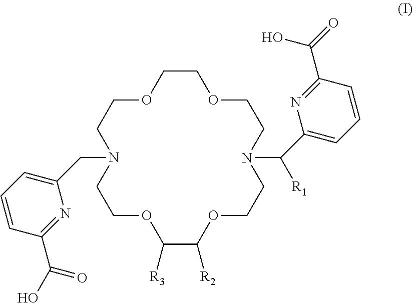
or a pharmaceutically acceptable salt thereof, wherein:
R₁ is hydrogen and R₂ is -L₁-R₄;
alternatively, R₁ is -L₁-R₄ and R₂ is hydrogen;
R₃ is hydrogen;
alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
L₁ is absent or a linker; and
R₄ is a nucleophilic moiety, an electrophilic moiety, or a targeting ligand.

2. A compound of claim 1, of formula (II): 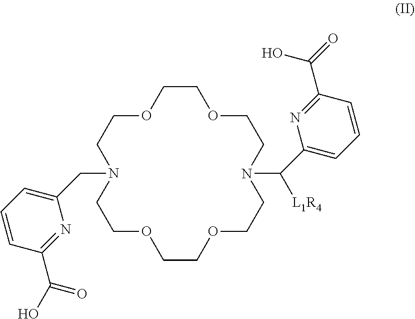
or a pharmaceutically acceptable salt thereof, wherein:
L₁ is absent or a linker; and
R₄ is a nucleophilic moiety, an electrophilic moiety, or a targeting ligand.

3. A compound of claim 1, of formula (III): 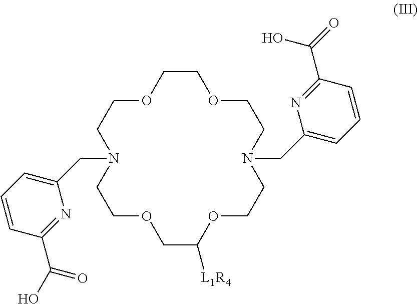
or a pharmaceutically acceptable salt thereof, wherein:
L₁ is absent or a linker; and
R₄ is a nucleophilic moiety, an electrophilic moiety, or a targeting ligand.

4. A compound of claim 1, wherein:
R₁ is -L₁-R₄;
R₂ and R₃ are taken together with the carbon atoms to which they are attached to form a 5- or 6-membered cycloalkyl;
L₁ is absent or a linker; and
R₄ is a nucleophilic moiety, an electrophilic moiety, or a targeting ligand;
or a pharmaceutically acceptable salt thereof.

5. A compound of claim 1, wherein
R₁ is H;
R₂ and R₃ are taken together with the carbon atoms to which they are attached to form a 5- or 6-membered cycloalkyl substituted with -L₁-R₄;
L₁ is absent or a linker; and
R₄ is a nucleophilic moiety, an electrophilic moiety, or a targeting ligand;
or a pharmaceutically acceptable salt thereof:

6. A compound of claim 1, wherein R₄ is selected from the group consisting of —NH₂, —NCS, —NCO, —N₃, alkynyl, cycloalkynyl, —C(O)R₁₃, —COOR₁₃, —CON(R₁₃)₂, maleimido, acyl halide, tetrazine, and trans-cyclooctene.

7. A compound of claim 1, wherein R₄ is selected from the group consisting of cyclooctynyl, bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

8. A compound of claim 7, wherein R₄ is DBCO or BCN.

9. A compound of claim 1 wherein R₄ comprises a targeting ligand, wherein the targeting ligand is selected from the group consisting of an antibody, antigen binding fragment of an antibody, scaffold protein, and aptamer.

10. The compound of claim 1, wherein L₁ is selected from the group consisting of: 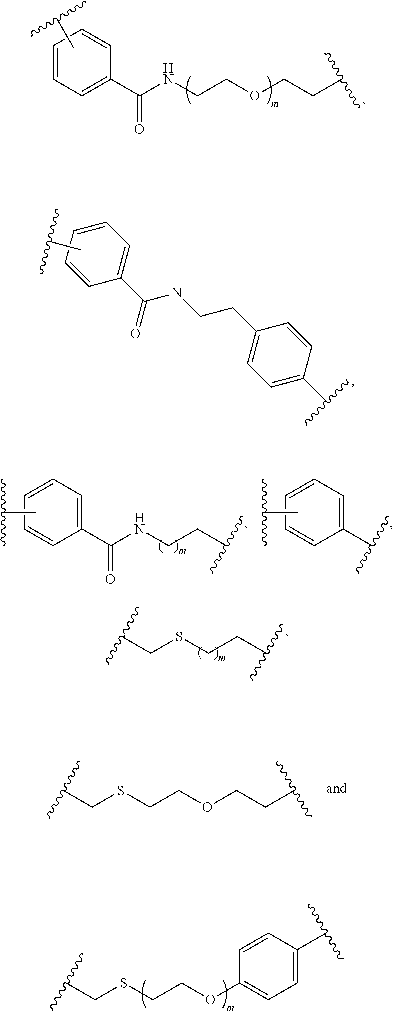
wherein m is an integer of 0 to 12.

11. A compound of claim 1, selected from the group consisting of: 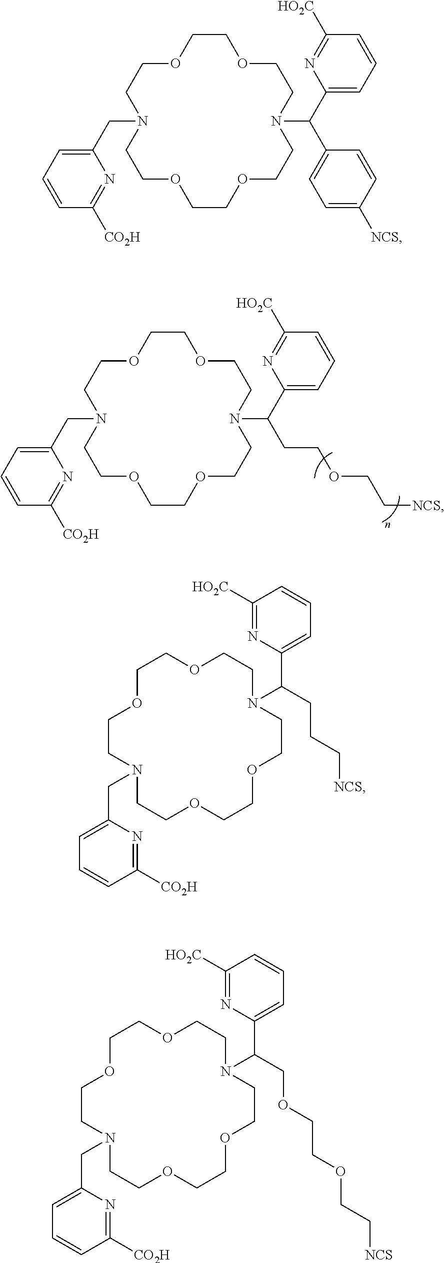 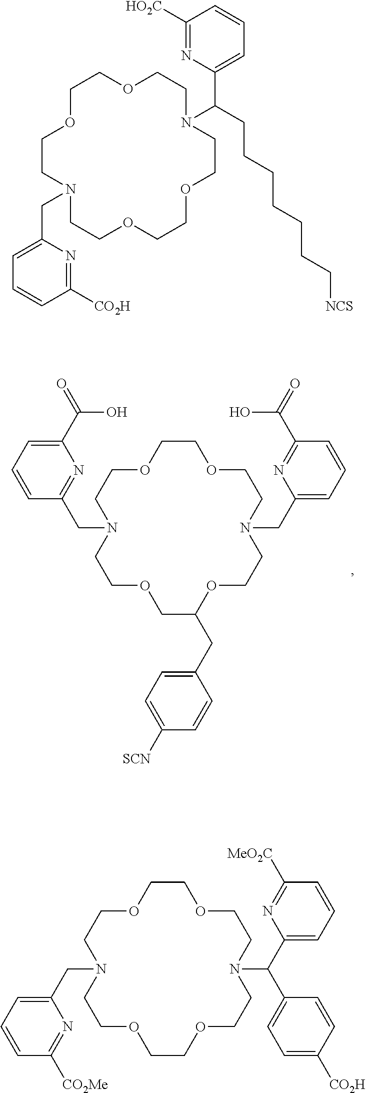 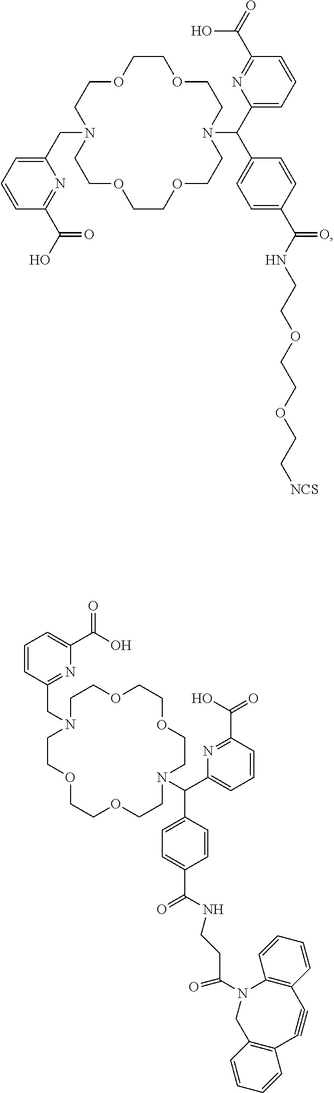 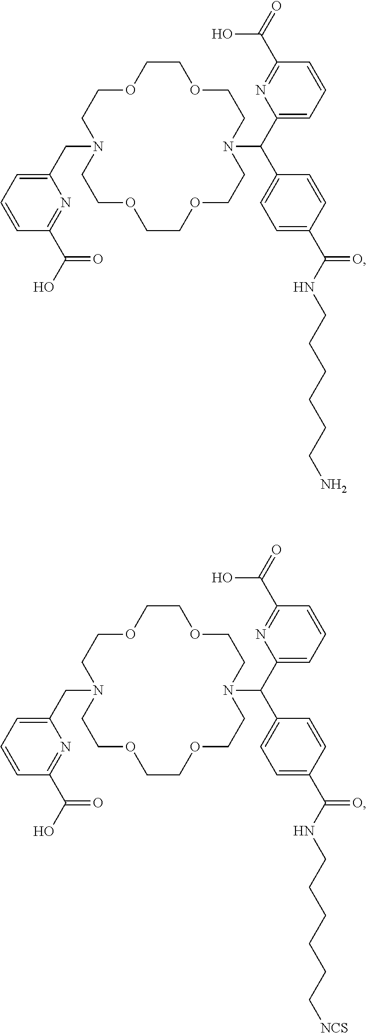 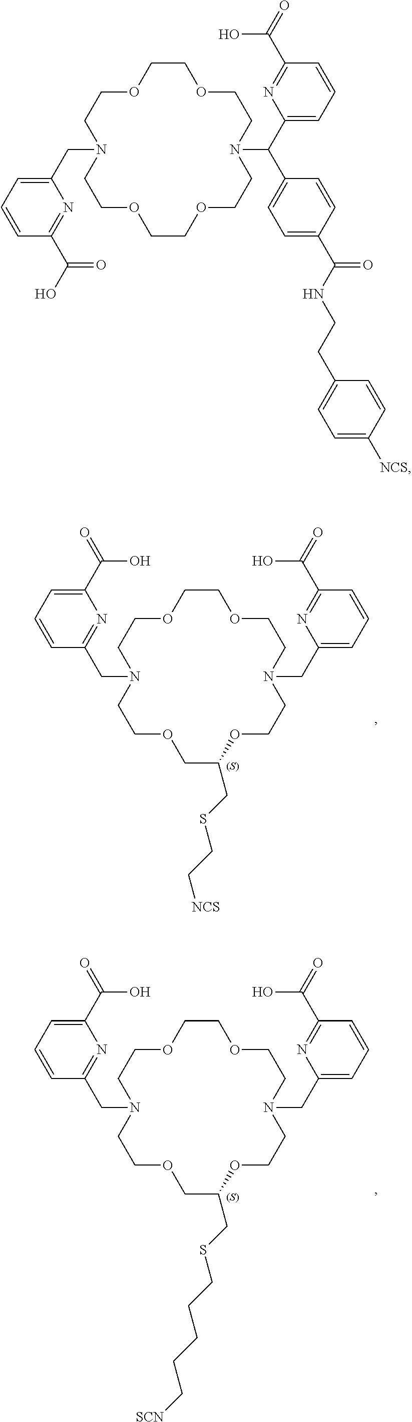 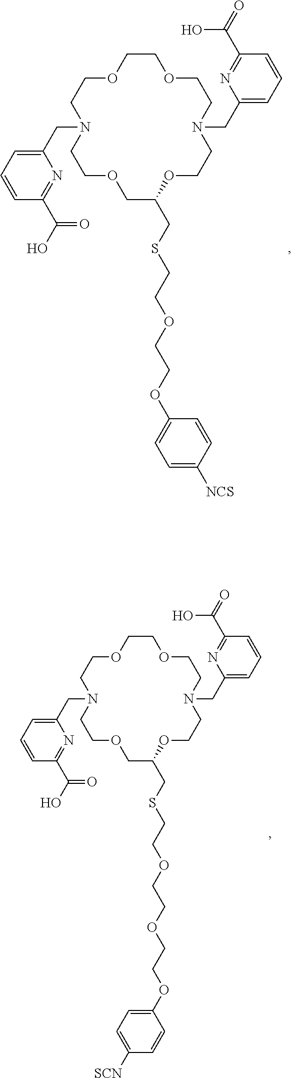 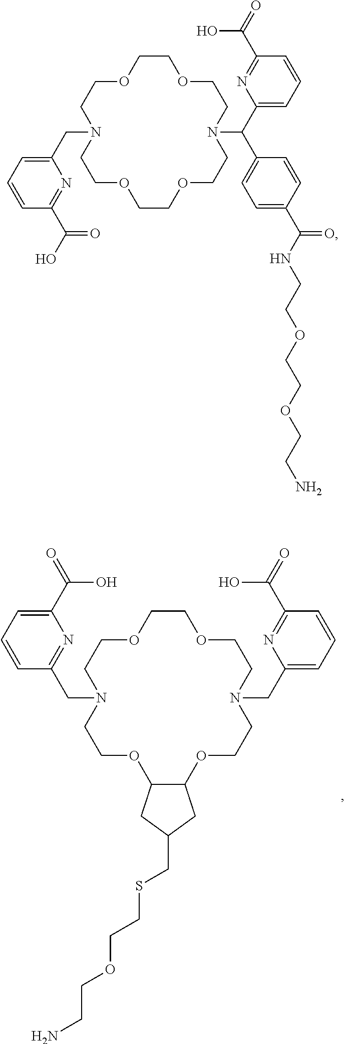 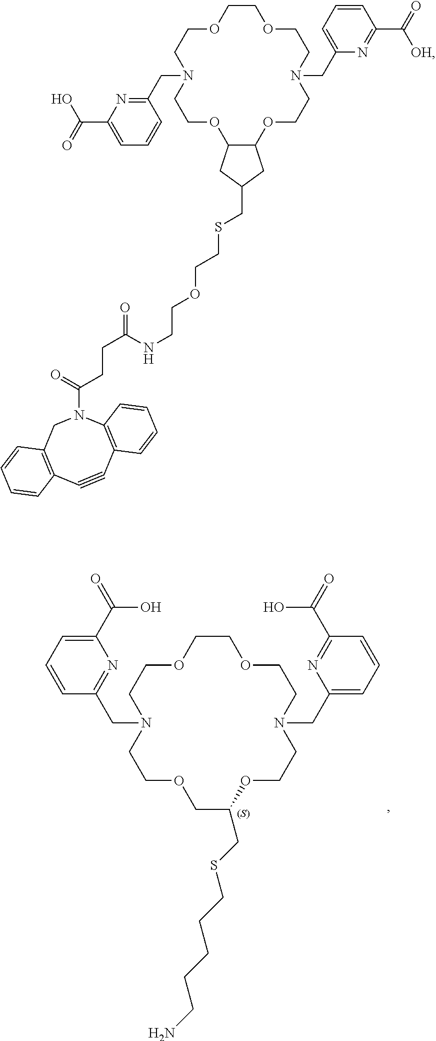 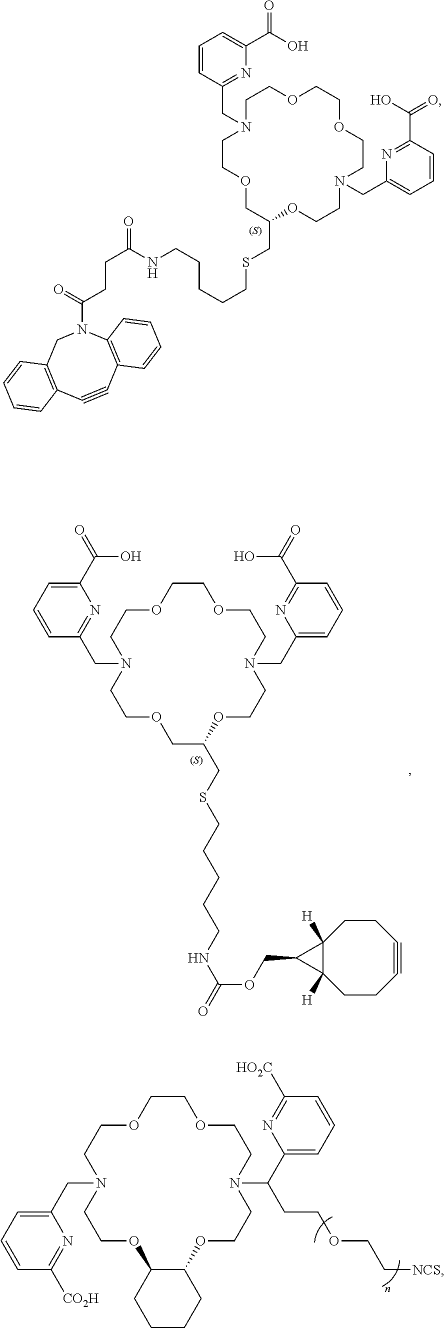 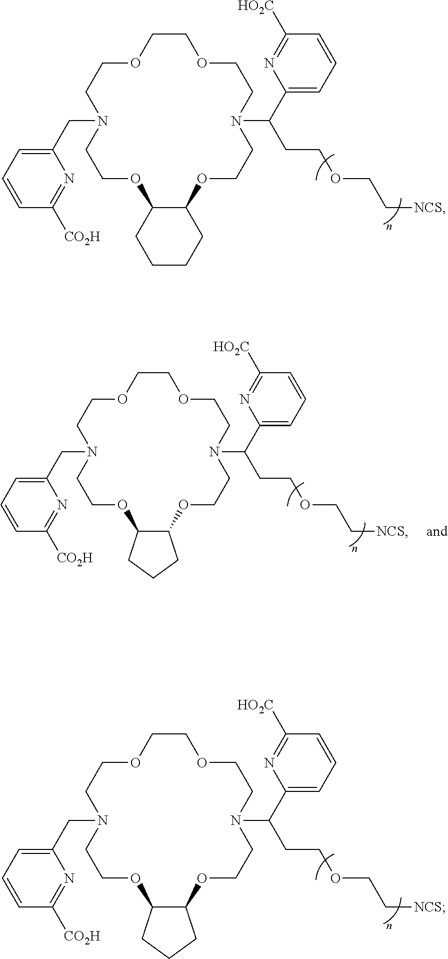
wherein n is 1-10.

12. The compound of claim 1, wherein the compound is bound to a radiometal ion forming a radiometal complex.

13. A radiometal complex of formula (I-M⁺): 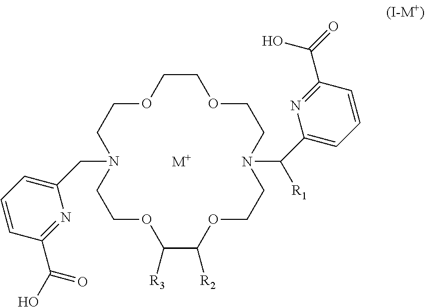
or a pharmaceutically acceptable salt thereof, wherein:
M⁺ is a radiometal ion selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
R₁ is hydrogen and R₂ is -L₁-R₄;
alternatively, R₁ is -L₁-R₄ and R₂ is hydrogen;
R₃ is hydrogen;
alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
L₁ is absent or a linker;
R₄ is a nucleophilic moiety, an electrophilic moiety, or a targeting ligand.

14. A radiometal complex of claim 13, of formula (II-M⁺): 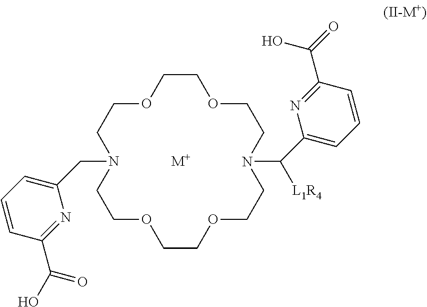
or a pharmaceutically acceptable salt thereof, wherein:
M⁺ is a radiometal ion selected from the group consisting of actinium-225(²²⁵Ac), radium-223 (²³³Ra), bismuth-213 (213Bi), lead-212 (212Pb(II) and/or ²¹²Pb(IV)), terbium-149 (149Tb), terbium-152 (¹⁵²Tb), terbium-155 (155Tb), fermium-255 (²⁵⁵Fm), thorium-227 (²²⁷Th), thorium-226 (²²⁶Th⁴⁺), astatine-211 (²¹¹At), cerium-134 (134Ce), neodymium-144 (144Nd), lanthanum-132 (132La), lanthanum-135 (135La) and uranium-230 (²³⁰U);
L₁ is absent or a linker;
R₄ is a nucleophilic moiety, an electrophilic moiety, or a targeting ligand.

15. A radiometal complex of claim 13, of formula (III-M⁺): 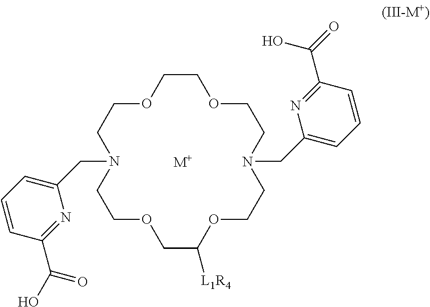
or a pharmaceutically acceptable salt thereof, wherein:
M⁺ is a radiometal ion selected from the group consisting of actinium-225(²²⁵Ac), radium-223 (²³³Ra), bismuth-213 (213Bi), lead-212 (212Pb(II) and/or ²¹²Pb(IV)), terbium-149 (149Tb), terbium-152 (¹⁵²Tb), terbium-155 (155Tb), fermium-255 (²⁵⁵Fm), thorium-227 (²²⁷Th), thorium-226 (²²⁶Th⁴⁺), astatine-211 (²¹¹At), cerium-134 (134Ce), neodymium-144 (144Nd), lanthanum-132 (132La), lanthanum-135 (135La) and uranium-230 (²³⁰U);
L₁ is absent or a linker; and
R₄ is a nucleophilic moiety, an electrophilic moiety, or a targeting ligand.

16. A radiometal complex of claim 13, wherein:
M⁺ is a radiometal ion selected from the group consisting of actinium-225(²²⁵Ac), radium-223 (²³³Ra), bismuth-213 (213Bi), lead-212 (212Pb(II) and/or ²¹²Pb(IV)), terbium-149 (149Tb), terbium-152 (¹⁵²Tb), terbium-155 (155Tb), fermium-255 (²⁵⁵Fm), thorium-227 (²²⁷Th), thorium-226 (²²⁶Th⁴⁺), astatine-211 (²¹¹At), cerium-134 (134Ce), neodymium-144 (144Nd), lanthanum-132 (132La), lanthanum-135 (135La) and uranium-230 (²³⁰U);
R₁ is -L₁-R₄;
R₂ and R₃ are taken together with the carbon atoms to which they are attached to form a 5-membered cycloalkyl;
L₁ is absent or a linker; and
R₄ is a nucleophilic moiety, an electrophilic moiety, or a targeting ligand;
or a pharmaceutically acceptable salt thereof.

17. A radiometal complex of claim 13, wherein
M⁺ is a radiometal ion selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
R₁ is H;
R₂ and R₃ are taken together with the carbon atoms to which they are attached to form a 6-membered cycloalkyl substituted with -L₁-R₄;
L₁ is absent or a linker; and
R₄ is a nucleophilic moiety, an electrophilic moiety, or a targeting ligand;
or a pharmaceutically acceptable salt thereof.

18. A radiometal complex of claim 13, selected from the group consisting of: 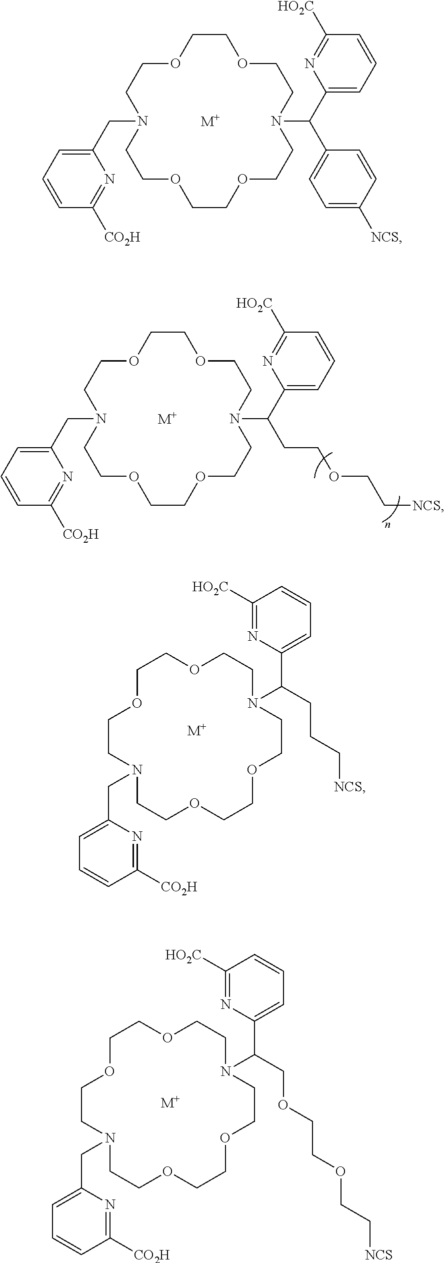 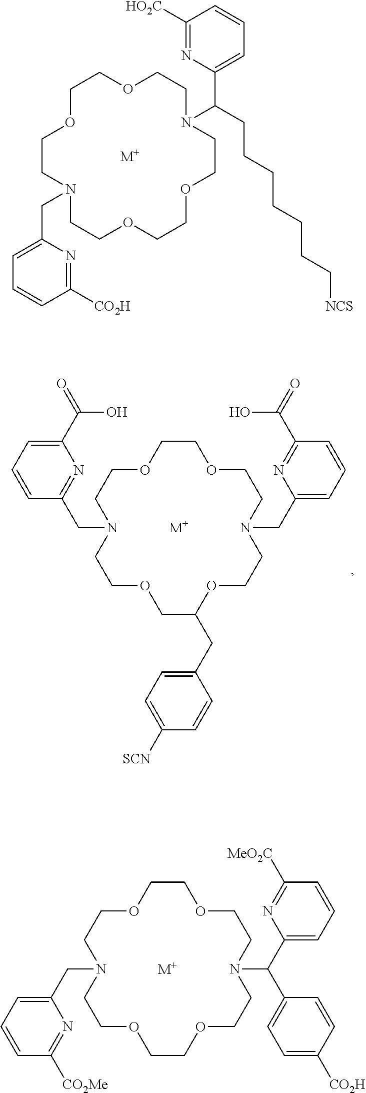 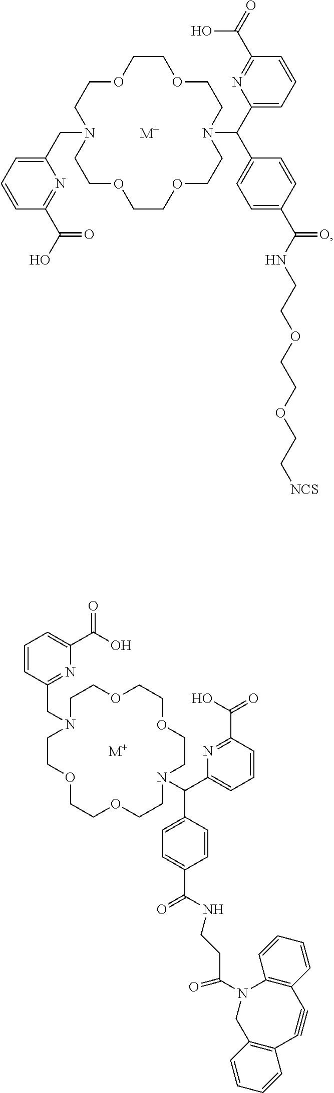 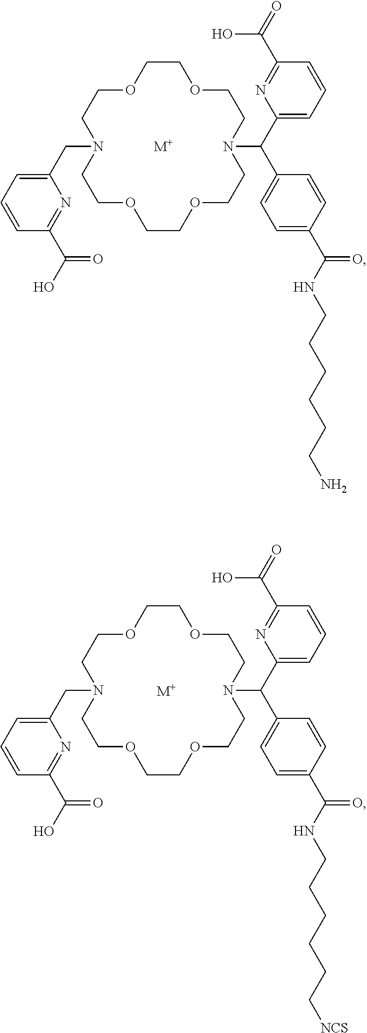 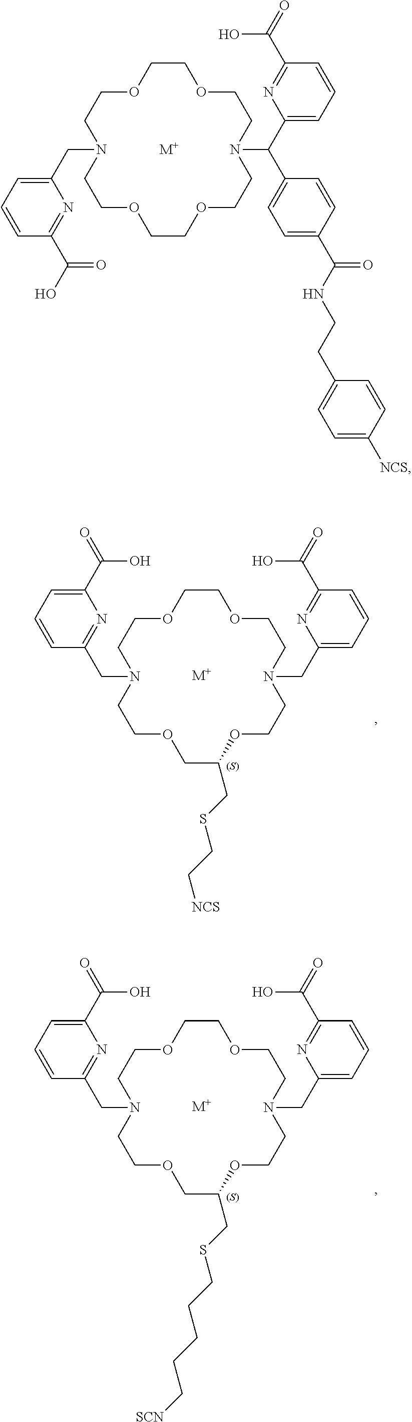 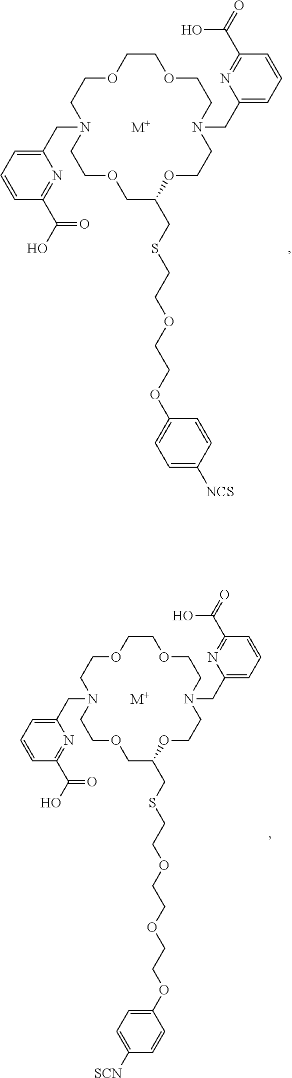 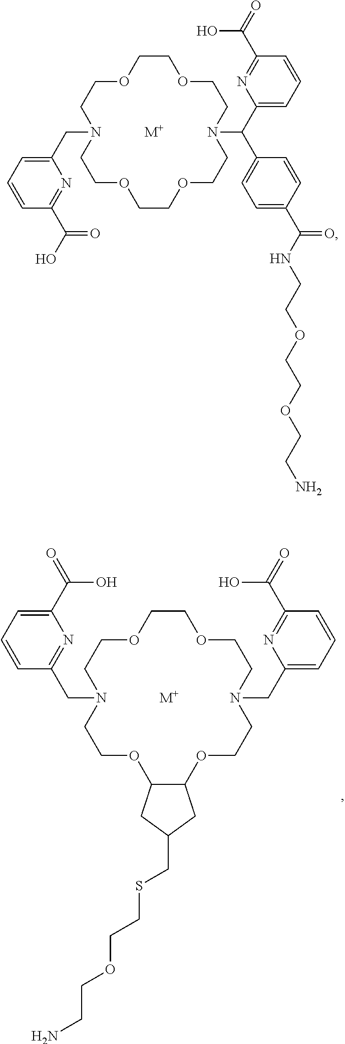 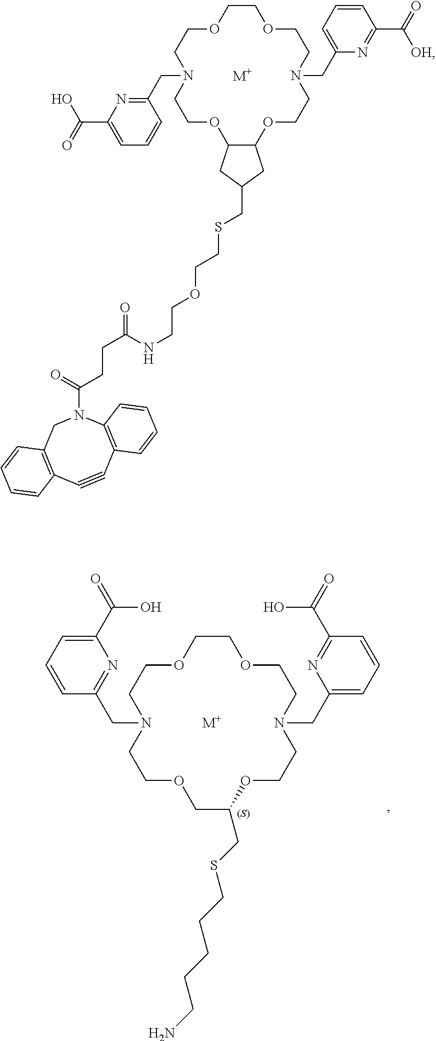 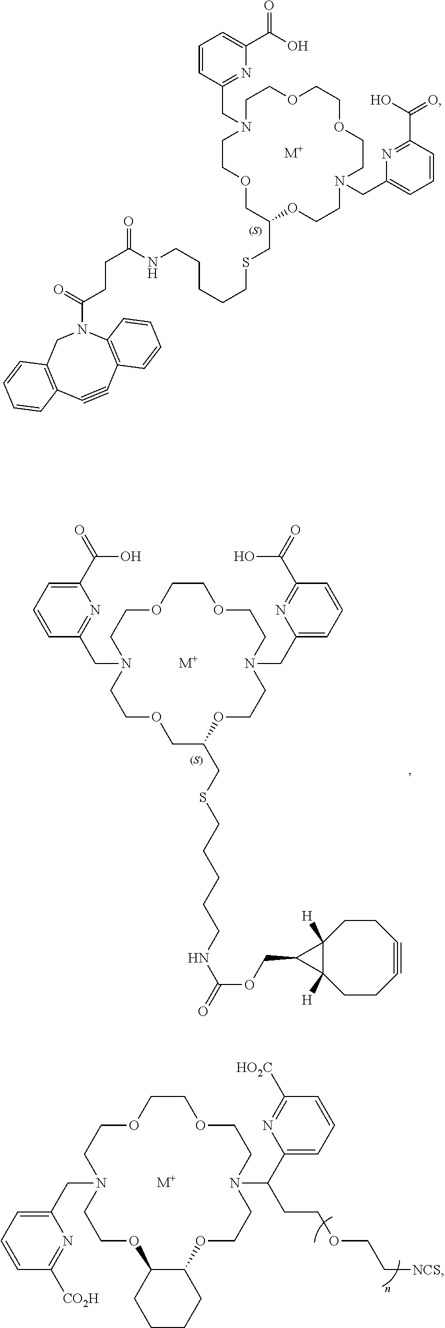 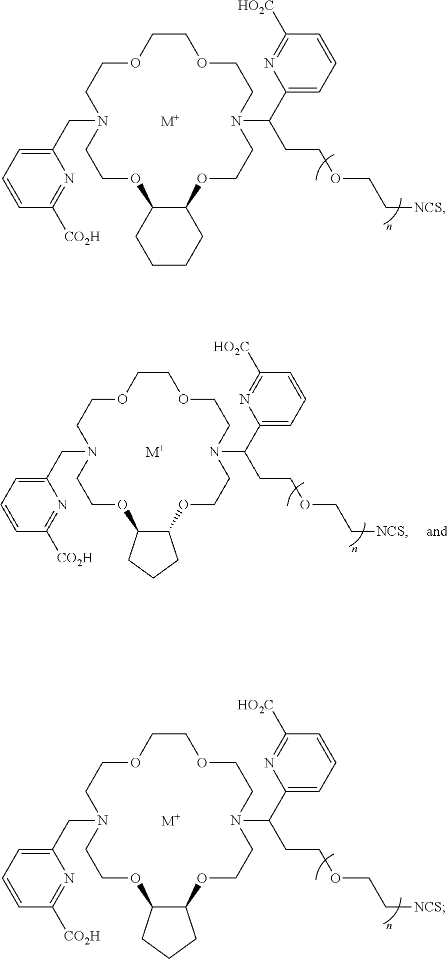
wherein n is 1-10;
and M⁺ is a radiometal ion selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U).

19. An immunoconjugate comprising the compound of claim 9 conjugated to an antibody or antigen binding fragment thereof.

20. An immunoconjugate of claim 19, wherein the antibody or antigen binding fragment thereof is linked to R₄ via a triazole moiety.

21. An immunoconjugate selected from the group consisting of: 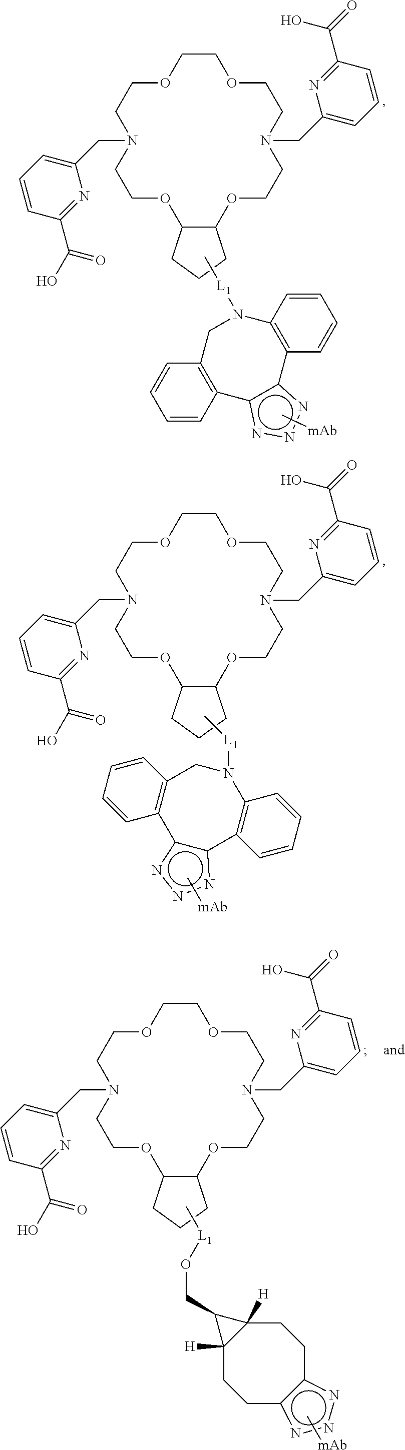 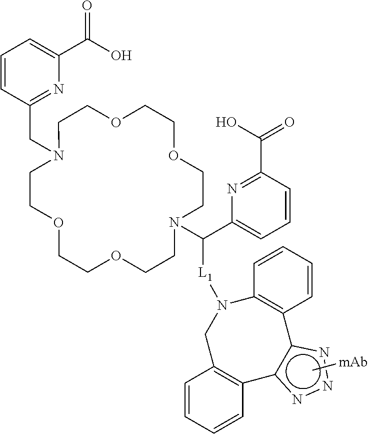
wherein:
L₁ is absent or a linker; and
mAb is an antibody or antigen binding fragment thereof.

22. An immunoconjugate of claim 21, wherein the mAb is h11B6 or PSMB-127.

23. An immunoconjugate of claim 21, selected from the group consisting of: 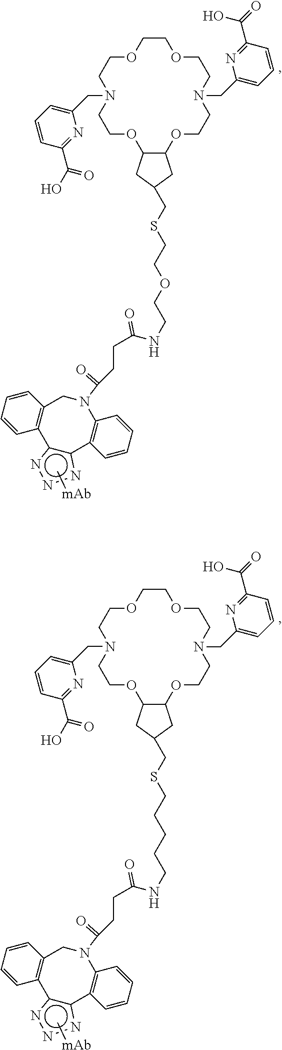 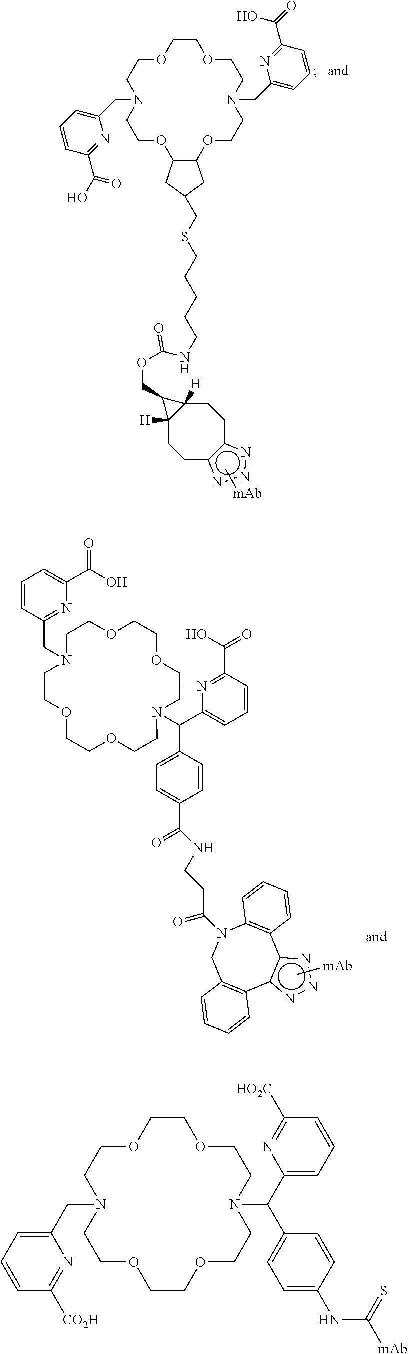
wherein mAb is an antibody or antigen binding fragment thereof.

24. An immunoconjugate of claim 23, wherein the mAb is h11B6 or PSMB-127.

25. A radioimmunoconjugate wherein the radiometal complex of claim 13 is conjugated to an antibody or antigen binding fragment thereof.

26. A radioimmunoconjugate of claim 25, wherein the antibody or antigen binding fragment thereof is linked to R₄ of the radiometal complex via a triazole moiety.

27. A radioimmunoconjugate of claim 25, wherein the antibody is h11B6 or PSMB-127.

28. A radioimmunoconjugate selected from the group consisting of: 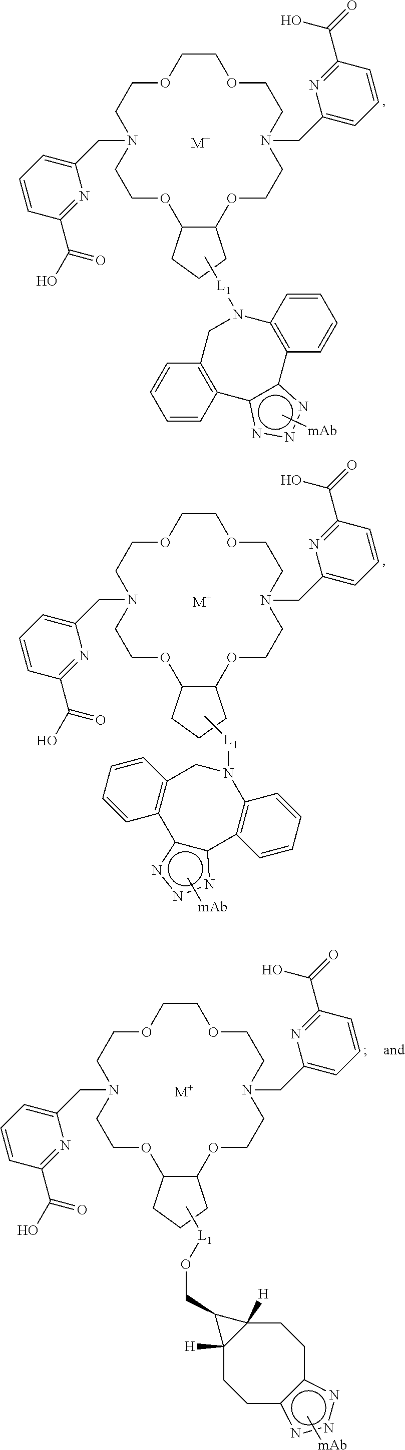 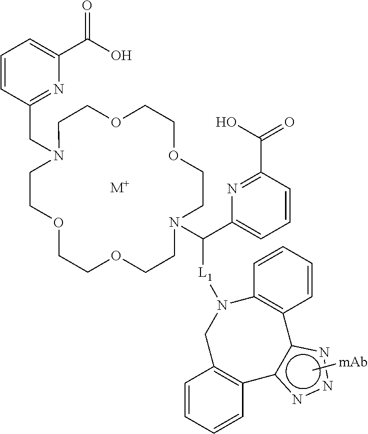
wherein:
M⁺ is a radiometal ion is selected from the group consisting of actinium-225(²²⁵Ac), radium-223 (²³³Ra), bismuth-213 (213Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
L₁ is absent or a linker; and
mAb is an antibody or antigen binding fragment thereof.

29. A radioimmunoconjugate of claim 28, wherein the mAb is h11B6 or PSMB-127.

30. A radioimmunoconjugate of claim 28, selected from the group consisting of: 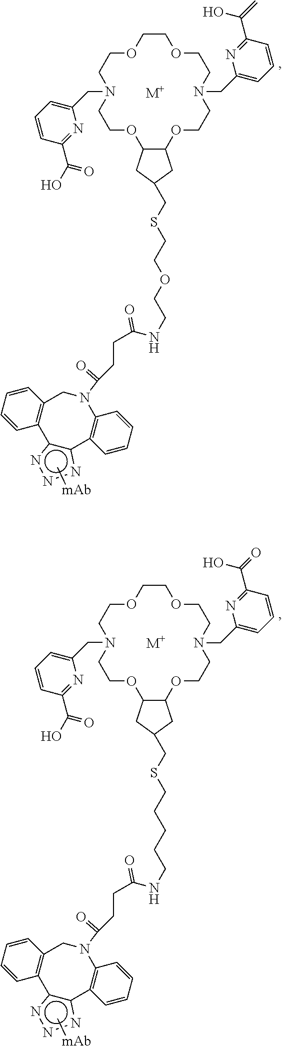 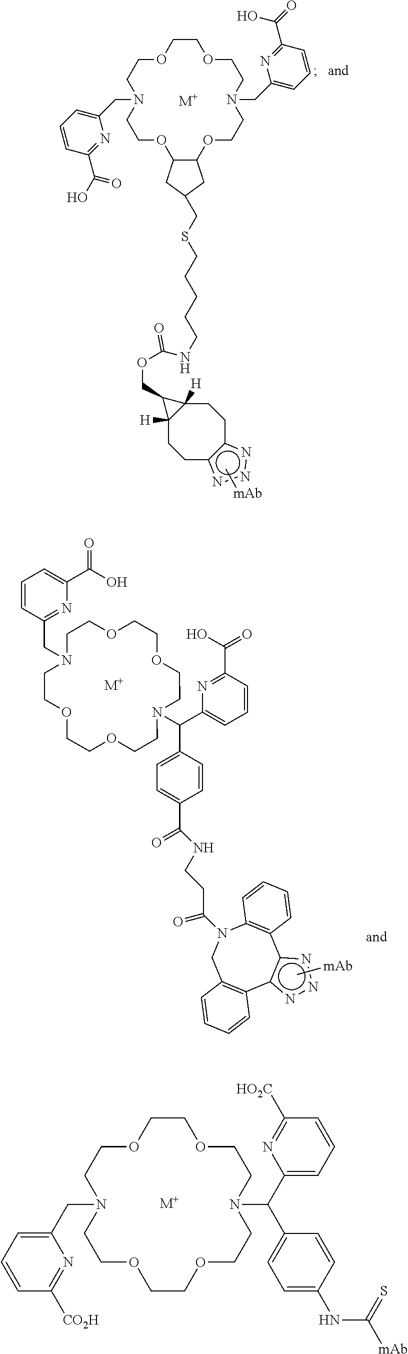
wherein:
M⁺ is a radiometal ion is selected from the group consisting of actinium-225(²²⁵Ac), radium-223 (²³³Ra), bismuth-213 (213Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U); and
mAb is an antibody or antigen binding fragment thereof.

31. An immunoconjugate of claim 30, wherein the mAb is h11B6.

32. A method of preparing a radioimmunoconjugate as in claim 25, comprising: reacting an immunoconjugate of claim 19 with a radiometal ion.

33. The method of claim 32, wherein the targeting ligand is an antibody or antigen binding fragment thereof.

34. The method of claim 33, wherein the antibody is h11B6 or PSMB-127.

35. A method of preparing a radioimmunoconjugate of formula (I-M⁺), 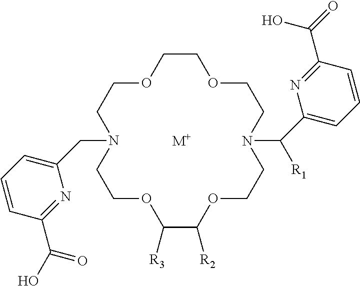
wherein:
M⁺ is a radiometal ion selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
R₁ is hydrogen and R₂ is -L₁-R₄;
alternatively, R₁ is -L₁-R₄ and R₂ is hydrogen;
R₃ is hydrogen;
alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
L₁ is absent or a linker;
R₄ is an alkynyl or cycloalkynyl;
comprising:
(i) reacting a modified polypeptide with a compound of claim 1, wherein the modified polypeptide is an antibody or antigen binding fragment thereof consisting of an azido group to yield an immunoconjugate; and
(ii) reacting the immunoconjugate with a radiometal ion to yield the radioimmunoconjugate of formula (I-M⁺).

36. The method of claim 35, wherein the antibody is h11B6.

37. A method of preparing a radioimmunoconjugate of formula (I-M⁺), 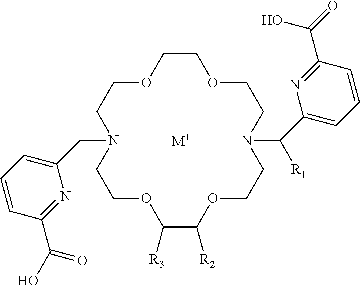
wherein:
M⁺ is a radiometal ion selected from the group consisting of actinium-225(²²⁵Ac), radium-223 (²³³Ra), bismuth-213 (²¹³Bi), lead-212 (²¹²Pb(II) and/or ²¹²Pb(IV)), terbium-149 (¹⁴⁹Tb), terbium-152 (¹⁵²Tb), terbium-155 (¹⁵⁵Tb), fermium-255 (²⁵⁵Fm), thorium-227 (²²⁷Th), thorium-226 (²²⁶Th⁴⁺), astatine-211 (²¹¹At), cerium-134 (¹³⁴Ce), neodymium-144 (¹⁴⁴Nd), lanthanum-132 (¹³²La), lanthanum-135 (¹³⁵La) and uranium-230 (²³⁰U);
R₁ is hydrogen and R₂ is -L₁-R₄;
alternatively, R₁ is -L₁-R₄ and R₂ is hydrogen;
R₃ is hydrogen;
alternatively, R₂ and R₃ are taken together with the carbon atoms to which they are attached to form a 5- or 6-membered cycloalkyl, wherein the 5- or 6-membered cycloalkyl is optionally substituted with -L₁-R₄;
L₁ is absent or a linker;
R₄ is an alkynyl or cycloalkynyl;
comprising:
(i) reacting a modified antibody or antigen binding fragment thereof consisting of an azido group with a compound of claim 1 to yield an immunoconjugate; and
(ii) reacting the immunoconjugate with a radiometal ion to yield a radioimmunoconjugate of formula (I-M⁺).

38. The method of claim 25, wherein R₄ is selected from the group consisting of cyclooctynyl, bicyclononynyl (BCN), difluorinated cyclooctynyl (DIFO), dibenzocyclooctynyl (DIBO), keto-DIBO, biarylazacyclooctynonyl (BARAC), dibenzoazacyclooctynyl (DIBAC, DBCO, ADIBO), dimethoxyazacyclooctynyl (DIMAC), difluorobenzocyclooctynyl (DIFBO), monobenzocyclooctynyl (MOBO), and tetramethoxy dibenzocyclooctynyl (TMDIBO).

39. The method of claim 37, wherein the antibody is h11B6 or PSMB-127.

40. A pharmaceutical composition comprising an immunoconjugate of claim 19, and a pharmaceutically acceptable carrier.

41. A method of selectively targeting neoplastic cells for radiotherapy in a subject in need thereof, comprising administering to the subject a therapeutically effective amount of the pharmaceutical composition of claim 40.

42. A method of treating a neoplastic disease or disorder in a subject in need thereof, comprising administering to the subject a therapeutically effective amount of the pharmaceutical composition of claim 40.

43. A method of treating cancer in a subject in need thereof, comprising administering to the subject a therapeutically effective amount of the pharmaceutical composition of claim 40.

44. A pharmaceutical composition comprising the radioimmunoconjugate of claim 25, and a pharmaceutically acceptable carrier.

45. A method of selectively targeting neoplastic cells for radiotherapy in a subject in need thereof, comprising administering to the subject a therapeutically effective amount of the pharmaceutical composition of claim 44.

46. A method of treating a neoplastic disease or disorder in a subject in need thereof, comprising administering to the subject a therapeutically effective amount of the pharmaceutical composition of claim 44.

47. A method of treating cancer in a subject in need thereof, comprising administering to the subject a therapeutically effective amount of the pharmaceutical composition of claim 44.
